SEQUENCE LISTING (3 sequences)

SEQ ID NO: 1 - protein, 366 aa, Artificial Sequence
  Synthetic Construct
DAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 2 - DNA, 45 bp, Artificial Sequence
  Synthetic Construct
tacagatctactagtgatctatgactgatctgtacatgatctaca

SEQ ID NO: 3 - DNA, 45 bp, Artificial Sequence
  Synthetic Construct
tgtagatcatgtacagatcagtcatagatcactagtagatctgta

(19) United States
(12) Patent Application Publication
(10) Pub. No.: US 2021/0246122 A1
(43) Pub. Date: Aug. 12, 2021

(54) Triazine Compounds and Uses Thereof

(71) Applicant: Aduro Biotech, Inc., Berkeley, CA (US)

(72) Inventors: George Edwin Katibah, San Carlos, CA (US); Jung Yun Kim, Mississuaga (CA); Chudi Obioma Ndubaku, Oakland, CA (US); Meiliana Tjandra, Dublin, CA (US); Tucker Curran Roberts, Berkeley, CA (US)

(73) Assignee: Aduro Biotech, Inc., Berkeley, CA (US)

(21) Appl. No.: 15/734,293

(22) PCT Filed: Jun. 14, 2019
(86) PCT No.: PCT/US2019/037252

§ 371 Date: Dec. 2, 2020

Related U.S. Application Data
(60) Provisional application No. 62/688,543, filed on Jun. 22, 2018.

(51) Int. Cl. C07D 403/14; C07D 403/12; C07D 417/14; C07D 405/14; C07D 251/70

(52) U.S. Cl. CPC C07D 403/14; C07D 403/12; C07D 251/70; C07D 405/14; C07D 417/14

(57) ABSTRACT

The present invention relates to triazine compounds. The present invention also relates to pharmaceutical compositions containing these compounds and methods of treating autoimmune, inflammatory, and neurodegenerative diseases by administering these compounds and pharmaceutical compositions to subjects in need thereof. The present invention also relates to the use of such compounds for research or other non-therapeutic purposes.

### BACKGROUND

The enzyme cyclic GMP-AMP Synthase (cGAS) catalyzes the synthesis of cyclic GMP-AMP (cGAMP) from ATP and GTP in the presence of DNA. This cGAMP then functions as a second messenger that binds to and activates STimulator of INterferon Genes (STING). The activation of IRF3 and the NF-κB signaling by this pathway results in the production of cytokines and type I interferons, which triggers an innate immune response to bacterial or viral infection. Genetic mutations that alter the balance of this pathway may result in an increased activation of the STING pathway, resulting in autoimmune and inflammatory diseases. For example, a loss of function mutation of TREX1 exonuclease, which digests DNA, can result in an accumulation of self-DNA in the cytosol, leading to excessive levels of cGAMP produced by cGAS and elevated expression of interferon induced genes in this pathway. Mutations in TREX1 are associated with systemic inflammatory diseases such as Aicardi-Goutieres Syndrome, familial chilblain lupus and systemic lupus erythematosus. Trex^{-/-} mice were shown to exhibit autoimmune and inflammatory phenotypes which are eliminated with genetic deletion of cGas in these mice (Gao et al., PNAS 112(42):E5699-705, 2015; Gray et al., The Journal of Immunology 195:1939-1943, 2015).

### SUMMARY

In one aspect, the present invention features a triazine compound of Formula (I) below or a pharmaceutically acceptable salt thereof.

In this formula,

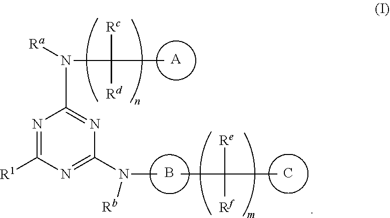

each of R^{a} and R^{b} independently is H or C₁-C₆ alkyl, wherein C₁-C₆ alkyl is optionally substituted with one or more substituents selected from the group consisting of cyano, halo, —OR, —S(═O)ₓR, and —NRR′;
each of R^{c} and R^{d} independently is, at each occurrence, H, halo, —C(═O)R², or C₁-C₃ alkyl, wherein the C₁-C₃ alkyl is optionally substituted with one or more substituents independently selected from the group consisting of cyano, halo, —OR, —S(═O)ₓR, —NRR′; or R^{c} and R^{d} attached to the same carbon is oxo; or R^{c} and R^{d} attached to the same carbon forms a C₃-C₈ cycloalkyl, or a 3- to 8-membered heterocycloalkyl, wherein the C₃-C₈ cycloalkyl, or 3- to 8-membered heterocycloalkyl is optionally substituted with one or more substituents independently selected from the group consisting of cyano, halo, oxo, —OR, —S(═O)ₓR, —NRR′, C₁-C₃ alkyl and C₁-C₃ haloalkyl; and wherein at least one occurrence of R^{c} is —C(═O)R²;
R² is —OR^{g} or —NR^{g}R^{h};
each of R^{g} and R^{h} is independently H, C₁-C₆ alkyl, (CH₂CH₂O)ᵤ—H, or (CH₂CH₂O)ᵤ—C₁-C₆ alkyl;
each of R^{e} and R^{f} independently is H, halo, or C₁-C₃ alkyl, wherein the C₁-C₃ alkyl is optionally substituted with one or more substituents independently selected from the group consisting of cyano, halo, —OR, —S(═O)ₓR, and —NRR′; or R^{e} and R^{f} attached to the same carbon is oxo; or R^{e} and R^{f} attached to the same carbon forms a C₃-C₈ cycloalkyl, or a 3- to 8-membered heterocycloalkyl, wherein the C₃-C₈ cycloalkyl, or 3- to 8-membered heterocycloalkyl is optionally substituted with one or more substituents independently selected from the group consisting of cyano, halo, oxo, —OR, —S(═O)ₓR, —NRR′, C₁-C₃ alkyl and C₁-C₃ haloalkyl;
R¹ is H, halo, cyano, —OR¹, —NRⁱR^{j}, or C₁-C₆ alkyl, wherein C₁-C₆ alkyl is optionally substituted with one or more R^{S1};
each R^{S1} is independently halo, cyano, oxo, —OR¹, —NRⁱR^{j}, C₃-C₈ cycloalkyl, C₆-C₁₀ aryl, 3- to 8-membered heterocycloalkyl, or 5- to 10-membered heteroaryl, wherein the C₃-C₈ cycloalkyl, C₆-C₁₀ aryl, 3- to 8-membered heterocycloalkyl, or 5- to 10-membered heteroaryl is optionally substituted with one or more substituents independently selected from the group consisting of cyano, halo, oxo, —OR, —S(═O)ₓR, —NRR′, C₁-C₆ alkyl and C₁-C₆ haloalkyl;
each of Rⁱ and R^{j} independently is H, C₁-C₆ alkyl, C₃-C₈ cycloalkyl, 3- to 10-membered heterocycloalkyl, C₆-C₁₀ aryl or 5- to 10-membered heteroaryl wherein the C₁-C₆ alkyl is optionally substituted with one or more R^{S2}, and the C₃-C₈ cycloalkyl, 3- to 10-membered heterocycloalkyl, C₆-C₁₀ aryl or 5- to 10-membered heteroaryl is optionally substituted with one or more substituents independently selected from the group consisting of cyano, halo, oxo, —OR, —S(═O)ₓR, —NRR′, C₁-C₆ alkyl and C₁-C₆ haloalkyl;
each R^{S2} is independently halo, cyano, —OR, —NRR^{′}, C₃-C₈ cycloalkyl, 3- to 10-membered heterocycloalkyl, C₆-C₁₀ aryl or 5- to 10-membered heteroaryl, wherein the C₃-C₈ cycloalkyl, 3- to 10-membered heterocycloalkyl, C₆-C₁₀ aryl or 5- to 10-membered heteroaryl is optionally substituted with one or more substituents independently selected from the group consisting of cyano, halo, oxo, —OR, —S(═O)ₓR, —NRR′, and C₁-C₆ alkyl, wherein the C₁-C₆ alkyl is optionally substituted with one or more substituents independently selected from the group consisting of cyano, halo, oxo, —OR, —S(═O)ₓR, and —NRR′;
each of R and R′ independently is, at each occurrence, H, C₁-C₆ alkyl, C₁-C₆ haloalkyl, C₀₋₃alkylene-C₃-C₅ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein the C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of cyano, halo, oxo, —OR^{p}, —S(═O)ₓR^{p}, —NR^{p}R^{q}, and C₁-C₆ alkyl, wherein the C₁-C₆ alkyl is optionally substituted with one or more substituents independently selected from the group consisting of cyano, halo, oxo, —OR^{p}, —S(═O)ₓR^{p}, and —NR^{p}R^{q};
A is a 3- to 14-membered ring optionally containing 1-4 heteroatoms selected from N, O, and S and optionally substituted with one or more R^{S3};
B is a 3- to 14-membered ring optionally containing 1-4 heteroatoms selected from N, O, and S and optionally substituted with one or more R^{S4};
C is a 3- to 14-membered ring optionally containing 1-4 heteroatoms selected from N, O, and S and optionally substituted with one or more R^{S5};
each of R^{S3}, and R^{S4} is independently selected from the group consisting of cyano, halo, oxo, —OR, —S(═O)ₓR, —NRR′, and C₁-C₆ alkyl, wherein the C₁-C₆ alkyl is optionally substituted with one or more substituents independently selected from the group consisting of cyano, halo, oxo, —OR, —S(═O)ₓR, and —NRR′;
each R^{S5} independently is halo, cyano, —C(═O)R^{k}, —S(═O)ₓR^{k}, a nitrogen protecting group bound to a suitable nitrogen of ring C, or C₁-C₆ alkyl, wherein the alkyl is optionally substituted with one or more R^{S6};
R^{k} is H, C₁-C₆ alkyl, C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein C₁-C₆ alkyl is optionally substituted with one or more R^{S6}, and wherein C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of R^{S6}, C₁-C₆ alkyl, and C₁-C₆ alkyl substituted with one or more R^{S6};
each R^{S6} independently is halo, cyano, oxo, —OR^{m}, —OC(═O)R^{m}, —C(═O)R^{m}, —C(═O)OR^{m}, —S(═O)ₓR^{m}, —S(═O)₂OR^{m}, —OS(═O)₂R^{m}, —NR^{m}Rⁿ, —C(═O)NR^{m}Rⁿ, —C(═NR^{m})NR^{m}Rⁿ, —S(═O)₂NR^{m}Rⁿ, —NR^{m}C(═O)R^{m}, —NR^{m}C(═NR^{m})R^{m}, —NR^{m}S(═O)₂R^{m}, —NR^{m}C(═O)NR^{m}Rⁿ, —NR^{m}C(═NR^{m})NR^{m}Rⁿ, —NR^{m}S(═O)₂NR^{m}Rⁿ, —NR^{m}C(═O)OR^{m}, —OC(═O)NR^{m}Rⁿ, C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein the C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of cyano, halo, oxo, OR^{m}, OC(═O)R^{m}, C(═O)R^{m}, C(═O)OR^{m}, S(═O)ₓR^{m}, S(═O)₂OR^{m}, OS(═O)₂R^{m}, NR^{m}Rⁿ, C(═O)NR^{m}Rⁿ, C(═NR^{m})NR^{m}Rⁿ, S(═O)₂NR^{m}Rⁿ, NR^{m}C(═O)R^{m}, NR^{m}C(═NR^{m})R^{m}, NR^{m}S(═O)₂R^{m}, NR^{m}C(═O)NR^{m}Rⁿ, NR^{m}C(═NR^{m})NR^{m}Rⁿ, NR^{m}S(═O)₂NR^{m}Rⁿ, NR^{m}C(═O)OR^{m}, OC(═O)NR^{m}Rⁿ, and C₁-C₆ alkyl, wherein the C₁-C₆ alkyl is optionally substituted with one or more substituents independently selected from the group consisting of cyano, halo, oxo, OR^{m}, OC(═O)R^{m}, C(═O)R^{m}, C(═O)OR^{m}, S(═O)ₓR^{m}, S(═O)₂OR^{m}, OS(═O)₂R^{m}, NR^{m}Rⁿ, C(═O)NR^{m}Rⁿ, C(═NR^{m})NR^{m}Rⁿ, S(═O)₂NR^{m}Rⁿ, NR^{m}C(═O)R^{m}, NR^{m}C(═NR^{m})R^{m}, NR^{m}S(═O)₂R^{m}, NR^{m}C(═O)NR^{m}Rⁿ, NR^{m}C(═NR^{m})NR^{m}Rⁿ, NR^{m}S(═O)₂NR^{m}Rⁿ, NR^{m}C(═O)OR^{m}, OC(═O)NR^{m}Rⁿ;
each of R^{m} and Rⁿ independently is, at each occurrence, H, C₁-C₆ alkyl, C₁-C₆ haloalkyl, C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein the C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of cyano, halo, oxo, —OR^{p}, —OC(═O)R^{p}, —C(═O)R^{p}, —C(═O)OR^{p}, —S(═O)ₓR^{p}, —S(═O)₂OR^{p}, —OS(═O)₂R^{p}, —NR^{p}R^{q}, —C(═O)NR^{p}R^{q}, —C(═NR^{p})NR^{p}R^{q}, —S(═O)₂NR^{p}R^{q}, —NR^{p}C(═O)R^{p}, —NR^{p}C(═NR^{p})R^{p}, —NR^{p}S(═O)₂R^{p}, —NR^{p}C(═O)NR^{p}R^{q}, —NR^{p}C(═NR^{p})NR^{p}R^{q}, —NR^{p}S(═O)₂NR^{p}R^{q}, —NR^{p}C(═O)OR^{p}, —OC(═O)NR^{p}R^{q}, and C₁-C₆ alkyl, wherein the C₁-C₆ alkyl is optionally substituted with one or more substituents independently selected from the group consisting of cyano, halo, oxo, —OR^{p}, —OC(═O)R^{p}, —C(═O)R^{p}, —C(═O)OR^{p}, —S(═O)ₓR^{p}, —S(═O)₂OR^{p}, —OS(═O)₂R^{p}, —NR^{p}R^{q}, —C(═O)NR^{p}R^{q}, —C(═NR^{p})NR^{p}R^{q}, —S(═O)₂NR^{p}R^{q}, —NR^{p}C(═O)R^{p}, —NR^{p}C(═NR^{p})R^{p}, —NR^{p}S(═O)₂R^{p}, —NR^{p}C(═O)NR^{p}R^{q}, —NR^{p}C(═NR^{p})NR^{p}R^{q}, —NR^{p}S(═O)₂NR^{p}R^{q}, —NR^{p}C(═O)OR^{p}, —OC(═O)NR^{p}R^{q};
each R^{p} and R^{q} are independently H or C₁-C₆ alkyl;
m is 0, 1, 2, or 3;
n is 1, 2, 3, or 4;
each occurrence of x is independently 0, 1, or 2; and
each u independently is 1, 2, or 3.

One subset of the compounds of Formula (I) includes those of Formula (Ia):

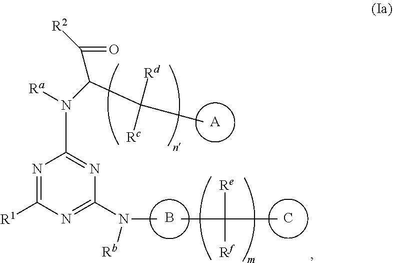

wherein n′ is 0, 1, 2, or 3.

Another subset of the compounds of Formula (I) includes those of Formula (Ib):

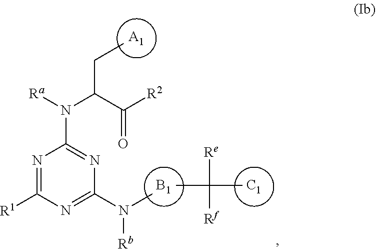

wherein
A₁ is a 3- to 14-membered ring optionally containing 1-4 heteroatoms selected from N, O, and S and optionally substituted with one or more R^{S3};
B₁ is a 3- to 14-membered ring optionally containing 1-4 heteroatoms selected from N, O, and S and optionally substituted with one or more R^{S4}; and
C₁ is a 3- to 14-membered ring optionally containing 1-4 heteroatoms selected from N, O, and S and optionally substituted with one or more R^{S5}.

Yet another subset of the compounds of Formula (I) includes those of Formula (Ic):

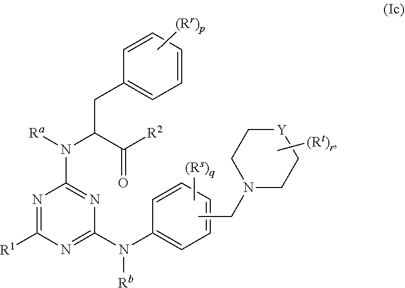

wherein
each R^{r} independently is cyano, halo, oxo, —OR, —S(═O)ₓR, —NRR′, and C₁-C₆ alkyl;
each R^{s} independently is cyano, halo, oxo, —OR, —S(═O)ₓR, —NRR′, and C₁-C₆ alkyl;
each R^{L} independently is halo, cyano, —C(═O)R^{k}, —S(═O)ₓR^{k}, a nitrogen protecting group bound to a suitable nitrogen in the ring, or C₁-C₆ alkyl, wherein the alkyl is optionally substituted with one or more R^{S6};
Y is O, CH₂, CHR^{t}, NH, or NR^{t};
p is 0, 1, 2, 3, 4, or 5; and
each of q and r independently is 0, 1, 2, 3, or 4.

The compounds of Formula (I), (Ia), (Ib), or (Ic) can include one or more of the following features:

Each R^{c} is H.

Each R^{d} is H.

n is 2 or n′ is 1.

A is a 5- to 6-membered ring optionally containing 1-4 heteroatoms selected from N, O, and S and optionally substituted with one or more R^{S3}.

A is phenyl.

A is 4-cyanophenyl.

B is a 5- to 6-membered ring optionally containing 1-4 heteroatoms selected from N, O, and S and optionally substituted with one or more R^{S4}.

B is phenyl.

R^{e} is H.

R^{f} is H.

m is 1.

C is a 5- to 6-membered ring optionally containing 1-4 heteroatoms selected from N, O, and S and optionally substituted with one or more R^{S5}.

C is a 6-membered ring optionally containing 1-2 heteroatoms selected from N, O, and S and optionally substituted with one or more R^{S5}.

C is

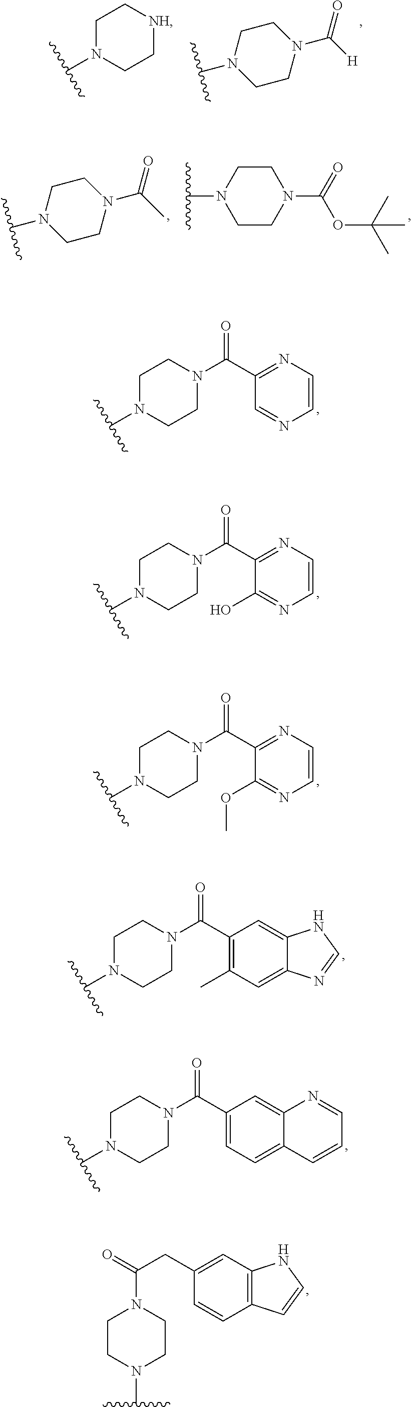

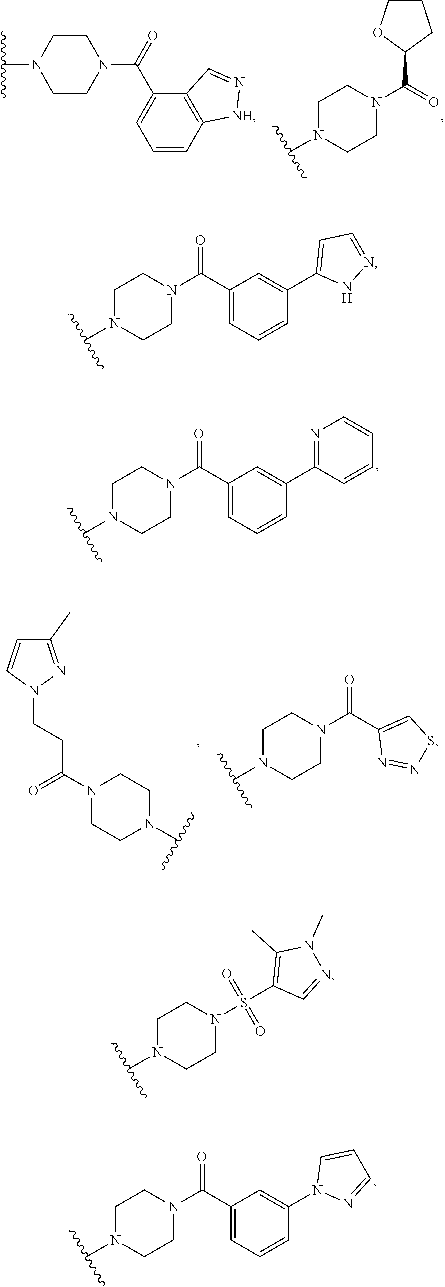

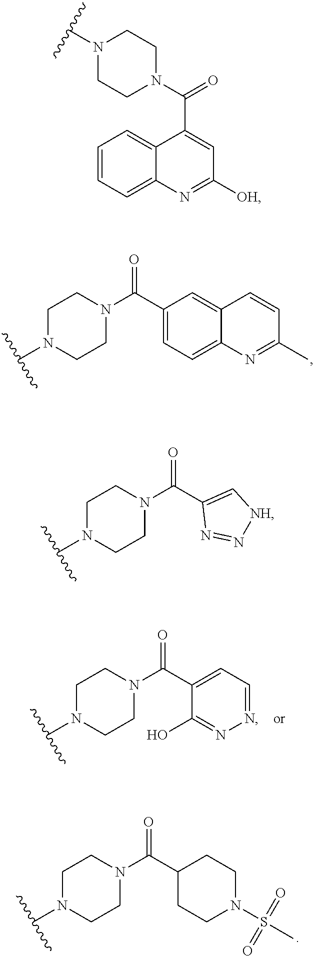 [SMILES: *N1CCN(C(=O)c2cc(O)nc3ccccc23)CC1 ; *N1CCN(C(=O)c2ccc3nc(C)ccc3c2)CC1 ; *N1CCN(C(=O)c2c[nH]nn2)CC1 ; *N1CCN(C(=O)c2ccnnc2O)CC1 ; *N1CCN(C(=O)C2CCN(S(C)(=O)=O)CC2)CC1]

A₁ is phenyl.

A₁ is 4-cyanophenyl.

B₁ is a 5- to 6-membered ring optionally containing 1∝heteroatoms selected from N, O, and S and optionally substituted with one or more R^{S4}.

B₁ is phenyl.

C₁ is a 5- to 6-membered ring optionally containing 1-4 heteroatoms selected from N, O, and S and optionally substituted with one or more R^{S5}.

C₁ is a 6-membered ring optionally containing 1-2 heteroatoms selected from N, O, and S and optionally substituted with one or more R^{S5}.

C₁ is

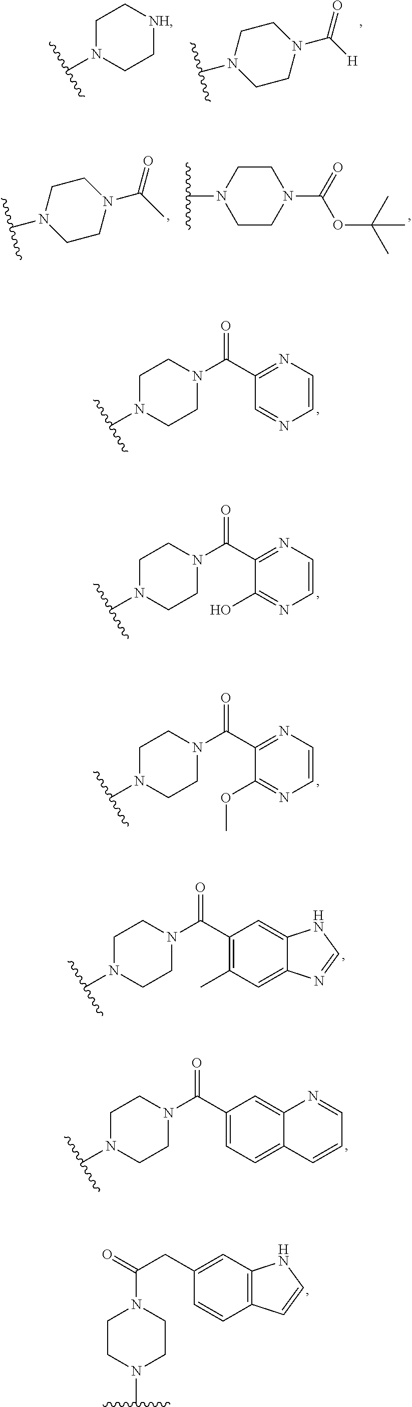

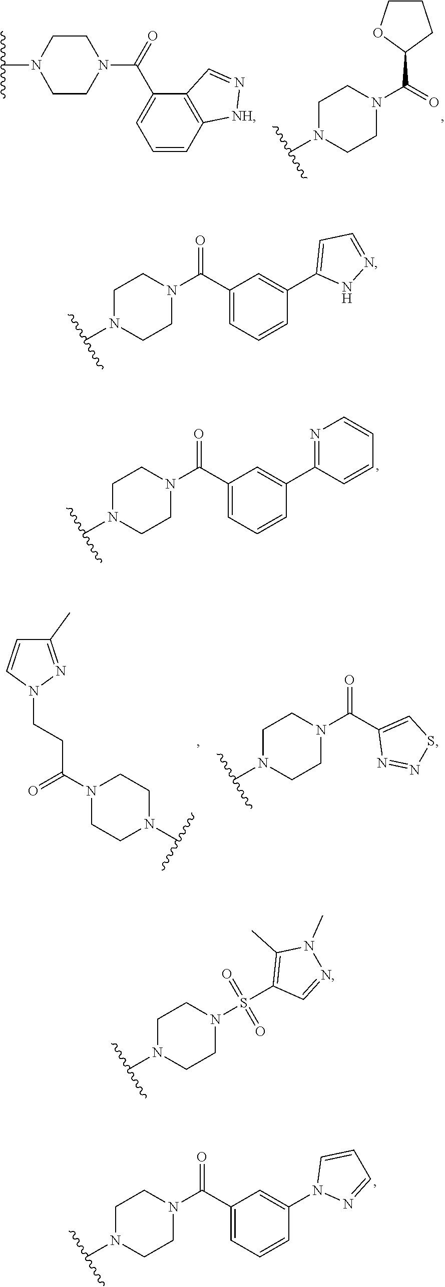

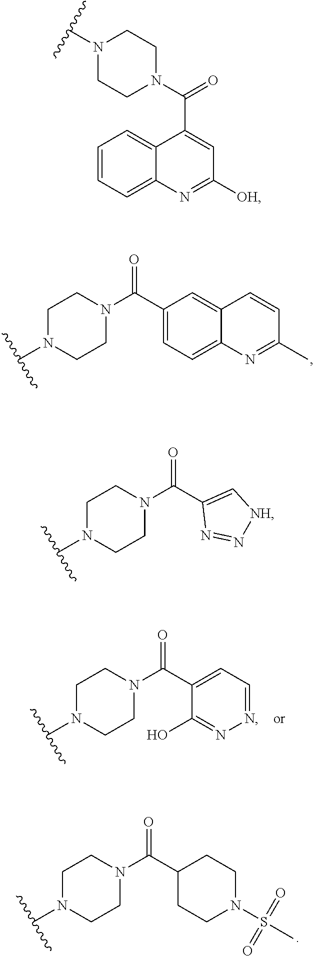 [SMILES: *N1CCN(C(=O)c2cc(O)nc3ccccc23)CC1 ; *N1CCN(C(=O)c2ccc3nc(C)ccc3c2)CC1 ; *N1CCN(C(=O)c2c[nH]nn2)CC1 ; *N1CCN(C(=O)c2ccnnc2O)CC1 ; *N1CCN(C(=O)C2CCN(S(C)(=O)=O)CC2)CC1]

p is 1.

R^{r} is cyano.

q is 0.

r is 1.

r is 0.

Y is NH.

Y is NR^{t}.

R^{k} is H.

R^{t} is C(═O)R^{k}.

R^{t} is a nitrogen protecting group.

R^{t} is —C(═O)C₂-C₄ alkoxy.

R^{t} is —C(═O)O-t-butyl.

R^{k} is 5- to 6-membered heteroaryl.

R^{k} is pyrazinyl.

R^{k} is

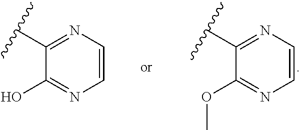

R^{k} is 9- to 10-membered heteroaryl.

R^{k} is benzimidazolyl.

R^{k} is

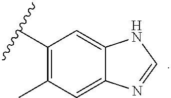 [SMILES: *c1cc2[nH]cnc2cc1C]

R¹ is H.

R¹ is halo.

R¹ is Cl.

R¹ is OR¹.

R¹ is C₁-C₆ alkyl.

R¹ is methyl.

R¹ is NRⁱR^{j}.

R¹ is C₁-C₆ alkyl and R^{j} is H.

each of Rⁱ and R^{j} is C₁-C₆ alkyl.

R¹ is NHCH₃,

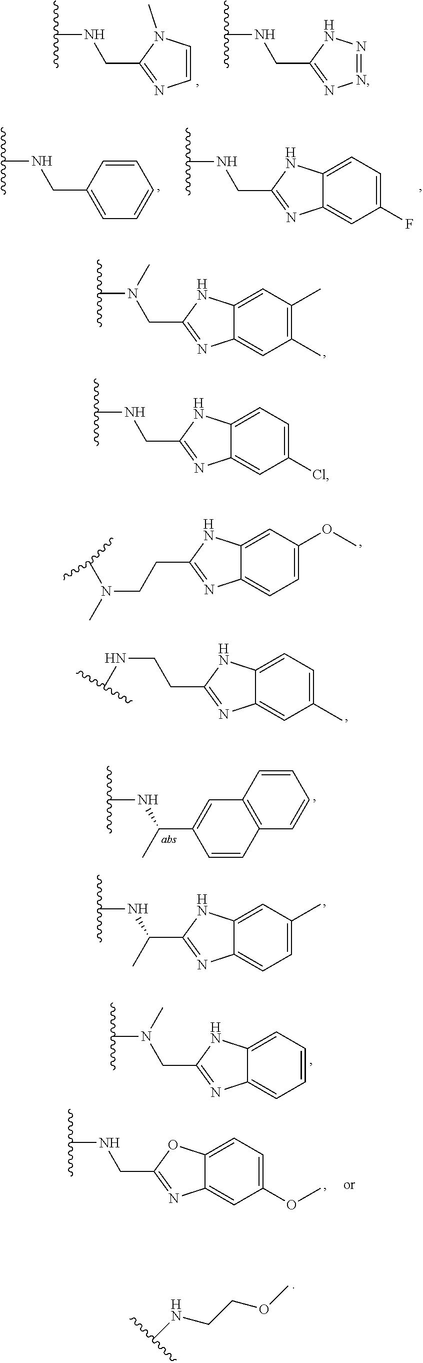

R² is OR^{g}.

R^{g} is C₁-C₃ alkyl.

R^{g} is methyl.

R² is NR^{g}R^{h}.

R^{g} is C₁-C₆ alkyl and R^{h} is H.

R^{g} is methyl.

each of R^{g} and R^{h} is C₁-C₆ alkyl.

each of R^{g} and R^{h} is methyl.

R^{g} is (CH₂CH₂O)ᵤ—C₁-C₆ alkyl and R^{g} is H.

R^{g} is (CH₂CH₂O)ᵤ—CH₃.

u is 1.

u is 2.

R^{a} is H.

R^{b} is H.

The present invention also provides pharmaceutical compositions comprising a compound disclosed herein or a tautomer, enantiomer, or salt thereof together with a pharmaceutically acceptable diluent or carrier.

Another aspect of the invention relates to a method of inhibiting cGAS in a cell, comprising contacting the cell with the compound or composition disclosed herein.

Yet another aspect of the invention is a method of treating a cGAS-mediated condition, comprising administering to a patient in need thereof an effective amount of a compound disclosed herein or a tautomer, enantiomer, or salt thereof, or a composition disclosed herein. For example, the cGAS-mediated condition is an autoimmune, inflammatory, or neurodegenerative condition.

Still another aspect of the invention is a method of treating an autoimmune disease in a subject, comprising administering to the subject a therapeutically effective amount of a compound disclosed herein or a tautomer, enantiomer, or salt thereof, or a composition disclosed herein. For example, the autoimmune disease is SIRS, sepsis, septic shock, atherosclerosis, celiac disease, interstitial cystitis, transplant rejection, Aicardi-Goutieres Syndrome, chilblain lupus erythematosus, systemic lupus erythematosus, idiopathic thrombocytopenic purpura, thrombotic thrombocytopenic purpura, autoimmune thrombocytopenia, spondyloenchondrodysplasia, psoriasis, Type 1 diabetes, Type 2 diabetes, or Sjogren's syndrome.

Yet another aspect of the invention features a method of treating an inflammatory disease in a subject, comprising administering to the subject a therapeutically effective amount of a compound disclosed herein or a tautomer, enantiomer, or salt thereof, or a composition disclosed herein. For example, the inflammatory disease is rheumatoid arthritis, juvenile rheumatoid arthritis, inflammatory bowel disease (ulcerative colitis, Crohn's disease), age-related macular degeneration, IgA nephropathy, glomerulonephritis, vasculitis, polymyositis, or Wegener's disease.

Still another aspect of the invention relates to a method of treating neurodegenerative diseases in a subject, comprising administering to the subject a therapeutically effective amount of a compound disclosed herein or a tautomer, enantiomer, or salt thereof, or a composition disclosed herein. For example, the neurodegenerative disease is Alzheimer's disease, Parkinson's disease, multiple sclerosis, IgM polyneuropathies, or myasthenia gravis.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the disclosure will be apparent from the following detailed description and claims.

### DETAILED DESCRIPTION

STING (STimulator of INterferon Genes) is a central mediator for a cytosolic pathway that triggers type 1 interferon, in response to sensing cytosolic double-stranded (ds) DNA from infectious pathogens or aberrant host cells (Danger Associated Molecular Patterns, DAMPS) (Barber, Immunol. Rev 243: 99-108, 2011). Alternatively known as TMEM173, MITA, ERIS, and MPYS, STING was discovered using cDNA expression cloning methods as a MyD88-independent host cell defense factor expressed in macrophages, dendritic cells (DCs) and fibroblasts was found to induce expression of IFN-β and NF-κB dependent pro-inflammatory cytokines in response to sensing cytoplasmic DNA, in response to infection with herpes simplex vims (Ishikawa and Barber, Nature 455: 674-79, 2008).

While STING was discovered as being the critical sensor for inducing the production of IFN-β in response to infection with herpes simplex vims, the mechanism for this sensing function initially remained elusive. This conundrum was solved with the discovery of cyclic GMP-AMP synthase (cGAS), a host cell nucleotidyl transferase that directly binds dsDNA, and in response synthesizes a second messenger, c[G(2′,5′)pA(3′,5′)p] (cyclic GMP-AMP or 2′3′-cGAMP), which activates the STING pathway and induces IFN-β expression (Sun et al., Science 339: 786-91, 2013; Wu et al., Science 339: 826-30, 2013). This 2′3′-cGAMP product differed from bacterial-derived canonical cyclic dinucleotides, which were shown to respond differently to single nucleotide polymorphisms in the hSTING gene (Diner et al., Cell Reports 3:1355-1361, 2013; Gao et al., Cell 154:748-762, 2013; Conlon et. al., J Immunol 190:5216-5225, 2013). It was demonstrated that, while the bacterial-derived cyclic dinucleotides contained bis-3′-5′ linkages, cGAS produces a non-canonical, i.e., mixed linkage, CDN represented as c[G(2′,5′)pA(3′,5′)p] (Diner et al., Cell Reports 3:1355-1361, 2013; Gao et al., Cell 153:1094-1107, 2013; Ablasser et al., Nature 498: 380-84, 2013; Kranzusch et al., Cell Reports 3: 1362-68, 2013; Zhang et al., Mol. Cell. 51: 226-35, 2013). Cells without a functional cGAS are unable to express IFN-β in response to stimulation with cytosolic DNA.

Given the role of cGAS in the STING pathway and the role of type I interferons in various diseases, treatment with a cGAS inhibitor may have therapeutic benefit in a number of inflammatory, autoimmune, and neurodegenerative diseases, including, but are not limited to, systemic inflammatory response syndrome (SIRS), sepsis, septic shock, atherosclerosis, celiac disease, interstitial cystitis, transplant rejection, Aicardi-Goutieres Syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, myasthenia gravis, vasculitis, Type 1 diabetes, Type 2 diabetes, Sjorgen's syndrome, polymyositis, spondyloenchondrodysplasia, age-related macular degeneration, Alzheimer's disease and Parkinson's disease.

The present invention provides novel triazine compounds, synthetic methods for making the compounds, pharmaceutical compositions containing them and various uses of the compounds.

### Triazine Compounds

The present invention provides compounds of Formula (I):

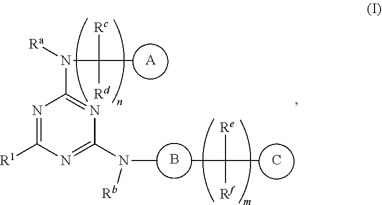

or a pharmaceutically acceptable salt thereof. In this formula:
each of R^{a} and R^{b} independently is H or C₁-C₆ alkyl, wherein C₁-C₆ alkyl is optionally substituted with one or more substituents selected from the group consisting of cyano, halo, —OR, —S(═O)ₓR, and —NRR′;
each of R^{c} and R^{d} independently is, at each occurrence, H, halo, —C(═O)R², or C₁-C₃ alkyl, wherein the C₁-C₃ alkyl is optionally substituted with one or more substituents independently selected from the group consisting of cyano, halo, —OR, —S(═O)ₓR, —NRR′; or R^{c} and R^{d} attached to the same carbon is oxo; or R^{c} and R^{d} attached to the same carbon forms a C₃-C₈ cycloalkyl, or a 3- to 8-membered heterocycloalkyl, wherein the C₃-C₈ cycloalkyl, or 3- to 8-membered heterocycloalkyl is optionally substituted with one or more substituents independently selected from the group consisting of cyano, halo, oxo, —OR, —S(═O)ₓR, —NRR′, C₁-C₃ alkyl and C₁-C₃ haloalkyl; and wherein at least one occurrence of R^{c} is —C(═O)R²;
R² is —OR^{g} or —NR^{g}R^{h};
each of R^{g} and R^{h} is independently H, C₁-C₆ alkyl, (CH₂CH₂O)ᵤ—H, or (CH₂CH₂O)ᵤ—C₁-C₆ alkyl;
each of R^{e} and R^{f} independently is H, halo, or C₁-C₃ alkyl, wherein the C₁-C₃ alkyl is optionally substituted with one or more substituents independently selected from the group consisting of cyano, halo, —OR, —S(═O)ₓR, and —NRR′; or R^{e} and R^{f} attached to the same carbon is oxo; or R^{e} and R^{f} attached to the same carbon forms a C₃-C₈ cycloalkyl, or a 3- to 8-membered heterocycloalkyl, wherein the C₃-C₈ cycloalkyl, or 3- to 8-membered heterocycloalkyl is optionally substituted with one or more substituents independently selected from the group consisting of cyano, halo, oxo, —OR, —S(═O)ₓR, —NRR′, C₁-C₃ alkyl and C₁-C₃ haloalkyl;
R¹ is H, halo, cyano, —OR¹, —NRⁱR^{j}, or C₁-C₆ alkyl, wherein C₁-C₆ alkyl is optionally substituted with one or more R^{S1};
each R^{S1} is independently halo, cyano, oxo, —OR¹, —NRⁱR^{j}, C₃-C₈ cycloalkyl, C₆-C₁₀ aryl, 3- to 8-membered heterocycloalkyl, or 5- to 10-membered heteroaryl, wherein the C₃-C₈ cycloalkyl, C₆-C₁₀ aryl, 3- to 8-membered heterocycloalkyl, or 5- to 10-membered heteroaryl is optionally substituted with one or more substituents independently selected from the group consisting of cyano, halo, oxo, —OR, —S(═O)ₓR, —NRR′, C₁-C₆ alkyl and C₁-C₆ haloalkyl;
each of Rⁱ and R^{j} independently is H, C₁-C₆ alkyl, C₃-C₈ cycloalkyl, 3- to 10-membered heterocycloalkyl, C₆-C₁₀ aryl or 5- to 10-membered heteroaryl wherein the C₁-C₆ alkyl is optionally substituted with one or more R^{S2}, and the C₃-C₈ cycloalkyl, 3- to 10-membered heterocycloalkyl, C₆-C₁₀ aryl or 5- to 10-membered heteroaryl is optionally substituted with one or more substituents independently selected from the group consisting of cyano, halo, oxo, —OR, —S(═O)ₓR, —NRR′, C₁-C₆ alkyl and C₁-C₆ haloalkyl;
each R^{S2} is independently halo, cyano, —OR, —NRR, C₃-C₈ cycloalkyl, 3- to 10-membered heterocycloalkyl, C₆-C₁₀ aryl or 5- to 10-membered heteroaryl, wherein the C₃-C₈ cycloalkyl, 3- to 10-membered heterocycloalkyl, C₆-C₁₀ aryl or 5- to 10-membered heteroaryl is optionally substituted with one or more substituents independently selected from the group consisting of cyano, halo, oxo, —OR, —S(═O)ₓR, —NRR′, and C₁-C₆ alkyl, wherein the C₁-C₆ alkyl is optionally substituted with one or more substituents independently selected from the group consisting of cyano, halo, oxo, —OR, —S(═O)ₓR, and —NRR′;
each of R and R′ independently is, at each occurrence, H, C₁-C₆ alkyl, C₁-C₆ haloalkyl, C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein the C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of cyano, halo, oxo, —OR^{p}, —S(═O)ₓR^{p}, —NR^{p}R^{q}, and C₁-C₆ alkyl, wherein the C₁-C₆ alkyl is optionally substituted with one or more substituents independently selected from the group consisting of cyano, halo, oxo, —OR^{p}, —S(═O)ₓR^{p}, and —NR^{p}R^{q};
A is a 3- to 14-membered ring optionally containing 1-4 heteroatoms selected from N, O, and S and optionally substituted with one or more R^{S3};
B is a 3- to 14-membered ring optionally containing 1-4 heteroatoms selected from N, O, and S and optionally substituted with one or more R^{S4};
C is a 3- to 14-membered ring optionally containing 1-4 heteroatoms selected from N, O, and S and optionally substituted with one or more R^{S5};
each of R^{S3}, and R^{S4} is independently selected from the group consisting of cyano, halo, oxo, —OR, —S(═O)ₓR, —NRR′, and C₁-C₆ alkyl, wherein the C₁-C₆ alkyl is optionally substituted with one or more substituents independently selected from the group consisting of cyano, halo, oxo, —OR, —S(═O)ₓR, and —NRR′;
each R^{S5} independently is halo, cyano, —C(═O)R^{k}, —S(═O)ₓR^{k}, a nitrogen protecting group bound to a suitable nitrogen of ring C, or C₁-C₆ alkyl, wherein the alkyl is optionally substituted with one or more R^{S6};
R^{k} is H, C₁-C₆ alkyl, C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein C₁-C₆ alkyl is optionally substituted with one or more R^{S6}, and wherein C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of R^{S6}, C₁-C₆ alkyl, and C₁-C₆ alkyl substituted with one or more R^{S6};
each R^{S6} independently is halo, cyano, oxo, —OR^{m}, —OC(═O)R^{m}, —C(═O)R^{m}, —C(═O)OR^{m}, —S(═O)ₓR^{m}, —S(═O)₂OR^{m}, —OS(═O)₂R^{m}, —NR^{m}Rⁿ, —C(═O)NR^{m}Rⁿ, —C(═NR^{m})NR^{m}Rⁿ, —S(═O)₂NR^{m}Rⁿ, —NR^{m}C(═O)R^{m}, —NR^{m}C(═NR^{m})R^{m}, —NR^{m}S(═O)₂R^{m}, —NR^{m}C(═O)NR^{m}Rⁿ, —NR^{m}C(═NR^{m})NR^{m}Rⁿ, —NR^{m}S(═O)₂NR^{m}Rⁿ, —NR^{m}C(═O)OR^{m}, —OC(═O)NR^{m}Rⁿ, C₀₋₃alkylene-CV C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein the C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of cyano, halo, oxo, OR^{m}, OC(═O)R^{m}, C(═O)R^{m}, C(═O)OR^{m}, S(═O)ₓR^{m}, S(═O)₂OR^{m}, OS(═O)₂R^{m}, NR^{m}Rⁿ, C(═O)NR^{m}Rⁿ, C(═NR^{m})NR^{m}Rⁿ, S(═O)₂NR^{m}Rⁿ, NR^{m}C(═O)R^{m}, NR^{m}C(═NR^{m})R^{m}, NR^{m}S(═O)₂R^{m}, NR^{m}C(═O)NR^{m}Rⁿ, NR^{m}C(═NR^{m})NR^{m}Rⁿ, NR^{m}S(═O)₂NR^{m}Rⁿ, NR^{m}C(═O)OR^{m}, OC(═O)NR^{m}Rⁿ, and C₁-C₆ alkyl, wherein the C₁-C₆ alkyl is optionally substituted with one or more substituents independently selected from the group consisting of cyano, halo, oxo, OR^{m}, OC(═O)R^{m}, C(═O)R^{m}, C(═O)OR^{m}, S(═O)ₓR^{m}, S(═O)₂OR^{m}, OS(═O)₂R^{m}, NR^{m}Rⁿ, C(═O)NR^{m}Rⁿ, C(═NR^{m})NR^{m}Rⁿ, S(═O)₂NR^{m}Rⁿ, NR^{m}C(═O)R^{m}, NR^{m}C(═NR^{m})R^{m}, NR^{m}S(═O)₂R^{m}, NR^{m}C(═O)NR^{m}Rⁿ, NR^{m}C(═NR^{m})NR^{m}Rⁿ, NR^{m}S(═O)₂NR^{m}Rⁿ, NR^{m}C(═O)OR^{m}, OC(═O)NR^{m}Rⁿ;
each of R^{m} and Rⁿ independently is, at each occurrence, H, C₁-C₆ alkyl, C₁-C₆ haloalkyl, C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein the C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of cyano, halo, oxo, —OR^{p}, —OC(═O)R^{p}, —C(═O)R^{p}, —C(═O)OR^{p}, —S(═O)ₓR^{p}, —S(═O)₂OR^{p}, —OS(═O)₂R^{p}, —NR^{p}R^{q}, —C(═O)NR^{p}R^{q}, —C(═NR^{p})NR^{p}R^{q}, —S(═O)₂NR^{p}R^{q}, —NR^{p}C(═O)R^{p}, —NR^{p}C(═NR^{p})R^{p}, —NR^{p}S(═O)₂R^{p}, —NR^{p}C(═O)NR^{p}R^{q}, —NR^{p}C(═NR^{p})NR^{p}R^{q}, —NR^{p}S(═O)₂NR^{p}R^{q}, —NR^{p}C(═O)OR^{p}, —OC(═O)NR^{p}R^{q}, and C₁-C₆ alkyl, wherein the C₁-C₆ alkyl is optionally substituted with one or more substituents independently selected from the group consisting of cyano, halo, oxo, —OR^{p}, —OC(═O)R^{p}, —C(═O)R^{p}, —C(═O)OR^{p}, —S(═O)ₓR^{p}, —S(═O)₂OR^{p}, —OS(═O)₂R^{p}, —NR^{p}R^{q}, —C(═O)NR^{p}R^{q}, —C(═NR^{p})NR^{p}R^{q}, —S(═O)₂NR^{p}R^{q}, —NR^{p}C(═O)R^{p}, —NR^{p}C(═NR^{p})R^{p}, —NR^{p}S(═O)₂R^{p}, —NR^{p}C(═O)NR^{p}R^{q}, —NR^{p}C(═NR^{p})NR^{p}R^{q}, —NR^{p}S(═O)₂NR^{p}R^{q}, —NR^{p}C(═O)OR^{p}, —OC(═O)NR^{p}R^{q};
each R^{p} and R^{q} are independently H or C₁-C₆ alkyl;
m is 0, 1, 2, or 3;
n is 1, 2, 3, or 4;
each occurrence of x is independently 0, 1, or 2; and
each u independently is 1, 2, or 3.

For example, n is 2.

For example, one occurrence of R^{c} is —C(O)OR and the other occurrences, if present, are H.

For example, one occurrence of R^{c} is —C(O)OCH₃ and the other occurrences, if present, are H.

For example, one occurrence of R^{c} is —C(O)NRR′ and the other occurrences, if present, are H.

For example, one occurrence of R^{c} is —C(O)NHCH₃, —C(O)N(CH₃)₂, —C(O)NHCH₂CH₂OCH₃, or —C(O)NHCH₂CH₂OCH₂CH₂OCH₃ and the other occurrences, if present, are H.

The present invention provides compounds of Formula (I) having the structure of Formula (Ia):

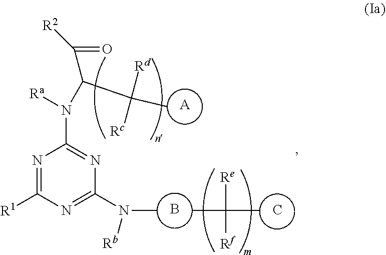

or a pharmaceutically acceptable salt thereof, wherein n′ is 0, 1, 2, or 3.

For example, each R^{c} is H.

For example, each R^{d} is H.

For example, nisi.

For example, A is a 5- to 6-membered ring optionally containing 1-4 heteroatoms selected from N, O, and S and optionally substituted with one or more R^{S3}.

For example, A is phenyl.

For example, A is 4-cyanophenyl.

For example, B is a 5- to 6-membered ring optionally containing 1-4 heteroatoms selected from N, O, and S and optionally substituted with one or more R^{S4}.

For example, B is phenyl.

For example, each R^{e} is H.

For example, each R^{f} is H.

For example, m is 1.

For example, C is a 5- to 6-membered ring optionally containing 1-4 heteroatoms selected from N, O, and S and optionally substituted with one or more R^{S5}.

For example, C is a 6-membered ring optionally containing 1-2 heteroatoms selected from N, O, and S and optionally substituted with one or more R^{S5}.

For example, C is

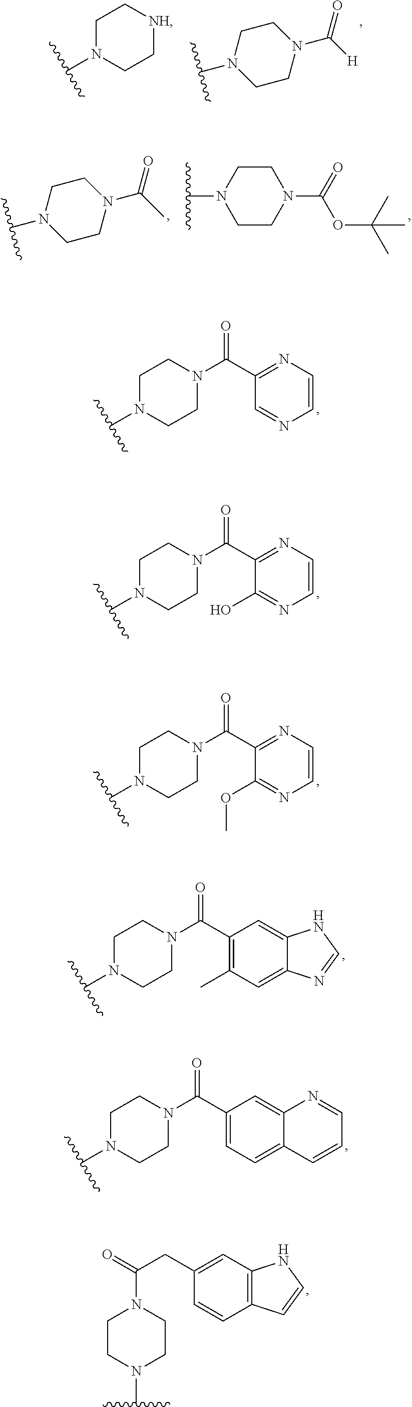

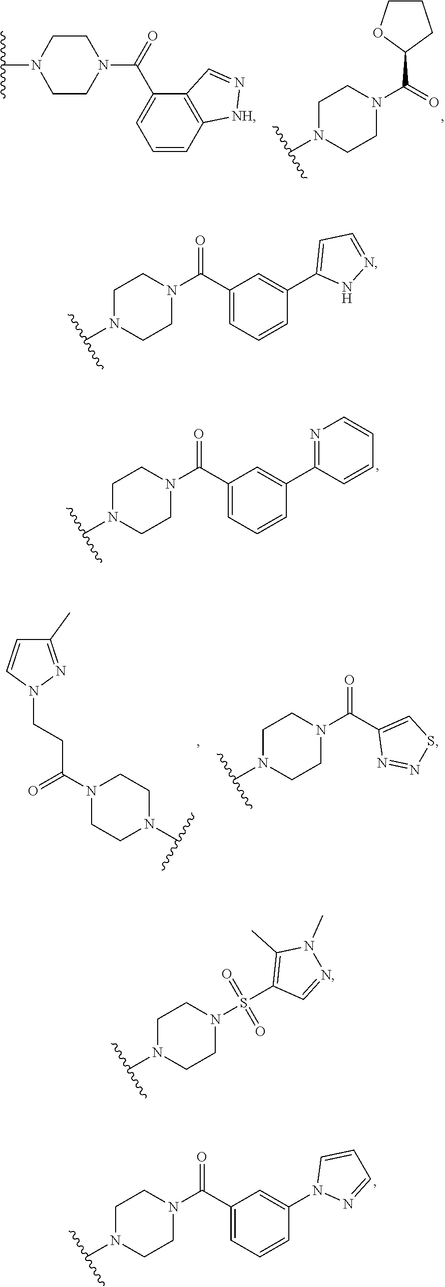

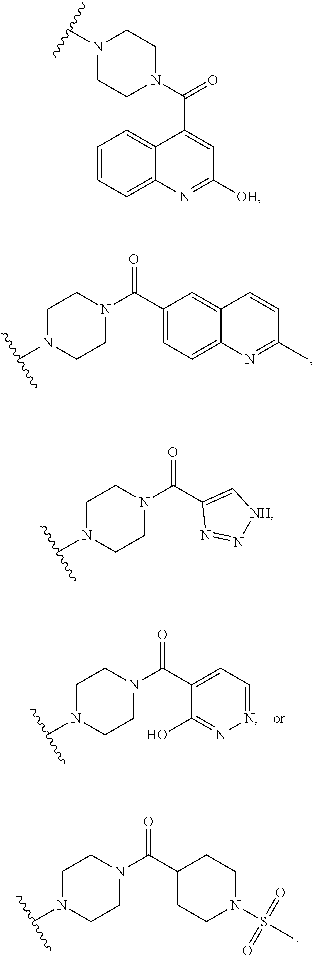 [SMILES: *N1CCN(C(=O)c2cc(O)nc3ccccc23)CC1 ; *N1CCN(C(=O)c2ccc3nc(C)ccc3c2)CC1 ; *N1CCN(C(=O)c2c[nH]nn2)CC1 ; *N1CCN(C(=O)c2ccnnc2O)CC1 ; *N1CCN(C(=O)C2CCN(S(C)(=O)=O)CC2)CC1]

The present invention provides compounds of Formula (I) having the structure of Formula (Ib):

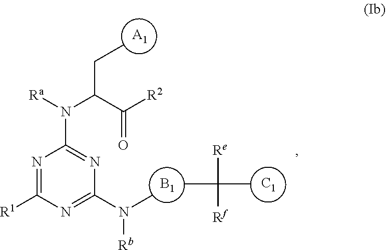

or a pharmaceutically acceptable salt wherein
A₁ is a 3- to 14-membered ring optionally containing 1-4 heteroatoms selected from N, O, and S and optionally substituted with one or more R^{S3};
B₁ is a 3- to 14-membered ring optionally containing 1-4 heteroatoms selected from N, O, and S and optionally substituted with one or more R^{S4}; and
C₁ is a 3- to 14-membered ring optionally containing 1-4 heteroatoms selected from N, O, and S and optionally substituted with one or more R^{S5}.

For example, A₁ is phenyl.

For example, A₁ is 4-cyanophenyl.

For example, B₁ is a 5- to 6-membered ring optionally containing 1∝heteroatoms selected from N, O, and S and optionally substituted with one or more R^{S4}.

For example, B₁ is phenyl.

For example, C₁ is a 5- to 6-membered ring optionally containing 1-4 heteroatoms selected from N, O, and S and optionally substituted with one or more R^{S5}.

For example, C₁ is a 6-membered ring optionally containing 1-2 heteroatoms selected from N, O, and S and optionally substituted with one or more R^{S5}.

For example, C₁ is

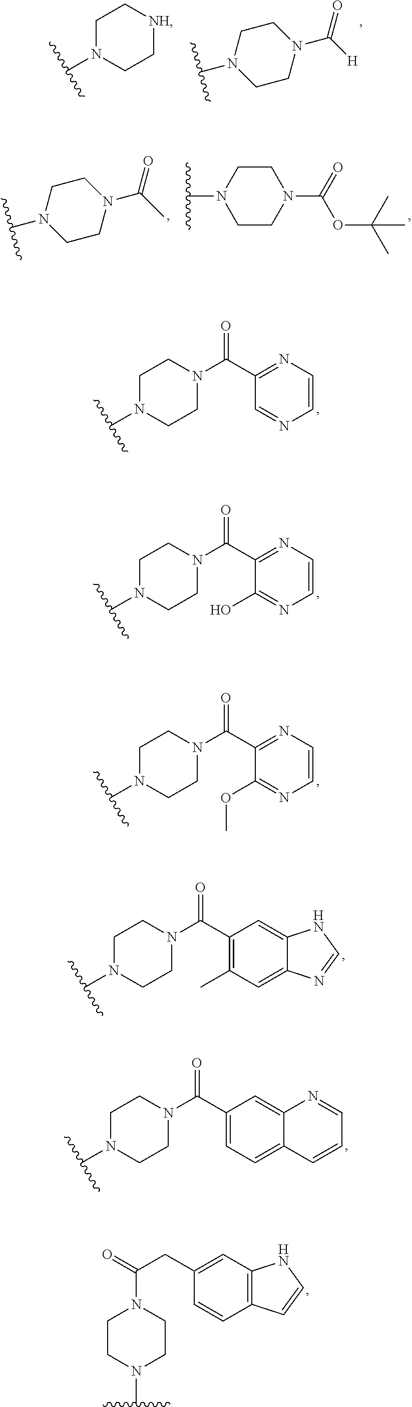

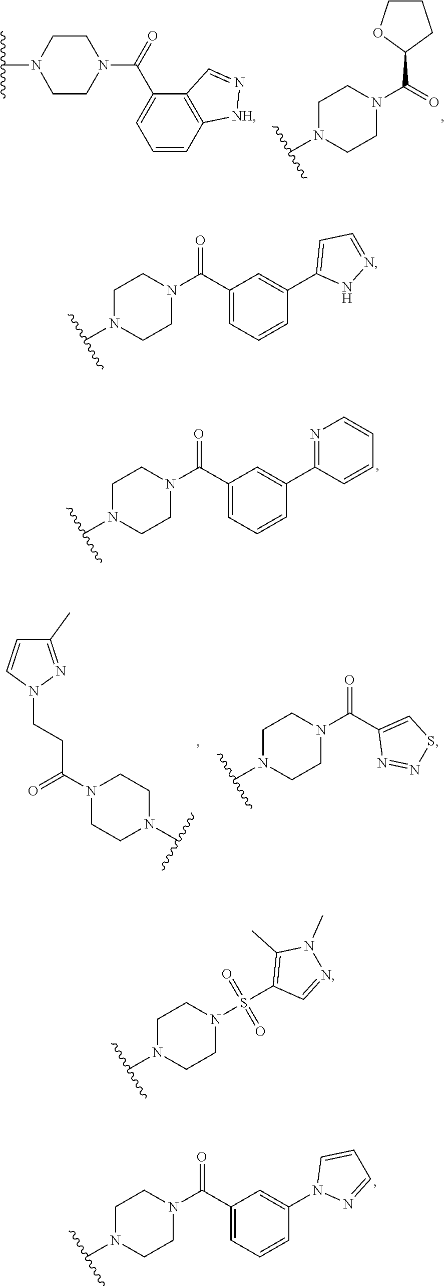

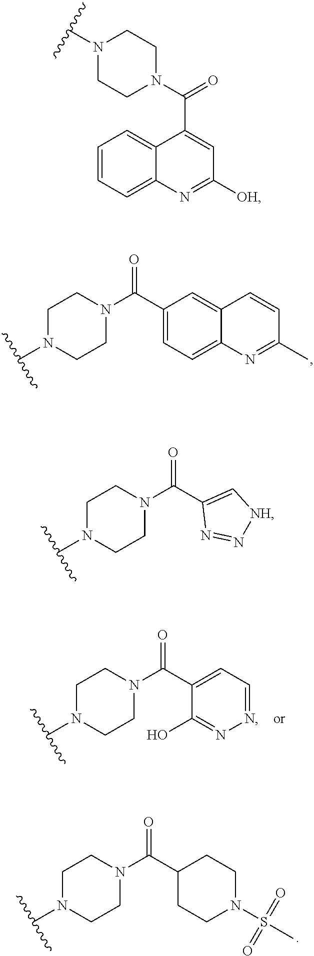 [SMILES: *N1CCN(C(=O)c2cc(O)nc3ccccc23)CC1 ; *N1CCN(C(=O)c2ccc3nc(C)ccc3c2)CC1 ; *N1CCN(C(=O)c2c[nH]nn2)CC1 ; *N1CCN(C(=O)c2ccnnc2O)CC1 ; *N1CCN(C(=O)C2CCN(S(C)(=O)=O)CC2)CC1]

The present invention provides compounds of Formula (I) having the structure of Formula (Ic):

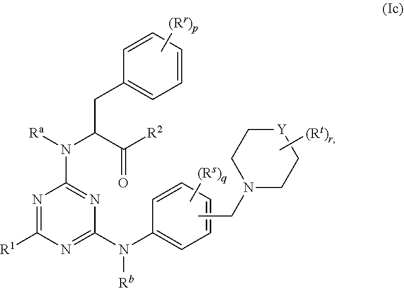

or a pharmaceutically acceptable salt thereof, wherein
each R^{r} independently is cyano, halo, oxo, —OR, —S(═O)ₓR, —NRR′, and C₁-C₆ alkyl;
each R^{s} independently is cyano, halo, oxo, —OR, —S(═O)ₓR, —NRR′, and C₁-C₆ alkyl;
each R^{L} independently is halo, cyano, —C(═O)R^{k}, —S(═O)ₓR^{k}, a nitrogen protecting group bound to a suitable nitrogen in the ring, or C₁-C₆ alkyl, wherein the alkyl is optionally substituted with one or more R^{S6};
Y is O, CH₂, CHR^{t}, NH, or NR^{t};
p is 0, 1, 2, 3, 4, or 5; and
each of q and r independently is 0, 1, 2, 3, or 4.

For example, p is 1.

For example, R^{r} is cyano.

For example, q is 0.

For example, r is 1.

For example, r is 0.

For example, Y is NH.

For example, Y is NR^{L}.

For example, R¹ is C(═O)R^{k}.

For example, R^{k} is H.

For example, R^{L} is a nitrogen protecting group.

For example, R^{L} is —C(═O)C₂-C₄ alkoxy.

For example, R^{L} is —C(═O)O-t-butyl.

For example, R^{k} is 5- to 6-membered heteroaryl.

For example, R^{k} is pyrazinyl.

For example, R^{k} is

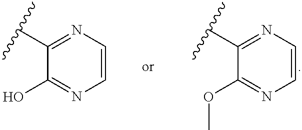

For example, R^{k} is 9- to 10-membered heteroaryl.

For example, R^{k} is benzimidazolyl.

For example, R^{k} is

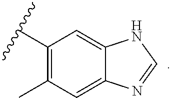 [SMILES: *c1cc2[nH]cnc2cc1C]

For example, R¹ is H.

For example, R¹ is halo.

For example, R¹ is Cl.

For example, R¹ is OR¹.

For example, R¹ is C₁-C₆ alkyl.

For example, R¹ is methyl.

For example, R¹ is NRⁱR^{j}.

For example, R¹ is C₁-C₆ alkyl and R^{j} is H.

For example, each of Rⁱ and R^{j} is C₁-C₆ alkyl.

For example, R¹ is NHCH₃,

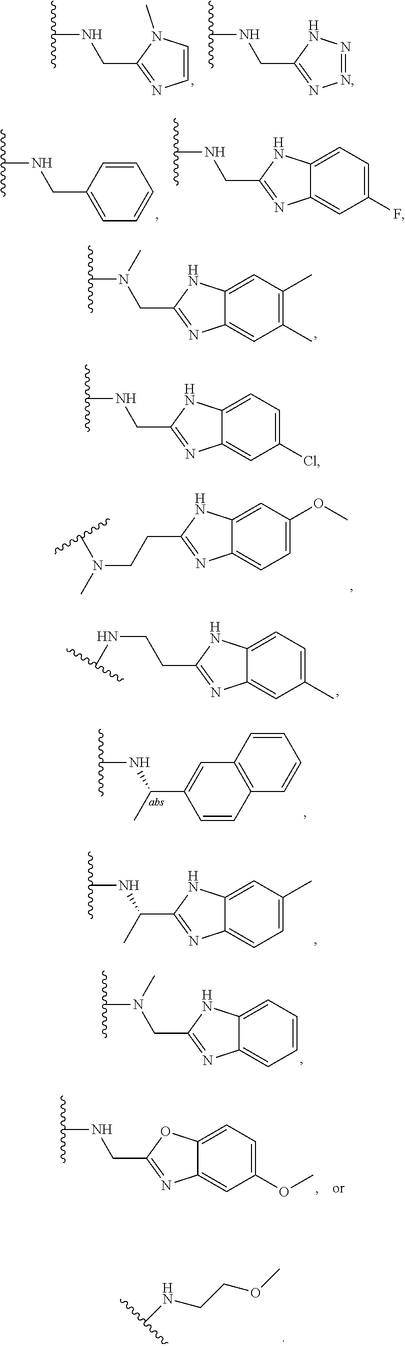

For example, R² is OR^{g}.

For example, R^{g} is C₁-C₃ alkyl.

For example, R^{g} is methyl.

For example, R² is NR^{g}R^{h}.

For example, R^{g} is C₁-C₆ alkyl and R^{h} is H.

For example, R^{g} is methyl.

For example, each of R^{g} and R^{h} is C₁-C₆ alkyl.

For example, each of R^{g} and R^{h} is methyl.

For example, R^{g} is (CH₂CH₂O)ᵤ—C₁-C₆ alkyl and R^{g} is H.

For example, R^{g} is (CH₂CH₂O)ᵤ—CH₃.

For example, u is 1.

For example, u is 2.

For example, R^{a} is H.

For example, R^{b} is H.

Representative compounds of the present invention are listed in Table 1A below followed by their compound number:

TABLE 1A
tert-butyl (S)-4-(3-((4-chloro-6-((3-(4-cyanophenyl)-1-methoxy-1-oxopropan-2-yl)amino)-
1,3,5-triazin-2-yl)amino)benzyl)piperazine-1-carboxylate (TA1002);
tert-butyl (S)-4-(3-((4-((3-(4-cyanophenyl)-1-methoxy-1-oxopropan-2-yl)amino)-6-(((5-
fluoro-1H-benzo[d]imidazol-2-yl)methyl)amino)-1,3,5-triazin-2-yl)amino)benzyl)piperazine-
1-carboxylate (TA1003);
tert-butyl (S)-4-(3-((4-chloro-6-((3-(4-cyanophenyl)-1-(dimethylamino)-1-oxopropan-2-
yl)amino)-1,3,5-triazin-2-yl)amino)benzyl)piperazine-1-carboxylate (TA1008);
(S)-2-((4-chloro-6-((3-(piperazin-1-ylmethyl)phenyl)amino)-1,3,5-triazin-2-yl)amino)-3-(4-
cyanophenyl)-N,N-dimethylpropanamide (TA1009);
tert-butyl (S)-4-(3-((4-((3-(4-cyanophenyl)-1-(dimethylamino)-1-oxopropan-2-yl)amino)-
1,3,5-triazin-2-yl)amino)benzyl)piperazine-1-carboxylate (TA1010);
(S)-3-(4-cyanophenyl)-N,N-dimethyl-2-((4-((3-(piperazin-1-ylmethyl)phenyl)amino)-1,3,5-
triazin-2-yl)amino)propanamide (TA1011);
methyl (S)-3-(4-cyanophenyl)-2-((4-(((5-fluoro-1H-benzo[d]imidazol-2-yl)methyl)amino)-6-
((3-(piperazin-1-ylmethyl)phenyl)amino)-1,3,5-triazin-2-yl)amino)propanoate (TA1012);
(S)-3-(4-cyanophenyl)-2-((4-(((5-fluoro-1H-benzo[d]imidazol-2-yl)methyl)amino)-6-((3-
(piperazin-1-ylmethyl)phenyl)amino)-1,3,5-triazin-2-yl)amino)-N,N-dimethylpropanamide
(TA1013);
(S)-3-(4-cyanophenyl)-2-((4-(((5,6-dimethyl-1H-benzo[d]imidazol-2-
yl)methyl)(methyl)amino)-6-((3-(piperazin-1-ylmethyl)phenyl)amino)-1,3,5-triazin-2-
yl)amino)-N,N-dimethylpropanamide (TA1014);
(S)-3-(4-cyanophenyl)-2-((4-(((5,6-dimethyl-1H-benzo[d]imidazol-2-
yl)methyl)(methyl)amino)-6-((3-((4-(5-methyl-1H-benzo[d]imidazole-6-carbonyl)piperazin-1-
yl)methyl)phenyl)amino)-1,3,5-triazin-2-yl)amino)-N,N-dimethylpropanamide (TA1015);
(S)-3-(4-cyanophenyl)-2-((4-(((5-fluoro-1H-benzo[d]imidazol-2-yl)methyl)amino)-6-((3-
((4-formylpiperazin-1-yl)methyl)phenyl)amino)-1,3,5-triazin-2-yl)amino)-N,N-
dimethylpropanamide (TA1016);
(S)-3-(4-cyanophenyl)-2-((4-(((5-fluoro-1H-benzo[d]imidazol-2-yl)methyl)amino)-6-((3-
(piperazin-1-ylmethyl)phenyl)amino)-1,3,5-triazin-2-yl)amino)-N-(2-
methoxyethyl)propanamide (TA1017);
(S)-3-(4-cyanophenyl)-2-((4-(((5-fluoro-1H-benzo[d]imidazol-2-yl)methyl)amino)-6-((3-((4-
(3-hydroxypyrazine-2-carbonyl)piperazin-1-yl)methyl)phenyl)amino)-1,3,5-triazin-2-
yl)amino)-N-(2-methoxyethyl)propanamide (TA1018);
methyl (S)-3-(4-cyanophenyl)-2-((4-(((5-fluoro-1H-benzo[d]imidazol-2-yl)methyl)amino)-6-
((3-(piperazin-1-ylmethyl)phenyl)amino)-1,3,5-triazin-2-yl)amino)propanoate (TA1019);
methyl (S)-3-(4-cyanophenyl)-2-((4-(((5-fluoro-1H-benzo[d]imidazol-2-yl)methyl)amino)-6-
((3-((4-(3-hydroxypyrazine-2-carbonyl)piperazin-1-yl)methyl)phenyl)amino)-1,3,5-triazin-2-
yl)amino)propanoate (TA1020);
(S)-3-(4-cyanophenyl)-2-((4-(((5-fluoro-1H-benzo[d]imidazol-2-yl)methyl)amino)-6-((3-((4-
(3-hydroxypyrazine-2-carbonyl)piperazin-1-yl)methyl)phenyl)amino)-1,3,5-triazin-2-
yl)amino)-N,N-dimethylpropanamide (TA1021);
(S)-3-(4-cyanophenyl)-2-((4-(((5-fluoro-1H-benzo[d]imidazol-2-yl)methyl)amino)-6-((3-
(piperazin-1-ylmethyl)phenyl)amino)-1,3,5-triazin-2-yl)amino)-N-methylpropanamide
(TA1022);
(S)-3-(4-cyanophenyl)-2-((4-(((5-fluoro-1H-benzo[d]imidazol-2-yl)methyl)amino)-6-((3-
(piperazin-1-ylmethyl)phenyl)amino)-1,3,5-triazin-2-yl)amino)-N-(2-(2-
methoxyethoxy)ethyl)propanamide (TA1023);
(S)-3-(4-cyanophenyl)-2-((4-(((5-fluoro-1H-benzo[d]imidazol-2-yl)methyl)amino)-6-((3-((4-
(3-hydroxypyrazine-2-carbonyl)piperazin-1-yl)methyl)phenyl)amino)-1,3,5-triazin-2-
yl)amino)-N-methylpropanamide (TA1024);
(S)-3-(4-cyanophenyl)-2-((4-(((5-fluoro-1H-benzo[d]imidazol-2-yl)methyl)amino)-6-((3-((4-
(3-hydroxypyrazine-2-carbonyl)piperazin-1-yl)methyl)phenyl)amino)-1,3,5-triazin-2-
yl)amino)-N-(2-(2-methoxyethoxy)ethyl)propanamide (TA1025);
methyl (S)-3-(4-cyanophenyl)-2-((4-((3-((4-(3-hydroxypyrazine-2-carbonyl)piperazin-1-
yl)methyl)phenyl)amino)-1,3,5-triazin-2-yl)amino)propanoate (TA1026);
methyl (R)-3-(4-cyanophenyl)-2-((4-(((5-fluoro-1H-benzo[d]imidazol-2-yl)methyl)amino)-6-
((3-((4-(3-hydroxypyrazine-2-carbonyl)piperazin-1-yl)methyl)phenyl)amino)-1,3,5-triazin-2-
yl)amino)propanoate (TA1027);
methyl (S)-3-(4-cyanophenyl)-2-((4-methoxy-6-((3-(piperazin-1-ylmethyl)phenyl)amino)-
1,3,5-triazin-2-yl)amino)propanoate (TA1028);
(S)-3-(4-cyanophenyl)-2-((4-(((5-fluoro-1H-benzo[d]imidazol-2-yl)methyl)amino)-6-((3-((4-
(3-methoxypyrazine-2-carbonyl)piperazin-1-yl)methyl)phenyl)amino)-1,3,5-triazin-2-
yl)amino)-N-(2-(2-methoxyethoxy)ethyl)propanamide (TA1029);
(S)-3-(4-cyanophenyl)-2-((4-(((5-fluoro-1H-benzo[d]imidazol-2-yl)methyl)amino)-6-((3-((4-
formylpiperazin-1-yl)methyl)phenyl)amino)-1,3,5-triazin-2-yl)amino)-N-methylpropanamide
(TA1030);
methyl (S)-3-(4-cyanophenyl)-2-((4-((2-methoxyethyl)amino)-6-((3-(piperazin-1-
ylmethyl)phenyl)amino)-1,3,5-triazin-2-yl)amino)propanoate (TA1031);
methyl (S)-3-(4-cyanophenyl)-2-((4-(((1-methyl-1H-imidazol-2-yl)methyl)amino)-6-((3-
(piperazin-1-ylmethyl)phenyl)amino)-1,3,5-triazin-2-yl)amino)propanoate (TA1032);
methyl (S)-3-(4-cyanophenyl)-2-((4-((3-((4-(3-hydroxypyrazine-2-carbonyl)piperazin-1-
yl)methyl)phenyl)amino)-6-((2-methoxyethyl)amino)-1,3,5-triazin-2-yl)amino)propanoate
(TA1033);
methyl (S)-3-(4-cyanophenyl)-2-((4-((3-((4-(3-hydroxypyrazine-2-carbonyl)piperazin-1-
yl)methyl)phenyl)amino)-6-(((1-methyl-1H-imidazol-2-yl)methyl)amino)-1,3,5-triazin-2-
yl)amino)propanoate (TA1034);
methyl (S)-3-(4-cyanophenyl)-2-((4-((3-((4-(3-hydroxypyrazine-2-carbonyl)piperazin-1-
yl)methyl)phenyl)amino)-6-(methylamino)-1,3,5-triazin-2-yl)amino)propanoate (TA1035);
tert-butyl (S)-4-(3-((4-((3-(4-cyanophenyl)-1-(dimethylamino)-1-oxopropan-2-yl)amino)-6-
(((5-fluoro-1H-benzo[d]imidazol-2-yl)methyl)amino)-1,3,5-triazin-2-
yl)amino)benzyl)piperazine-1-carboxylate (TA1036);
tert-butyl (S)-4-(3-((4-((3-(4-cyanophenyl)-1-((2-methoxyethyl)amino)-1-oxopropan-2-
yl)amino)-6-(((5-fluoro-1H-benzo[d]imidazol-2-yl)methyl)amino)-1,3,5-triazin-2-
yl)amino)benzyl)piperazine-1-carboxylate (TA1037);
tert-butyl (S)-4-(3-((4-((3-(4-cyanophenyl)-1-(methylamino)-1-oxopropan-2-yl)amino)-6-(((5-
fluoro-1H-benzo[d]imidazol-2-yl)methyl)amino)-1,3,5-triazin-2-yl)amino)benzyl)piperazine-
1-carboxylate (TA1038);
tert-butyl (S)-4-(3-((4-((3-(4-cyanophenyl)-1-((2-(2-methoxyethoxy)ethyl)amino)-1-
oxopropan-2-yl)amino)-6-(((5-fluoro-1H-benzo[d]imidazol-2-yl)methyl)amino)-1,3,5-triazin-
2-yl)amino)benzyl)piperazine-1-carboxylate (TA1039);
tert-butyl (R)-4-(3-((4-((3-(4-cyanophenyl)-1-methoxy-1-oxopropan-2-yl)amino)-6-(((5-
fluoro-1H-benzo[d]imidazol-2-yl)methyl)amino)-1,3,5-triazin-2-yl)amino)benzyl)piperazine-
1-carboxylate (TA1040);
tert-butyl (S)-4-(3-((4-((3-(4-cyanophenyl)-1-methoxy-1-oxopropan-2-yl)amino)-6-((2-
methoxyethyl)amino)-1,3,5-triazin-2-yl)amino)benzyl)piperazine-1-carboxylate (TA1041);
tert-butyl (S)-4-(3-((4-((3-(4-cyanophenyl)-1-methoxy-1-oxopropan-2-yl)amino)-6-(((1-
methyl-1H-imidazol-2-yl)methyl)amino)-1,3,5-triazin-2-yl)amino)benzyl)piperazine-1-
carboxylate (TA1042);
tert-butyl (S)-4-(3-((4-((3-(4-cyanophenyl)-1-methoxy-1-oxopropan-2-yl)amino)-6-
(methylamino)-1,3,5-triazin-2-yl)amino)benzyl)piperazine-1-carboxylate (TA1043);
methyl (S)-3-(4-cyanophenyl)-2-((4-(methylamino)-6-((3-(piperazin-1-
ylmethyl)phenyl)amino)-1,3,5-triazin-2-yl)amino)propanoate (TA1044);
tert-butyl (S)-4-(3-((4-((3-(4-cyanophenyl)-1-(dimethylamino)-1-oxopropan-2-yl)amino)-6-
(((5,6-dimethyl-1H-benzo[d]imidazol-2-yl)methyl)(methyl)amino)-1,3,5-triazin-2-
yl)amino)benzyl)piperazine-1-carboxylate (TA1045);
tert-butyl (S)-4-(3-((4-((3-(4-cyanophenyl)-1-methoxy-1-oxopropan-2-yl)amino)-1,3,5-triazin-
2-yl)amino)benzyl)piperazine-1-carboxylate (TA1046);
methyl (S)-3-(4-cyanophenyl)-2-((4-((3-(piperazin-1-ylmethyl)phenyl)amino)-1,3,5-triazin-2-
yl)amino)propanoate (TA1047);
tert-butyl (S)-4-(3-((4-(benzylamino)-6-((3-(4-cyanophenyl)-1-methoxy-1-oxopropan-2-
yl)amino)-1,3,5-triazin-2-yl)amino)benzyl)piperazine-1-carboxylate (TA1048);
methyl (S)-2-((4-(benzylamino)-6-((3-(piperazin-1-ylmethyl)phenyl)amino)-1,3,5-triazin-2-
yl)amino)-3-(4-cyanophenyl)propanoate (TA1049);
methyl (S)-2-((4-(benzylamino)-6-((3-((4-(3-hydroxypyrazine-2-carbonyl)piperazin-1-
yl)methyl)phenyl)amino)-1,3,5-triazin-2-yl)amino)-3-(4-cyanophenyl)propanoate (TA1050);
tert-butyl (S)-4-(3-((4-((3-(4-cyanophenyl)-1-methoxy-1-oxopropan-2-yl)amino)-6-((2-(6-
methoxy-1H-benzo[d]imidazol-2-yl)ethyl)(methyl)amino)-1,3,5-triazin-2-
yl)amino)benzyl)piperazine-1-carboxylate (TA1051);
methyl (S)-3-(4-cyanophenyl)-2-((4-((2-(6-methoxy-1H-benzo[d]imidazol-2-
yl)ethyl)(methyl)amino)-6-((3-(piperazin-d-ylmethyl)phenyl)amino)-1,3,5-triazin-2-
yl)amino)propanoate (TA1052);
methyl (S)-3-(4-cyanophenyl)-2-((4-((3-((4-(3-hydroxypyrazine-2-carbonyl)piperazin-1-
yl)methyl)phenyl)amino)-6-((2-(6-methoxy-1H-benzo[d]imidazol-2-yl)ethyl)(methyl)amino)-
1,3,5-triazin-2-yl)amino)propanoate (TA1053);
methyl (S)-3-(4-cyanophenyl)-2-((4-((2-(6-methoxy-1H-benzo[d]imidazol-2-
yl)ethyl)(methyl)amino)-6-((3-((4-(pyrazine-2-carbonyl)piperazin-1-yl)methyl)phenyl)amino)-
1,3,5-triazin-2-yl)amino)propanoate (TA1054);
methyl (S)-2-((4-((3-((4-(1,2,3-thiadiazole-4-carbonyl)piperazin-1-yl)methyl)phenyl)amino)-
6-((2-(6-methoxy-1H-benzo[d]imidazol-2-yl)ethyl)(methyl)amino)-1,3,5-triazin-2-yl)amino)-
3-(4-cyanophenyl)propanoate (TA1055);
methyl (S)-3-(4-cyanophenyl)-2-((4-((2-(6-methoxy-1H-benzo[d]imidazol-2-
yl)ethyl)(methyl)amino)-6-((3-((4-((S)-tetrahydrofuran-2-carbonyl)piperazin-1-
yl)methyl)phenyl)amino)-1,3,5-triazin-2-yl)amino)propanoate (TA1056);
tert-butyl (S)-4-(3-((4-(((5-chloro-1H-benzo[d]imidazol-2-yl)methyl)amino)-6-((3-(4-
cyanophenyl)-1-methoxy-1-oxopropan-2-yl)amino)-1,3,5-triazin-2-
yl)amino)benzyl)piperazine-1-carboxylate (TA1057);
methyl (S)-2-((4-(((5-chloro-1H-benzo[d]imidazol-2-yl)methyl)amino)-6-((3-(piperazin-1-
ylmethyl)phenyl)amino)-1,3,5-triazin-2-yl)amino)-3-(4-cyanophenyl)propanoate (TA1058);
methyl (S)-2-((4-(((5-chloro-1H-benzo[d]imidazol-2-yl)methyl)amino)-6-((3-((4-(3-
hydroxypyrazine-2-carbonyl)piperazin-1-yl)methyl)phenyl)amino)-1,3,5-triazin-2-yl)amino)-
3-(4-cyanophenyl)propanoate (TA1059);
methyl (S)-2-((4-((3-((4-(3-(1H-pyrazol-1-yl)benzoyl)piperazin-1-yl)methyl)phenyl)amino)-6-
(((5-chloro-1H-benzo[d]imidazol-2-yl)methyl)amino)-1,3,5-triazin-2-yl)amino)-3-(4-
cyanophenyl)propanoate (TA1060);
methyl (S)-2-((4-((3-((4-(1H-1,2,3-triazole-4-carbonyl)piperazin-1-yl)methyl)phenyl)amino)-
6-(((5-chloro-1H-benzo[d]imidazol-2-yl)methyl)amino)-1,3,5-triazin-2-yl)amino)-3-(4-
cyanophenyl)propanoate (TA1061);
methyl (S)-2-((4-(((5-chloro-1H-benzo[d]imidazol-2-yl)methyl)amino)-6-((3-((4-
formylpiperazin-1-yl)methyl)phenyl)amino)-1,3,5-triazin-2-yl)amino)-3-(4-
cyanophenyl)propanoate (TA1062);
tert-butyl (S)-4-(3-((4-(((5-fluoro-1H-benzo[d]imidazol-2-yl)methyl)amino)-6-((1-methoxy-1-
oxo-3-phenylpropan-2-yl)amino)-1,3,5-triazin-2-yl)amino)benzyl)piperazine-1-carboxylate
(TA1063);
methyl (4-(((5-fluoro-1H-benzo[d]imidazol-2-yl)methyl)amino)-6-((3-(piperazin-1-
ylmethyl)phenyl)amino)-1,3,5-triazin-2-yl)phenylalaninate (TA1064);
methyl (4-(((5-fluoro-1H-benzo[d]imidazol-2-yl)methyl)amino)-6-((3-((4-(3-
hydroxypyrazine-2-carbonyl)piperazin-1-yl)methyl)phenyl)amino)-1,3,5-triazin-2-yl)-L-
phenylalaninate (TA1065);
methyl (S)-3-(4-cyanophenyl)-2-((4-(((5-fluoro-1H-benzo[d]imidazol-2-yl)methyl)amino)-6-
((3-((4-(3-hydroxypyridazine-4-carbonyl)piperazin-1-yl)methyl)phenyl)amino)-1,3,5-triazin-
2-yl)amino)propanoate (TA1066);
tert-butyl (S)-4-(3-((4-(((1H-tetrazol-5-yl)methyl)amino)-6-((3-(4-cyanophenyl)-1-methoxy-1-
oxopropan-2-yl)amino)-1,3,5-triazin-2-yl)amino)benzyl)piperazine-1-carboxylate (TA1067);
methyl (S)-2-((4-(((1H-tetrazol-5-yl)methyl)amino)-6-((3-(piperazin-1-
ylmethyl)phenyl)amino)-1,3,5-triazin-2-yl)amino)-3-(4-cyanophenyl)propanoate (TA1068);
methyl (S)-2-((4-(((1H-tetrazol-5-yl)methyl)amino)-6-((3-((4-(3-hydroxypyrazine-2-
carbonyl)piperazin-1-yl)methyl)phenyl)amino)-1,3,5-triazin-2-yl)amino)-3-(4-
cyanophenyl)propanoate (TA1069);
2-(2-methoxyethoxy)ethyl (S)-3-(4-cyanophenyl)-2-((4-(((5-fluoro-1H-benzo[d]imidazol-2-
yl)methyl)amino)-6-((3-((4-(3-hydroxypyrazine-2-carbonyl)piperazin-1-
yl)methyl)phenyl)amino)-1,3,5-triazin-2-yl)amino)propanoate (TA1070);
(S)-3-(4-cyanophenyl)-2-((4-(((5-fluoro-1H-benzo[d]imidazol-2-yl)methyl)amino)-6-((3-((4-
(3-hydroxypyrazine-2-carbonyl)piperazin-1-yl)methyl)phenyl)amino)-1,3,5-triazin-2-
yl)amino)propanoic acid (TA1071);
methyl (S)-3-cyclohexyl-2-((4-(((5-fluoro-1H-benzo[d]imidazol-2-yl)methyl)amino)-6-((3-((4-
(3-hydroxypyrazine-2-carbonyl)piperazin-1-yl)methyl)phenyl)amino)-1,3,5-triazin-2-
yl)amino)propanoate (TA1072);
(S)-3-(4-cyanophenyl)-2-((4-(((5,6-dimethyl-1H-benzo[d]imidazol-2-
yl)methyl)(methyl)amino)-6-((3-((4-((1,5-dimethyl-1H-pyrazol-4-yl)sulfonyl)piperazin-1-
yl)methyl)phenyl)amino)-1,3,5-triazin-2-yl)amino)-N,N-dimethylpropanamide (TA1073);
methyl (S)-3-(4-cyanophenyl)-2-((4-((3-((4-(3-hydroxypyrazine-2-carbonyl)piperazin-1-
yl)methyl)phenyl)amino)-6-methoxy-1,3,5-triazin-2-yl)amino)propanoate (TA1074);
methyl (S)-2-((4-((3-((4-acetylpiperazin-1-yl)methyl)phenyl)amino)-6-(((5-fluoro-1H-
benzo[d]imidazol-2-yl)methyl)amino)-1,3,5-triazin-2-yl)amino)-3-(4-cyanophenyl)propanoate
(TA1075);
methyl (S)-2-((4-((3-((4-acetylpiperazin-1-yl)methyl)phenyl)amino)-6-(N-((5-fluoro-1H-
benzo[d]imidazol-2-yl)methyl)acetamido)-1,3,5-triazin-2-yl)amino)-3-(4-
cyanophenyl)propanoate (TA1076);
methyl (S)-3-(4-cyanophenyl)-2-((4-((3-((4-formylpiperazin-1-yl)methyl)phenyl)amino)-6-
methoxy-1,3,5-triazin-2-yl)amino)propanoate (TA1077);
methyl (S)-3-(4-cyanophenyl)-2-((4-(((5-fluoro-1H-benzo[d]imidazol-2-yl)methyl)amino)-6-
((3-((4-(3-methoxypyrazine-2-carbonyl)piperazin-1-yl)methyl)phenyl)amino)-1,3,5-triazin-2-
yl)amino)propanoate (TA1078); and
methyl (S)-3-(4-cyanophenyl)-2-((4-(((5-fluoro-1H-benzo[d]imidazol-2-yl)methyl)amino)-6-
((3-((4-(1-(methylsulfonyl)piperidine-4-carbonyl)piperazin-1-yl)methyl)phenyl)amino)-1,3,5-
triazin-2-yl)amino)propanoate (TA1079).

The representative compounds have the following structures as shown in Table IB below:

TABLE 1B

| Compound | |
|---|---|
| ID | Structure |
| TA1002 | 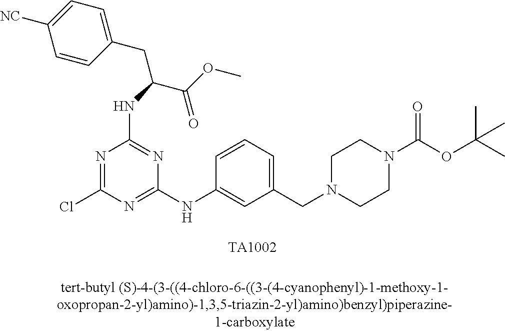 |
| TA1003 | 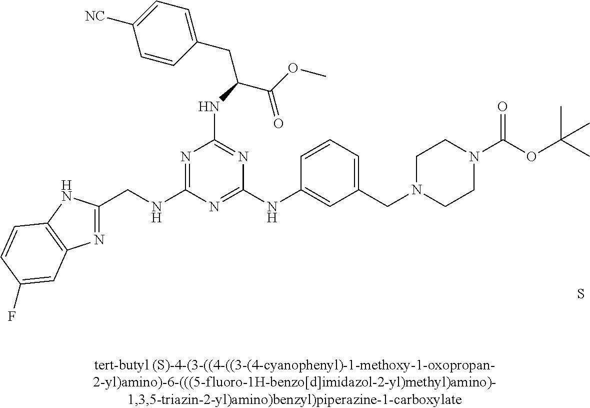 |
| TA1008 | 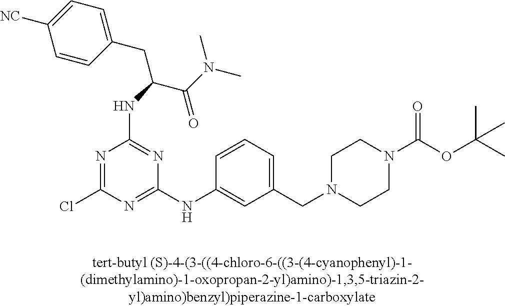 [SMILES: CN(C)C(=O)[C@H](Cc1ccc(C#N)cc1)Nc1nc(Cl)nc(Nc2cccc(CN3CCN(C(=O)OC(C)(C)C)CC3)c2)n1] |
| TA1009 | 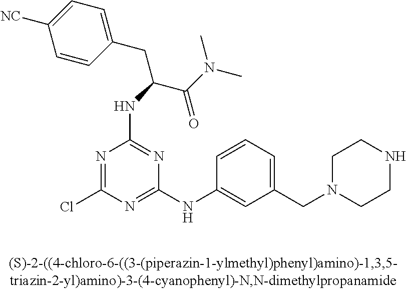 [SMILES: CN(C)C(=O)[C@H](Cc1ccc(C#N)cc1)Nc1nc(Cl)nc(Nc2cccc(CN3CCNCC3)c2)n1] |
| TA1010 | 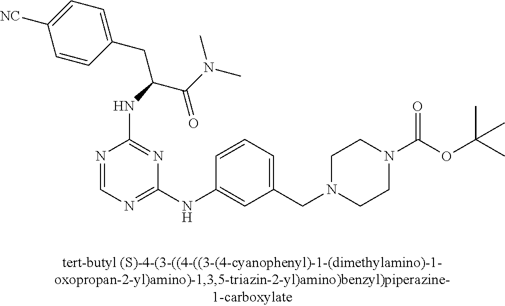 [SMILES: CN(C)C(=O)[C@H](Cc1ccc(C#N)cc1)Nc1ncnc(Nc2cccc(CN3CCN(C(=O)OC(C)(C)C)CC3)c2)n1] |
| TA1011 | 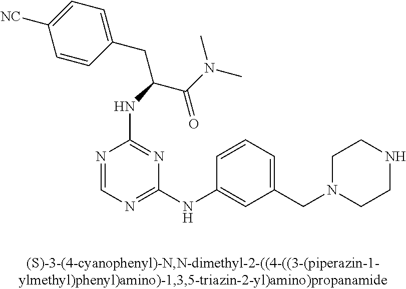 [SMILES: CN(C)C(=O)[C@H](Cc1ccc(C#N)cc1)Nc1ncnc(Nc2cccc(CN3CCNCC3)c2)n1] |
| TA1012 | 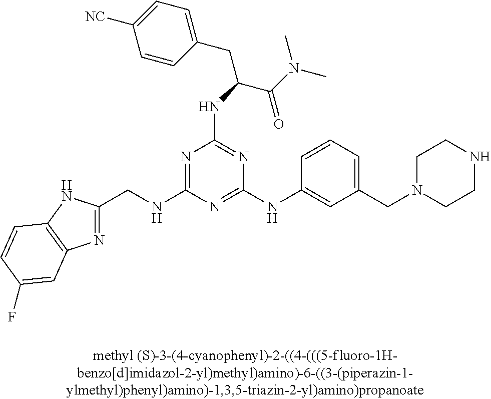 [SMILES: CN(C)C(=O)[C@H](Cc1ccc(C#N)cc1)Nc1nc(NCc2nc3cc(F)ccc3[nH]2)nc(Nc2cccc(CN3CCNCC3)c2)n1] |
| TA1013 | 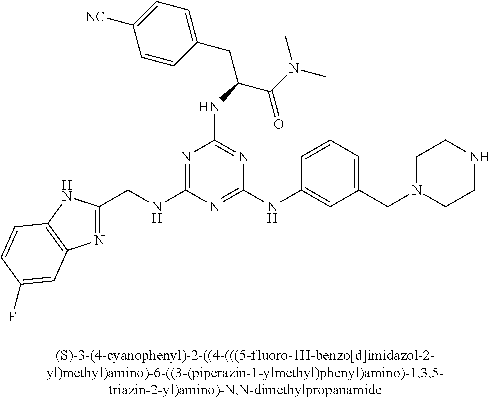 [SMILES: CN(C)C(=O)[C@H](Cc1ccc(C#N)cc1)Nc1nc(NCc2nc3cc(F)ccc3[nH]2)nc(Nc2cccc(CN3CCNCC3)c2)n1] |
| TA1014 | 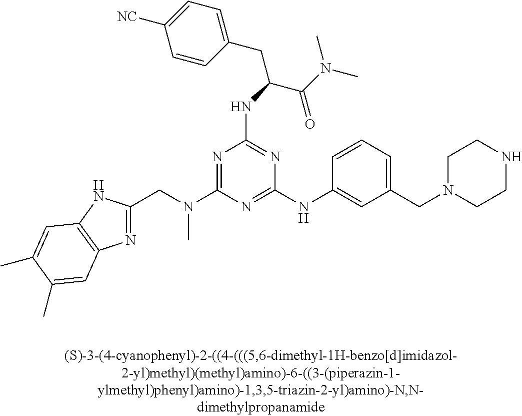 [SMILES: Cc1cc2nc(CN(C)c3nc(Nc4cccc(CN5CCNCC5)c4)nc(N[C@@H](Cc4ccc(C#N)cc4)C(=O)N(C)C)n3)[nH]c2cc1C] |
| TA1015 | 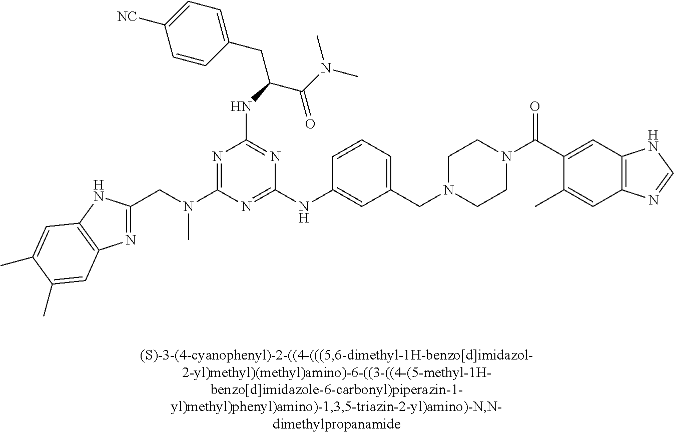 [SMILES: Cc1cc2nc(CN(C)c3nc(Nc4cccc(CN5CCN(C(=O)c6cc7[nH]cnc7cc6C)CC5)c4)nc(N[C@@H](Cc4ccc(C#N)cc4)C(=O)N(C)C)n3)[nH]c2cc1C] |
| TA1016 | 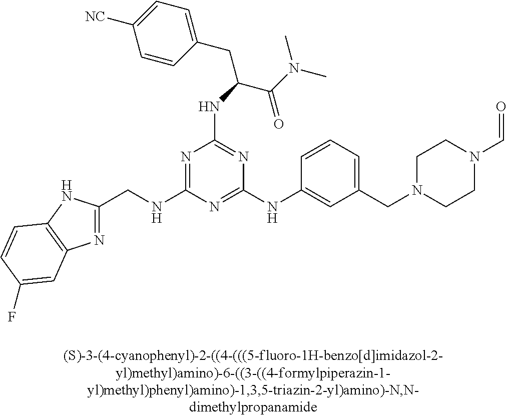 [SMILES: CN(C)C(=O)[C@H](Cc1ccc(C#N)cc1)Nc1nc(NCc2nc3cc(F)ccc3[nH]2)nc(Nc2cccc(CN3CCN(C=O)CC3)c2)n1] |
| TA1017 | 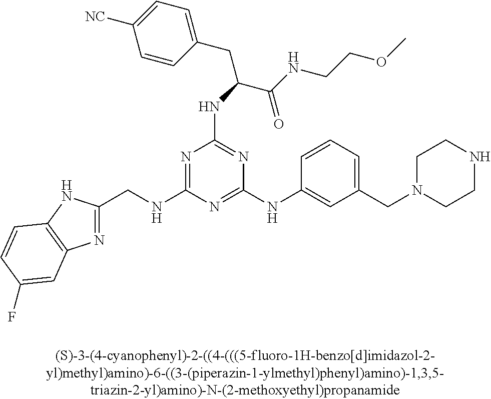 [SMILES: COCCNC(=O)[C@H](Cc1ccc(C#N)cc1)Nc1nc(NCc2nc3cc(F)ccc3[nH]2)nc(Nc2cccc(CN3CCNCC3)c2)n1] |
| TA1018 | 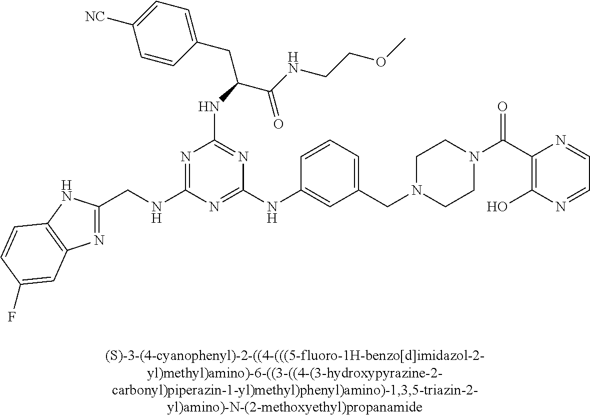 [SMILES: COCCNC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| TA1019 | 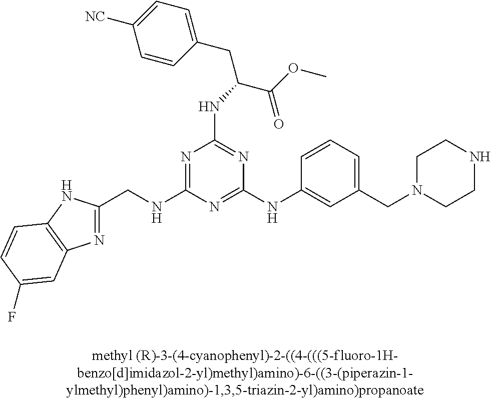 [SMILES: COC(=O)[C@@H](Cc1ccc(C#N)cc1)Nc1nc(NCc2nc3cc(F)ccc3[nH]2)nc(Nc2cccc(CN3CCNCC3)c2)n1] |
| TA1020 | 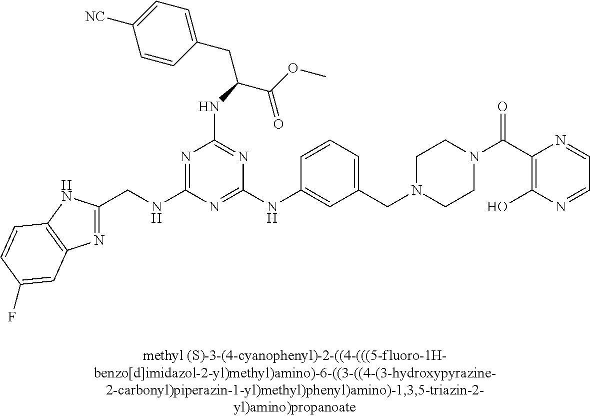 [SMILES: COC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| TA1021 | 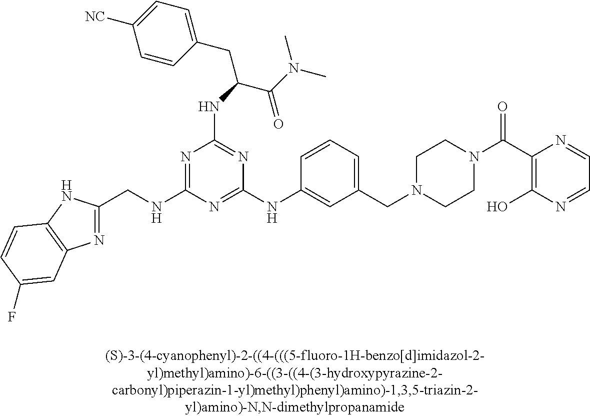 [SMILES: CN(C)C(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| TA1022 | 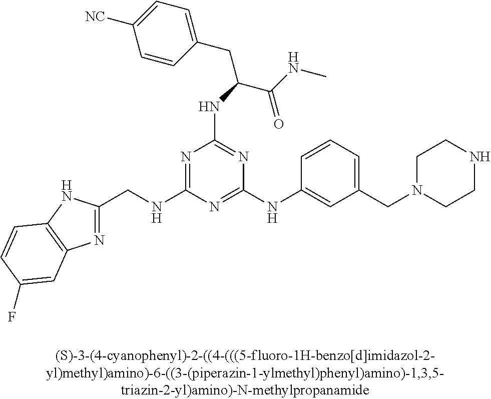 [SMILES: CNC(=O)[C@H](Cc1ccc(C#N)cc1)Nc1nc(NCc2nc3cc(F)ccc3[nH]2)nc(Nc2cccc(CN3CCNCC3)c2)n1] |
| TA1023 | 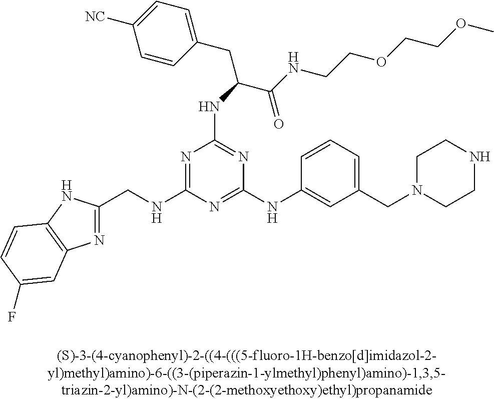 [SMILES: COCCOCCNC(=O)[C@H](Cc1ccc(C#N)cc1)Nc1nc(NCc2nc3cc(F)ccc3[nH]2)nc(Nc2cccc(CN3CCNCC3)c2)n1] |
| TA1024 | 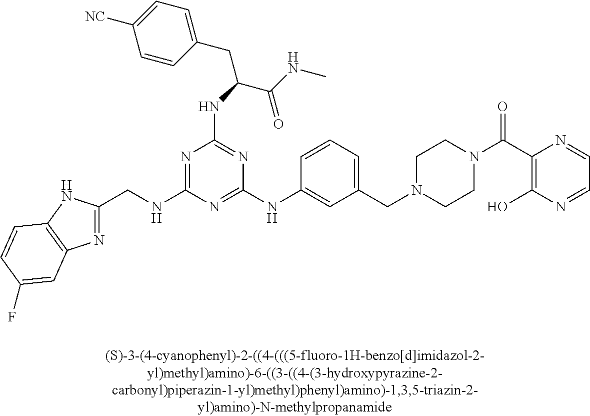 [SMILES: CNC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| TA1025 | 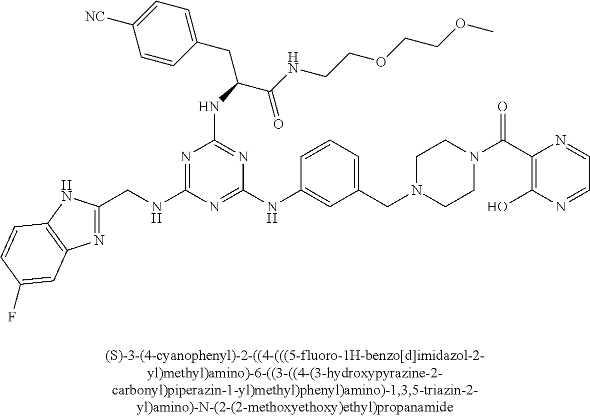 [SMILES: COCCOCCNC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| TA1026 | 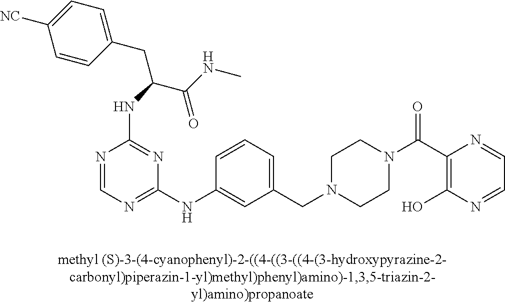 [SMILES: CNC(=O)[C@H](Cc1ccc(C#N)cc1)Nc1ncnc(Nc2cccc(CN3CCN(C(=O)c4nccnc4O)CC3)c2)n1] |
| TA1027 | 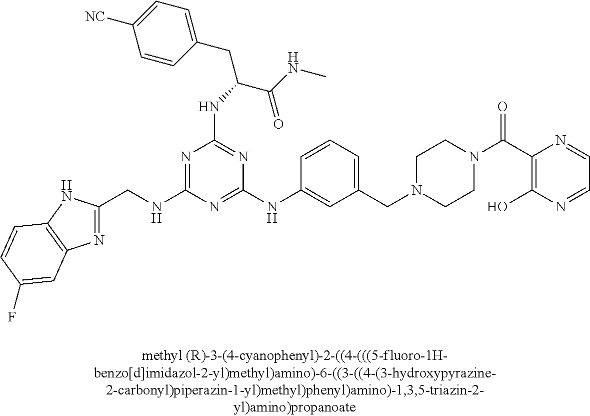 [SMILES: CNC(=O)[C@@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| TA1028 | 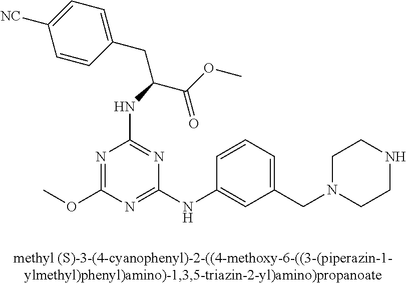 [SMILES: COC(=O)[C@H](Cc1ccc(C#N)cc1)Nc1nc(Nc2cccc(CN3CCNCC3)c2)nc(OC)n1] |
| TA1029 | 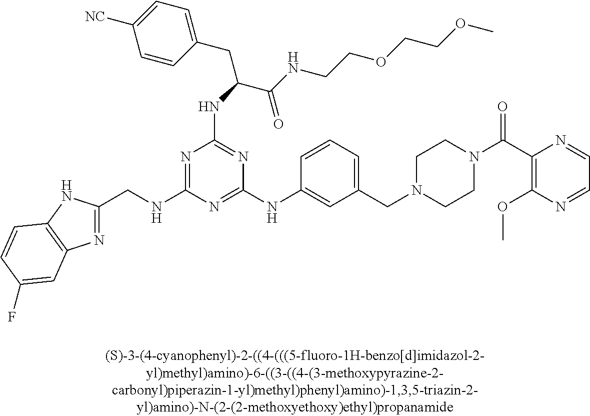 [SMILES: COCCOCCNC(=O)[C@H](Cc1ccc(C#N)cc1)Nc1nc(NCc2nc3cc(F)ccc3[nH]2)nc(Nc2cccc(CN3CCN(C(=O)c4nccnc4OC)CC3)c2)n1] |
| TA1030 | 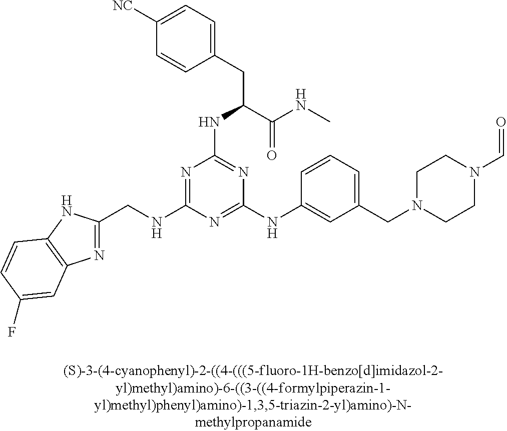 [SMILES: CNC(=O)[C@H](Cc1ccc(C#N)cc1)Nc1nc(NCc2nc3cc(F)ccc3[nH]2)nc(Nc2cccc(CN3CCN(C=O)CC3)c2)n1] |
| TA1031 | 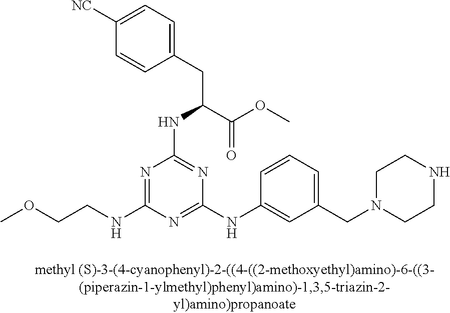 [SMILES: COCCNc1nc(Nc2cccc(CN3CCNCC3)c2)nc(N[C@@H](Cc2ccc(C#N)cc2)C(=O)OC)n1] |
| TA1032 | 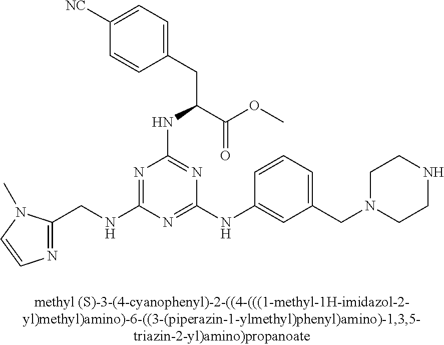 [SMILES: COC(=O)[C@H](Cc1ccc(C#N)cc1)Nc1nc(NCc2nccn2C)nc(Nc2cccc(CN3CCNCC3)c2)n1] |
| TA1033 | 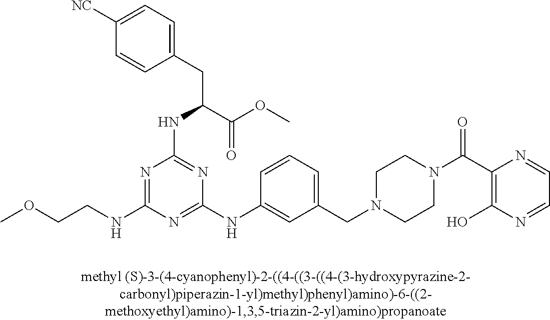 [SMILES: COCCNc1nc(Nc2cccc(CN3CCN(C(=O)c4nccnc4O)CC3)c2)nc(N[C@@H](Cc2ccc(C#N)cc2)C(=O)OC)n1] |
| TA1034 | 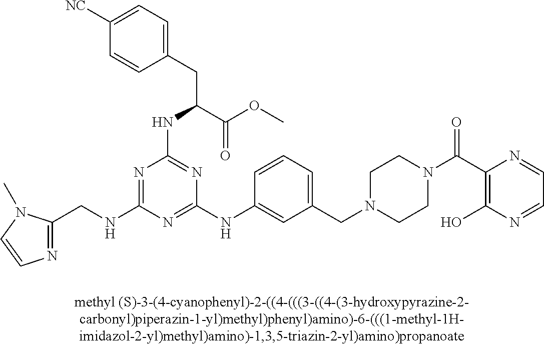 [SMILES: COC(=O)[C@H](Cc1ccc(C#N)cc1)Nc1nc(NCc2nccn2C)nc(Nc2cccc(CN3CCN(C(=O)c4nccnc4O)CC3)c2)n1] |
| TA1035 | 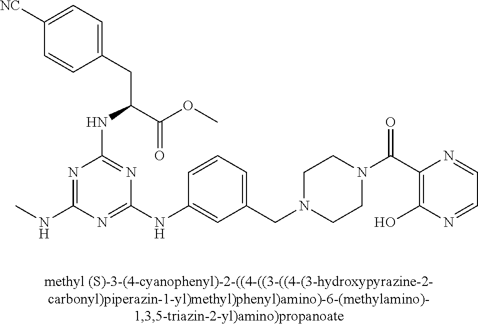 [SMILES: CNc1nc(Nc2cccc(CN3CCN(C(=O)c4nccnc4O)CC3)c2)nc(N[C@@H](Cc2ccc(C#N)cc2)C(=O)OC)n1] |
| TA1036 | 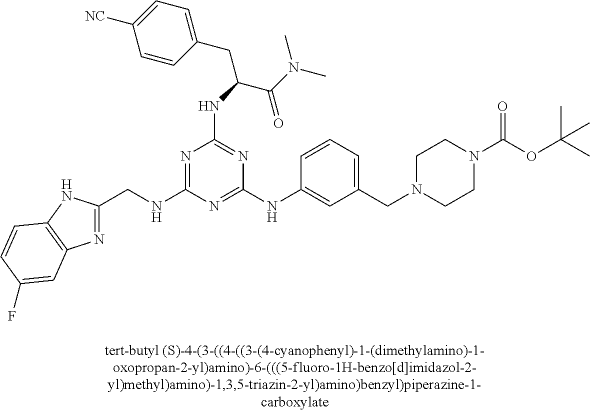 [SMILES: CN(C)C(=O)[C@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1] |
| TA1037 | 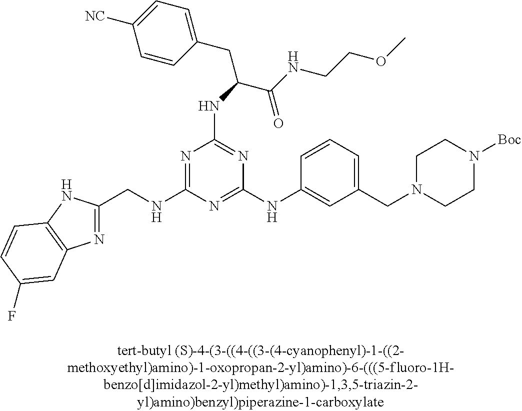 [SMILES: COCCNC(=O)[C@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1] |
| TA1038 | 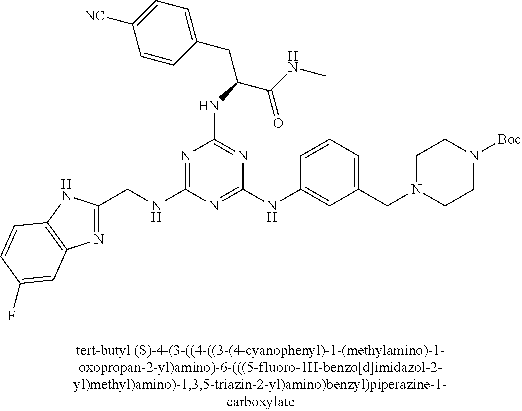 [SMILES: CNC(=O)[C@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1] |
| TA1039 | 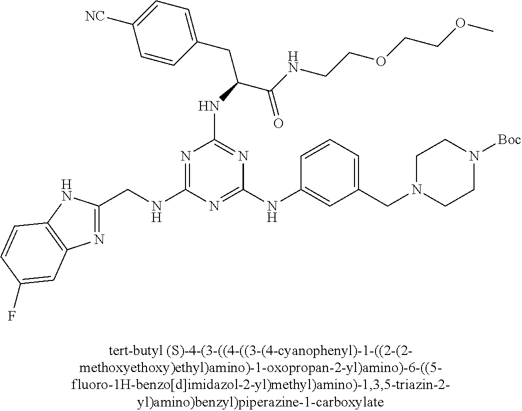 [SMILES: COCCOCCNC(=O)[C@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1] |
| TA1040 | 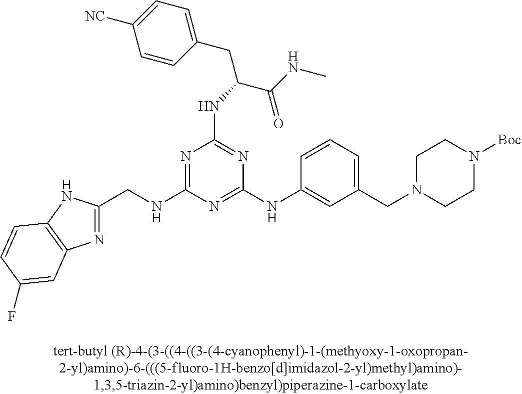 [SMILES: CNC(=O)[C@@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1] |
| TA1041 | 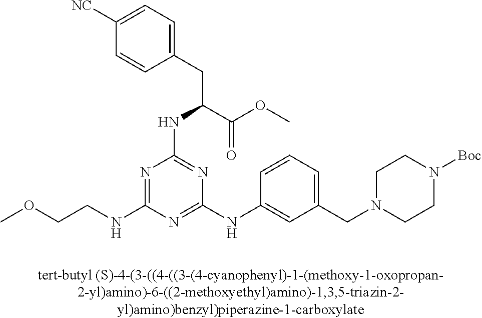 [SMILES: COCCNc1nc(Nc2cccc(CN3CCN(C(=O)OC(C)(C)C)CC3)c2)nc(N[C@@H](Cc2ccc(C#N)cc2)C(=O)OC)n1] |
| TA1042 | 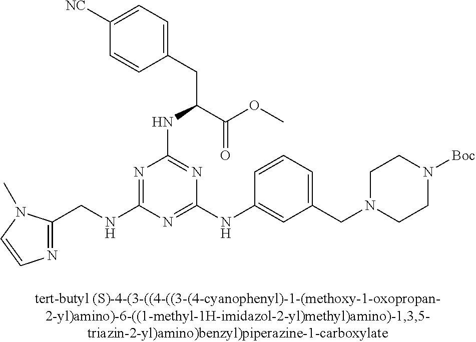 [SMILES: COC(=O)[C@H](Cc1ccc(C#N)cc1)Nc1nc(NCc2nccn2C)nc(Nc2cccc(CN3CCN(C(=O)OC(C)(C)C)CC3)c2)n1] |
| TA1043 | 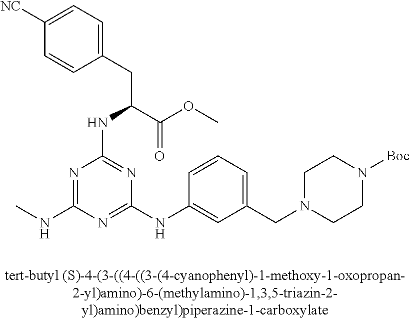 [SMILES: CNc1nc(Nc2cccc(CN3CCN(C(=O)OC(C)(C)C)CC3)c2)nc(N[C@@H](Cc2ccc(C#N)cc2)C(=O)OC)n1] |
| TA1044 | 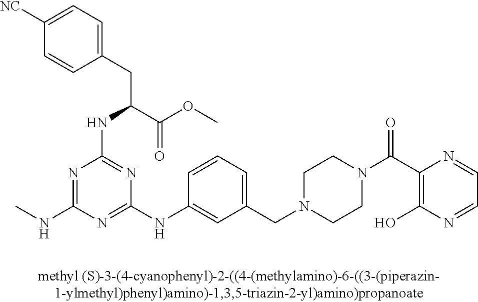 [SMILES: CNc1nc(Nc2cccc(CN3CCN(C(=O)c4nccnc4O)CC3)c2)nc(N[C@@H](Cc2ccc(C#N)cc2)C(=O)OC)n1] |
| TA1045 | 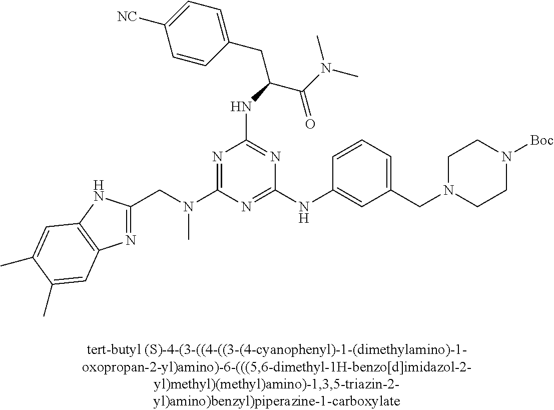 [SMILES: Cc1cc2nc(CN(C)c3nc(Nc4cccc(CN5CCN(C(=O)OC(C)(C)C)CC5)c4)nc(N[C@@H](Cc4ccc(C#N)cc4)C(=O)N(C)C)n3)[nH]c2cc1C] |
| TA1046 | 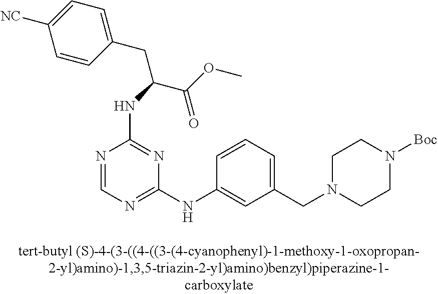 [SMILES: COC(=O)[C@H](Cc1ccc(C#N)cc1)Nc1ncnc(Nc2cccc(CN3CCN(C(=O)OC(C)(C)C)CC3)c2)n1] |
| TA1047 | 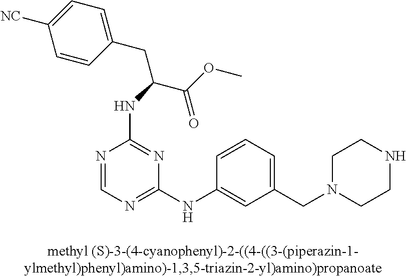 [SMILES: COC(=O)[C@H](Cc1ccc(C#N)cc1)Nc1ncnc(Nc2cccc(CN3CCNCC3)c2)n1] |
| TA1048 | 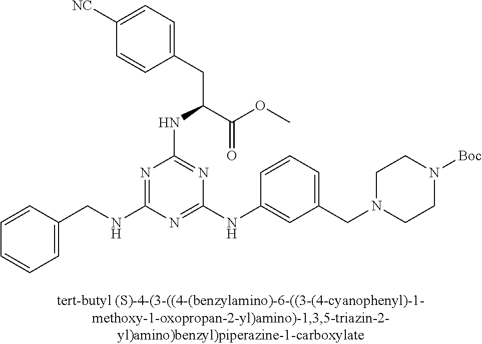 [SMILES: COC(=O)[C@H](Cc1ccc(C#N)cc1)Nc1nc(NCc2ccccc2)nc(Nc2cccc(CN3CCN(C(=O)OC(C)(C)C)CC3)c2)n1] |
| TA1049 | 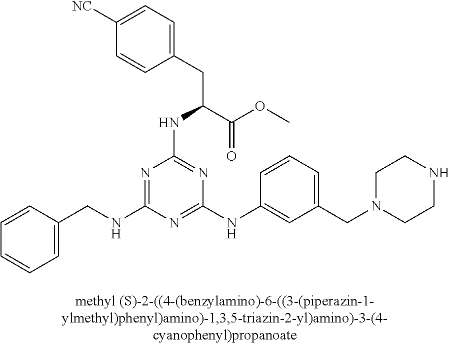 [SMILES: COC(=O)[C@H](Cc1ccc(C#N)cc1)Nc1nc(NCc2ccccc2)nc(Nc2cccc(CN3CCNCC3)c2)n1] |
| TA1050 | 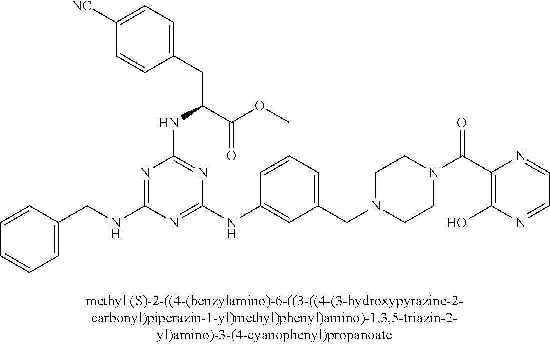 [SMILES: COC(=O)[C@H](Cc1ccc(C#N)cc1)Nc1nc(NCc2ccccc2)nc(Nc2cccc(CN3CCN(C(=O)c4nccnc4O)CC3)c2)n1] |
| TA1051 | 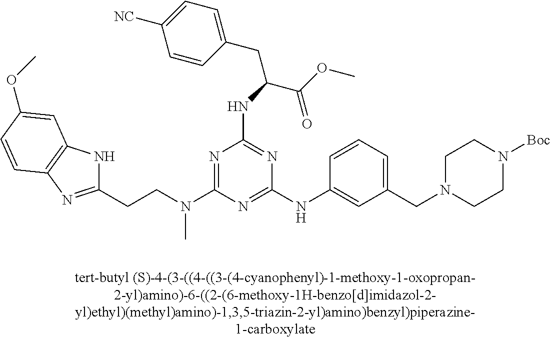 [SMILES: COC(=O)[C@H](Cc1ccc(C#N)cc1)Nc1nc(Nc2cccc(CN3CCN(C(=O)OC(C)(C)C)CC3)c2)nc(N(C)CCc2nc3ccc(OC)cc3[nH]2)n1] |
| TA1052 | 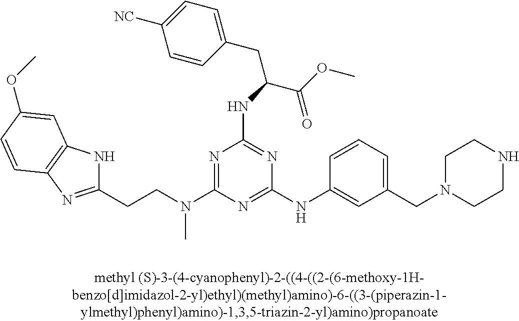 [SMILES: COC(=O)[C@H](Cc1ccc(C#N)cc1)Nc1nc(Nc2cccc(CN3CCNCC3)c2)nc(N(C)CCc2nc3ccc(OC)cc3[nH]2)n1] |
| TA1053 | 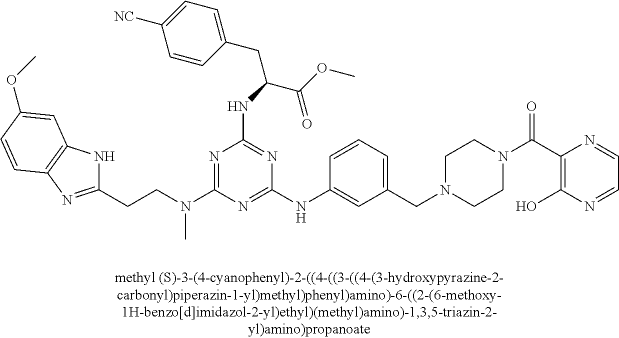 [SMILES: COC(=O)[C@H](Cc1ccc(C#N)cc1)Nc1nc(Nc2cccc(CN3CCN(C(=O)c4nccnc4O)CC3)c2)nc(N(C)CCc2nc3ccc(OC)cc3[nH]2)n1] |
| TA1054 | 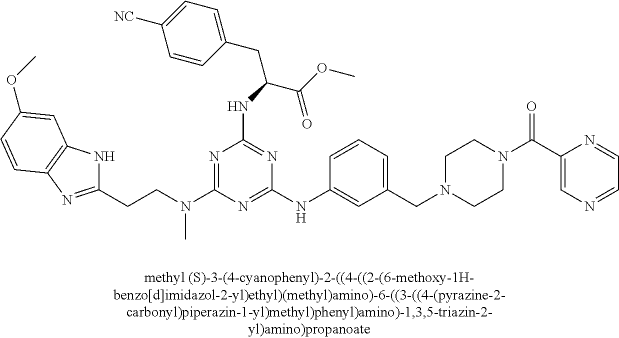 [SMILES: COC(=O)[C@H](Cc1ccc(C#N)cc1)Nc1nc(Nc2cccc(CN3CCN(C(=O)c4cnccn4)CC3)c2)nc(N(C)CCc2nc3ccc(OC)cc3[nH]2)n1] |
| TA1055 | 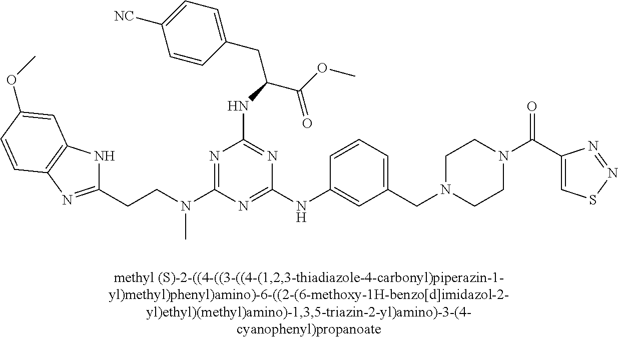 [SMILES: COC(=O)[C@H](Cc1ccc(C#N)cc1)Nc1nc(Nc2cccc(CN3CCN(C(=O)c4csnn4)CC3)c2)nc(N(C)CCc2nc3ccc(OC)cc3[nH]2)n1] |
| TA1056 | 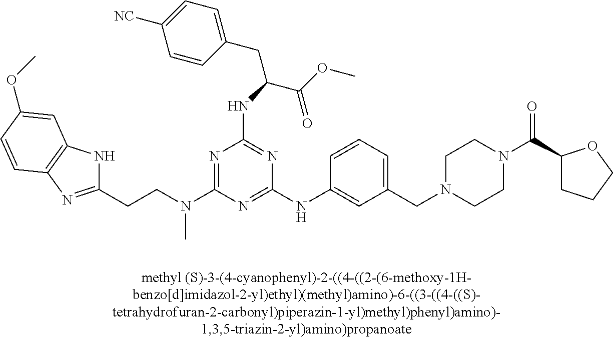 [SMILES: COC(=O)[C@H](Cc1ccc(C#N)cc1)Nc1nc(Nc2cccc(CN3CCN(C(=O)[C@@H]4CCCO4)CC3)c2)nc(N(C)CCc2nc3ccc(OC)cc3[nH]2)n1] |
| TA1057 | 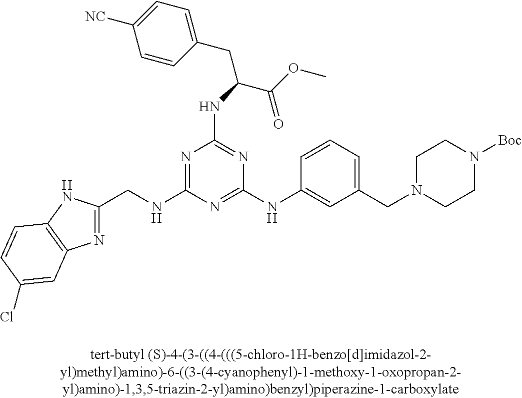 [SMILES: COC(=O)[C@H](Cc1ccc(C#N)cc1)Nc1nc(NCc2nc3cc(Cl)ccc3[nH]2)nc(Nc2cccc(CN3CCN(C(=O)OC(C)(C)C)CC3)c2)n1] |
| TA1058 | 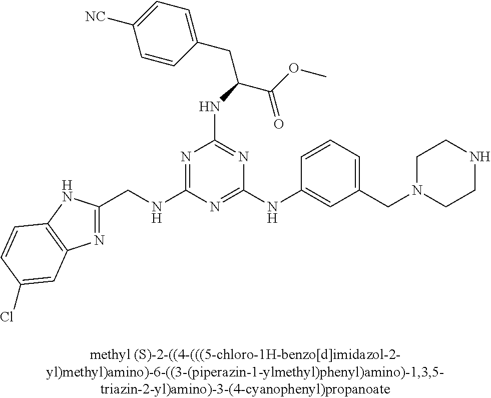 [SMILES: COC(=O)[C@H](Cc1ccc(C#N)cc1)Nc1nc(NCc2nc3cc(Cl)ccc3[nH]2)nc(Nc2cccc(CN3CCNCC3)c2)n1] |
| TA1059 | 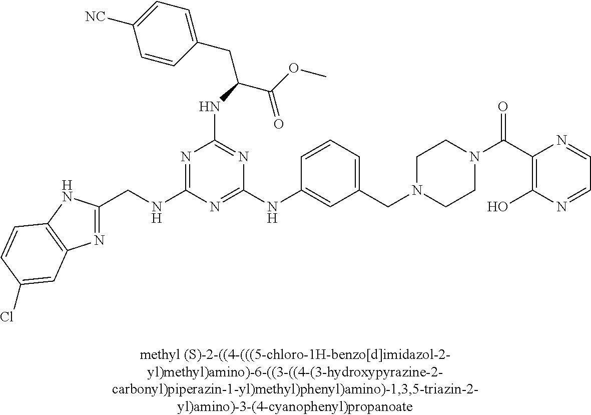 [SMILES: COC(=O)[C@H](Cc1ccc(C#N)cc1)Nc1nc(NCc2nc3cc(Cl)ccc3[nH]2)nc(Nc2cccc(CN3CCN(C(=O)c4nccnc4O)CC3)c2)n1] |
| TA1060 | 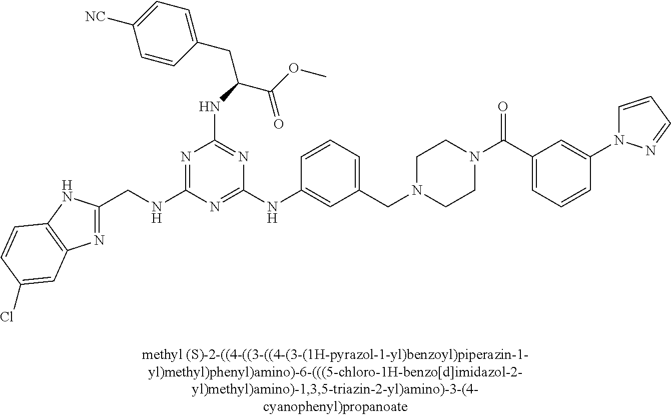 [SMILES: COC(=O)[C@H](Cc1ccc(C#N)cc1)Nc1nc(NCc2nc3cc(Cl)ccc3[nH]2)nc(Nc2cccc(CN3CCN(C(=O)c4cccc(-n5cccn5)c4)CC3)c2)n1] |
| TA1061 | 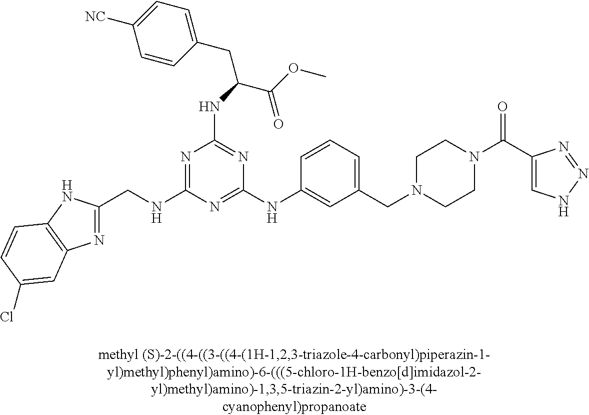 [SMILES: COC(=O)[C@H](Cc1ccc(C#N)cc1)Nc1nc(NCc2nc3cc(Cl)ccc3[nH]2)nc(Nc2cccc(CN3CCN(C(=O)c4c[nH]nn4)CC3)c2)n1] |
| TA1062 | 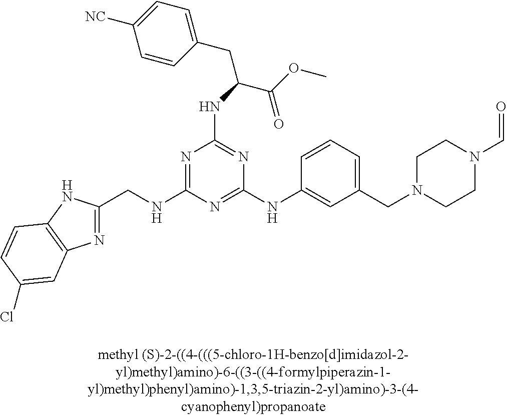 [SMILES: COC(=O)[C@H](Cc1ccc(C#N)cc1)Nc1nc(NCc2nc3cc(Cl)ccc3[nH]2)nc(Nc2cccc(CN3CCN(C=O)CC3)c2)n1] |
| TA1063 | 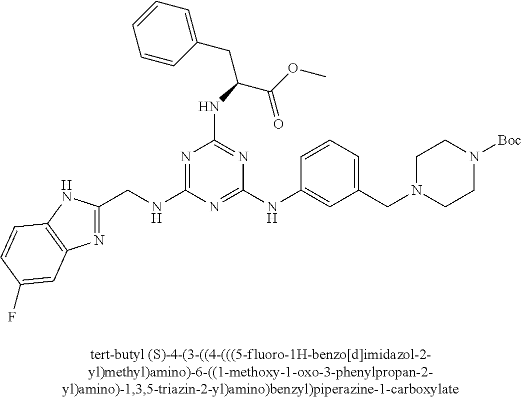 [SMILES: COC(=O)[C@H](Cc1ccccc1)Nc1nc(NCc2nc3cc(F)ccc3[nH]2)nc(Nc2cccc(CN3CCN(C(=O)OC(C)(C)C)CC3)c2)n1] |
| TA1064 | 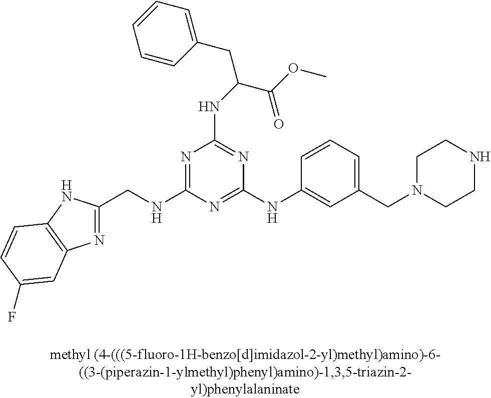 [SMILES: COC(=O)C(Cc1ccccc1)Nc1nc(NCc2nc3cc(F)ccc3[nH]2)nc(Nc2cccc(CN3CCNCC3)c2)n1] |
| TA1065 | 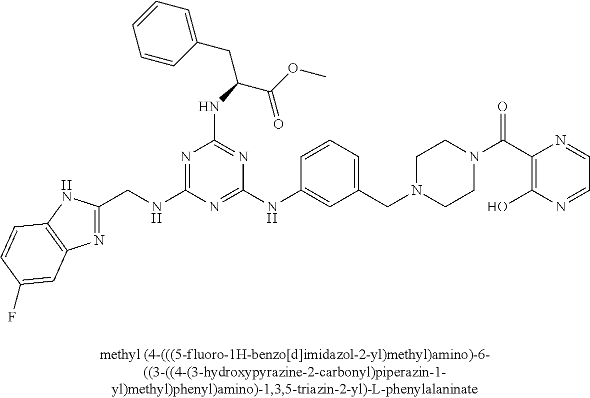 [SMILES: COC(=O)[C@H](Cc1ccccc1)Nc1nc(NCc2nc3cc(F)ccc3[nH]2)nc(Nc2cccc(CN3CCN(C(=O)c4nccnc4O)CC3)c2)n1] |
| TA1066 | 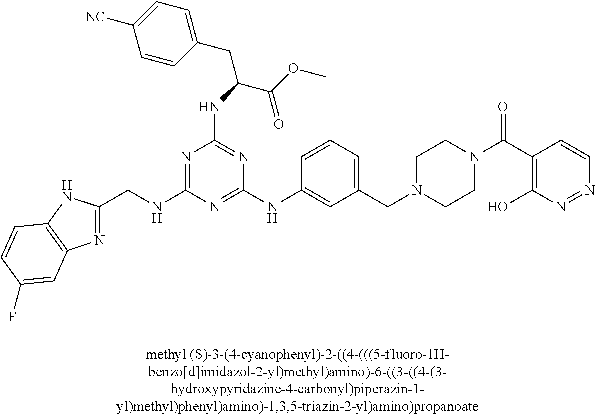 [SMILES: COC(=O)[C@H](Cc1ccc(C#N)cc1)Nc1nc(NCc2nc3cc(F)ccc3[nH]2)nc(Nc2cccc(CN3CCN(C(=O)c4ccnnc4O)CC3)c2)n1] |
| TA1067 | 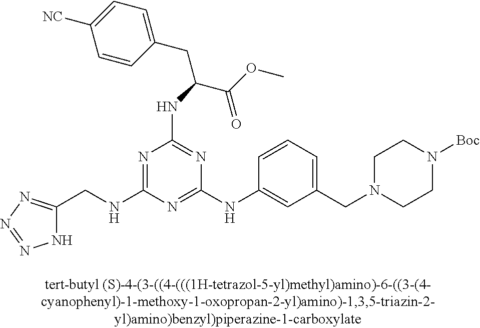 [SMILES: COC(=O)[C@H](Cc1ccc(C#N)cc1)Nc1nc(NCc2nnn[nH]2)nc(Nc2cccc(CN3CCN(C(=O)OC(C)(C)C)CC3)c2)n1] |
| TA1068 | 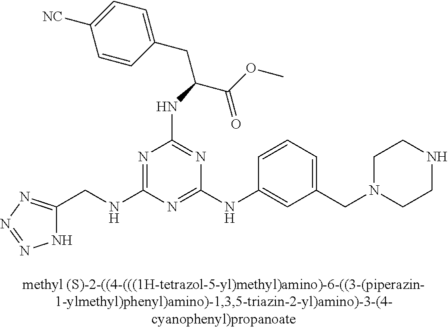 [SMILES: COC(=O)[C@H](Cc1ccc(C#N)cc1)Nc1nc(NCc2nnn[nH]2)nc(Nc2cccc(CN3CCNCC3)c2)n1] |
| TA1069 | 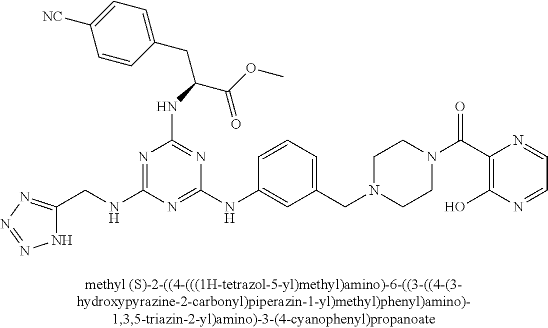 [SMILES: COC(=O)[C@H](Cc1ccc(C#N)cc1)Nc1nc(NCc2nnn[nH]2)nc(Nc2cccc(CN3CCN(C(=O)c4nccnc4O)CC3)c2)n1] |
| TA1070 | 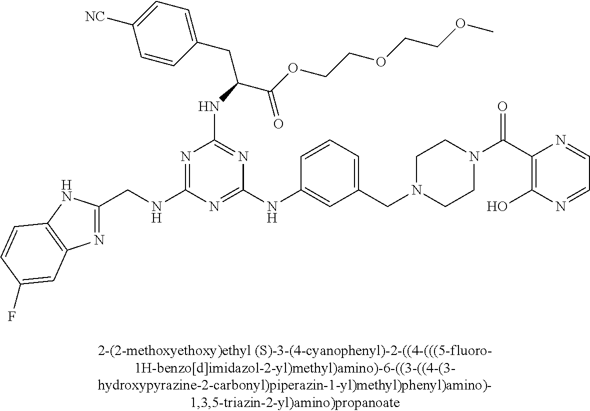 [SMILES: COCCOCCOC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| TA1071 | 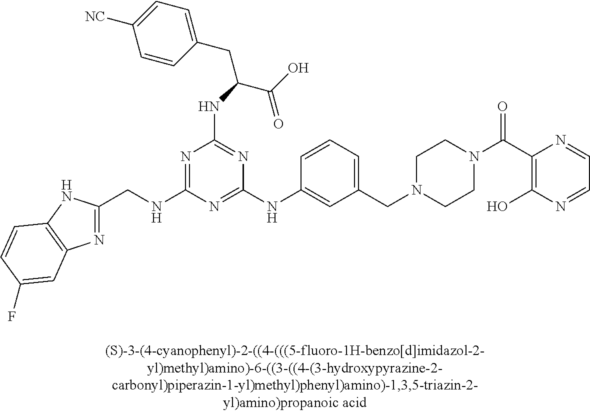 [SMILES: N#Cc1ccc(C[C@H](Nc2nc(NCc3nc4cc(F)ccc4[nH]3)nc(Nc3cccc(CN4CCN(C(=O)c5nccnc5O)CC4)c3)n2)C(=O)O)cc1] |
| TA1072 | 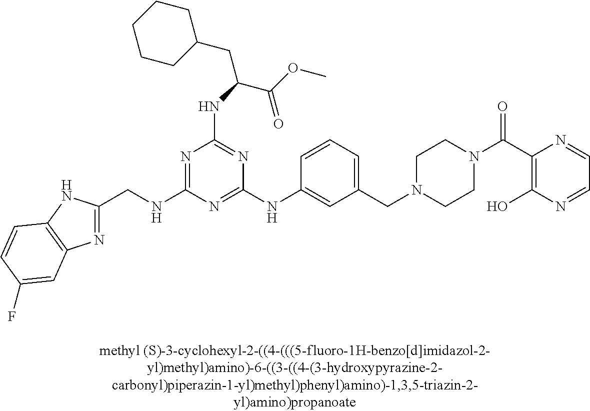 [SMILES: COC(=O)[C@H](CC1CCCCC1)Nc1nc(NCc2nc3cc(F)ccc3[nH]2)nc(Nc2cccc(CN3CCN(C(=O)c4nccnc4O)CC3)c2)n1] |
| TA1073 | 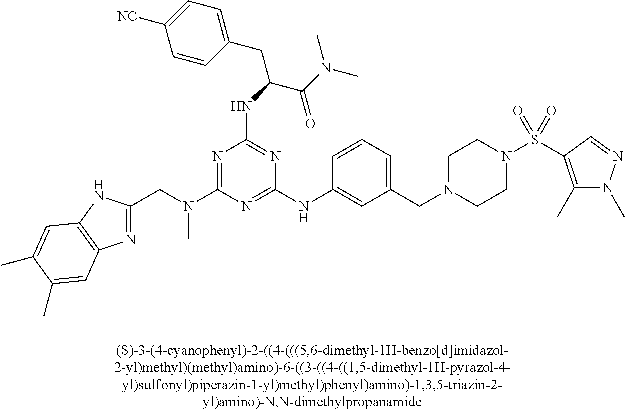 |
| TA1074 | 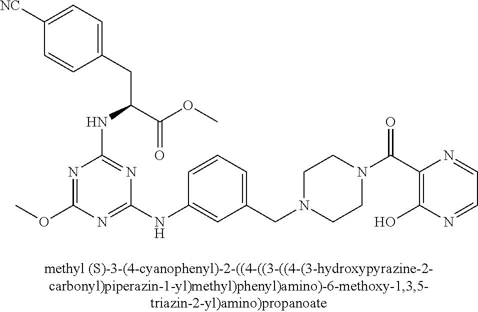 [SMILES: COC(=O)[C@H](Cc1ccc(C#N)cc1)Nc1nc(Nc2cccc(CN3CCN(C(=O)c4nccnc4O)CC3)c2)nc(OC)n1] |
| TA1075 | 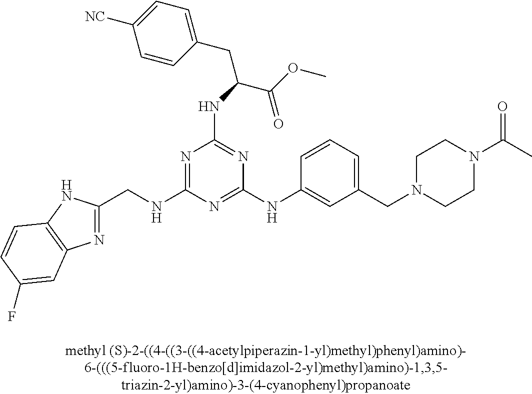 [SMILES: COC(=O)[C@H](Cc1ccc(C#N)cc1)Nc1nc(NCc2nc3cc(F)ccc3[nH]2)nc(Nc2cccc(CN3CCN(C(C)=O)CC3)c2)n1] |
| TA1076 | 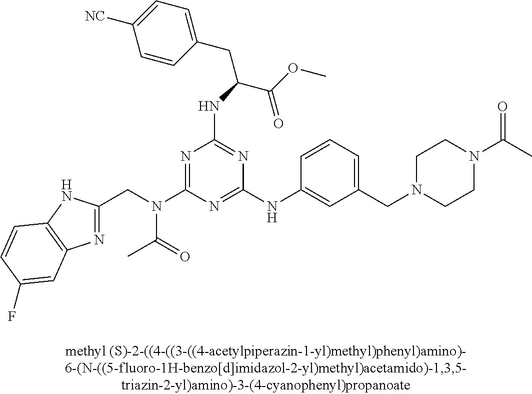 [SMILES: COC(=O)[C@H](Cc1ccc(C#N)cc1)Nc1nc(Nc2cccc(CN3CCN(C(C)=O)CC3)c2)nc(N(Cc2nc3cc(F)ccc3[nH]2)C(C)=O)n1] |
| TA1077 | 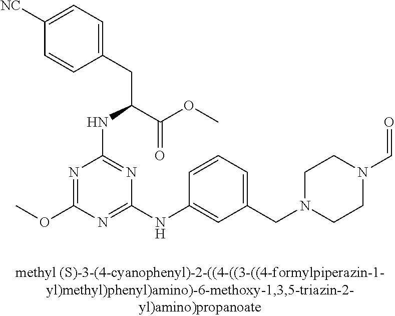 [SMILES: COC(=O)[C@H](Cc1ccc(C#N)cc1)Nc1nc(Nc2cccc(CN3CCN(C=O)CC3)c2)nc(OC)n1] |
| TA1078 | 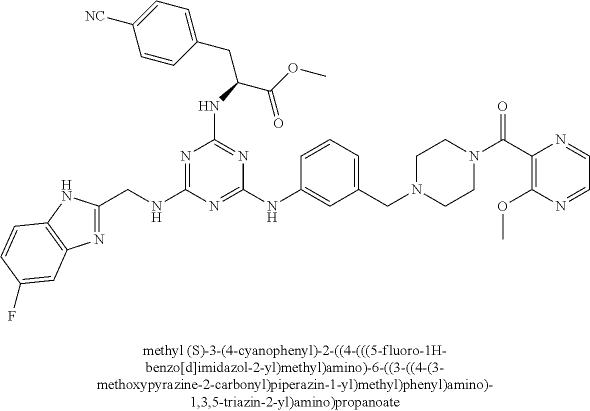 [SMILES: COC(=O)[C@H](Cc1ccc(C#N)cc1)Nc1nc(NCc2nc3cc(F)ccc3[nH]2)nc(Nc2cccc(CN3CCN(C(=O)c4nccnc4OC)CC3)c2)n1] |
| TA1079 | 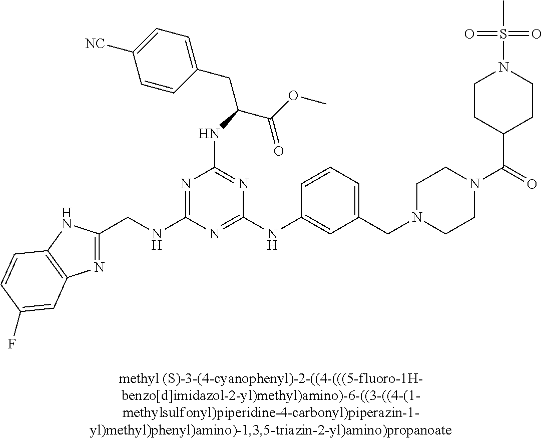 [SMILES: COC(=O)[C@H](Cc1ccc(C#N)cc1)Nc1nc(NCc2nc3cc(F)ccc3[nH]2)nc(Nc2cccc(CN3CCN(C(=O)C4CCN(S(C)(=O)=O)CC4)CC3)c2)n1] |

As used herein, “alkyl”, “C1, C2, C3, C4, C5 or C6 alkyl” or “C₁-C₆ alkyl” or “C₁₋₆alkyl” is intended to include C1, C2, C3, C4, C5 or C6 straight chain (linear) saturated aliphatic hydrocarbon groups and C3, C4, C5 or C6 branched saturated aliphatic hydrocarbon groups. For example, C₁-C₆ alkyl is intended to include C1, C2, C3, C4, C5 and C6 alkyl groups. Examples of alkyl include, moieties having from one to six carbon atoms, such as, but not limited to, methyl, ethyl, n-propyl, i-propyl, n-butyl, s-butyl, t-butyl, n-pentyl, s-pentyl or n-hexyl. The term Cₘ₋ₙ means the alkyl group has “m” to “n” carbon atoms. The term “alkylene” refers to an alkyl group having a substituent. In some embodiments, for example, C₀₋₃alkylene within a substituent represents a 0, 1, 2 or 3 carbon linker, preferably linear, and optionally substituted where indicated.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

The term “3- to 14-membered ring” refers to a cycloalkyl, heterocycloalkyl, aryl, or heteroaryl group having 3 to 14 atoms. The 3-to 14-membered ring can have one or more heteroatoms (such as O, N, S, or Se). For example, the 3- to 14-membered ring can have 1-4 heteroatoms, 1-3 heteroatoms, or 1-2 heteroatoms. Examples of 3- to 14-membered rings include, but are not limited to, C₃-C₈ cycloalkyl, 3- to 10-membered heterocycloalkyl, C₆-C₁₀ aryl or 5- to 10-membered heteroaryl.

As used herein, the term “cycloalkyl” refers to a saturated or unsaturated nonaromatic hydrocarbon mono- or multi-ring (e.g., fused, bridged, or spiro rings) system having 3 to 30 carbon atoms (e.g., C₃-C₁₀). For example, a C₃-C₈ cycloalkyl is intended to include a monocyclic, bicyclic or tricyclic ring having 3, 4, 5, 6, 7, or 8 carbon atoms. Examples of cycloalkyl rings include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of cycloalkyl, including, for example, [3.3.0]bicyclooctane, [4.3.0]bicyclononane, [4.4.0]bicyclodecane and [2.2.2]bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included. In the case of multicyclic rings, none of the rings is aromatic.

The term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, or Se), unless specified otherwise. For example, a 3 to 12-membered heterocycloalkyl ring is intended to include a monocyclic, bicyclic, or tricyclic ring having 3, 4, 5, 6, 7, 8, 9, 10, 11, or 12 atoms selected from C, O, N, S, and Se. In the case of multicyclic rings, none of the rings is aromatic. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, azocinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furazanyl, imidazolidinyl, imidazolinyl, 1H-indazolyl, indolenyl, isatinoyl, isobenzofuranyl, isochromanyl, isoindazolyl, isoindolinyl, methylenedioxyphenyl, morpholinyl, naphthyridinyl, octahydroisoquinolinyl, oxazolidinyl, oxindolyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pyranyl, pyrazolidinyl, pyrazolinyl, pyrrolidinyl, pyrrolinyl, 2H-pyrrolyl, quinuclidinyl, tetrazolyl, 6H-1,2,5-thiadiazinyl, 1,2,3-thiadiazolyl, thianthrenyl, thienyl, thienothiazolyl, thienooxazolyl, thienoimidazolyl and xanthenyl and the like.

Substituted alkyl is alkyl in which the designated substituents replace one or more hydrogen atoms on one or more carbons of the hydrocarbon backbone. Such substituents can include, for example, alkyl, alkenyl, alkynyl, halogen, hydroxyl, oxo, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups.

In certain embodiments, a straight chain or branched alkenyl group has six or fewer carbon atoms in its backbone (e.g., C2-C6 for straight chain, C3-C6 for branched chain). The term “C2-C6” includes alkenyl groups containing two to six carbon atoms. The term or “C3-C6” includes alkenyl groups containing three to six carbon atoms.

Substituted alkenyl is alkenyl in which the designated substituents replace one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyl groups described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C2-C6 for straight chain, C₃-C₆ for branched chain). The term “C2-C6” includes alkynyl groups containing two to six carbon atoms. The term “C3-C6” includes alkynyl groups containing three to six carbon atoms.

Substituted alkynyl is alkynyl in which the designated substituents replace one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

“Aryl” includes groups with aromaticity, including “conjugated,” or multicyclic systems with at least one aromatic ring and do not contain any heteroatom in the ring structure. For example, a C6-C10aryl is intended to include a monocyclic, bicyclic or tricyclic ring having 6, 7, 8, 9, or 10 carbon atoms. Examples include phenyl, 1,2,3,4-tetrahydronaphthalenyl, naphthalene, etc.

“Heteroaryl” groups are aryl groups, as defined above, except having from one to four heteroatoms in the ring structure, and may also be referred to as “aryl heterocycles” or “heteroaromatics.” For example, a 5- to 10-membered heterocycloalkyl ring is intended to include a stable 5-, 6-, 7-, 8-, or 9-membered monocyclic or 5-, 6-, 7-, 8-, 9-, or 10-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen, sulfur, selenium, and boron. The nitrogen atom may be substituted or unsubstituted (i.e., N or NR wherein R is H or other substituents, as defined). The nitrogen and sulfur heteroatoms may optionally be oxidized (i.e., N→0 and S(O)p, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, furanyl, oxazolyl, imidazolyl, indolyl, 3H-indolyl, isoindolyl, isoquinolinyl, isothiazolyl, isoxazolyl, pyrazolyl, pyridazinyl, pyridooxazole, pyridoimidazole, pyridothiazole, pyridinyl, pyridyl, pyrimidinyl, oxadiazolyl, pyrazolopyridyl, benzimidazolyl, benzothiazolyl, benzofuranyl, pteridinyl, purinyl, pyrazinyl, benzothiofuranyl, benztriazolyl, benztetrazolyl, benzisoxazolyl, benzisothiazolyl, benzothiophenyl, benzoxazolyl, azabenzimidazolyl, azabenzoxazolyl, azabenzothiazolyl, 1,2,4-thiadiazolyl, 1,2,5-thiadiazolyl, 1,3,4-thiadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, thiophenyl, triazinyl, 1,2,3-triazolyl, 1,2,4-triazolyl, 1,2,5-triazolyl, 1,3,4-triazolyl, tetrahydroisoquinolinyl, tetrahydroquinolinyl, benzoxazolinyl, benzimidazolinyl, indolinyl, indolizinyl, quinazolinyl, quinolinyl, 4H-quinolizinyl, quinoxalinyl, pyrrolyl, thiazolyl, benzoxazole, benzoxadiazole, benzothiazole, benzoimidazole, benzothiophene, 4,5,6,7-tetrahydrobenzo[d]oxazole, 4,5,6,7-tetrahydro-1H-benzo[d]imidazole, methylenedioxyphenyl, quinoline, isoquinoline, 1,2,3,4-tetrahydroquinoline, 1,2,3,4-tetrahydroisoquinoline, naphthrydine, indole, deazapurine, indolizine, and the like.

Furthermore, the terms “aryl” and “heteroaryl” include multicyclic aryl and heteroaryl groups, e.g., tricyclic, bicyclic, e.g.,

In the case of multicyclic aromatic rings, only one of the rings needs to be aromatic (e.g., 2,3-dihydroindole), although all of the rings may be aromatic (e.g., quinoline). The second ring can also be fused or bridged.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, oxo, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl).

The term “nitrogen protecting group” generally comprises any group that is capable of reversibly protecting a nitrogen functionality, e.g., an amino and/or amide functionality.

For example, the nitrogen protecting group can be an amine protecting group and/or an amide protecting group. Suitable nitrogen protecting groups are described, e.g., in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, and in “Methoden der organischen Ckemie” (Methods of Organic Chemistry), Houben Weyl, 4th edition, Volume 15/1, Georg Thieme Verlag, Stuttgart 1974.

For example, the nitrogen protecting group can be C₁-C₆ alkyl, C₁-C₄ alkyl, C₁-C₂ alkyl, or C₁ alkyl, which is mono-, di- or tri-substituted by trialkylsilyl C₁-C₇-alkoxy (e.g., trimethylsilyethoxy)aryl, e.g., phenyl, or an heterocyclic group, e.g., pyrrolidinyl, wherein the aryl ring or the heterocyclic group is unsubstituted or substituted by one or more, e.g., two or three, residues, e.g., selected from the group consisting of C₁-C₇ alkyl, hydroxy, C₁-C₇ alkoxy, —C(═O)C₂-C₈ alkoxy, halogen, nitro, cyano, and CF₃; aryl-C₁-C₂-alkoxycarbonyl (e.g., phenyl-C₁-C₂-alkoxycarbonyl, e.g., benzyloxycarbonyl); C₁₋₁₀alkenyloxycarbonyl; C₁₋₆alkylcarbonyl (eg. acetyl or pivaloyl); C₆₋₁₀arylcarbonyl; C₁₋₆alkoxycarbonyl (eg. t-butoxycarbonyl); C₆₋₁₀arylC₁₋₆alkoxycarbonyl; allyl or cinnamyl; sulfonyl or sulfenyl; succinimidyl group, silyl, e.g. triarylsilyl or trialkylsilyl (eg. triethylsilyl).

Examples of nitrogen protecting groups include, but are not limited to, acetyl, benzyl, cumyl, benzhydryl, trityl, benzyloxycarbonyl (Cbz), 9-fluorenylmethyloxycarbony (Fmoc), benzyloxymethyl (BOM), pivaloyl-oxy-methyl (POM), trichloroethxoycarbonyl (Troc), 1-adamantyloxycarbonyl (Adoc), allyl, allyloxycarbonyl, trimethylsilyl, tert.-butyl-dimethylsilyl, triethylsilyl (TES), triisopropylsilyl, trimethylsilyethoxymethyl (SEM), t-butoxycarbonyl (BOC), t-butyl, 1-methyl-1,1-dimethylbenzyl, (phenyl)methyl benzene, pyrridinyl and pivaloyl. Most preferred nitrogen protecting groups are acetyl, benzyl, benzyloxycarbonyl (Cbz), triethylsilyl (TES), trimethylsilyethoxymethyl (SEM), t-butoxycarbonyl (BOC), pyrrolidinylmethyl and pivaloyl.

The term “substituted,” as used herein, means that any one or more hydrogen atoms on the designated atom is replaced with a selection from the indicated groups, provided that the designated atom's normal valency is not exceeded, and that the substitution results in a stable compound. When a moiety is indicated as substituted with one or more substituents, this typically indicates substitution with 1, 2, 3, 4, 5, or more, including 1 to 5, 1 to 4, 1 to 3, 1 to 2 or 1 substituents independently selected from an indicated group. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula.

Combinations of substituents and/or variables are permissible, but only if such combinations result in stable compounds.

When any variable (e.g., R^{e}) occurs more than one time in any constituent or formula for a compound, its definition at each occurrence is independent of its definition at every other occurrence. Thus, for example, if a group is shown to be substituted with 0-2 R¹ moieties, then the group may optionally be substituted with up to two R¹ moieties and R¹ at each occurrence is selected independently from the definition of R¹. Also, combinations of substituents and/or variables are permissible, but only if such combinations result in stable compounds.

The term “hydroxy” or “hydroxyl” includes groups with an —OH or —O—.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

The term “carboxyl” refers to —COOH or its C1-C6 alkyl ester.

“Acyl” includes moieties that contain the acyl radical (R—C(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

### Synthesis of Triazine Compounds of the Invention

The present invention provides methods for the synthesis of the compounds of any Formula disclosed herein. The present invention also provides detailed methods for the synthesis of various disclosed compounds of the present invention according to the following schemes as shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the invention can tolerate a wide variety of functional groups, therefore various substituted starting materials can be used. The processes generally provide the desired final compound at or near the end of the overall process, although it may be desirable in certain instances to further convert the compound to a pharmaceutically acceptable salt, ester, or prodrug thereof.

Compounds of the present invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present invention.

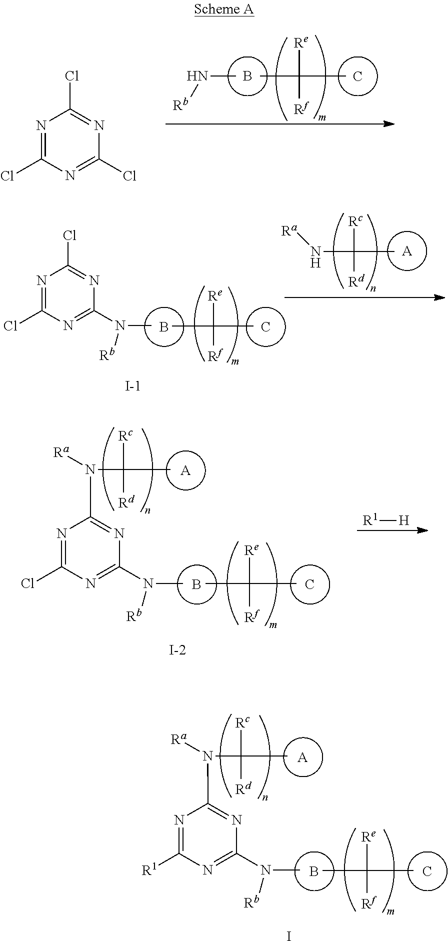

Scheme A shows the synthesis of formula (I), wherein A, B, C, R¹, R^{a}, R^{b}, R^{c}, R^{d}, R^{e}, R^{f}, n, and m are as defined above. Cyanuric chloride can react with an appropriate amine to form dichlorinated intermediate I-1. I-1 can then react with an appropriate amine in the presence of a suitable base, such as K₂CO₃, to form mono-chlorinated intermediate I-2. Finally, I-2 can react with R¹—H in the presence of a suitable base, such as DIPEA, and optionally in the presence of a coupling agent, such as Pd(OAc)₂, to form a compound of formula (I).

### Pharmaceutical Compositions

The present invention also provides pharmaceutical compositions comprising a compound of any Formula disclosed herein in combination with at least one pharmaceutically acceptable excipient or carrier.

A “pharmaceutical composition” is a formulation containing the compounds of the present invention in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the invention can be administered to a subject in many of the well-known methods currently used for chemotherapeutic treatment. The dose chosen should be sufficient to constitute effective treatment but not so high as to cause unacceptable side effects. The state of the disease condition and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives.

Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It can be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the progression of the autoimmune, neurodegenerative, or inflammatory disease. Dosages can be in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. As used herein, the term “dosage effective manner” refers to amount of an active compound to produce the desired biological effect in a subject or cell.

The pharmaceutical compositions can be included in a container, pack, or dispenser together with instructions for administration.

The compounds of the present invention are capable of further forming salts. All of these forms are also contemplated within the scope of the claimed invention.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, bisulfate, bitartric, boric, bromic, butyric, calcium, calcium edetic, camsylate, carbonic, chloric, citric, clavularic, dihydrochloric, edetic, ethane disulfonic, 1,2-ethane sulfonic, estolate, esylate, fumaric, glucoheptonic, gluconic, glutamic, glycolic, glycollyarsanilic, hexafluorophosphoric, hexylresorcinic, hydrabamic, hydrobromic, hydrochloric, hydroiodic, hydroxymaleic, hydroxynaphthoic, iodic, isethionic, lactic, lactobionic, lauryl sulfonic, maleic, malic, mandelic, methane sulfonic, methylbromic, methylnitric, napsylic, nitric, N-methylglucamine ammonium salt, 3-hydroxy-2-naphthoic, oleic, oxalic, pamoic, pantothenic, phenylacetic, phosphoric, polygalacturonic, propionic, salicyclic, stearic, subacetic, succinic, sulfamic, sulfanilic, sulfuric, sulfosalicylic, suramic, tannic, tartaric, toluene sulfonic, tosyl, triethiodic, trifluoroacetic, and valeric and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

The compounds of the present invention can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds of the present invention can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs in the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl)N-Mannich bases, Schiff bases and enaminones of amino functional groups, oximes, acetals, ketals and enol esters of ketone and aldehyde functional groups in compounds of the invention, and the like, See Bundegaard, H., Design of Prodrugs, p 1-92, Elesevier, N.Y.-Oxford (1985).

The compounds, or pharmaceutically acceptable salts, esters or prodrugs thereof, are administered by a route selected from the group consisting of enterally, orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the invention can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

In the synthetic schemes described herein, compounds may be drawn with one particular configuration for simplicity. Such particular configurations are not to be construed as limiting the invention to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity. A

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

### Methods

The phrase “cGAS-mediated condition,” as used herein, comprises autoimmune, inflammatory, and neurodegenerative conditions. For example, the autoimmune disorder is selected from SIRS, sepsis, septic shock, atherosclerosis, celiac disease, interstitial cystitis, transplant rejection, Aicardi-Goutieres Syndrome, chilblain lupus erythematosus, systemic lupus erythematosus, idiopathic thrombocytopenic purpura, thrombotic thrombocytopenic purpura, autoimmune thrombocytopenia, spondyloenchondrodysplasia, psoriasis, Type 1 diabetes, Type 2 diabetes, and Sjogren's syndrome. For example, the inflammatory disorder is selected from rheumatoid arthritis, juvenile rheumatoid arthritis, inflammatory bowel disease (ulcerative colitis, Crohn's disease), age-related macular degeneration, IgA nephropathy, glomerulonephritis, vasculitis, polymyositis, or Wegener's disease. For example, the neurodegenerative disorder is selected from Alzheimer's disease, Parkinson's disease, multiple sclerosis, IgM polyneuropathies, or myasthenia gravis.

As used herein, “treating” or “treat” describes the management and care of a patient for the purpose of combating a disease, condition, or disorder and includes the administration of a compound of the present invention, or a pharmaceutically acceptable salt thereof, to alleviate the symptoms or complications of a disease, condition or disorder, or to eliminate the disease, condition or disorder. The term “treat” can also include treatment of a cell in vitro or an animal model.

A compound of the present invention, or a pharmaceutically acceptable salt thereof, can also be used to prevent a disease, condition or disorder, or used to identify suitable candidates for such purposes. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as an autoimmune, inflammatory, or neurodegenerative disease, which can occur in multiple locations, is alleviated if the severity of the disease is decreased within at least one of multiple locations.

Compounds of the present invention inhibit cGAS and, accordingly, in one aspect of the invention, certain compounds disclosed herein are candidates for treating, or preventing certain conditions and diseases. The present invention provides methods for treating conditions and diseases wherein the course of the condition or disease can be influenced by the STING pathway. The method includes administering to a subject in need of such treatment, a therapeutically effective amount of a compound of the present invention, or a pharmaceutically acceptable salt, metabolite, solvate, or stereoisomer thereof.

The present invention provides a method of inhibiting cGAS in a cell, comprising contacting the cell with one or more compounds or compositions of the present invention.

The present invention also provides a method of treating a cGAS-mediated condition, comprising administering to a patient in need thereof an effective amount of one or more compounds or compositions of the present invention. In some embodiments, the cGAS-mediated condition is an autoimmune, inflammatory, or neurodegenerative condition or cancer (see Rayburn, E. R. et al., Mol Cell Pharmacol. 2009; 1(1): 29-43 and Urbanska, A. M. et al., Cell BiochemBiophys. 2015 July; 72(3):757-69).

The present invention also provides a method of inhibiting type I interferon production mediated by the cGAS-STING pathway comprising: administering to the subject a therapeutically effective amount of one or more compounds or compositions of the present invention. The cGAS-STING pathway of cytosolic DNA sensing as that phrase is used herein comprises the following proteins: SAMHD1, DNase II, STAT1, STAT2, TREX1, ENPP1, cGAS, STING, IRF3, TBK1, IKK, and NF-κB. Such a method may be practiced in vitro, in a cell, or in an organism (e.g., in a human).

The present invention provides a method of treating an autoimmune disease in a subject, comprising administering to the subject a therapeutically effective amount of one or more compounds or compositions of the present invention. In some embodiments, the autoimmune disease can be a type I interferonopathy (e.g., Aicardi-Goutieres Syndrome, Sjogren's syndrome, Singleton-Merten Syndrome, proteasome-associated autoinflammatory syndrome, SAVI (STING-associated vasculopathy with onset in infancy), CANDLE syndrome, chilblain lupus erythematosus, systemic lupus erythematosus, spondyloenchondrodysplasia), rheumatoid arthritis, juvenile rheumatoid arthritis, idiopathic thrombocytopenic purpura, autoimmune myocarditis, thrombotic thrombocytopenic purpura, autoimmune thrombocytopenia, psoriasis, Type 1 diabetes, or Type 2 diabetes.

The present invention provides a method of treating an inflammatory disease in a subject, comprising administering to the subject a therapeutically effective amount of one or more compounds or compositions of the present invention. For example, the inflammatory disease can be atherosclerosis, dermatomyositis, SIRS, sepsis, septic shock, atherosclerosis, celiac disease, interstitial cystitis, transplant rejection, inflammatory bowel disease (ulcerative colitis, Crohn's disease), age-related macular degeneration, IgA nephropathy, glomerulonephritis, vasculitis, polymyositis, or Wegener's disease.

The present invention further provides a method of treating neurodegenerative diseases in a subject, comprising administering to the subject a therapeutically effective amount of one or more compounds or compositions of the present invention. For example, the neurodegenerative disease can be Alzheimer's disease, Parkinson's disease, multiple sclerosis, IgM polyneuropathies, or myasthenia gravis.

The present invention further provides the use of one or more compounds or compositions of the present invention for inhibiting cGAS in a cell.

The present invention further provides the use of one or more compounds or compositions of the present invention for the treatment of a cGAS-mediated condition.

The present invention further provides the use of one or more compounds or compositions of the present invention for the treatment of an autoimmune disease. In some embodiments, the autoimmune disease can be Aicardi-Goutieres Syndrome, chilblain lupus erythematosus, systemic lupus erythematosus, idiopathic thrombocytopenic purpura, thrombotic thrombocytopenic purpura, autoimmune thrombocytopenia, spondyloenchondrodysplasia, psoriasis, Type 1 diabetes, Type 2 diabetes, or Sjogren's syndrome.

The present invention further provides the use of one or more compounds or compositions of the present invention for the treatment of an inflammatory disease. For example, the inflammatory disease can be SIRS, sepsis, septic shock, atherosclerosis, celiac disease, interstitial cystitis, transplant rejection, rheumatoid arthritis, juvenile rheumatoid arthritis, inflammatory bowel disease (ulcerative colitis, Crohn's disease), age-related macular degeneration, IgA nephropathy, glomerulonephritis, vasculitis, polymyositis, or Wegener's disease.

The present invention further provides the use of one or more compounds or compositions of the present invention for the treatment of a neurodegenerative disease. For example, the neurodegenerative disease can be Alzheimer's disease, Parkinson's disease, multiple sclerosis, IgM polyneuropathies, or myasthenia gravis.

The present invention further provides the use of one or more compounds or compositions of the present invention in the manufacture of a medicament for inhibiting cGAS in a cell.

The present invention further provides the use of one or more compounds or compositions of the present invention in the manufacture of a medicament for the treatment of a cGAS-mediated condition.

The present invention further provides the use of one or more compounds or compositions of the present invention in the manufacture of a medicament for the treatment of an autoimmune disease.

The present invention further provides the use of one or more compounds or compositions of the present invention in the manufacture of a medicament for the treatment of an inflammatory disease.

The present invention further provides the use of one or more compounds or compositions of the present invention in the manufacture of a medicament for the treatment of a neurodegenerative disease.

cGAS inhibitory activity of any of the compounds disclosed herein can be determined by reacting the compound in a properly buffered environment with a DNA-activated cGAS in the presence of ATP and GTP. Antagonist activity can then be quantified by measuring the amount of ATP and/or GTP remaining after reaction is halted. Human cGAS sequence encoding amino acids 155-522 (DAAPGASKLRAVLEKLKLSRDDISTAAGMVKGVVDHLLLRLKCDSAFRGVGLLNTGS YYEHVKISAPNEFDVMFKLEVPRIQLEEYSNTRAYYFVKFKRNPKENPLSQFLEGEILSA SKMLSKFRKIIKEEINDIKDTDVIMKRKRGGSPAVTLLISEKISVDITLALESKSSWPASTQ EGLRIQNWLSAKVRKQLRLKPFYLVPKHAKEGNGFQEETWRLSFSHIEKEILNNHGKSK TCCENKEEKCCRKDCLKLMKYLLEQLKERFKDKKHLDKFSSYHVKTAFFHVCTQNPQD SQWDRKDLGLCFDNCVTYFLQCLRTEKLENYFIPEFNLFSSNLIDKRSKEFLTKQIEYER NNEFPVFDEF, SEQ. ID No. 1) can be cloned into an expression plasmid to create a construct containing codes for the appropriate proteins and tags (e.g., hexahistidine tag, maltose binding protein fusion, and a cleavable linker) preceding the cGAS sequence. The protein can then be expressed and purified using standard techniques.

The cGAS inhibitory activity of any of the compounds disclosed herein can also be determined by measuring changes in the type I interferon signature resulting from administration of the compound(s).

Potential cGAS antagonists, e.g., the triazine compounds disclosed herein, can be made to react, in a properly buffered environment, with a DNA-activated cGAS in the presence of ATP and GTP. Antagonist activity can then be quantified by measuring the amount of ATP and/or GTP remaining after reaction is halted.

The disclosure having been described, the following examples are offered by way of illustration and not limitation.

### EXAMPLES

NMR spectra were recorded on a Bruker Avance III HD spectrometer (400 MHz). UPLCMS were acquired on a Shimadzu LCMS 2020 equipped with a Shimadzu PDA (190-700 nm) UV detector and a Shimadzu ESI (ES+, 200-800 amu) MS Detector.

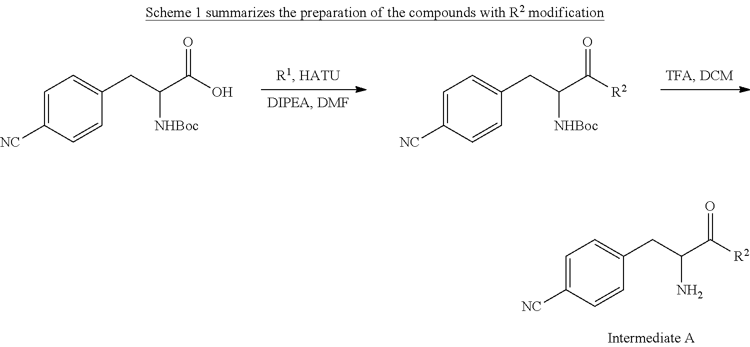

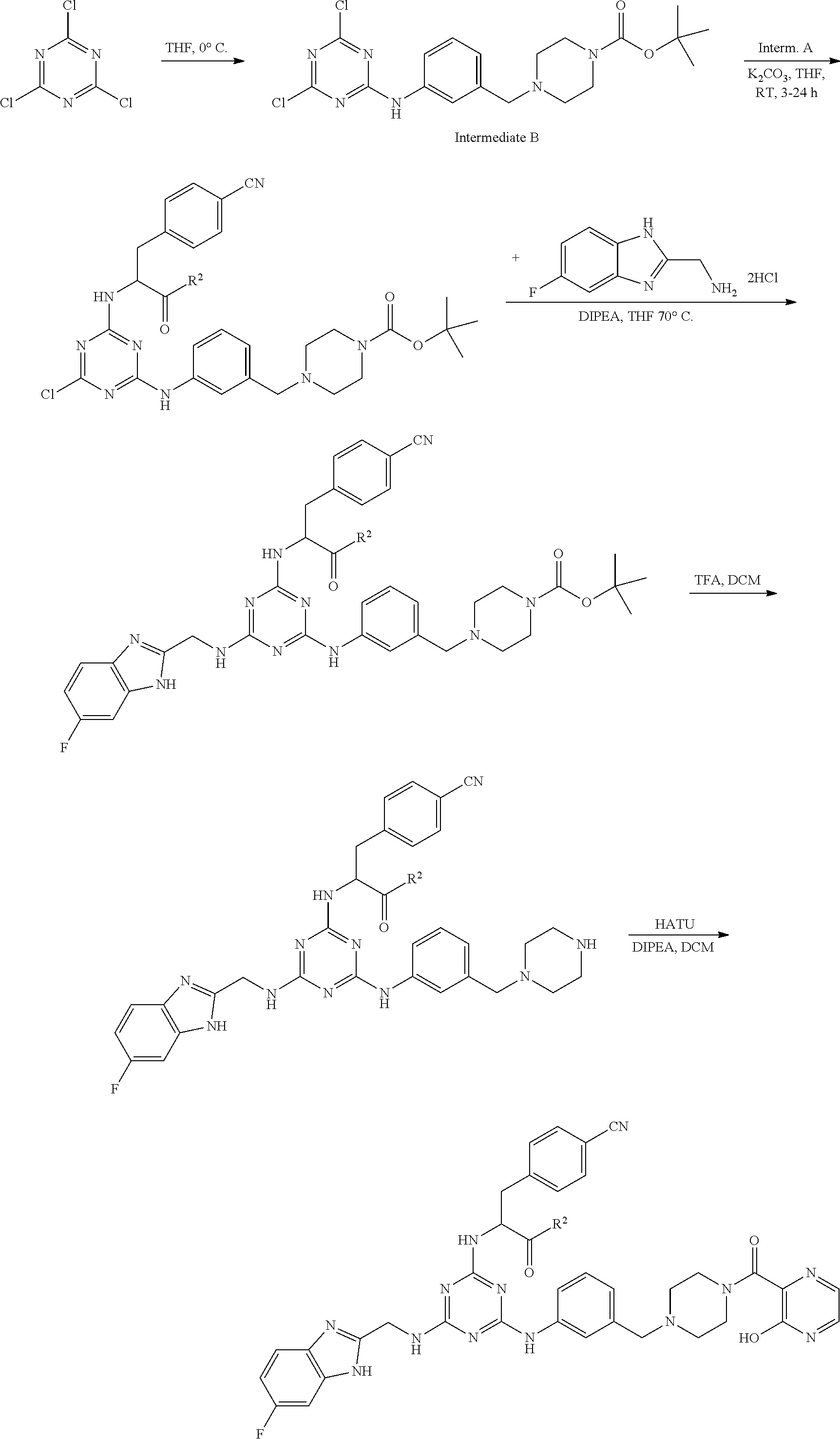

### General Procedure A: Key Intermediate a Synthesis

### Step 1

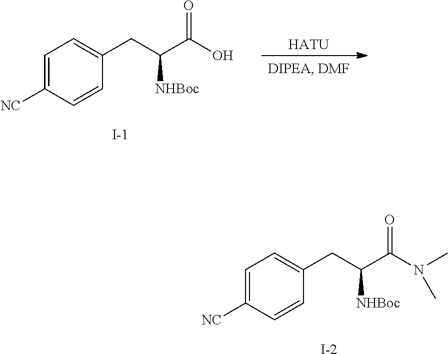

Preparation of tert-butyl (S)-(3-(4-cyanophenyl)-1-(dimethylamino)-1-oxopropan-2-yl)carbamate (I-2; R²=—N(CH₃)₂). HATU (1.2 equiv., 1.57 g, 4.13 mmol) was added to a stirring solution of (2S)-2-(tert-butoxycarbonylamino)-3-(4-cyanophenyl)propanoic acid (I-1, 1 equiv., 1 g, 3.44 mmol, Alfa Aesar, Tewksbury, Mass., USA) in N,N-dimethylformamide (14 mL) and N,N-diisopropylamine (3 equiv., 1.47 mL, 10.33 mmol). After being stirred at r.t. for 10 minutes, 2.0M dimethylamine in tetrahydrofuran (3 equiv., 5.2 mL, 10.33 mmol, Sigma-Aldrich, St. Louis, Mo., USA) was added. The reaction was stirred for 2 h. at r.t. and was partitioned between water and ethyl acetate. The organic phase was washed with saturated brine, dried over sodium sulfate, filtered and concentrated. The crude material was purified by ISCO Teledyne Combi-flash (DCM/MeOH=99:1 to 90:10) to afford the desired compound (I-2, 1.02 g, 94%).

### Step 2

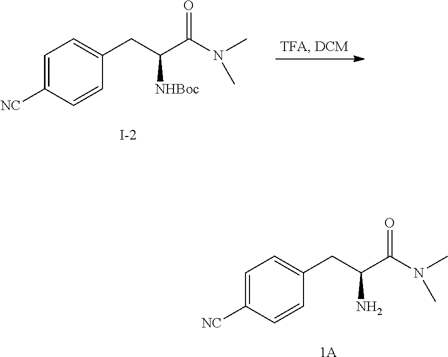

Preparation of (S)-2-amino-3-(4-cyanophenyl)-N,N-dimethylpropanamide (Intermediate 1A). To a solution of compound I-2 (0.8 g, 2.52 mmol) in DCM (0.08-0.50M) was added dropwise TFA (TFA/DCM=1:1) at 0° C. The mixture was stirred for 30 minutes and concentrated in vacuo. The residue was re-dissolved in DCM and very carefully neutralized using saturated NaHCO₃(aq.). The organic phase was washed with brine, dried over sodium sulfate and concentrated to afford the intermediate 1A which was used for the next step directly.

Additional intermediate compounds synthesized using General Procedure A are shown in Table 2 below, where dimethylamine is replaced with methylamine (Spectrum Chemical, New Brunswick, N.J., USA), 2-methoxyethan-1-amine (TCI America, Portland, Oreg., USA), or 2-(2-methoxyethoxy)ethan-1-amine (Ark Pharm, Arlington Heights, Ill., USA) in Step 1 above.

TABLE 2

| | Structure | Prepared as in |
|---|---|---|
| Intermediate 2A | 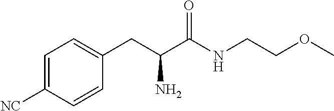 [SMILES: COCCNC(=O)[C@@H](N)Cc1ccc(C#N)cc1] | General procedure A |
| Intermediate 3A | 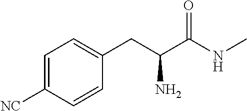 | General procedure A |
| Intermediate 4A | 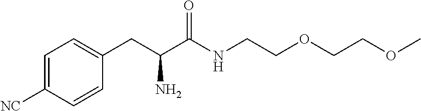 [SMILES: COCCOCCNC(=O)[C@@H](N)Cc1ccc(C#N)cc1] | General procedure A |

### General Procedure B: Key Intermediate B Synthesis

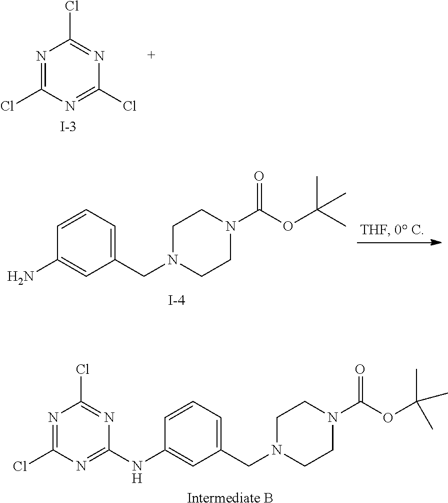

Preparation of tert-butyl 4-(3-((4,6-dichloro-1,3,5-triazin-2-yl)amino)benzyl)piperazine-1-carboxylate (Intermediate B). To a stirring solution of cyanuric chloride (I-3, 1.2 equiv., 0.66 g, 3.6 mmol, Acros Organics, Fisher Scientific) in anhydrous THF (3 mL) was added dropwise a solution of tert-butyl 4-[(3-aminophenyl)methyl]piperazine-1-carboxylate (I-4, 1 equiv., 0.87 g, 3 mmol, Maybridge, Fisher Scientific) in THF (3 mL) at 0° C. The resulting mixture was stirred at 0° C. for 2 h and concentrated under reduced pressure. The crude residue was purified by ISCO Teledyne combi-flash (DCM/MeOH 99:1 to 90:10) to afford the desired Intermediate B (1.25 g, 95%) as a white solid.

### Example 1—(S)-3-(4-cyanophenyl)-2-((4-(((5-fluoro-1H-benzo[d]imidazol-2-yl)methyl)amino)-6-((3-((4-(3-hydroxypyrazine-2-carbonyl)piperazin-1-yl)methyl)phenyl)amino)-1,3,5-triazin-2-yl)amino)-N,N-dimethylpropanamide (TA1021)

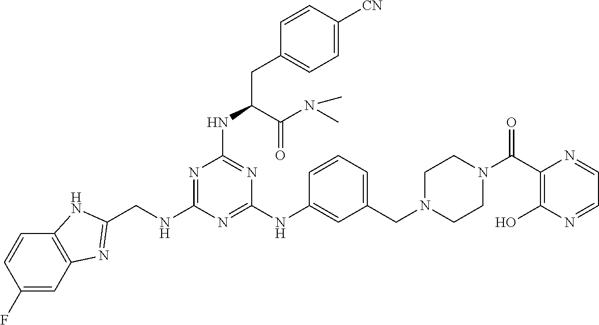 [SMILES: CN(C)C(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1]

### Step 1

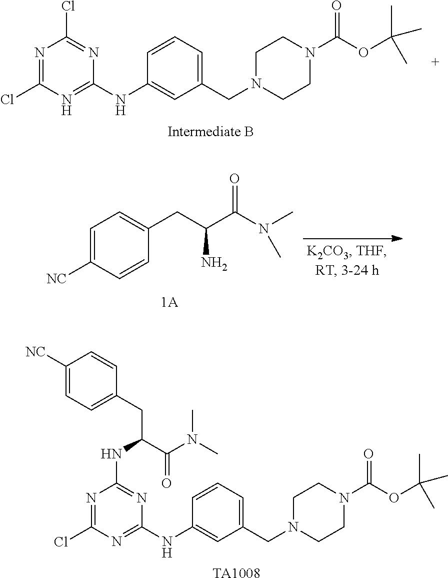

Preparation of tert-butyl (S)-4-(3-((4-chloro-6-((3-(4-cyanophenyl)-1-(dimethylamino)-1-oxopropan-2-yl)amino)-1,3,5-triazin-2-yl)amino)benzyl)piperazine-1-carboxylate (TA1008). To a stirred solution of intermediate 1A (1 equiv., 0.90 g, 2.1 mmol) in anhydrous THF (17 mL) was added intermediate B (1.2 equiv., 0.53 g, 2.5 mmol) and K₂CO₃ (1.5 equiv., 0.43 g, 3.1 mmol). The mixture was stirred at r.t. for overnight. The resultant mixture was concentrated and purified by ISCO Teledyne combi-flash (DCM/MeOH=99:1 to 90:10) to afford the desired compound TA1008 (1.61 g, 58%).

### Step 2

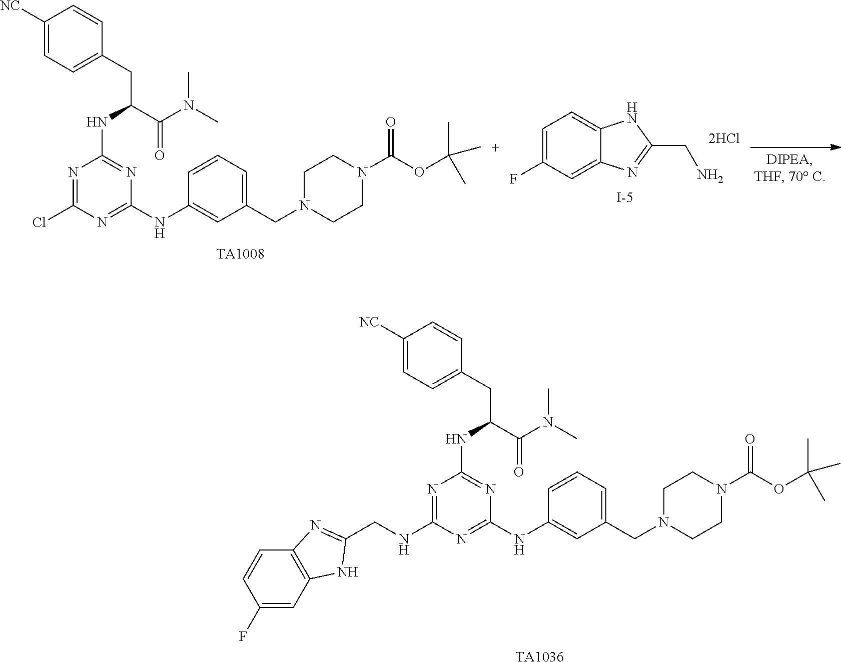

Preparation of tert-butyl (S)-4-(3-((4-((3-(4-cyanophenyl)-1-(dimethylamino)-1-oxopropan-2-yl)amino)-6-(((5-fluoro-1H-benzo[d]imidazol-2-yl)methyl)amino)-1,3,5-triazin-2-yl)amino)benzyl)piperazine-1-carboxylate (TA1036). To a solution of TA1008 (1 equiv., 0.2 g, 0.32 mmol) in anhydrous THF (3.2 mL) was added (5-fluoro-1H-benzimidazol-2-yl)methanamine dihydrochloride salt (I-5, 0.23 g, 0.97 mmol, Enamine LLC, Monmouth Jet, N.J., USA)), followed by adding DIPEA (20 equiv. 6.5 mmol, 0.92 mL). The reaction was stirred at 70° C. overnight. The solvent was evaporated and the residue was washed with water and brine. The organic phase was dried over sodium sulfate, the desiccant was filtered off, and the solvent was concentrated to give the crude compound TA1036 which was used directly for the next step without further purification.

### Step 3

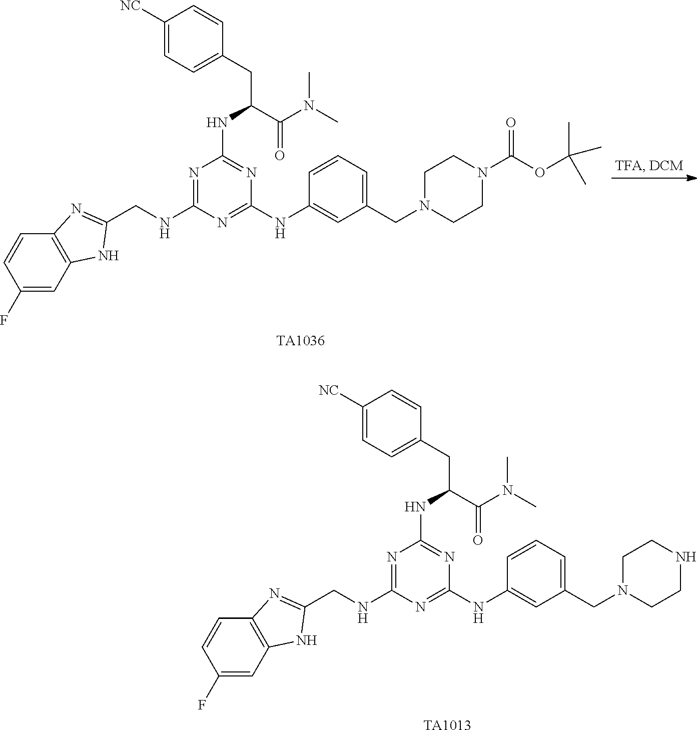

Preparation of (S)-3-(4-cyanophenyl)-2-((4-(((5-fluoro-1H-benzo[d]imidazol-2-yl)methyl)amino)-6-((3-(piperazin-1-ylmethyl)phenyl)amino)-1,3,5-triazin-2-yl)amino)-N,N-dimethylpropanamide (TA1013). To a solution of TA1036 (1 equiv., 0.15 g, 0.20 mmol) in DCM (0.08-0.50M) was added dropwise TFA (TFA/DCM=1:1) at 0° C. The mixture was stirred for 30 minutes and concentrated in vacuo. The crude residue was purified by reverse phase preparative HPLC (XBridge BEH, 19×150 mm, 5 μm, C18 column; ACN/water with 0.1% formic acid modifier, 20 mL/min), affording desired compound TA1013 (109.9 mg, 85%) as an off-white solid. MS (m/z): 649 [M+1]⁺, purity: 99%. Compound (S)-2-((4-chloro-6-((3-(piperazin-1-yl methyl)phenyl)amino)-1,3,5-triazin-2-yl)amino)-3-(4-cyanophenyl)-N,N-dimethylpropanamide TA1009 is prepared similarly by reacting TA1008 with TFA/DCM according to this step.

### Step 4

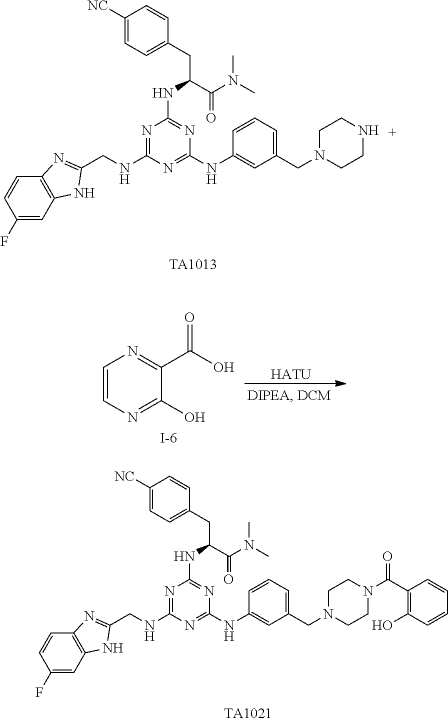

Preparation of (S)-3-(4-cyanophenyl)-2-((4-(((5-fluoro-1H-benzo[d]imidazol-2-yl)methyl)amino)-6-((3-((4-(3-hydroxypyrazine-2-carbonyl)piperazin-1-yl)methyl)phenyl)amino)-1,3,5-triazin-2-yl)amino)-N,N-dimethylpropanamide (TA1021). HATU (1.2 equiv., 34 mg, 0.093 mmol) was added to a solution of 3-hydroxypyrazine-2-carboxylic acid (I-6, 1.1 equiv., 12 mg, 0.080 mmol, Synthonix, Fisher Scientific) in anhydrous DCM (0.70 mL) and DIPEA (3 equiv., 0.040 mL, 0.23 mmol). After being stirred at r.t. for 10 minutes, TA1013 (50 mg, 0.080 mmol) was added. The reaction was stirred at r.t. until the LCMS analysis showed complete consumption of the starting material (3-24 h). The crude residue was then purified by reverse phase preparative HPLC (XBridge BEH, 19×150 mm, 5 μm, C18 column; ACN/water with 0.1% formic acid modifier, 20 mL/min), affording Compound TA1021 (17.3 mg, 29%) as an orange solid. ¹H NMR (400 MHz, DMSO-d₆) δ 12.20 (s, 1H), 8.95 (d, J=24.1 Hz, 1H), 8.07-7.87 (m, 1H), 7.84-7.68 (m, 1H), 7.68-7.41 (m, 4H), 7.40-7.22 (m, 2H), 7.22-6.78 (m, 3H), 6.56 (s, 1H), 5.18-4.91 (m, 1H), 4.79-4.50 (m, 2H), 3.74-3.39 (m, 7H), 3.28-3.13 (m, 4H), 3.12-2.78 (m, 5H), 2.75-2.66 (m, 1H), 2.61 (s, 1H), 2.48-2.02 (m, 4H). MS (m/z): 771 [M+1]⁺, LCMS purity: 99%.

The compounds (S)-3-(4-cyanophenyl)-2-((4-(((5-fluoro-1H-benzo[<7]imidazol-2-yl)methyl)amino)-6-((3-(piperazin-1-ylmethyl)phenyl)amino)-1,3,5-triazin-2-yl)amino)-N-(2-methoxyethyl)propanamide (TA1017), (5)-3-(4-cyanophenyl)-2-((4-(((5-fluoro-1H-benzo[d]imidazol-2-yl) methyl)amino)-6-((3-((4-(3-hydroxypyrazine-2-carbonyl)piperazin-1-yl)methyl)phenyl)amino)-1,3,5-triazin-2-yl)amino)-N-(2-methoxyethyl)propanamide (TA1018) and tert-butyl (S)-4-(3-((4-((3-(4-cyanophenyl)-1-((2-methoxyethyl)amino)-1-oxopropan-2-yl)amino)-6-(((5-fluoro-1H-benzo[d]imidazol-2-yl)methyl)amino)-1,3,5-triazin-2-yl)amino)benzyl)piperazine-1-carboxylate (TA1037):

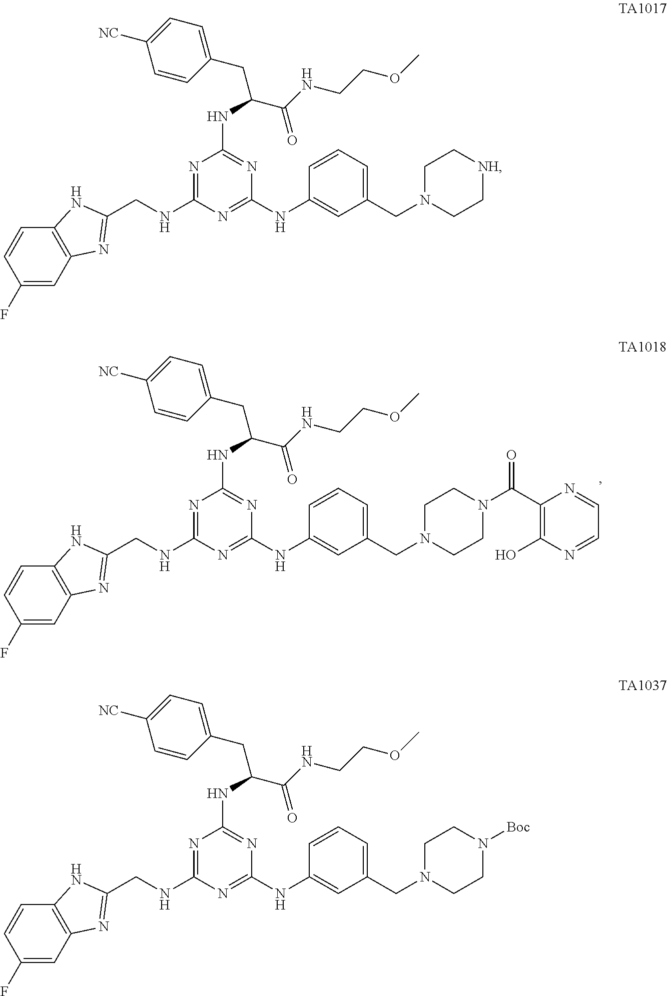 [SMILES: COCCNC(=O)[C@H](Cc1ccc(C#N)cc1)Nc1nc(NCc2nc3cc(F)ccc3[nH]2)nc(Nc2cccc(CN3CCNCC3)c2)n1 ; COCCNC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1 ; COCCNC(=O)[C@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1]

were prepared similarly to these methods, replacing Int. 1A with Int. 2A in step 1.

TA1018: ¹H NMR (400 MHz, DMSO-d₆) δ 12.57 (s, 1H), 12.27-12.13 (m, 1H), 9.06-8.90 (m, 1H), 8.19 (d, 7=32.2 Hz, 1H), 8.04-7.87 (m, 1H), 7.82-7.70 (m, 1H), 7.70-7.61 (m, 1H), 7.61-7.44 (m, 3H), 7.43-7.28 (m, 2H), 7.25-6.95 (m, 3H), 6.92-6.79 (m, 1H), 4.67 (s, 2H), 4.39-4.26 (m, 1H), 3.65-3.46 (m, 5H), 3.31-3.13 (m, 7H), 3.10-2.82 (m, 3H), 2.73-2.65 (m, 1H), 2.46-2.18 (m, 4H). MS (m/z): 801 [M+1]⁺, LCMS purity: 97%.

The compounds (5)-3-(4-cyanophenyl)-2-((4-(((5-fluoro-1H-benzo[d]imidazol-2-yl)methyl)amino)-6-((3-(piperazin-1-ylmethyl)phenyl)amino)-1,3,5-triazin-2-yl)amino)-N-methylpropanamide (TA1022), (S)-3-(4-cyanophenyl)-2-((4-(((5-fluoro-1H-benzo[d]imidazol-yl)methyl)amino)-6-((3-(hydroxypyrazine-2-carbonyl)piperazin-1-yl)methyl)phenyl)amino)-1,3,5-triazin-2-yl)amino)-N-methylpropanamide (TA1024) and tert-butyl (S)-4-(3-((4-((3-(4-cyanophenyl)-1-(methylamino)-1-oxopropan-2-yl)amino)-6-(((5-fluoro-1H-benzo[d]imidazol-2-yl)methyl)amino)-1,3,5-triazin-2-yl)amino)benzyl)piperazine-1-carboxylate (TA1038):

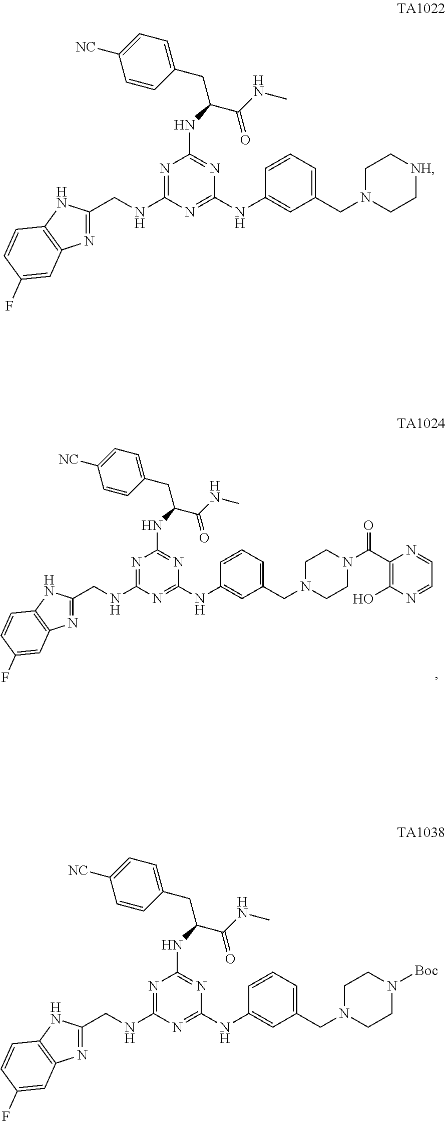 [SMILES: CNC(=O)[C@H](Cc1ccc(C#N)cc1)Nc1nc(NCc2nc3cc(F)ccc3[nH]2)nc(Nc2cccc(CN3CCNCC3)c2)n1 ; CNC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1 ; CNC(=O)[C@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1]

were prepared similarly to these methods, replacing Int. 1A with Int. 3A in step 1.

TA1024: ¹H NMR (400 MHz, DMSO-d₆) δ 12.79 (s, 1H), 11.62-10.95 (m, 1H), 9.65-9.21 (m, 1H), 8.30-7.94 (m, 2H), 7.87-7.61 (m, 3H), 7.52 (dt, J=25.5, 11.4 Hz, 3H), 7.43-7.36 (m, 1H), 7.31 (q, J=8.3 z, 1H), 7.29-7.04 (m, 2H), 4.93-4.69 (m, 2H), 4.69-4.48 (m, 1H), 4.43-4.18 (m, 2H), 3.88-2.82 (m, 14H), 2.65 (m, 2H), 2.44 (d, J=4.0 Hz, 1H). MS (m/z): 757 [M+1]⁺, LCMS purity: 98%.

The compounds (S)-3-(4-cyanophenyl)-2-((4-(((5-fluoro-1H-benzo[d]imidazol-2-yl)methyl)amino)-6-((3-(piperazin-1-yl methyl)phenyl)amino)-1,3,5-triazin-2-yl)amino)-N-(2-(2-methoxyethoxy)ethyl)propanamide (TA1023), (S)-3-(4-cyanophenyl)-2-((4-(((5-fluoro-1H-benzo[₆/]imidazol-2-yl) methyl)amino)-6-((3-((4-(3-hydroxypyrazine-2-carbonyl)piperazin-1-yl)methyl)phenyl)amino)-1,3,5-triazin-2-yl)amino)-N-(2-(2-methoxyethoxy)ethyl)propanamide (TA1025) and tert-butyl (S)-4-(3-((4-((3-(4-cyanophenyl)-1-((2-(2-methoxyethoxy)ethyl)amino)-1-oxopropan-2-yl)amino)-6-(((5-fluoro-1H-benzo[d]imidazol-2-yl)methyl)amino)-1,3,5-triazin-2-yl)amino)benzyl)piperazine-1-carboxylate (TA1039):

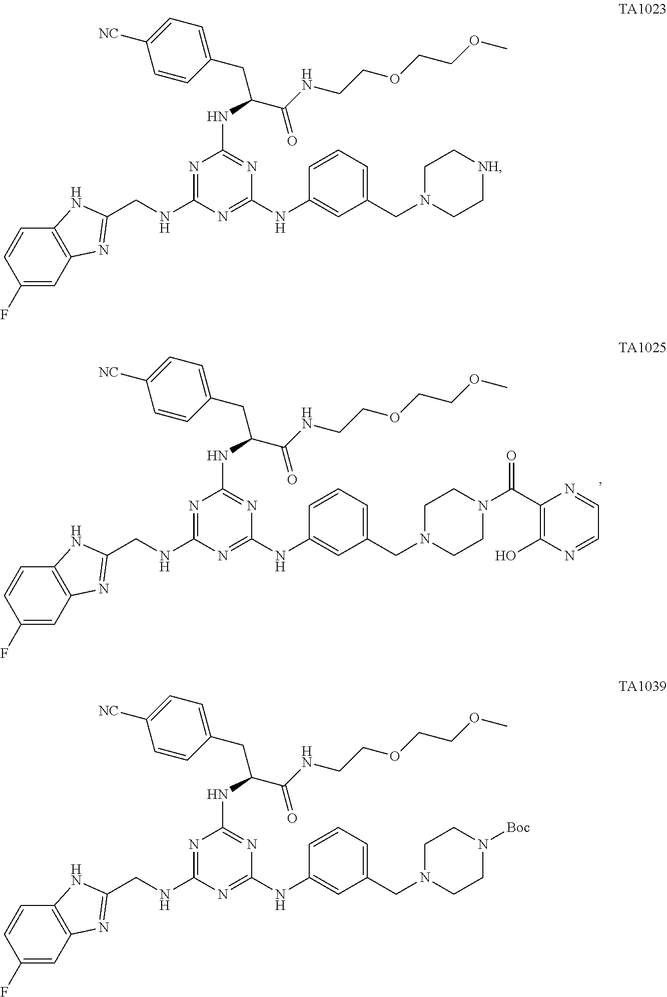 [SMILES: COCCOCCNC(=O)[C@H](Cc1ccc(C#N)cc1)Nc1nc(NCc2nc3cc(F)ccc3[nH]2)nc(Nc2cccc(CN3CCNCC3)c2)n1 ; COCCOCCNC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1 ; COCCOCCNC(=O)[C@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1]

were prepared similarly to these methods, replacing Int. 1A with Int. 4A in step 1.

TA1025: ¹H NMR (400 MHz, DMSO-d₆) δ 12.75 (s, 1H), 11.47-10.76 (m, 1H), 9.80-9.29 (m, 1H), 8.34-7.92 (m, 2H), 7.91-7.63 (m, 3H), 7.65-7.46 (m, 3H), 7.37 (ddd, J=30.4, 18.7, 6.1 Hz, 3H), 7.25-7.05 (m, 1H), 4.98-4.75 (m, 2H), 4.74-4.45 (m, 2H), 4.44-4.22 (m, 2H), 4.21-3.48 (m, 9H), 3.47-3.31 (m, 6H), 3.31-3.23 (m, 2H), 3.23-3.16 (m, 3H), 3.16-2.80 (m, 4H). MS (m/z): 845 [M+1]⁺, LCMS purity: 97%.

The compounds (S)-3-(4-cyanophenyl)-2-((4-(((5-fluoro-1H-benzo[d]imidazol-2-yl)methyl)amino)-6-((3-methoxypyrazine-2-carbonyl)piperazin-1-yl)methyl)phenyl)amino)-1,3,5-triazin-2-yl)amino)-N-(2-(2-methoxyethoxy)ethyl)propanamide (TA1029),

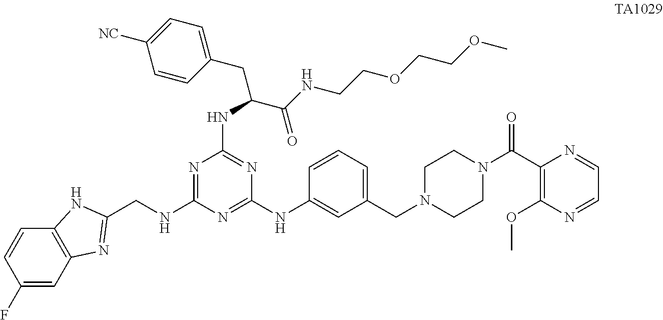 [SMILES: COCCOCCNC(=O)[C@H](Cc1ccc(C#N)cc1)Nc1nc(NCc2nc3cc(F)ccc3[nH]2)nc(Nc2cccc(CN3CCN(C(=O)c4nccnc4OC)CC3)c2)n1]

was prepared similarly, where 3-hydroxypyrazine-2-carboxylic acid I-6 is also replaced with 3-methoxypyrazine-2-carboxylic acid (Ark Pharm, Arlington Heights, Ill., USA) in step 4.

TA1029: ¹H NMR (400 MHz, DMSO-d₆) 8.28 (d, J=2.7 Hz, 1H), 8.19 (d, J=2.7 Hz, 1H), 7.73-7.55 (m, 3H), 7.55-7.35 (m, 4H), 7.30-7.12 (m, 3H), 7.07-6.90 (m, 2H), 4.77 (m, 3H), 4.60 (s, 3H), 4.01 (s, 3H), 3.88-3.68 (m, 3H), 3.64-3.57 (m, 1H), 3.53-3.38 (m, 7H), 3.27-2.99 (m, 5H), 2.79-2.23 (m, 6H). MS (m/z): 859 [M+1]⁺, LCMS purity: 98%.

The compounds (S)-3-(4-cyanophenyl)-2-((4-(((5,6-dimethyl-1H-benzo[d]imidazol-2-yl)methyl)(methyl)amino)-6-((3-(piperazin-1-ylmethyl)phenyl)amino)-1,3,5-triazin-2-yl)amino)-N,N-dimethylpropanamide (TA1014), (5)-3-(4-cyanophenyl)-2-((4-(((5,6-dimethyl-1H-benzo[₆/]imidazol-2-yl) methyl)(methyl)amino)-6-((3-((4-(5-methyl-1H-benzo[d]imidazole-6-carbonyl)piperazin-1-yl)methyl)phenyl)amino)-1,3,5-triazin-2-yl)amino)-N,N-dimethylpropanamide (TA1015) tert-butyl (S)-4-(3-((4-((3-(4-cyanophenyl)-1-(dimethylamino)-1-oxopropan-2-yl)amino)-6-(((5,6-dimethyl-1H-benzo[d]imidazol-2-yl)methyl)(methyl)amino)-1,3,5-triazin-2-yl)amino)benzyl)piperazine-1-carboxylate (TA1045), and (S)-3-(4-cyanophenyl)-2-((4-(((5,6-dimethyl-1H-benzo[d]imidazol-2-yl)methyl)(methyl)amino)-6-((3-((4-((1,5-dimethyl-1H-pyrazol-4-yl)sulfonyl)piperazin-1-yl)methyl)phenyl)amino)-1,3,5-triazin-2-yl)amino)-N,N-dimethylpropanamide (TA1073):

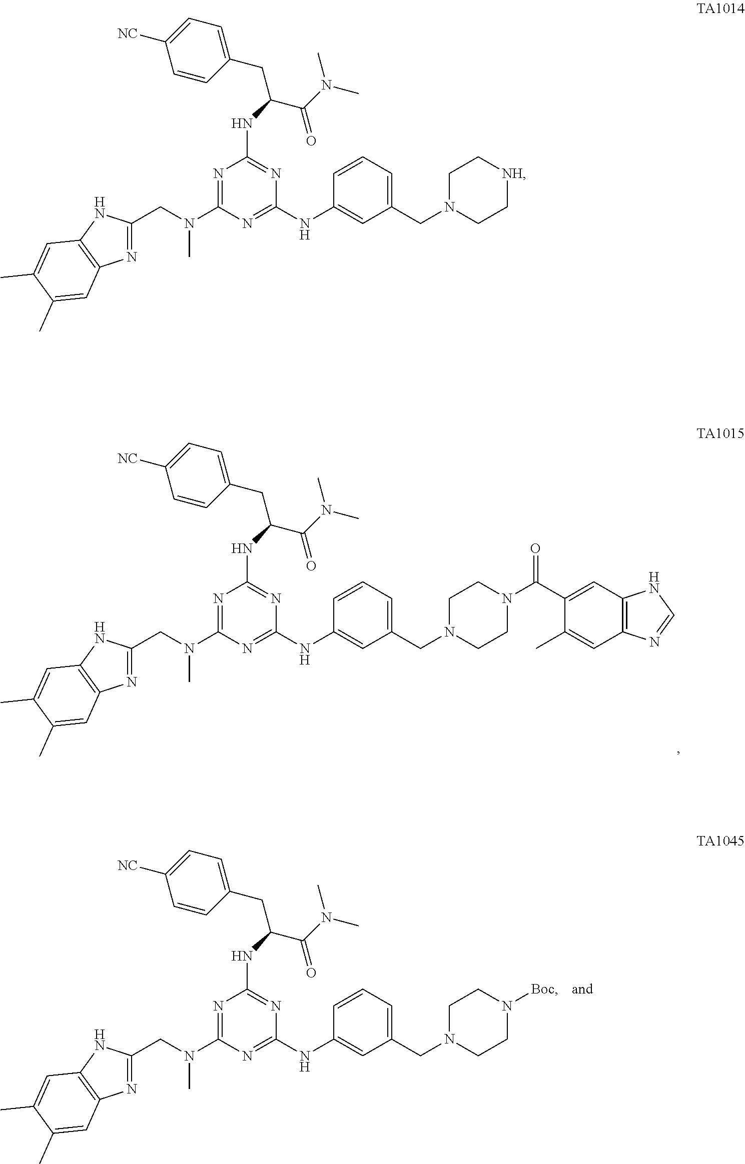 [SMILES: Cc1cc2nc(CN(C)c3nc(Nc4cccc(CN5CCNCC5)c4)nc(N[C@@H](Cc4ccc(C#N)cc4)C(=O)N(C)C)n3)[nH]c2cc1C ; Cc1cc2nc(CN(C)c3nc(Nc4cccc(CN5CCN(C(=O)c6cc7[nH]cnc7cc6C)CC5)c4)nc(N[C@@H](Cc4ccc(C#N)cc4)C(=O)N(C)C)n3)[nH]c2cc1C ; Cc1cc2nc(CN(C)c3nc(Nc4cccc(CN5CCN(C(=O)OC(C)(C)C)CC5)c4)nc(N[C@@H](Cc4ccc(C#N)cc4)C(=O)N(C)C)n3)[nH]c2cc1C]

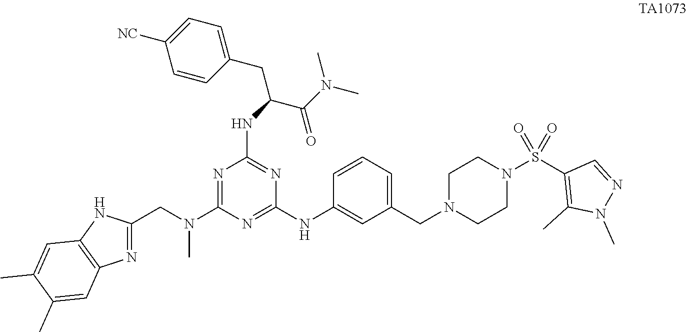 [SMILES: Cc1cc2nc(CN(C)c3nc(Nc4cccc(CN5CCN(S(=O)(=O)c6cnn(C)c6C)CC5)c4)nc(N[C@@H](Cc4ccc(C#N)cc4)C(=O)N(C)C)n3)[nH]c2cc1C]

were prepared similarly to these methods, replacing (5-fluoro-1H-benzimidazol-2-yl)methanamine dihydrochloride salt I-5 with l-(5,6-dimethyl-1H-benzo[d]imidazol-2-yl)-N-methylmethanamine (Combi-Blocks, Sigma-Aldrich) in step 2 and replacing 3-hydroxypyrazine-2-carboxylic acid I-6 with 5-methyl-1H-benzo[d]imidazole-6-carboxylic acid (Alfa Aesar, Tewksbury, Mass., USA) for TA1015 or with 1,5-dimethyl-1H-pyrazole-4-sulfonic acid for TA1073 (SOURCE) in step 4.

TA1015: ¹H NMR (400 MHz, DMSO-d₆) δ 12.42 (s, 1H), 11.86 (s, 1H), 8.99 (d, J=22.1 Hz, 1H), 8.20 (s, 1H), 7.81-7.71 (m, 1H), 7.69-7.60 (m, 1H), 7.58-7.38 (m, 4H), 7.38-7.25 (m, 3H), 7.25-7.05 (m, 3H), 6.92-6.77 (m, 1H), 5.14 (dq, J=15.1, 8.7 Hz, 1H), 5.06-4.80 (m, 3H), 3.75-3.54 (m, 2H), 3.54-3.38 (m, 2H), 3.22-3.12 (m, 3H), 3.00 (m, 7H), 2.90-2.73 (m, 4H), 2.64 (s, 1H), 2.44 (s, 1H), 2.29 (s, 10H). MS (m/z): 859 [M+1]⁺, LCMS purity: 99%.

TA1073: MS (m/z): 831 [M+1]⁺, LCMS purity: 99.9%.

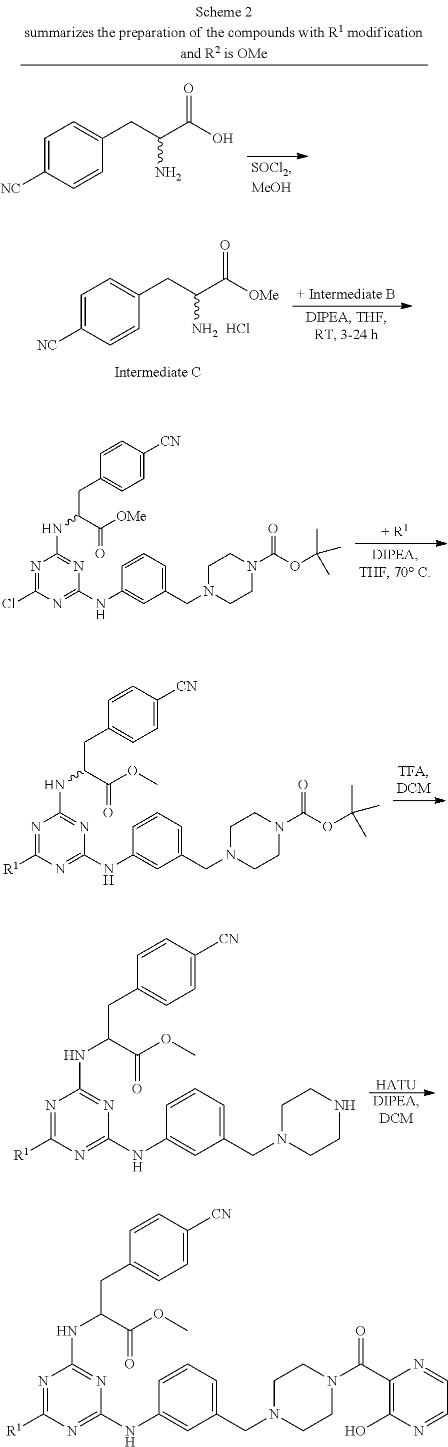

### General Procedure C: Key Intermediate C Synthesis

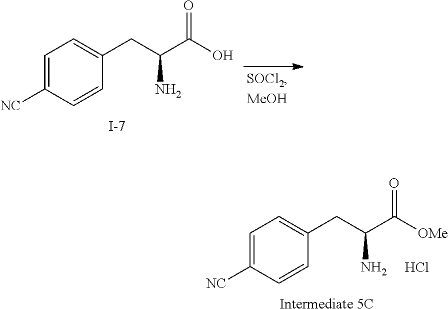

Preparation of methyl (S)-2-amino-3-(4-cyanophenyl)propanoate (Intermediate 5C). Prepared according to Nitsche et al. J. Med. Chem. 2017, 60, 511-516. To a solution of L-4-cyanophenylalanine (I-7, 1.3 g, 6.83 mmol, Alfa Aesar, Tewksbury, Mass., USA) in methanol (20 mL) was added dropwise thionyl chloride (2.48 mL, 34.17 mmol) at 0° C. The reaction mixture was warmed to r.t. and stirred overnight. The mixture was then concentrated under reduced pressure to afford crude compound as a white solid. Characterization data were consistent to that reported in the literature. The R isomer is prepared similarly starting with R-4-cyanophenylalanine (Alfa Aesar) to provide methyl (S)-2-amino-3-(4-cyanophenyl)propanoate (Intermediate 6C):

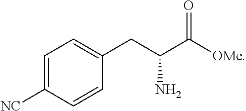 [SMILES: COC(=O)[C@H](N)Cc1ccc(C#N)cc1]

Intermediate 7C is prepared similarly by reacting I-7 with 2-(2-methoxyethoxy)-ethanol in place of methanol:

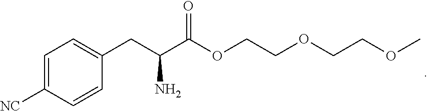 [SMILES: COCCOCCOC(=O)[C@@H](N)Cc1ccc(C#N)cc1]

### Example 2—Methyl (S)-3-(4-cyanophenyl)-2-((4-(((5-fluoro-1H-benzo[d]imidazol-2-yl)methyl)amino)-6-((3-((4-(3-hydroxypyrazine-2-carbonyl)piperazin-1-yl)methyl)phenyl)amino)-1,3,5-triazin-2-yl)amino)propanoate (TA1020)

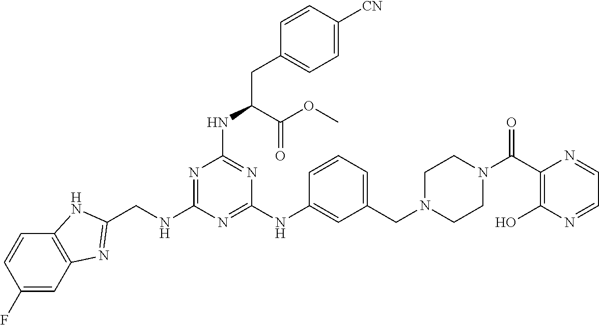 [SMILES: COC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1]

### Step 1

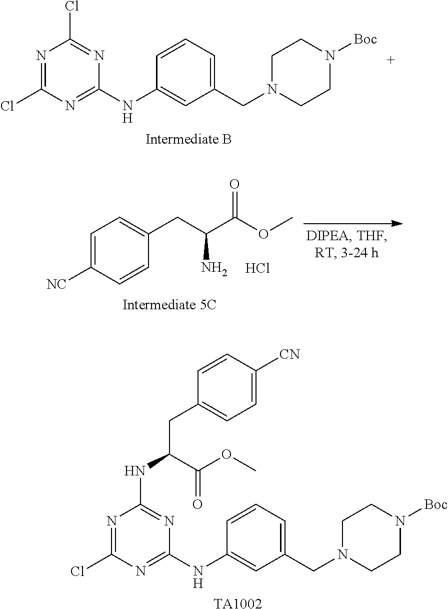

Preparation of tert-butyl (S)-4-(3-((4-chloro-6-((3-(4-cyanophenyl)-1-methoxy-1-oxopropan-2-yl)amino)-1,3,5-triazin-2-yl)amino)benzyl)piperazine-1-carboxylate (TA1002). Prepared in an analogous manner to example 1, step 1 using Intermediate B, Intermediate 5C and DIPEA as a base. To a stirred solution of Intermediate B (1 equiv., 0.25 g, 0.57 mmol) in anhydrous THF (3.8 mL) was added Intermediate 5C (4 equiv., 0.63 g, 2.28 mmol) and DIPEA (6 equiv., 0.60 mL, 3.42 mmol). The mixture was stirred at r.t. for 2 h. The resultant mixture was concentrated and purified by ISCO Teledyne combi-flash (EtOAc/Heptane=4:6 to 7:3) to afford the desired compound TA1002 (0.17 g, 50%).

### Step 2

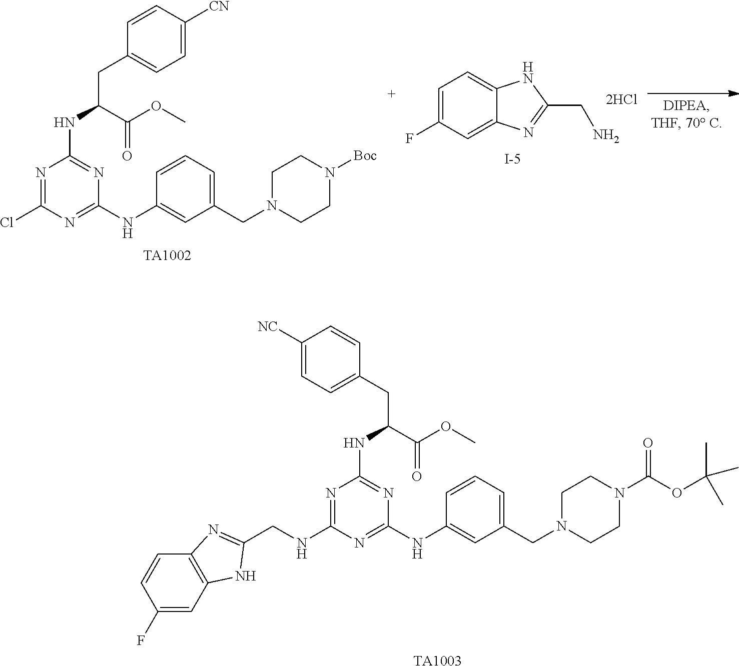

Preparation of tert-butyl (S)-4-(3-((4-((3-(4-cyanophenyl)-1-methoxy-1-oxopropan-2-yl)amino)-6-(((5-fluoro-1H-benzo[d]imidazol-2-yl)methyl)amino)-1,3,5-triazin-2-yl)amino)benzyl)piperazine-1-carboxylate (TA1003). Prepared in an analogous manner to example 1, step 2. To a solution of TA1002 (1 equiv., 0.12 g, 0.20 mmol) in anhydrous THF (1.8 mL) was added (5-fluoro-1H-benzimidazol-2-yl)methanamine dihydrochloride salt (I-5, 3 equiv., 0.14 g, 0.60 mmol), followed by adding DIPEA (20 equiv. 4.0 mmol, 0.56 mL). The reaction was stirred at 70° C. overnight. The solvent was evaporated and the residue was washed with water and brine. The organic phase was dried over sodium sulfate, the desiccant was filtered off, and the solvent was concentrated to give the crude compound TA1003 which was used directly for the next step without further purification.

### Step 3

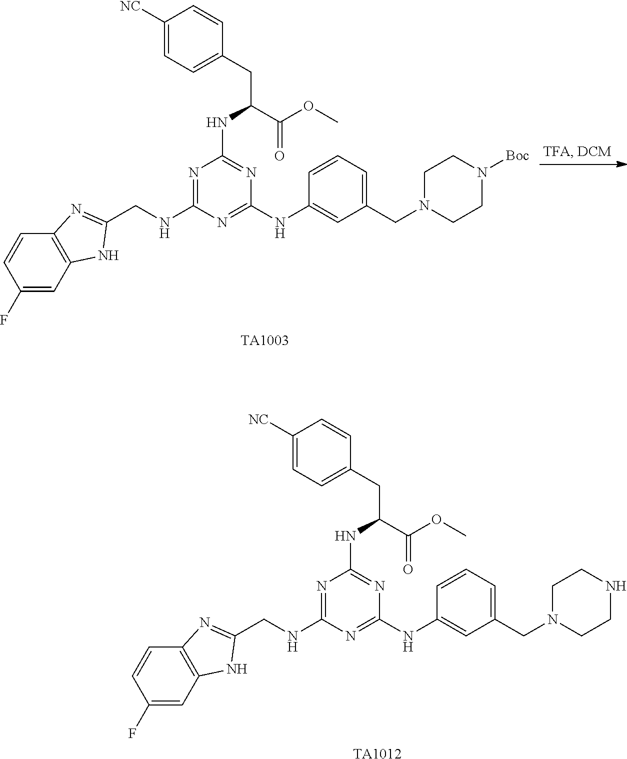

Preparation of methyl (S)-3-(4-cyanophenyl)-2-((4-(((5-fluoro-1H-benzo[d]imidazol-2-yl)methyl)amino)-6-((3-(piperazin-1-ylmethyl)phenyl)amino)-1,3,5-triazin-2-yl)amino)propanoate (TA1012). Prepared in an analogous manner to example 1, step 3. To a solution of TA1003 (1 equiv., 77 mg, 0.10 mmol) in DCM (0.08-0.50M) was added dropwise TFA (TFA/DCM=1:1) at 0° C. The mixture was stirred for 30 minutes and concentrated in vacuo. The crude residue was purified by reverse phase preparative HPLC (XBridge BEH, 19×150 mm, 5 μm, C18 column; ACN/water with 0.1% formic acid modifier, 20 mL/min), affording desired compound TA1012 (109.9 mg, 85%) as a pale yellow solid.

### Step 4

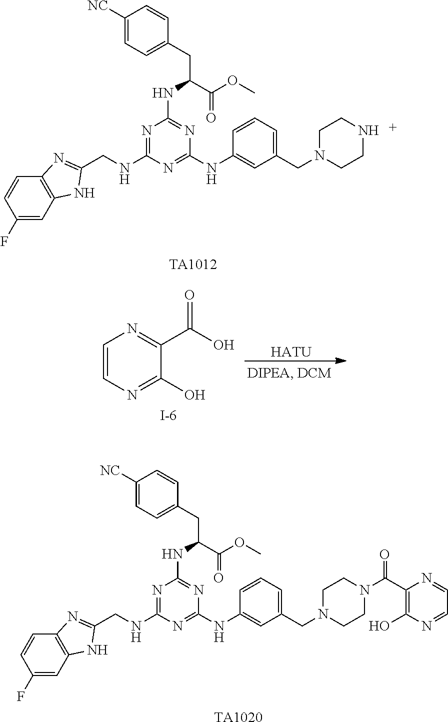

Preparation of methyl (S)-3-(4-cyanophenyl)-2-((4-(((5-fluoro-1H-benzo[d]imidazol-2-yl)methyl)amino)-6-((3-((4-(3-hydroxypyrazine-2-carbonyl)piperazin-1-yl)methyl)phenyl)amino)-1,3,5-triazin-2-yl)amino)propanoate (TA1020). Prepared in an analogous manner to example 001, step 4. HATU (1.2 equiv., 36 mg, 0.094 mmol) was added to a solution of 3-hydroxypyrazine-2-carboxylic acid (I-6, 1.1 equiv., 12 mg, 0.087 mmol) in anhydrous DCM (1 mL) and DIPEA (3 equiv., 0.040 mL, 0.23 mmol). After being stirred at r.t. for 10 minutes, TA1012 (1 equiv., 50 mg, 0.079 mmol) was added. The reaction was stirred at r.t. until the LCMS analysis showed complete consumption of the starting material (3-24 h). The crude residue was then purified by reverse phase preparative HPLC (XBridge BEH, 19×150 mm, 5 μm, C18 column; ACN/water with 0.1% formic acid modifier, 20 mL/min), affording Compound TA1020 (15.1 mg, 25%) as a yellow solid. ¹H NMR (400 MHz, Methanol-d₄) δ 8.25 (s, 1H), 7.74-7.54 (m, 3H), 7.53-7.31 (m, 5H), 7.31-7.13 (m, 3H), 7.09-6.88 (m, 3H), 4.77 (d, J=9.7 Hz, 2H), 4.60 (s, 3H), 3.85-3.67 (m, 4H), 3.63 (s, 1H), 3.53-3.47 (m, 2H), 3.23 (dd, J=14.6, 7.3 Hz, 1H), 3.17-3.09 (m, 1H), 3.05-2.94 (m, 1H), 2.86 (d, J=19.2 Hz, 1H), 2.70 (d, J=16.5 Hz, 1H), 2.67-2.34 (m, 5H). MS (m/z): 758 [M+1]⁺, LCMS purity: 98%.

The compound TA1020 was further reacted with lithium hydroxide in water/THF to provide (S)-3-(4-cyanophenyl)-2-((4-(((5-fluoro-1H-benzo[d]imidazol-2-yl)methyl)amino)-6-((3-((4-(3-hydroxypyrazine-2-carbonyl)piperazin-1-yl)methyl)phenyl)amino)-1,3,5-triazin-2-yl)amino)propanoic acid (TA1071):

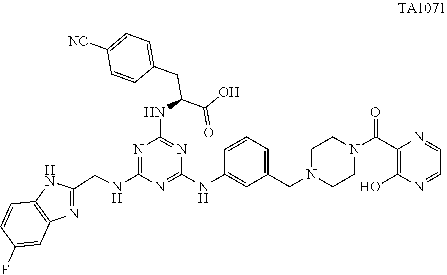 [SMILES: N#Cc1ccc(C[C@H](Nc2nc(NCc3nc4cc(F)ccc4[nH]3)nc(Nc3cccc(CN4CCN(C(=O)c5nccnc5O)CC4)c3)n2)C(=O)O)cc1]

TA1071: ¹H NMR (400 MHz, Methanol-d₄) δ 8.29-8.12 (m, 1H), 7.74-7.35 (m, 8H), 7.33-7.07 (m, 4H), 7.04-6.92 (m, 2H), 4.50-3.71 (m, 6H), 3.60-3.41 (m, 3H), 3.28-2.87 (m, 6H), 2.86-2.63 (m, 1H). MS (m/z): 744 [M+1]⁺, LCMS purity: 99%.

The compounds methyl (R)-3-(4-cyanophenyl)-2-((4-(((5-fluoro-1H-benzo[d]imidazol-2-yl)methyl)amino)-6-((3-(piperazin-1-ylmethyl)phenyl)amino)-1,3,5-triazin-2-yl)amino)propanoate (TA1019), methyl (R)-3-(4-cyanophenyl)-2-((4-(((5-fluoro-1H-benzo[d]imidazol-2-yl) methyl)amino)-6-((3-((4-(3-hydroxypyrazine-2-carbonyl)piperazin-1-yl)methyl)phenyl)amino)-1,3,5-triazin-2-yl)amino)propanoate (TA1027) and tert-butyl (R)-4-(3-((4-((3-(4-cyanophenyl)-1-methoxy-1-oxopropan-2-yl)amino)-6-(((5-fluoro-1H-benzo[d]imidazol-2-yl)methyl)amino)-1,3,5-triazin-2-yl)amino)benzyl)piperazine-1-carboxylate (TA1040):

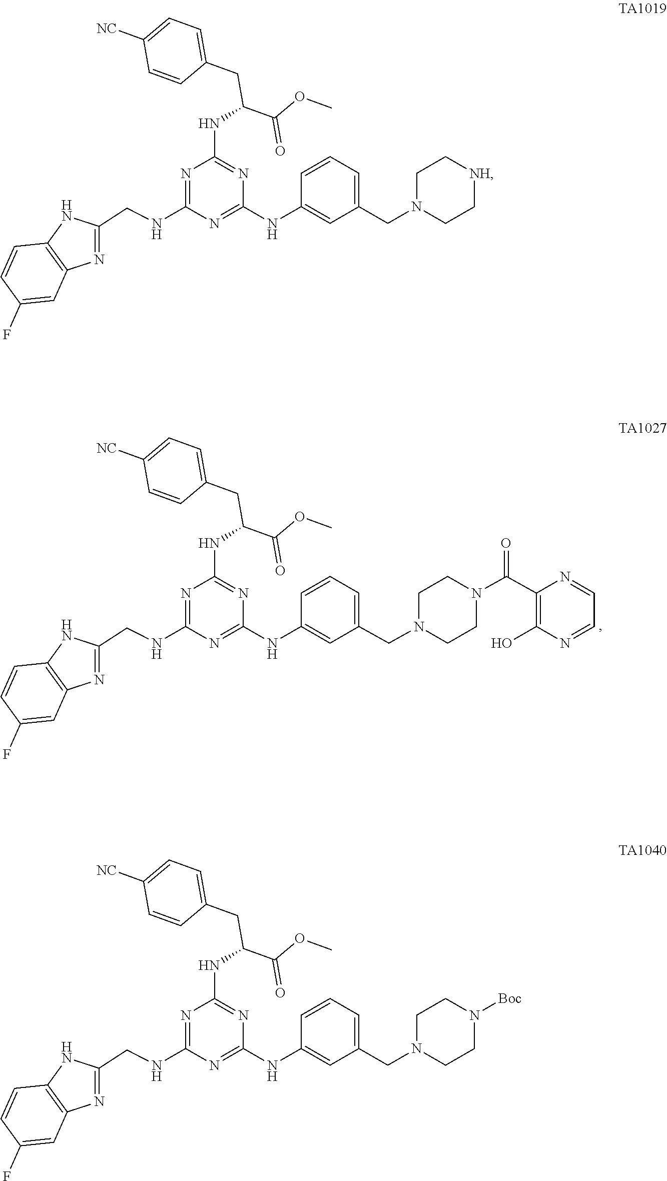 [SMILES: COC(=O)[C@@H](Cc1ccc(C#N)cc1)Nc1nc(NCc2nc3cc(F)ccc3[nH]2)nc(Nc2cccc(CN3CCNCC3)c2)n1 ; COC(=O)[C@@H](Cc1ccc(C#N)cc1)Nc1nc(NCc2nc3cc(F)ccc3[nH]2)nc(Nc2cccc(CN3CCN(C(=O)c4nccnc4O)CC3)c2)n1 ; COC(=O)[C@@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1]

were prepared similarly to these methods, replacing Int. 5C with Int. 6C in step 1.

TA1027: ¹H NMR (400 MHz, DMSO-d₆) δ 12.77 (s, 1H), 11.32 (s, 1H), 9.51-9.22 (m, 1H), 8.18-7.89 (m, 1H), 7.78 (d, J=7.6 Hz, 1H), 7.70 (d, J=7.3 Hz, 2H), 7.54 (ddd, J=33.9, 23.6, 8.1 Hz, 3H), 7.43-7.08 (m, 3H), 4.90-4.66 (m, 2H), 4.66-4.45 (m, 1H), 4.44-4.16 (m, 2H), 3.77-2.89 (m, 18H). MS (m/z): 758 [M+1]⁺, LCMS purity: 99%.

The compounds methyl (S)-3-(4-cyanophenyl)-2-((4-((2-methoxyethyl)amino)-6-((3-(piperazin-1-ylmethyl)phenyl)amino)-1,3,5-triazin-2-yl)amino)propanoate (TA1031), methyl (S)-3-(4-cyanophenyl)-2-((4-((3-((4-(3-hydroxypyrazine-2-carbonyl)piperazin-1-yl)methyl)phenyl)amino)-6-((2-methoxyethyl)amino)-1,3,5-triazin-2-yl)amino)propanoate (TA1033) and tert-butyl (S)-4-(3-((4-((3-(4-cyanophenyl)-1-methoxy-1-oxopropan-2-yl)amino)-6-((2-methoxyethyl)amino)-1,3,5-triazin-2-yl)amino)benzyl)piperazine-1-carboxylate (TA1041):

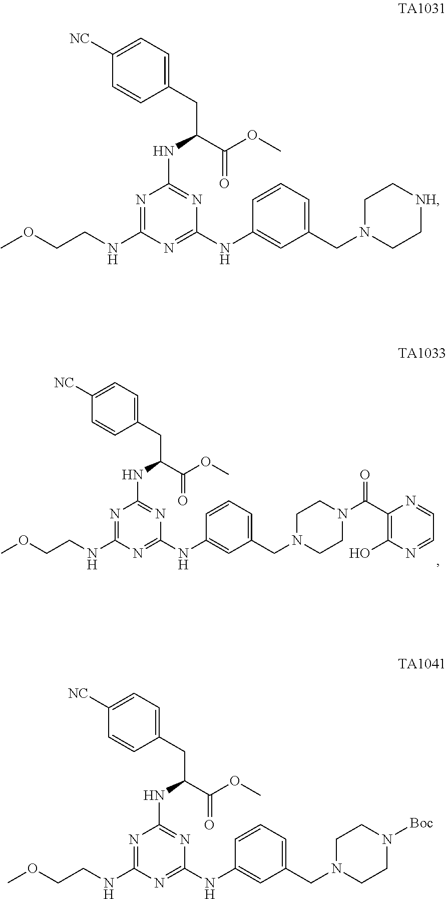 [SMILES: COCCNc1nc(Nc2cccc(CN3CCNCC3)c2)nc(N[C@@H](Cc2ccc(C#N)cc2)C(=O)OC)n1 ; COCCNc1nc(Nc2cccc(CN3CCN(C(=O)c4nccnc4O)CC3)c2)nc(N[C@@H](Cc2ccc(C#N)cc2)C(=O)OC)n1 ; COCCNc1nc(Nc2cccc(CN3CCN(C(=O)OC(C)(C)C)CC3)c2)nc(N[C@@H](Cc2ccc(C#N)cc2)C(=O)OC)n1]

were prepared similarly to these methods, replacing (5-fluoro-1H-benzimidazol-2-yl)methanamine dihydrochloride salt I-5 with 2-methoxyethan-1-amine in step 2.

TA1033: ¹H NMR (400 MHz, DMSO-d₆) δ 12.60 (s, 1H), 9.15-8.75 (m, 1H), 7.76 (d, J=7.9 Hz, 3H), 7.50 (d, J=8.0 Hz, 3H), 7.43-7.06 (m, 3H), 6.82 (dd, J=59.6, 13.2 Hz, 2H), 4.81-4.53 (m, 1H), 3.63 (m, 5H), 3.45 (m, 10H), 3.30-2.98 (m, 8H). MS (m/z): 669 [M+1]⁺, LCMS purity: 95%.

The compounds methyl (5)-3-(4-cyanophenyl)-2-((4-(((1-methyl-1H-imidazol-2-yl)methyl)amino)-6-((3-(piperazin-1-ylmethyl)phenyl)amino)-1,3,5-triazin-2-yl)amino)propanoate (TA1032), methyl (S)-3-(4-cyanophenyl)-2-((4-((3-((4-(3-hydroxypyrazine-2-carbonyl)piperazin-1-yl)methyl)phenyl)amino)-6-(((1-methyl-1H-imidazol-2-yl)methyl)amino)-1,3,5-triazin-2-yl)amino)propanoate (TA1034) and tert-butyl (S)-4-(3-((4-((3-(4-cyanophenyl)-1-methoxy-1-oxopropan-2-yl)amino)-6-(((1-methyl-1H-imidazol-2-yl)methyl)amino)-1,3,5-triazin-2-yl)amino)benzyl)piperazine-1-carboxylate (TA1042):

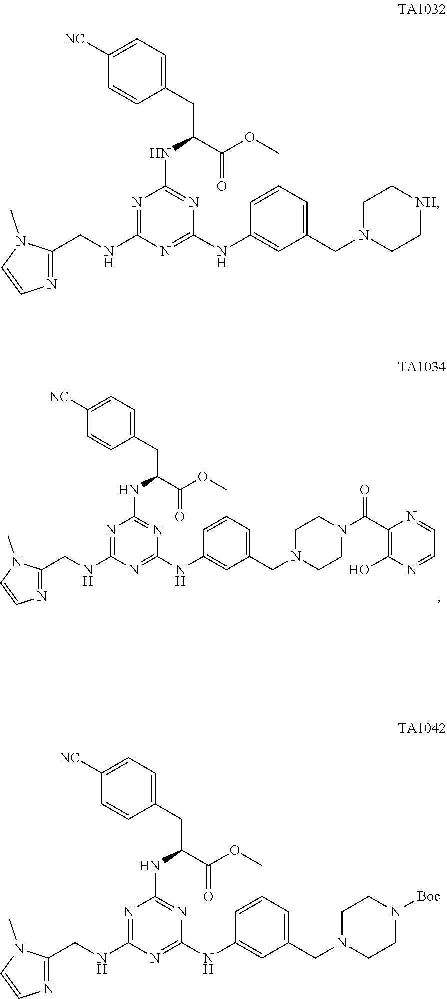 [SMILES: COC(=O)[C@H](Cc1ccc(C#N)cc1)Nc1nc(NCc2nccn2C)nc(Nc2cccc(CN3CCNCC3)c2)n1 ; COC(=O)[C@H](Cc1ccc(C#N)cc1)Nc1nc(NCc2nccn2C)nc(Nc2cccc(CN3CCN(C(=O)c4nccnc4O)CC3)c2)n1 ; COC(=O)[C@H](Cc1ccc(C#N)cc1)Nc1nc(NCc2nccn2C)nc(Nc2cccc(CN3CCN(C(=O)OC(C)(C)C)CC3)c2)n1]

were prepared similarly to these methods, replacing (5-fluoro-1H-benzimidazol-2-yl)methanamine dihydrochloride salt I-5 with (1-methyl-1H-imidazol-2-yl)methanamine (Accela ChemBio, Inc., San Diego, Calif., USA) in step 2.

TA1034: MS (m/z): 705 [M+1]⁺, LCMS purity: <50% (includes byproduct with MW of 610).

The compounds methyl (S)-3-(4-cyanophenyl)-2-((4-((3-((4-(3-hydroxypyrazine-2-carbonyl)piperazin-1-yl)methyl)phenyl)amino)-6-(methylamino)-1,3,5-triazin-2-yl)amino)propanoate (TA1035), tert-butyl (S)-4-(3-((4-((3-(4-cyanophenyl)-1-methoxy-1-oxopropan-2-yl)amino)-6-(methylamino)-1,3,5-triazin-2-yl)amino)benzyl)piperazine-1-carboxylate (TA1043) and methyl (S)-3-(4-cyanophenyl)-2-((4-(methylamino)-6-((3-(piperazin-1-ylmethyl)phenyl)amino)-1,3,5-triazin-2-yl)amino)propanoate (TA1044):

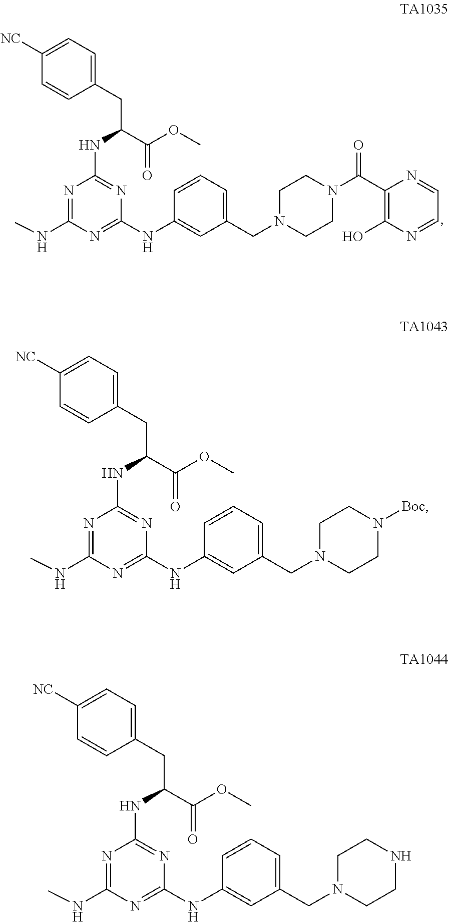 [SMILES: CNc1nc(Nc2cccc(CN3CCN(C(=O)c4nccnc4O)CC3)c2)nc(N[C@@H](Cc2ccc(C#N)cc2)C(=O)OC)n1 ; CNc1nc(Nc2cccc(CN3CCN(C(=O)OC(C)(C)C)CC3)c2)nc(N[C@@H](Cc2ccc(C#N)cc2)C(=O)OC)n1 ; CNc1nc(Nc2cccc(CN3CCNCC3)c2)nc(N[C@@H](Cc2ccc(C#N)cc2)C(=O)OC)n1]

were prepared similarly to these methods, replacing (5-fluoro-1H-benzimidazol-2-yl)methanamine dihydrochloride salt I-5 with methylamine in step 2.

TA1035: ¹H NMR (400 MHz, DMSO-d₆) δ 12.55 (s, 1H), 8.89 (dd, J=59.5, 33.9 Hz, 1H), 7.76 (d, J=8.0 Hz, 2H), 7.50 (s, 3H), 7.43-7.31 (m, 1H), 7.27-7.08 (m, 1H), 6.87 (d, J=7.5 Hz, 1H), 6.83-6.63 (m, 1H), 4.83-4.54 (m, 1H), 3.61 (s, 4H), 3.46 (s, 2H), 3.19 (m, 6H), 2.86-2.61 (m, 3H), 2.50 (m, 6H). MS (m/z): 625 [M+1]⁺, LCMS purity: 98%.

The compounds tert-butyl (S)-4-(3-((4-(benzylamino)-6-((3-(4-cyanophenyl)-1-methoxy-1-oxopropan-2-yl)amino)-1,3,5-triazin-2-yl)amino)benzyl)piperazine-1-carboxylate (TA1048), methyl (S)-2-((4-(benzylamino)-6-((3-(piperazin-1-ylmethyl)phenyl)amino)-1,3,5-triazin-2-yl)amino)-3-(4-cyanophenyl)propanoate (TA1049) and methyl (5)-2-((4-(benzylamino)-6-((3-((4-(3-hydroxypyrazine-2-carbonyl)piperazin-1-yl)methyl)phenyl)amino)-1,3,5-triazin-2-yl)amino)-3-(4-cyanophenyl)propanoate (TA1050):

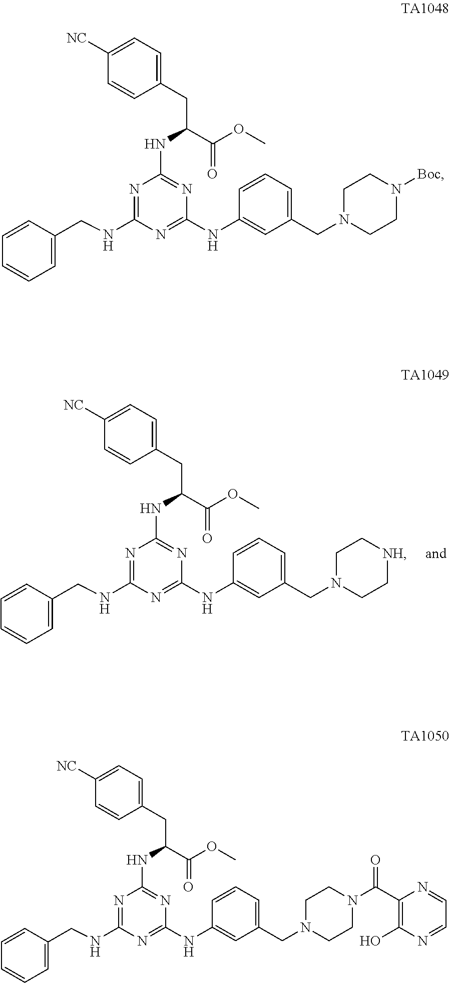

were prepared similarly to these methods, replacing (5-fluoro-1H-benzimidazol-2-yl)methanamine dihydrochloride salt I-5 with phenylmethanamine (SOURCE) in step 2.

TA1049: ¹H NMR (400 MHz, Methanol-d₄) δ 7.77-7.53 (m, 3H), 7.42 (d, J=6.8 Hz, 2H), 7.37-7.28 (m, 4H), 7.29-7.19 (m, 2H), 6.98 (s, 1H), 4.69-4.49 (m, 4H), 3.78-3.46 (m, 5H), 3.25-3.07 (m, 5H), 2.67 (d, J=20.7 Hz, 4H). MS (m/z): 578 [M+1]⁺, LCMS purity: 99.9%.

TA1050: ¹H NMR (400 MHz, Methanol-d₄) δ 8.05-7.66 (m, 2H), 7.59 (dd, J=20.9, 5.9 Hz, 2H), 7.49 (s, 2H), 7.45-7.39 (m, 1H), 7.38-7.27 (m, 4H), 7.27-7.10 (m, 2H), 7.05-6.92 (m, 1H), 5.10-4.90 (m, 2H), 4.57 (d, J=19.4 Hz, 2H), 3.72 (s, 3H), 3.66-3.36 (m, 5H), 3.28-3.00 (m, 2H), 2.69-2.30 (m, 4H). MS (m/z): 700 [M+1]⁺, LCMS purity: 98%.

The compounds tert-butyl (S)-4-(3-((4-((3-(4-cyanophenyl)-1-methoxy-1-oxopropan-2-yl)amino)-6-((2-(6-methoxy-1H-benzo[d]imidazol-2-yl)ethyl)(methyl)amino)-1.3.5-triazin-2-yl)amino)benzyl)piperazine-1-carboxylate (TA1051), methyl (5)-3-(4-cyanophenyl)-2-((4-((2-(6-methoxy-1H-benzo[d]imidazol-2-yl)ethyl)(methyl)amino)-6-((3-(piperazin-1-ylmethyl)phenyl)amino)-1,3,5-triazin-2-yl)amino)propanoate (TA1052), methyl (S)-3-(4-cyanophenyl)-2-((4-((3-((4-(3-hydroxypyrazine-2-carbonyl)piperazin-1-yl)methyl)phenyl)amino)-6-((2-(6-methoxy-1H-benzo[d]imidazol-2-yl)ethyl)(methyl)amino)-1.3.5-triazin-2-yl)amino)propanoate (TA1053), methyl (5)-3-(4-cyanophenyl)-2-((4-((2-(6-methoxy-1H-benzo[d]imidazol-2-yl)ethyl)(methyl)amino)-6-((3-((4-(pyrazine-2-carbonyl)piperazin-1-yl)methyl)phenyl)amino)-1,3,5-triazin-2-yl)amino)propanoate (TA1054), methyl (S)-2-((4-((3-((4-(1,2,3-thiadiazole-4-carbonyl)piperazin-1-yl)methyl)phenyl)amino)-6-((2-(6-methoxy-1H-benzo[d]imidazol-2-yl)ethyl)(methyl)amino)-1,3,5-triazin-2-yl)amino)-3-(4-cyanophenyl)propanoate (TA1055) and methyl (5)-3-(4-cyanophenyl)-2-((4-((2-(6-methoxy-1H-benzo[d]imidazol-2-yl)ethyl)(methyl)amino)-6-((3-((4-((S)-tetrahydrofuran-2-carbonyl)piperazin-1-yl)methyl)phenyl)amino)-1,3,5-triazin-2-yl)amino)propanoate (TA1056):

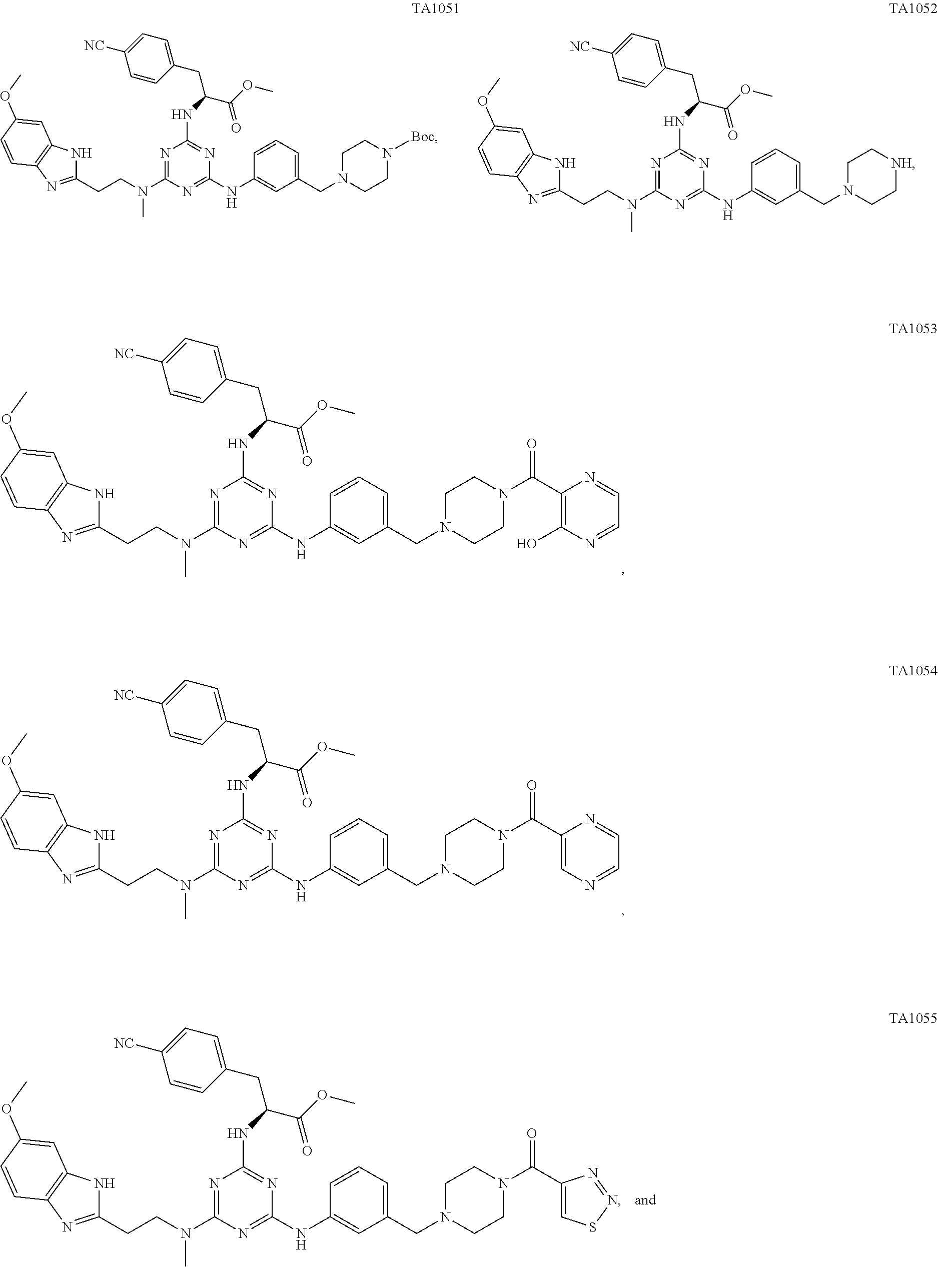

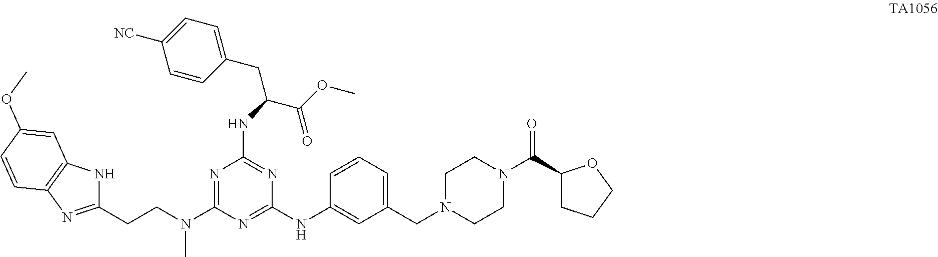 [SMILES: COC(=O)[C@H](Cc1ccc(C#N)cc1)Nc1nc(Nc2cccc(CN3CCN(C(=O)[C@@H]4CCCO4)CC3)c2)nc(N(C)CCc2nc3ccc(OC)cc3[nH]2)n1]

were prepared similarly to these methods, replacing (5-fluoro-1H-benzimidazol-2-yl)methanamine dihydrochloride salt I-5 with 2-(6-methoxy-1H-benzo[d]imidazol-2-yl)-N-methylethan-1-amine (SOURCE) in step 2. For TA1054, TA1055 and TA1056, 3-hydroxypyrazine-2-carboxylic acid I-6 was also replaced with pyrazine-2-carboxylic acid (SOURCE), 1,2,3-thiadiazole-4-carboxylic acid (SOURCE), and (S)-tetrahydrofuran-2-carboxylic acid (SOURCE), respectively, in step 4.

TA1052: ¹H NMR (400 MHz, Methanol-d₄) δ 7.63 (d, J=7.5 Hz, 3H), 7.56-7.36 (m, 4H), 7.25 (t, J=7.7 Hz, 2H), 7.13-6.94 (m, 3H), 4.09 (s, 2H), 3.96-3.73 (m, 4H), 3.71 (s, 3H), 3.59 (s, 2H), 3.38 (s, 3H), 3.32-3.26 (m, 1H), 3.26-3.20 (m, 4H), 3.15 (s, 4H), 2.71 (s, 4H). MS (m/z): 676 [M+1]⁺, LCMS purity: 99.9%.

TA1053: ¹H NMR (400 MHz, Methanol-d₄) δ 7.80 (s, 1H), 7.68-7.56 (m, 2H), 7.49 (s, 2H), 7.45-7.33 (m, 3H), 7.25-7.18 (m, 1H), 7.16-6.89 (m, 3H), 6.85 (d, J=8.4 Hz, 1H), 4.20-3.94 (m, 3H), 3.81 (s, 3H), 3.77-3.64 (m, 4H), 3.61-3.38 (m, 4H), 3.28-3.12 (m, 4H), 3.05 (s, 3H), 2.68-2.33 (m, 5H). MS (m/z): 799 [M+1]⁺, LCMS purity: 99.9%.

TA1054: ¹H NMR (400 MHz, Methanol-d₄) δ 8.85 (s, 1H), 8.70 (d, J=2.5 Hz, 1H), 8.66-8.62 (m, 1H), 7.79 (s, 1H), 7.65-7.56 (m, 2H), 7.46-7.31 (m, 4H), 7.27-7.19 (m, 1H), 7.03-6.91 (m, 2H), 6.85 (d, J=8.0 Hz, 1H), 4.13-4.00 (m, 2H), 3.87-3.65 (m, 9H), 3.63-3.48 (m, 4H), 3.28-3.12 (m, 4H), 3.06 (s, 3H), 2.64-2.42 (m, 4H). MS (m/z): 783 [M+1]⁺, LCMS purity: 100%.

TA1055: ¹H NMR (400 MHz, Methanol-d₄) δ 9.39 (s, 1H), 7.81 (s, 1H), 7.64 (d, J=6.7 Hz, 2H), 7.50-7.30 (m, 3H), 7.29-7.19 (m, 1H), 7.16-6.79 (m, 4H), 4.60 (s, 4H), 4.10 (s, 2H), 3.92-3.74 (m, 5H), 3.70 (s, 2H), 3.64-3.48 (m, 2H), 3.31-3.12 (m, 4H), 3.08 (s, 2H), 2.73-2.46 (m, 4H). MS (m/z): 789 [M+1]⁺, LCMS purity: 99.9%.

TA1056: ¹H NMR (400 MHz, Methanol-d₄) δ 7.80 (s, 1H), 7.70-7.55 (m, 2H), 7.53-7.12 (m, 5H), 7.10-6.81 (m, 3H), 4.75-4.52 (m, 3H), 4.17-3.99 (m, 2H), 3.97-3.90 (m, 1H), 3.90-3.78 (m, 3H), 3.70 (s, 5H), 3.62-3.48 (m, 2H), 3.32-3.13 (m, 4H), 3.09 (s, 2H), 2.65 (s, 4H), 2.17 (h, 7=7.8 Hz, 1H), 2.06 (dq, J=12.6, 7.1 Hz, 1H), 1.94 (ddt, J=14.6, 7.1, 4.3 Hz, 2H). MS (m/z): 775 [M+1]⁺, LCMS purity: 100%.

The compounds tert-butyl (5)-4-(3-((4-(((5-chloro-1H-benzo[₆/]imidazol-2-yl)methyl)amino)-6-((3-(4-cyanophenyl)-1-methoxy-1-oxopropan-2-yl)amino)-1,3,5-triazin-2-yl)amino)benzyl)piperazine-1-carboxylate (TA1057), methyl (5)-2-((4-(((5-chloro-1H-benzo[<7]imidazol-2-yl)methyl)amino)-6-((3-(piperazin-1-ylmethyl)phenyl)amino)-1,3,5-triazin-2-yl)amino)-3-(4-cyanophenyl)propanoate (TA1058), methyl (5)-2-((4-(((5-chloro-1H-benzo[<7]imidazol-2-yl)methyl)amino)-6-((3-((4-(3-hydroxypyrazine-2-carbonyl)piperazin-1-yl)methyl)phenyl)amino)-1,3,5-triazin-2-yl)amino)-3-(4-cyanophenyl)propanoate (TA1059), methyl (5)-2-((4-((3-((4-(3-(1H-pyrazol-1-yl)benzoyl)piperazin-1-yl)methyl)phenyl)amino)-6-(((5-chloro-1H-benzo[d]imidazol-2-yl)methyl)amino)-1,3,5-triazin-2-yl)amino)-3-(4-cyanophenyl)propanoate (TA1060), methyl (5)-2-((4-((3-((4-(1H-1,2,3-triazole-4-carbonyl)piperazin-1-yl)methyl)phenyl)amino)-6-(((5-chloro-1H-benzo[d]imidazol-2-yl)methyl)amino)-1,3,5-triazin-2-yl)amino)-3-(4-cyanophenyl)propanoate (TA1061), methyl (S)-2-((4-(((5-chloro-1H-benzo[d]imidazol-2-yl)methyl)amino)-6-((3-((4-formyl piperazin-1-yl)methyl)phenyl)amino)-1,3,5-triazin-2-yl)amino)-3-(4-cyanophenyl)propanoate and (TA1062):

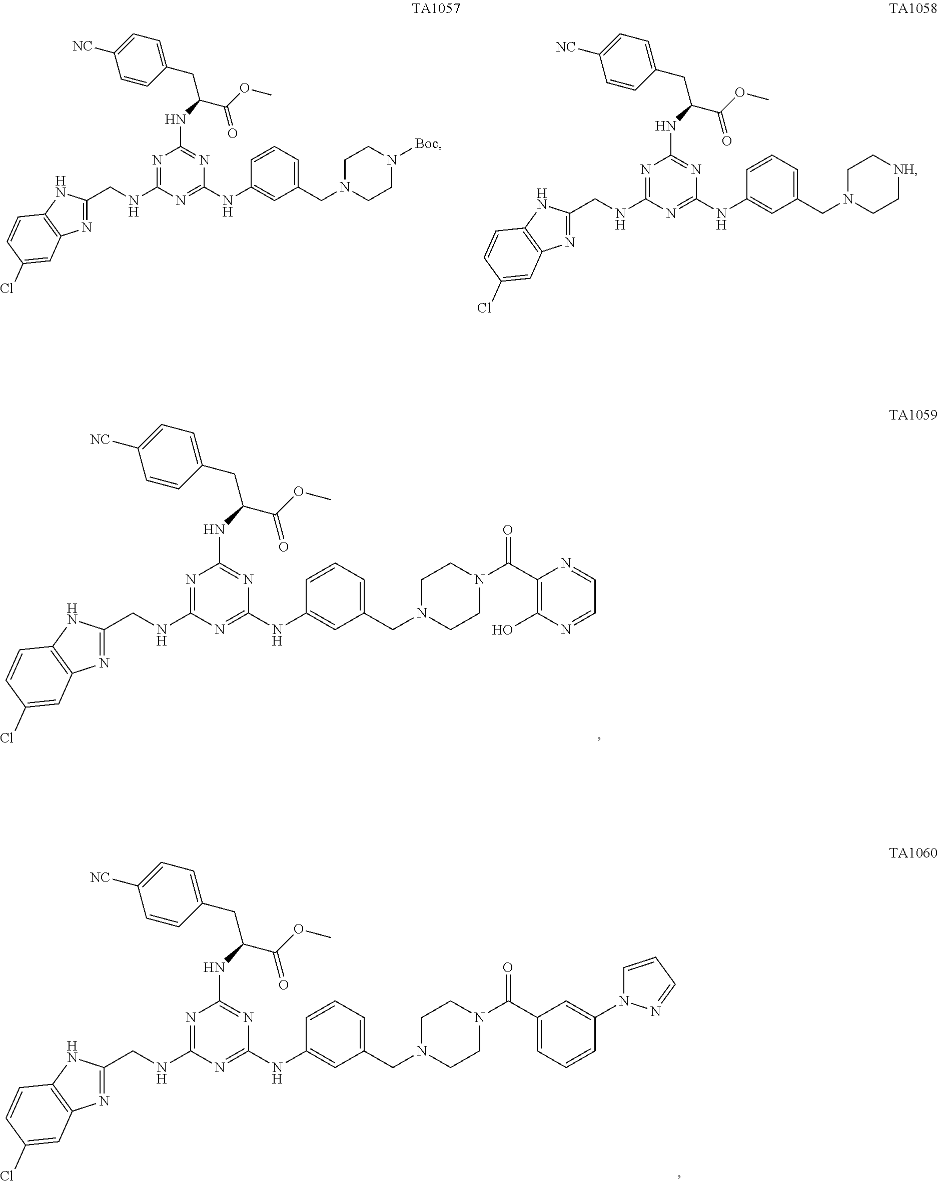 [SMILES: COC(=O)[C@H](Cc1ccc(C#N)cc1)Nc1nc(NCc2nc3cc(Cl)ccc3[nH]2)nc(Nc2cccc(CN3CCN(C(=O)OC(C)(C)C)CC3)c2)n1 ; COC(=O)[C@H](Cc1ccc(C#N)cc1)Nc1nc(NCc2nc3cc(Cl)ccc3[nH]2)nc(Nc2cccc(CN3CCNCC3)c2)n1 ; COC(=O)[C@H](Cc1ccc(C#N)cc1)Nc1nc(NCc2nc3cc(Cl)ccc3[nH]2)nc(Nc2cccc(CN3CCN(C(=O)c4nccnc4O)CC3)c2)n1 ; COC(=O)[C@H](Cc1ccc(C#N)cc1)Nc1nc(NCc2nc3cc(Cl)ccc3[nH]2)nc(Nc2cccc(CN3CCN(C(=O)c4cccc(-n5cccn5)c4)CC3)c2)n1]

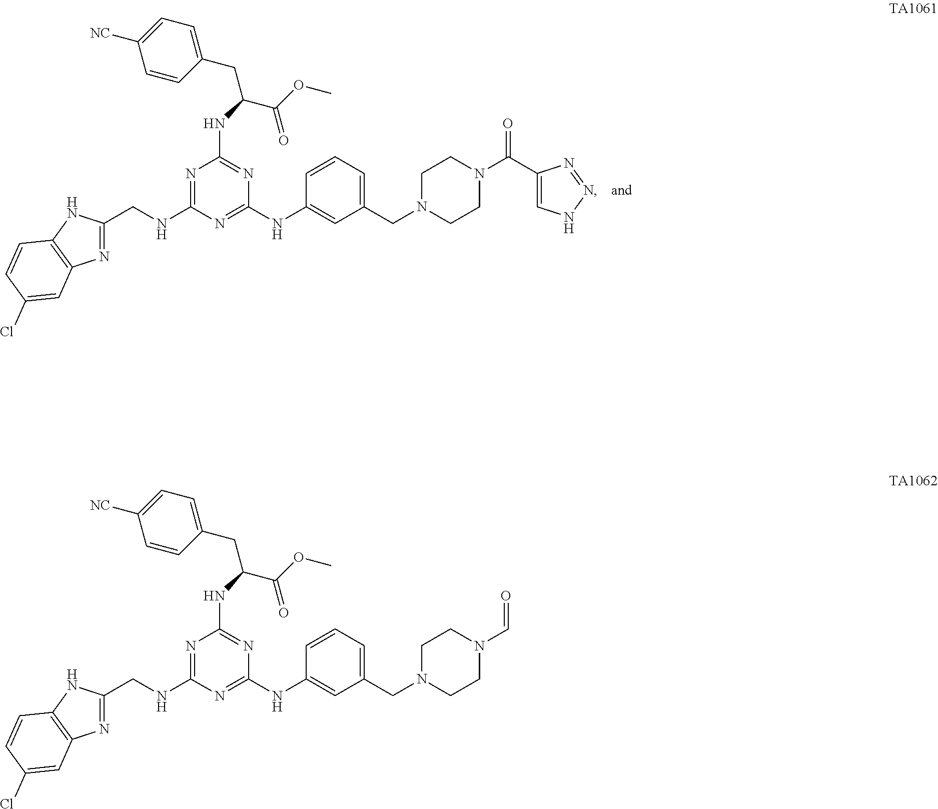 [SMILES: COC(=O)[C@H](Cc1ccc(C#N)cc1)Nc1nc(NCc2nc3cc(Cl)ccc3[nH]2)nc(Nc2cccc(CN3CCN(C(=O)c4c[nH]nn4)CC3)c2)n1 ; COC(=O)[C@H](Cc1ccc(C#N)cc1)Nc1nc(NCc2nc3cc(Cl)ccc3[nH]2)nc(Nc2cccc(CN3CCN(C=O)CC3)c2)n1]

were prepared similarly to these methods, replacing (5-fluoro-1H-benzimidazol-2-yl)methanamine dihydrochloride salt I-5 with (5-chloro-1H-benzimidazol-2-yl)methanamine (SOURCE) in step 2, and for TA1060 and TA1061, also replacing 3-hydroxypyrazine-2-carboxylic acid I-6 with 3-(1H-pyrazol-1-yl)benzoic acid (SOURCE), and 1H-1,2,3-triazole-4-carboxylic acid (SOURCE), respectively, in step 4. TA1062 was an isolated by-product of these reactions.

TA1058: ¹H NMR (400 MHz, Methanol-d₄) δ 8.54 (s, 1H), 7.76-7.32 (m, 7H), 7.32-7.05 (m, 3H), 7.03-6.88 (m, 1H), 5.09-4.93 (m, 2H), 4.83-4.70 (m, 2H), 3.72 (s, 2H), 3.64-3.41 (m, 3H), 3.26-3.06 (m, 4H), 3.03-2.82 (m, 1H), 2.76-2.43 (m, 4H). MS (m/z): 653 [M+1]⁺, LCMS purity: 99.9%,

TA1059: ¹H NMR (400 MHz, Methanol-d₄) δ 7.76-7.60 (m, 2H), 7.59-7.36 (m, 6H), 7.33-7.09 (m, 3H), 7.03-6.87 (m, 1H), 4.82-4.72 (m, 2H), 3.83-3.65 (m, 4H), 3.60-3.54 (m, 1H), 3.53-3.46 (m, 1H), 3.43-3.35 (m, 2H), 3.28-3.05 (m, 2H), 2.67-2.35 (m, 4H), 1.72-1.49 (m, 2H), 1.36-1.29 (m, 3H), 1.03-0.83 (m, 3H). MS (m/z): 774 [M+1]⁺, LCMS purity: 97%,

TA1060: ¹H NMR (400 MHz, Methanol-d₄) δ 8.40 (s, 1H), 8.33-8.26 (m, 1H), 7.99 (d, J=7.9 Hz, 1H), 7.93-7.82 (m, 2H), 7.80-7.72 (m, 1H), 7.72-7.31 (m, 9H), 7.28-7.07 (m, 3H), 7.05-6.88 (m, 1H), 6.56 (d, J=5.8 Hz, 1H), 4.99 (s, 1H), 4.77 (d, J=23.5 Hz, 2H), 4.02-3.35 (m, 10H), 3.25-2.89 (m, 2H), 2.80-2.17 (m, 5H). MS (m/z): 823 [M+1]⁺, LCMS purity: 85%,

TA1061: ¹H NMR (400 MHz, Methanol-d₄) δ 8.14 (s, 1H), 7.60 (m, 3H), 7.50 (m, 1H), 7.44 (m, 1H), 7.39 (m, 2H), 7.29-7.05 (m, 3H), 6.98 (m, 1H), 4.76 (m, 3H), 4.58 (s, 1H), 3.98 (m, 2H), 3.73 (m, 4H), 3.51 (m, 2H), 3.23-2.87 (m, 2H), 2.47 (m, 4H). MS (m/z): 747 [M+1]⁺, LCMS purity: 98.8%,

TA1062: ¹H NMR (400 MHz, Methanol-d₄) δ 8.00 (s, 1H), 7.61 (d, J=11.4 Hz, 2H), 7.53 (s, 1H), 7.48 (d, 7=8.3 Hz, 1H), 7.42 (m, 2H), 7.20 (d, 7=8.5 Hz, 2H), 7.17-7.06 (m, 1H), 6.98 (m, 1H), 4.98 (m, 2H), 4.85-4.54 (m, 3H), 3.71 (s, 2H), 3.57-3.42 (m, 4H), 3.27-2.87 (m, 2H), 2.59-2.17. MS (m/z): 680 [M+1]⁺, LCMS purity: 99.7%

The compounds tert-butyl (5)-4-(3-((4-(((5-fluoro-1H-benzo[7]imidazol-2-yl)methyl)amino)-6-((1-methoxy-1-oxo-3-phenylpropan-2-yl)amino)-1,3,5-triazin-2-yl)amino)benzyl)piperazine-1-carboxylate (TA1063), methyl (4-(((5-fluoro-1H-benzo[<7]imidazol-2-yl)methyl)amino)-6-((3-(piperazin-1-ylmethyl)phenyl)amino)-1,3,5-triazin-2-yl)phenylalaninate (TA1064) and methyl (4-(((5-fluoro-1H-benzo[d]imidazol-2-yl)methyl)amino)-6-((3-((4-(3-hydroxypyrazine-2-carbonyl)piperazin-1-yl)methyl)phenyl)amino)-1,3,5-triazin-2-yl)-L-phenylalaninate (TA1065):

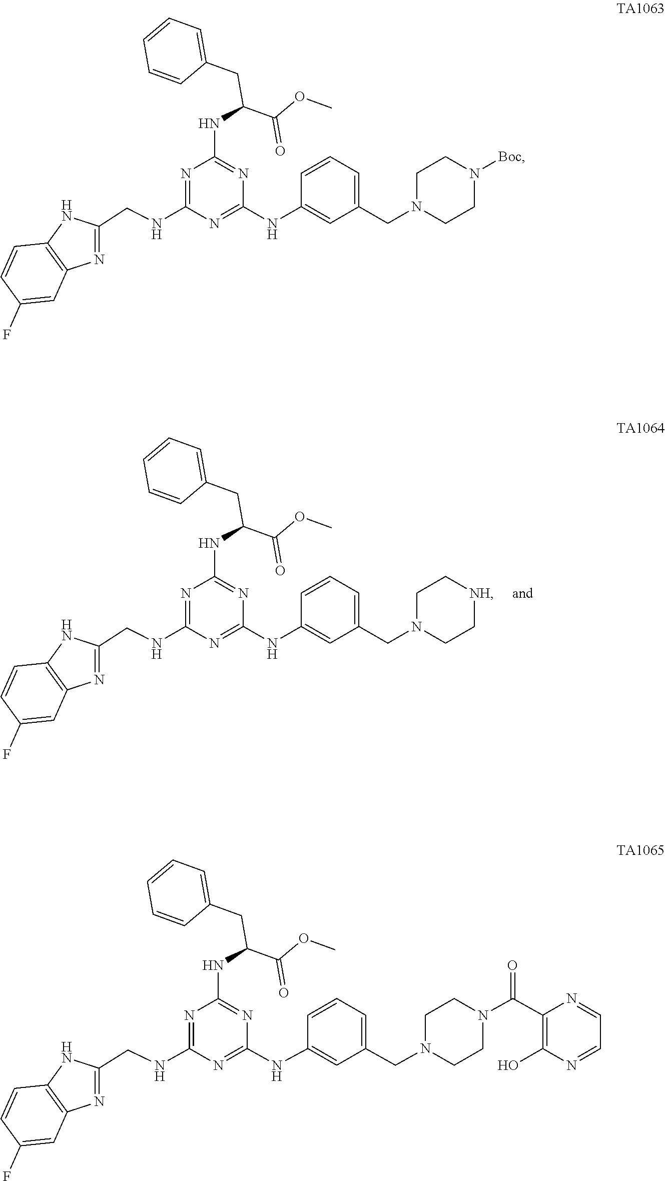

were prepared similarly to these methods, replacing Int. 5C with methyl L-phenylalaninate in step 1.

TA1064: ¹H NMR (400 MHz, Methanol-d₄) δ 7.78-7.54 (m, 2H), 7.50 (dd, J=8.7, 4.6 Hz, 1H), 7.44-7.09 (m, 8H), 7.07-6.90 (m, 2H), 3.92-3.56 (m, 4H), 3.55-3.34 (m, 3H), 3.32-3.06 (m, 6H), 3.04-2.85 (m, 1H), 2.77-2.52 (m, 4H). MS (m/z): 611 [M+1]⁺, LCMS purity: 98%,

TA1065: ¹H NMR (400 MHz, Methanol-d₄) δ 7.78-7.58 (m, 1H), 7.48 (s, 3H), 7.38-7.10 (m, 7H), 7.09-6.86 (m, 3H), 4.78 (s, 3H), 3.90-3.36 (m, 9H), 3.29-2.84 (m, 3H), 2.76-2.31 (m, 4H). MS (m/z): 733 [M+1]⁺, LCMS purity: 98%.

The compounds methyl (5)-3-(4-cyanophenyl)-2-((4-(((5-fluoro-1H-benzo[d]imidazol-2-yl) methyl)amino)-6-((3-((4-(3-hydroxypyridazine-4-carbonyl)piperazin-1-yl)methyl)phenyl)amino)-1,3,5-triazin-2-yl)amino)propanoate (TA1066), methyl (5)-3-(4-cyanophenyl)-2-((4-(((5-fluoro-1H-benzo[d]imidazol-2-yl)methyl)amino)-6-((3-((4-(3-methoxypyrazine-2-carbonyl)piperazin-1-yl)methyl)phenyl)amino)-1,3,5-triazin-2-yl)amino)propanoate (TA1078), and methyl (5)-3-(4-cyanophenyl)-2-((4-(((5-fluoro-1H-benzo[d]imidazol-2-yl)methyl)amino)-6-((3-((4-(1-(methylsulfonyl)piperidine-4-carbonyl)piperazin-1-yl)methyl)phenyl)amino)-1,3,5-triazin-2-yl)amino)propanoate (TA1079):

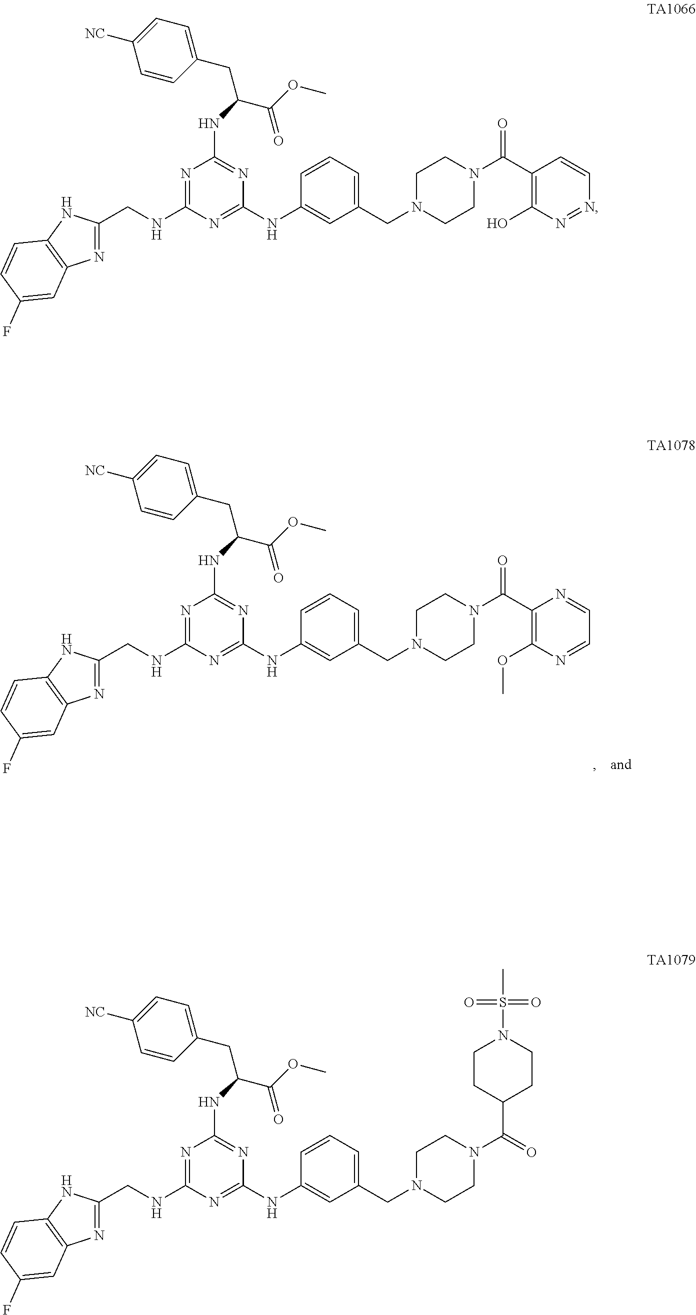 [SMILES: COC(=O)[C@H](Cc1ccc(C#N)cc1)Nc1nc(NCc2nc3cc(F)ccc3[nH]2)nc(Nc2cccc(CN3CCN(C(=O)c4ccnnc4O)CC3)c2)n1 ; COC(=O)[C@H](Cc1ccc(C#N)cc1)Nc1nc(NCc2nc3cc(F)ccc3[nH]2)nc(Nc2cccc(CN3CCN(C(=O)c4nccnc4OC)CC3)c2)n1 ; COC(=O)[C@H](Cc1ccc(C#N)cc1)Nc1nc(NCc2nc3cc(F)ccc3[nH]2)nc(Nc2cccc(CN3CCN(C(=O)C4CCN(S(C)(=O)=O)CC4)CC3)c2)n1]

were prepared similarly to these methods, replacing 3-hydroxypyrazine-2-carboxylic acid I-6 with 3-hydroxypyridazine-4-carboxylic acid (SOURCE), 3-methoxypyrazine-2-carboxylic acid (Ark Pharm, Arlington Heights, Ill., USA), and l-(methylsulfonyl)piperidine-4-carboxylic acid (SOURCE), respectively, in step 4.

TA1066: ¹H NMR (400 MHz, Methanol-d₄) δ 8.00 (d, J=4.0 Hz, 1H), 7.78-7.56 (m, 3H), 7.53-7.38 (m, 4H), 7.32-7.13 (m, 3H), 7.08-6.93 (m, 2H), 4.98 (s, 1H), 4.82-4.74 (m, 2H), 3.86-3.65 (m, 5H), 3.65-3.57 (m, 1H), 3.56-3.37 (m, 4H), 3.26-3.06 (m, 2H), 2.86-2.56 (m, 4H). MS (m/z): 758 [M+1]⁺, LCMS purity: 94%.

TA1078: ¹H NMR (400 MHz, Methanol-d₄). MS (m/z): [M+1]⁺, LCMS purity: %,

TA1079: ¹H NMR (400 MHz, Methanol-d₄). MS (m/z): [M+1]⁺, LCMS purity: %.

The compounds tert-butyl (5)-4-(3-((4-(((1H-tetrazol-5-yl)methyl)amino)-6-((3-(4-cyanophenyl)-1-methoxy-1-oxopropan-2-yl)amino)-1,3,5-triazin-2-yl)amino)benzyl)piperazine-1-carboxylate (TA1067), methyl (5)-2-((4-(((1H-tetrazol-5-yl)methyl)amino)-6-((3-(piperazin-1-ylmethyl)phenyl)amino)-1,3,5-triazin-2-yl)amino)-3-(4-cyanophenyl)propanoate (TA1068) and methyl (5)-2-((4-(((1H-tetrazol-5-yl)methyl)amino)-6-((3-((4-(3-hydroxypyrazine-2-carbonyl)piperazin-1-yl)methyl)phenyl)amino)-1,3,5-triazin-2-yl)amino)-3-(4-cyanophenyl)propanoate (TA1069):

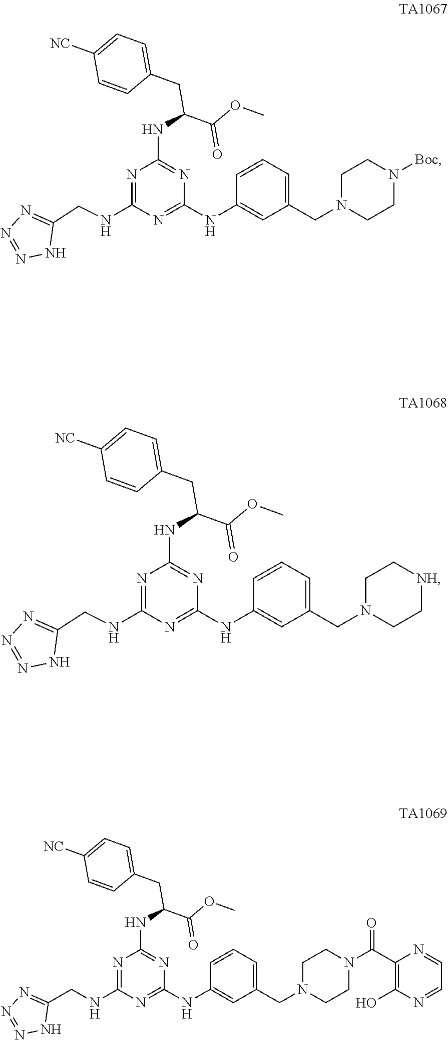 [SMILES: COC(=O)[C@H](Cc1ccc(C#N)cc1)Nc1nc(NCc2nnn[nH]2)nc(Nc2cccc(CN3CCN(C(=O)OC(C)(C)C)CC3)c2)n1 ; COC(=O)[C@H](Cc1ccc(C#N)cc1)Nc1nc(NCc2nnn[nH]2)nc(Nc2cccc(CN3CCNCC3)c2)n1 ; COC(=O)[C@H](Cc1ccc(C#N)cc1)Nc1nc(NCc2nnn[nH]2)nc(Nc2cccc(CN3CCN(C(=O)c4nccnc4O)CC3)c2)n1]

were prepared similarly to these methods, replacing (5-fluoro-1H-benzimidazol-2-yl)methanamine dihydrochloride salt I-5 with (1H-tetrazol-5-yl)methanamine (SOURCE) in step 2.

TA1068: MS (m/z): 570 [M+1]⁺, LCMS purity: 98.3%, TA1069: ¹H NMR (400 MHz, Methanol-d₄) δ 8.19 (s, 1H), 8.00-7.80 (m, 2H), 7.69-7.54 (m, 3H), 7.53-7.35 (m, 3H), 7.29-7.16 (m, 2H), 7.00-6.91 (m, 1H), 3.72 (s, 3H), 3.63-3.44 (m, 3H), 3.25-3.07 (m, 6H), 2.66 (s, 5H), 2.18 (s, 1H), 2.06 (s, 1H). MS (m/z): 692 [M+1]⁺, LCMS purity: 97.5%.

The compound 2-(2-methoxyethoxy)ethyl (5)-3-(4-cyanophenyl)-2-((4-(((5-fluoro-1H-benzo[d]imidazol-2-yl)methyl)amino)-6-((3-((4-(3-hydroxypyrazine-2-carbonyl)piperazin-1-yl)methyl)phenyl)amino)-1,3,5-triazin-2-yl)amino)propanoate (TA1070):

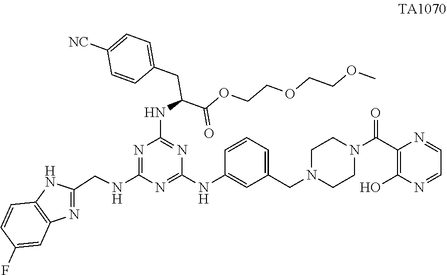 [SMILES: COCCOCCOC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1]

was prepared similarly to these methods, replacing Int. 5C with Int. 7C in step 1.

TA1066: ¹H NMR (400 MHz, Methanol-d₄) δ 7.76-7.54 (m, 3H), 7.54-7.39 (m, 4H), 7.23 (td, J=34.8, 32.3, 7.9 Hz, 4H), 7.05-6.93 (m, 2H), 4.82-4.70 (m, 3H), 4.59 (s, 1H), 4.36-3.97 (m, 5H), 3.83-3.63 (m, 4H), 3.61-3.52 (m, 3H), 3.52-3.41 (m, 4H), 3.29 (s, 2H), 3.27-3.20 (m, 1H), 3.16-3.08 (m, 1H), 3.05-2.95 (m, 1H), 2.65-2.32 (m, 5H). MS (m/z): 847 [M+1]⁺, LCMS purity: 96%.

The compound methyl (5)-3-cyclohexyl-2-((4-(((5-fluoro-1H-benzo[₆/]imidazol-2-yl)methyl)amino)-6-((3-((4-(3-hydroxypyrazine-2-carbonyl)piperazin-1-yl)methyl)phenyl)amino)-1,3,5-triazin-2-yl)amino)propanoate (TA1072):

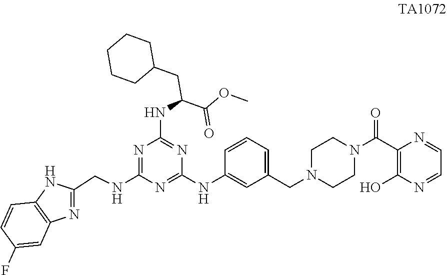 [SMILES: COC(=O)[C@H](CC1CCCCC1)Nc1nc(NCc2nc3cc(F)ccc3[nH]2)nc(Nc2cccc(CN3CCN(C(=O)c4nccnc4O)CC3)c2)n1]

was prepared similarly to these methods, replacing Int. 5C with methyl (5)-2-amino-3-cyclohexylpropanoate (SOURCE) in step 1.

TA1072: ¹H NMR (400 MHz, Methanol-d₄) δ 7.94 (s, 1H), 7.84-7.63 (m, 1H), 7.53 (q, J=4.0 Hz, 3H), 7.40-7.19 (m, 2H), 7.16-6.97 (m, 2H), 4.69 (s, 1H), 4.18-3.81 (m, 4H), 3.78-3.36 (m, 5H), 3.20-2.91 (m, 4H), 1.91-1.43 (m, 7H), 1.37-0.67 (m, 7H). MS (m/z): 739 [M+1]⁺, LCMS purity: 100%.

### Example 3—Methyl (S)-3-(4-cyanophenyl)-2-((4-((3-((4-(3-hydroxypyrazine-2-carbonyl)piperazin-1-yl)methyl)phenyl)amino)-1,3,5-triazin-2-yl)amino)propanoate (TA1026)

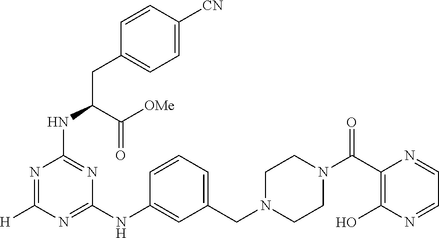 [SMILES: COC(=O)[C@H](Cc1ccc(C#N)cc1)Nc1ncnc(Nc2cccc(CN3CCN(C(=O)c4nccnc4O)CC3)c2)n1]

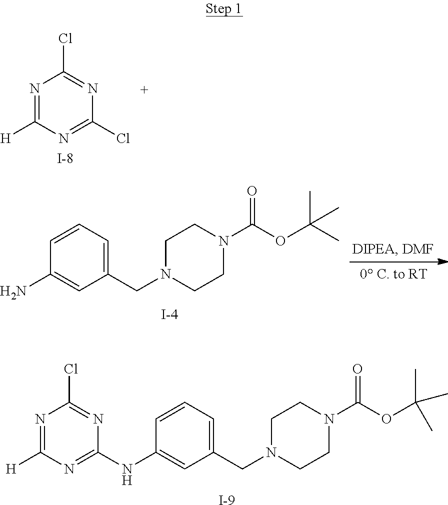

Preparation of tert-butyl 4-(3-((4-chloro-1,3,5-triazin-2-yl)amino)benzyl)piperazine-1-carboxylate (I-9). Prepared according to the procedure described in WO 02/083653. To a stirring solution of 2,4-dichloro-1,3,5-triazine (I-8, 1 equiv., 0.52 g, 3.50 mmol, AstaTech, Inc., Bristol, Pa., USA) in anhydrous DMF (10 mL) at 0° C. were added DIPEA (1.05 equiv., 0.65 mL, 3.68 mmol), followed by tert-butyl 4-[(3-aminophenyl)methyl]piperazine-1-carboxylate (I-4, 1 equiv., 1.02 g, 3.50 mmol). The reaction mixture was stirred at 0° C. for 30 minutes and warmed to r.t. After 12 h of reaction at r.t., the solution was concentrated to dryness, and the crude residue was purified on a combi-flash ISCO purified by column chromatography (EtOAc/heptane 5:5 to 7:3) to afford the desired compound I-9 (1.03 g, 73%) as a white solid.

### Step 2

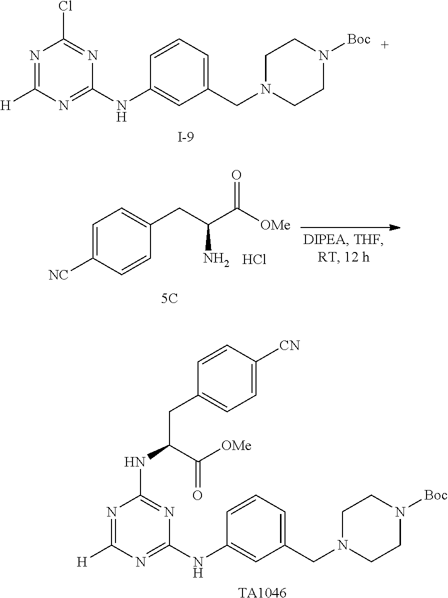

Preparation of tert-butyl (S)-4-(3-((4-((3-(4-cyanophenyl)-1-methoxy-1-oxopropan-2-yl)amino)-1,3,5-triazin-2-yl)amino)benzyl)piperazine-1-carboxylate (TA1046).

To a solution of tert-butyl 4-(3-((4-chloro-1,3,5-triazin-2-yl)amino)benzyl)piperazine-1-carboxylate (I-9, 1 equiv., 36.5 mg, 0.090 mmol), methyl (S)-2-amino-3-(4-cyanophenyl)propanoate hydrochloride (5C, 2 equiv., 43.4 mg, 0.18 mmol) in anhydrous THF (0.60 mL) was added DIPEA (86 μL) and the solution was stirred at 70° C. for 12 h. The reaction mixture was then concentrated under reduced pressure. The crude residue (TA1046) was used for the next step without further purification.

### Step 3

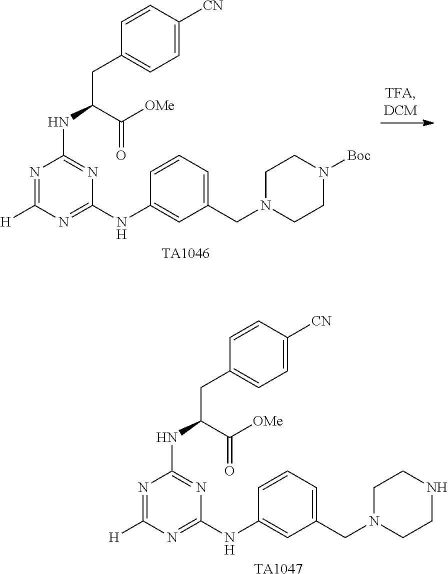

Preparation of methyl (S)-3-(4-cyanophenyl)-2-((4-((3-(piperazin-1-ylmethyl)phenyl)amino)-1,3,5-triazin-2-yl)amino)propanoate (TA1047). To a solution of TA1046 (1 equiv., 0.14 g, 0.24 mmol) in DCM (0.08-0.50M) was added dropwise TFA (TFA/DCM=1:1) at 0° C. The mixture was stirred for 30 minutes and concentrated in vacuo. The crude residue TA1047 was used for the next step without further purification.

The compounds TA1010 and TA1011 were prepared similarly to steps 1-3 replacing methyl (S)-2-amino-3-(4-cyanophenyl)propanoate hydrochloride 5C with (S)-2-amino-3-(4-cyanophenyl)-N,N-dimethylpropanamide 1A in step 2. TA1011: MS (m/z): 486 [M+1]⁺,

LCMS purity: 95%.

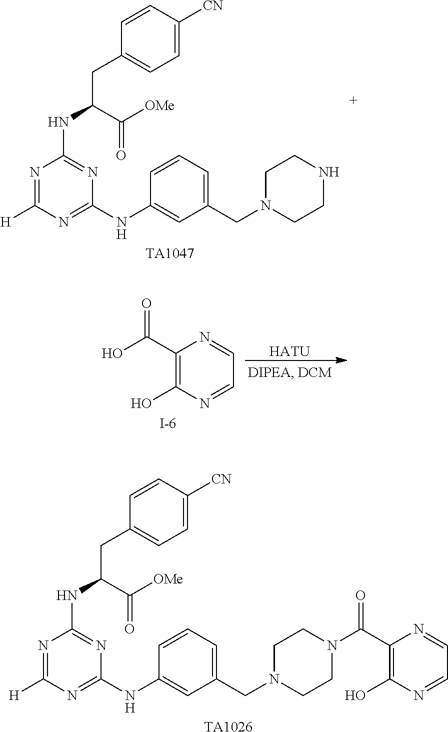

### Step 4

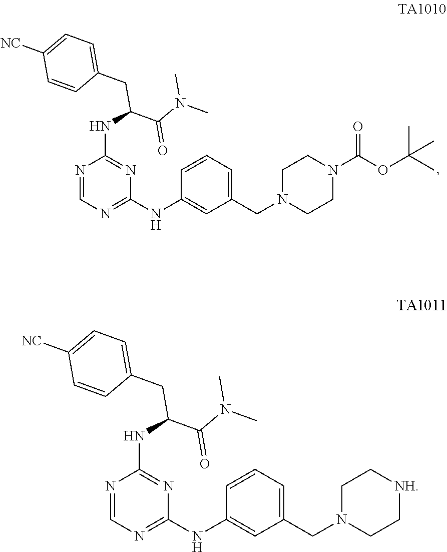 [SMILES: CN(C)C(=O)[C@H](Cc1ccc(C#N)cc1)Nc1ncnc(Nc2cccc(CN3CCN(C(=O)OC(C)(C)C)CC3)c2)n1 ; CN(C)C(=O)[C@H](Cc1ccc(C#N)cc1)Nc1ncnc(Nc2cccc(CN3CCNCC3)c2)n1]

Preparation of methyl (S)-3-(4-cyanophenyl)-2-((4-((3-((4-(3-hydroxypyrazine-2-carbonyl)piperazin-1-yl)methyl)phenyl)amino)-1,3,5-triazin-2-yl)amino)propanoate (TA1026). HATU (1.2 equiv., 45 mg, 0.12 mmol) was added to a solution of 3-hydroxypyrazine-2-carboxylic acid (I-6, 1.1 equiv., 15 mg, 0.11 mmol) in anhydrous DCM (1.5 mL) and DIPEA (3 equiv., 0.040 mL, 0.23 mmol). After being stirred at r.t. 10 minutes, TA1047 (1 equiv., 46 mg, 0.098 mmol) was added. The resulting suspension was stirred at r.t. 1 h, followed by DMF (0.1 mL). Then the reaction was stirred for another 2 h until the LCMS analysis showed complete consumption of the starting material. The crude residue was then purified by reverse phase preparative HPLC (XBridge BEH, 19×150 mm, 5 μm, C18 column; ACN/water with 0.1% formic acid modifier, 20 mL/min), affording Compound TA1026 (15.1 mg, 25%) as a yellow solid. ¹H NMR (400 MHz, Methanol-d₄) δ 8.12 (d, J=10.2 Hz, 1H), 7.71-7.65 (m, 2H), 7.61 (d, J=8.0 Hz, 2H), 7.49 (s, 2H), 7.43 (d, J=7.9 Hz, 3H), 7.34-7.27 (m, 1H), 7.08 (dd, J=17.7, 7.0 Hz, 1H), 4.96 (dd, J=8.6, 5.8 Hz, 1H), 3.83-3.77 (m, 2H), 3.71 (s, 2H), 3.60 (s, 2H), 3.43-3.39 (m, 3H), 3.25-3.14 (m, 3H), 2.58 (dt, J=36.9, 4.7 Hz, 5H). MS (m/z): 595 [M+1]⁺, LCMS purity: 99%.

### Example 4: Additional Compounds

Compounds of the invention as described herein can be prepared using the methods described in Examples 1-3, or similar methods with modifications readily available to one skilled in the art. For example, compounds (S)-3-(4-cyanophenyl)-2-((4-(((5-fluoro-1H-benzo[d]imidazol-2-yl)methyl)amino)-6-((3-((4-formylpiperazin-1-yl)methyl)phenyl)amino)-1,3,5-triazin-2-yl)amino)-N,N-dimethylpropanamide TA 1016, methyl (S)-3-(4-cyanophenyl)-2-((4-methoxy-6-((3-(piperazin-1-ylmethyl)phenyl)amino)-1,3,5-triazin-2-yl)amino)propanoate TA1028 (5)-3-(4-cyanophenyl)-2-((4-(((5-fluoro-1H-benzo[d]imidazol-2-yl)methyl)amino)-6-((3-((4-formylpiperazin-1-yl)methyl)phenyl)amino)-1,3,5-triazin-2-yl)amino)-N-methylpropanamide TA1030, methyl (S)-3-(4-cyanophenyl)-2-((4-((3-((4-(3-hydroxypyrazine-2-carbonyl)piperazin-1-yl)methyl)phenyl)amino)-6-methoxy-1,3,5-triazin-2-yl)amino)propanoate TA1074, methyl (5)-2-((4-((3-((4-acetylpiperazin-1-yl)methyl)phenyl)amino)-6-(((5-fluoro-1H-benzo[<7]imidazol-2-yl)methyl)amino)-1,3,5-triazin-2-yl)amino)-3-(4-cyanophenyl)propanoate TA1075, methyl (5)-2-((4-((3-((4-acetylpiperazin-1-yl)methyl)phenyl)amino)-6-(A-((5-fluoro-1H-benzo[d]imidazol-2-yl)methyl)acetamido)-1,3,5-triazin-2-yl)amino)-3-(4-cyanophenyl)propanoate TA1076, and methyl (S)-3-(4-cyanophenyl)-2-((4-((3-((4-formylpiperazin-1-yl)methyl)phenyl)amino)-6-methoxy-1,3,5-triazin-2-yl)amino)propanoate TA1077, were prepared following similar methods.

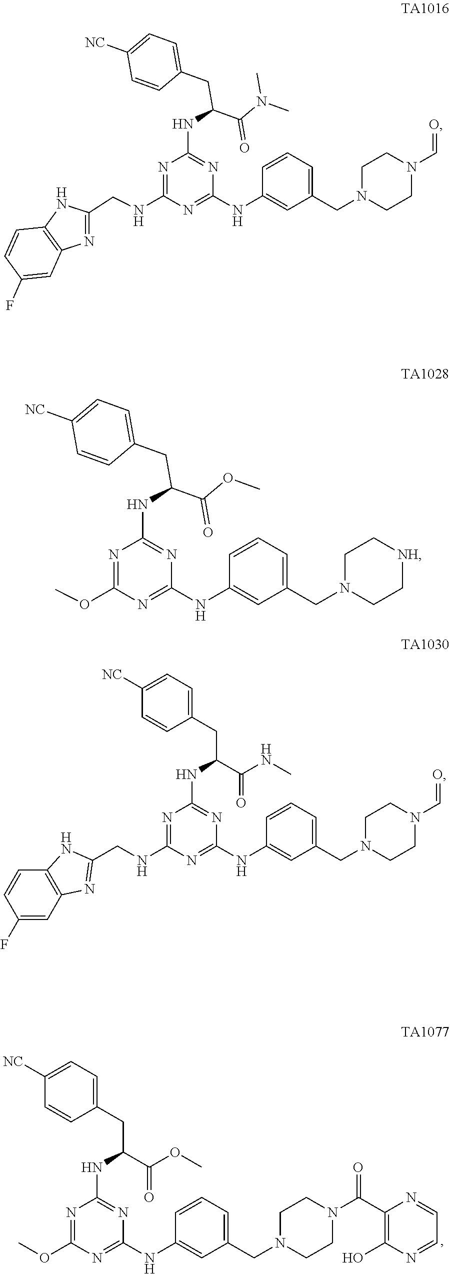 [SMILES: CN(C)C(=O)[C@H](Cc1ccc(C#N)cc1)Nc1nc(NCc2nc3cc(F)ccc3[nH]2)nc(Nc2cccc(CN3CCN(C=O)CC3)c2)n1 ; COC(=O)[C@H](Cc1ccc(C#N)cc1)Nc1nc(Nc2cccc(CN3CCNCC3)c2)nc(OC)n1 ; CNC(=O)[C@H](Cc1ccc(C#N)cc1)Nc1nc(NCc2nc3cc(F)ccc3[nH]2)nc(Nc2cccc(CN3CCN(C=O)CC3)c2)n1 ; COC(=O)[C@H](Cc1ccc(C#N)cc1)Nc1nc(Nc2cccc(CN3CCN(C(=O)c4nccnc4O)CC3)c2)nc(OC)n1]

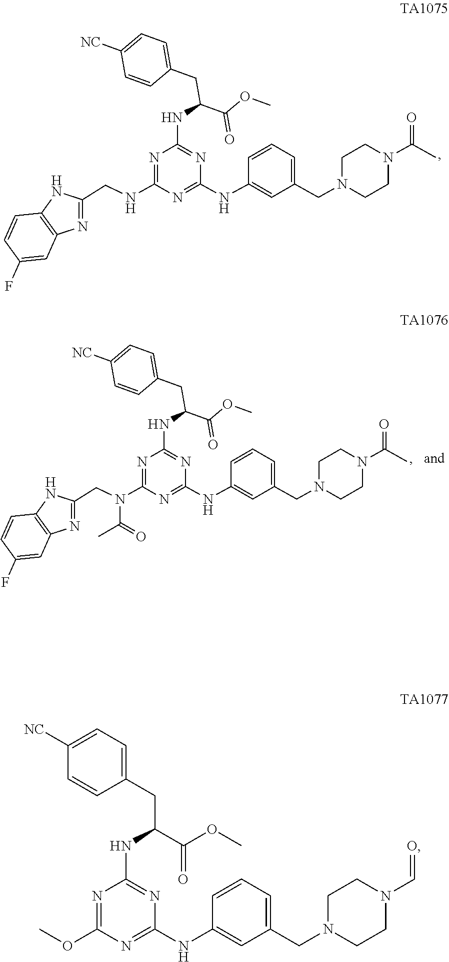

TA1074: ¹H NMR (400 MHz, Methanol-d₄) δ 7.73-7.58 (m, 4H), 7.55-7.41 (m, 4H), 7.33-7.24 (m, 2H), 7.12-7.01 (m, 2H), 3.91 (d, J=10.0 Hz, 2H), 3.83-3.75 (m, 2H), 3.75-3.70 (m, 2H), 3.62-3.52 (m, 3H), 3.52-3.45 (m, 2H), 3.42-3.37 (m, 2H), 3.23-3.13 (m, 3H), 2.65-2.57 (m, 2H), 2.49 (dt, J=19.9, 5.1 Hz, 3H). MS (m/z): 625 [M+1]⁺, LCMS purity: 98.5%.

### Example 5: Activity Assay

IC₅₀ values were determined as follows:

Human cGAS sequence encoding amino acids 155-522 was cloned into a pET (EMD Millipore) based expression plasmid. The resulting construct contained a tandem N-terminal hexahistidine tag, maltose binding protein fusion followed by a tobacco etch virus protease cleavable linker preceding cGAS amino acids 155-522.

Construct sequence: Amino acids 155-522, Human cGAS

SEQ. ID No. 1
DAAPGASKLRAVLEKLKLSRDDISTAAGMVKGVVDHLLLRLKCDSAFRG
VGLLNTGSYYEHVKISAPNEFDVMFKLEVPRIQLEEYSNTRAYYFVKFK
RNPKENPLSQFLEGEILSASKMLSKFRKIIKEEINDIKDTDVIMKRKRG
GSPAVTLLISEKISVDITLALESKSSWPASTQEGLRIQNWLSAKVRKQL
RLKPFYLVPKHAKEGNGFQEETWRLSFSHIEKEILNNHGKSKTCCENKE
EKCCRKDCLKLMKYLLEQLKERFKDKKHLDKFSSYHVKTAFFHVCTQNP
QDSQWDRKDLGLCFDNCVTYFLQCLRTEKLENYFIPEFNLFSSNLIDKR
SKEFLTKQIEYERNNEFPVFDEF,

Protein was expressed and purified from E. coli BL21 DE3 Rosetta 2 (EMD Millipore) cells using standard techniques. Cells were grown in 2× yeast extract tryptone medium and expression was initiated via the addition of isopropyl β-D-1-thiogalactopyranoside. Expression proceeded overnight at 18° C. Cells were harvested by centrifugation and subsequently lysed via sonication. Insoluble fraction was removed by centrifugation. Maltose binding protein (MBP) fusion proteins were purified on a dextrin sepharose column (GE Healthcare) and the MBP tag was removed using tobacco etch virus protease overnight during dialysis. Protein was further purified on a heparin column (GE Healthcare) and eluted using a NaCl gradient. Column fraction were pooled and further purified on a Superdex 75 gel filtration column (GE Healthcare). Protein was quantified using 280 nm absorbance. Protein was then flash frozen in liquid nitrogen and stored at −80° C. until use.

Potential antagonists were diluted in 100% dimethyl sulfoxide and added to the reaction. Final dimethyl sulfoxide concentration was 5%. The compounds were tested from 1 μM with either 3- or 4-fold serial dilutions down to 0.000051 or 0.000004 μM respectively.

Two complementary DNA oligos (IDT DNA) were annealed by slow cooling from 95° C. The resulting double stranded DNA was used to activate cGAS.

Top strand oligo:
SEQ. ID No. 2
5′-TACAGATCTACTAGTGATCTATGACTGATCTGTACATGATCTACA-3′
Bottom strand oligo:
SEQ. ID No. 3
3′-TGTAGATCATGTACAGATCAGTCATAGATCACTAGTAGATCTGTA-3′

Reactions were performed at 37° C. for 1.25 hours. Reaction buffer: 20 mM Tris HCl pH 9, 100 mM NaCl, 5 mM MgCl₂, 0.1 mg/ml bovine gamma globulin, 250 μM adenosine triphosphate, 100 μM guanosine triphosphate, 0.5 mM Tris(2-carboxyethyl)phosphine hydrochloride, 1 μM double stranded DNA and 300 nM purified cGAS protein.

Reactions were stopped and ATP levels in the reaction were measured using a luciferase based assay. Promega Kinase-Glo Max Assay. Luminescence was measured on a plate reader (Molecular Devices). Values were normalized to control wells lacking compound.

Table 7 below provides IC₅₀ data for certain compounds of the invention on cGAS, as determined using the assay described above. “A” indicates an IC₅₀ value less than 20 μM, “B” indicates an IC₅₀ value between 20 and 250 μM, and “C” indicates an IC₅₀ above the upper limit of the assay (250 μM), or where an IC₅₀ value could not be generated from the data.

TABLE 7

| | cGAS IC₅₀ |
|---|---|
| Compound ID | (μM) |
| TA1002 | C |
| TA1003 | C |
| TA1008 | C |
| TA1009 | C |
| TA1010 | C |
| TA1011 | C |
| TA1012 | B |
| TA1013 | C |
| TA1014 | C |
| TA1015 | C |
| TA1016 | B |
| TA1017 | C |
| TA1018 | A |
| TA1019 | C |
| TA1020 | A |
| TA1021 | A |
| TA1022 | C |
| TA1023 | C |
| TA1024 | A |
| TA1025 | A |
| TA1026 | C |
| TA1027 | A |
| TA1028 | C |
| TA1029 | C |
| TA1030 | C |
| TA1031 | C |
| TA1032 | C |
| TA1033 | C |
| TA1034 | C |
| TA1035 | C |
| TA1036 | C |
| TA1049 | C |
| TA1050 | B |
| TA1052 | B |
| TA1053 | A |
| TA1054 | C |
| TA1055 | C |
| TA1056 | C |
| TA1058 | B |
| TA1059 | A |
| TA1060 | C |
| TA1061 | A |
| TA1062 | B |
| TA1064 | C |
| TA1065 | A |
| TA1066 | A |
| TA1068 | C |
| TA1069 | C |
| TA1070 | A |
| TA1071 | A |
| TA1072 | A |
| TA1073 | C |
| TA1074 | C |
| TA1075 | C |
| TA1076 | C |
| TA1077 | C |
| TA1078 | C |
| TA1079 | C |

### Example 6: THP1 Cell-Based cGAS/STING Pathway Activity Assay

A cellular assay can be used to assess the compounds of the invention for their ability to inhibit the cGAS/STING pathway. Cells that express a luciferase-based reporter that is linked to IRF-3 activation are used to determine response as a function of compound concentration. Such an assay is described in Vincent et al., Nature Communications 2017, 8(1):750, doi: 10.1038/s41467-017-00833-9. Compounds of the invention were assessed using similar assay methods in a THP1 cell assay to generate IC₅₀ values as provided in the following Table 8. In this table, activity level “A” indicates an IC₅₀ value less than 20 μM, “B” indicates an IC₅₀ value between 20 and 100 μM, and “C” indicates an IC₅₀ value above the upper limit of the assay (100 μM), or where an IC₅₀ value could not be generated from the data.

TABLE 8

| | THP-1 IC₅₀ |
|---|---|
| Compound ID | (μM) |
| TA1012 | C |
| TA1018 | C |
| TA1020 | C |
| TA1021 | C |
| TA1024 | C |
| TA1025 | C |
| TA1027 | C |
| TA1053 | C |
| TA1059 | C |
| TA1061 | C |
| TA1065 | B |
| TA1066 | C |
| TA1070 | C |
| TA1071 | C |

### INCORPORATION BY REFERENCE

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EQUIVALENTS

The details of one or more embodiments of the invention are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the invention to the precise form disclosed, but by the claims appended hereto.

1. A compound of Formula (I) or a pharmaceutically acceptable salt thereof: 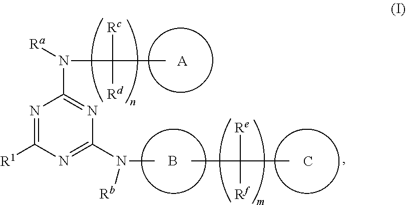
wherein
each of R^{a} and R^{b} independently is H or C₁-C₆ alkyl, wherein C₁-C₆ alkyl is optionally substituted with one or more substituents selected from the group consisting of cyano, halo, —OR, —S(═O)ₓR, and —NRR′;
each of R^{c} and R^{d} independently is, at each occurrence, H, halo, —C(═O)R², or C₁-C₃ alkyl, wherein the C₁-C₃ alkyl is optionally substituted with one or more substituents independently selected from the group consisting of cyano, halo, —OR, —S(═O)ₓR, —NRR′; or
R^{c} and R^{d} attached to the same carbon is oxo; or R^{c} and R^{d} attached to the same carbon forms a C₃-C₃ cycloalkyl, or a 3- to 8-membered heterocycloalkyl, wherein the C₃-C₈ cycloalkyl, or 3- to 8-membered heterocycloalkyl is optionally substituted with one or more substituents independently selected from the group consisting of cyano, halo, oxo, —OR, —S(═O)ₓR, —NRR′, C₁-C₃ alkyl and C₁-C₃ haloalkyl; and wherein at least one occurrence of R^{c} is —C(═O)R²;
R² is —OR^{g} or —NR^{g}R^{h};
each of R^{g} and R^{h} is independently H, C₁-C₆ alkyl, (CH₂CH₂O)ᵤ—H, or (CH₂CH₂O)ᵤ—C₁-C₆ alkyl;
each of R^{e} and R^{f} independently is H, halo, or C₁-C₃ alkyl, wherein the C₁-C₃ alkyl is optionally substituted with one or more substituents independently selected from the group consisting of cyano, halo, —OR, —S(═O)ₓR, and —NRR′; or R^{e} and R^{f} attached to the same carbon is oxo; or R^{e} and R^{f} attached to the same carbon forms a C₃-C₈ cycloalkyl, or a 3- to 8-membered heterocycloalkyl, wherein the C₃-C₈ cycloalkyl, or 3- to 8-membered heterocycloalkyl is optionally substituted with one or more substituents independently selected from the group consisting of cyano, halo, oxo, —OR, —S(═O)ₓR, —NRR′, C₁-C₃ alkyl and C₁-C₃ haloalkyl;
R¹ is H, halo, cyano, —OR¹, —NRⁱR^{j}, or C₁-C₆ alkyl, wherein C₁-C₆ alkyl is optionally substituted with one or more R^{S1};
each R^{S1} is independently halo, cyano, oxo, —OR¹, —NRⁱR^{j}, C₃-C₈ cycloalkyl, C₆-C₁₀ aryl, 3- to 8-membered heterocycloalkyl, or 5- to 10-membered heteroaryl, wherein the C₃-C₈ cycloalkyl, C₆-C₁₀ aryl, 3- to 8-membered heterocycloalkyl, or 5- to 10-membered heteroaryl is optionally substituted with one or more substituents independently selected from the group consisting of cyano, halo, oxo, —OR, —S(═O)ₓR, —NRR′, C₁-C₆ alkyl and C₁-C₆ haloalkyl;
each of Rⁱ and R^{j} independently is H, C₁-C₆ alkyl, C₃-C₈ cycloalkyl, 3- to 10-membered heterocycloalkyl, C₆-C₁₀ aryl or 5- to 10-membered heteroaryl wherein the C₁-C₆ alkyl is optionally substituted with one or more R^{S2}, and the C₃-C₈ cycloalkyl, 3- to 10-membered heterocycloalkyl, C₆-C₁₀ aryl or 5- to 10-membered heteroaryl is optionally substituted with one or more substituents independently selected from the group consisting of cyano, halo, oxo, —OR, —S(═O)ₓR, —NRR′, C₁-C₆ alkyl and C₁-C₆ haloalkyl;
each R^{S2} is independently halo, cyano, —OR, —NRR, C₃-C₈ cycloalkyl, 3- to 10-membered heterocycloalkyl, C₆-C₁₀ aryl or 5- to 10-membered heteroaryl, wherein the C₃-C₈ cycloalkyl, 3- to 10-membered heterocycloalkyl, C₆-C₁₀ aryl or 5- to 10-membered heteroaryl is optionally substituted with one or more substituents independently selected from the group consisting of cyano, halo, oxo, —OR, —S(═O)ₓR, —NRR′, and C₁-C₆ alkyl, wherein the C₁-C₆ alkyl is optionally substituted with one or more substituents independently selected from the group consisting of cyano, halo, oxo, —OR, —S(═O)ₓR, and —NRR′;
each of R and R′ independently is, at each occurrence, H, C₁-C₆ alkyl, C₁-C₆ haloalkyl, C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein the C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of cyano, halo, oxo, —OR^{p}, —S(═O)ₓR^{p}, —NR^{p}R^{q}, and C₁-C₆ alkyl, wherein the C₁-C₆ alkyl is optionally substituted with one or more substituents independently selected from the group consisting of cyano, halo, oxo, —OR^{p}, —S(═O)ₓR^{p}, and —NR^{p}R^{q};
A is a 3- to 14-membered ring optionally containing 1-4 heteroatoms selected from N, O, and S and optionally substituted with one or more R^{S3};
B is a 3- to 14-membered ring optionally containing 1-4 heteroatoms selected from N, O, and S and optionally substituted with one or more R^{S4};
C is a 3- to 14-membered ring optionally containing 1-4 heteroatoms selected from N, O, and S and optionally substituted with one or more R^{S5};
each of R^{S3}, and R^{S4} is independently selected from the group consisting of cyano, halo, oxo, —OR, —S(═O)ₓR, —NRR′, and C₁-C₆ alkyl, wherein the C₁-C₆ alkyl is optionally substituted with one or more substituents independently selected from the group consisting of cyano, halo, oxo, —OR, —S(═O)ₓR, and —NRR′;
each R^{S5} independently is halo, cyano, —C(═O)R^{k}, —S(═O)ₓR^{k}, a nitrogen protecting group bound to a suitable nitrogen of ring C, or C₁-C₆ alkyl, wherein the alkyl is optionally substituted with one or more R^{S6};
R^{k} is H, C₁-C₆ alkyl, C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein C₁-C₆ alkyl is optionally substituted with one or more R^{S6}, and wherein C₀₋₃alkylene-C₃-C₅ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of R^{S6}, C₁-C₆ alkyl, and C₁-C₆ alkyl substituted with one or more R^{S6};
each R^{S6} independently is halo, cyano, oxo, —OR^{m}, —OC(═O)R^{m}, —C(═O)R^{m}, —C(═O)OR^{m}, —S(═O)ₓR^{m}, —S(═O)₂OR^{m}, —OS(═O)₂R^{m}, —NR^{m}Rⁿ, —C(═O)NR^{m}Rⁿ, —C(═NR^{m})NR^{m}Rⁿ, —S(═O)₂NR^{m}Rⁿ, —NR^{m}C(═O)R^{m}, —NR^{m}C(═NR^{m})R^{m}, —NR^{m}S(═O)₂R^{m}, —NR^{m}C(═O)NR^{m}Rⁿ, —NR^{m}C(═NR^{m})NR^{m}Rⁿ, —NR^{m}S(═O)₂NR^{m}Rⁿ, —NR^{m}C(═O)OR^{m}, —OC(═O)NR^{m}Rⁿ, C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein the C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of cyano, halo, oxo, OR^{m}, OC(═O)R^{m}, C(═O)R^{m}, C(═O)OR^{m}, S(═O)ₓR^{m}, S(═O)₂OR^{m}, OS(═O)₂R^{m}, NR^{m}Rⁿ, C(═O)NR^{m}Rⁿ, C(═NR^{m})NR^{m}Rⁿ, S(═O)₂NR^{m}Rⁿ, NR^{m}C(═O)R^{m}, NR^{m}C(═NR^{m})R^{m}, NR^{m}S(═O)₂R^{m}, NR^{m}C(═O)NR^{m}Rⁿ, NR^{m}C(═NR^{m})NR^{m}Rⁿ, NR^{m}S(═O)₂NR^{m}Rⁿ, NR^{m}C(═O)OR^{m}, OC(═O)NR^{m}Rⁿ, and C₁-C₆ alkyl, wherein the C₁-C₆ alkyl is optionally substituted with one or more substituents independently selected from the group consisting of cyano, halo, oxo, OR^{m}, OC(═O)R^{m}, C(═O)R^{m}, C(═O)OR^{m}, S(═O)ₓR^{m}, S(═O)₂OR^{m}, OS(═O)₂R^{m}, NR^{m}Rⁿ, C(═O)NR^{m}Rⁿ, C(═NR^{m})NR^{m}Rⁿ, S(═O)₂NR^{m}Rⁿ, NR^{m}C(═O)R^{m}, NR^{m}C(═NR^{m})R^{m}, NR^{m}S(═O)₂R^{m}, NR^{m}C(═O)NR^{m}Rⁿ, NR^{m}C(═NR^{m})NR^{m}Rⁿ, NR^{m}S(═O)₂NR^{m}Rⁿ, NR^{m}C(═O)OR^{m}, OC(═O)NR^{m}Rⁿ;
each of R^{m} and Rⁿ independently is, at each occurrence, H, C₁-C₆ alkyl, C₁-C₆ haloalkyl, C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl, wherein the C₀₋₃alkylene-C₃-C₈ cycloalkyl, C₀₋₃alkylene-C₆-C₁₀ aryl, C₀₋₃alkylene-3- to 8-membered heterocycloalkyl, or C₀₋₃alkylene-5- to 10-membered heteroaryl is optionally substituted with one or more substituents independently selected from the group consisting of cyano, halo, oxo, —OR^{p}, —OC(═O)R^{p}, —C(═O)R^{p}, —C(═O)OR^{p}, —S(═O)ₓR^{p}, —S(═O)₂OR^{p}, —OS(═O)₂R^{p}, —NR^{p}R^{q}, —C(═O)NR^{p}R^{q}, —C(═NR^{p})NR^{p}R^{q}, —S(═O)₂NR^{p}R^{q}, —NR^{p}C(═O)R^{p}, —NR^{p}C(═NR^{p})R^{p}, —NR^{p}S(═O)₂R^{p}, —NR^{p}C(═O)NR^{p}R^{q}, —NR^{p}C(═NR^{p})NR^{p}R^{q}, —NR^{p}S(═O)₂NR^{p}R^{q}, —NR^{p}C(═O)OR^{p}, —OC(═O)NR^{p}R^{q}, and C₁-C₆ alkyl, wherein the C₁-C₆ alkyl is optionally substituted with one or more substituents independently selected from the group consisting of cyano, halo, oxo, —OR^{p}, —OC(═O)R^{p}, —C(═O)R^{p}, —C(═O)OR^{p}, —S(═O)ₓR^{p}, —S(═O)₂OR^{p}, —OS(═O)₂R^{p}, —NR^{p}R^{q}, —C(═O)NR^{p}R^{q}, —C(═NR^{p})NR^{p}R^{q}, —S(═O)₂NR^{p}R^{q}, —NR^{p}C(═O)R^{p}, —NR^{p}C(═NR^{p})R^{p}, —NR^{p}S(═O)₂R^{p}, —NR^{p}C(═O)NR^{p}R^{q}, —NR^{p}C(═NR^{p})NR^{p}R^{q}, —NR^{p}S(═O)₂NR^{p}R^{q}, —NR^{p}C(═O)OR^{p}, —OC(═O)NR^{p}R^{q};
each R^{p} and R^{q} are independently H or C₁-C₆ alkyl;
m is 0, 1, 2, or 3;
n is 1, 2, 3, or 4;
each occurrence of x is independently 0, 1, or 2; and
each u independently is 1, 2, or 3.

2. (canceled)

3. (canceled)

4. (canceled)

5. (canceled)

6. (canceled)

7. The compound of claim 1, wherein the compound is of Formula (Ia): (Ia); and 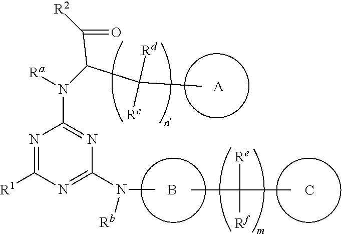
n′ is 0, 1, 2, or 3.

8. (canceled)

9. (canceled)

10. (canceled)

11. The compound of claim 7, wherein A is a 5- to 6-membered ring optionally containing 1-4 heteroatoms selected from N, O, and S and optionally substituted with one or more R^{S3}.

12. The compound of claim 11, wherein A is phenyl or 4-cyanophenyl.

13. (canceled)

14. The compound of claim 7, wherein B is a 5- to 6-membered ring optionally containing 1-4 heteroatoms selected from N, O, and S and optionally substituted with one or more R^{S4}.

15. The compound of claim 14, wherein B is phenyl.

16. The compound of claim 15, wherein m is 1, and each of R^{e} and R^{f} is H.

17. (canceled)

18. (canceled)

19. (canceled)

20. The compound of claim 7, wherein C is a 6-membered ring optionally containing 1-2 heteroatoms selected from N, O, and S and optionally substituted with one or more R^{S5}.

21. The compound of claim 8, wherein C is

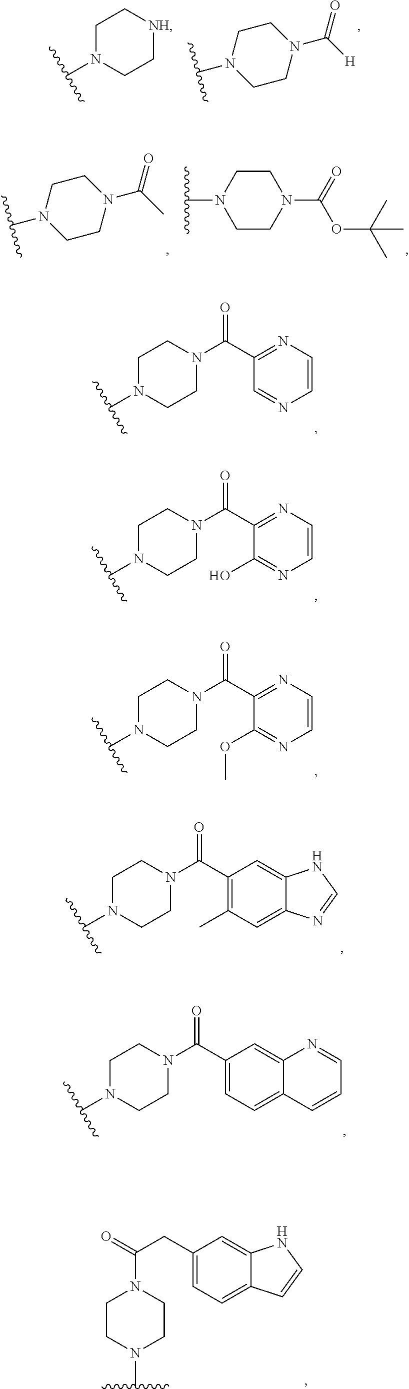

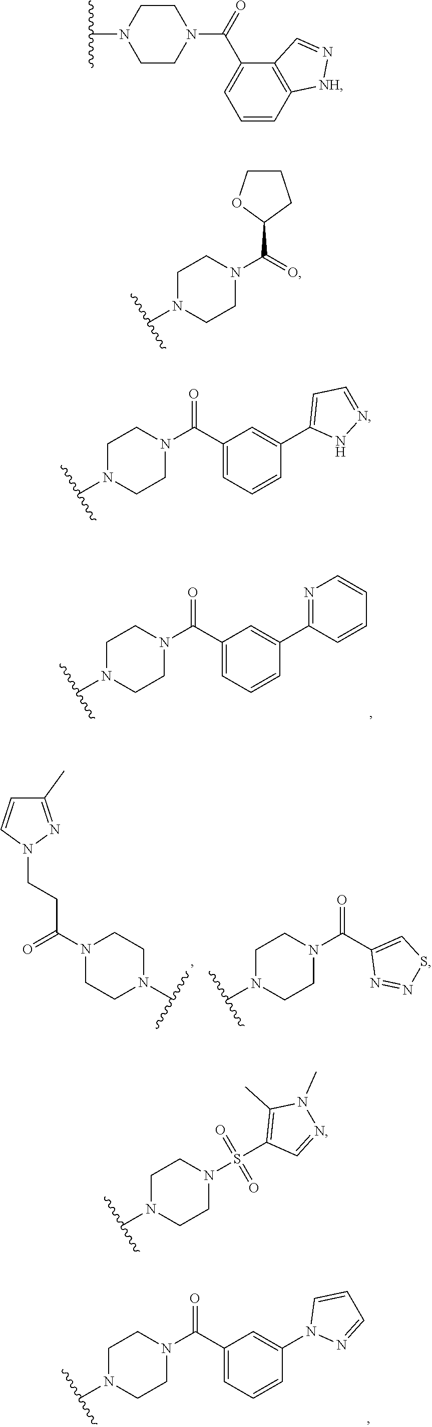

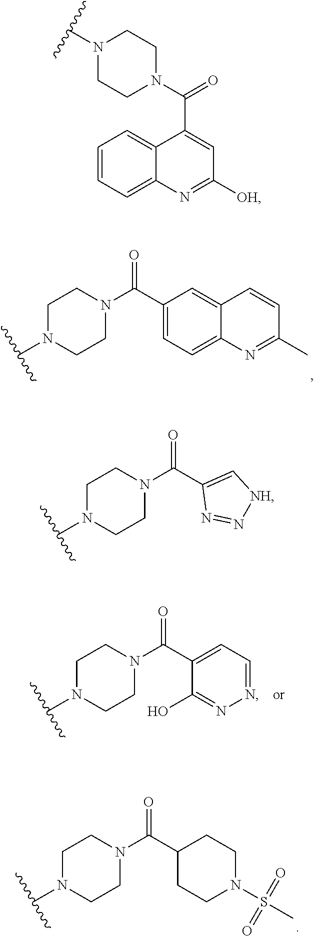 [SMILES: *N1CCN(C(=O)c2cc(O)nc3ccccc23)CC1 ; *N1CCN(C(=O)c2ccc3nc(C)ccc3c2)CC1 ; *N1CCN(C(=O)c2c[nH]nn2)CC1 ; *N1CCN(C(=O)c2ccnnc2O)CC1 ; *N1CCN(C(=O)C2CCN(S(C)(=O)=O)CC2)CC1]

22. The compound of claim 1, wherein the compound is of Formula (Ib): 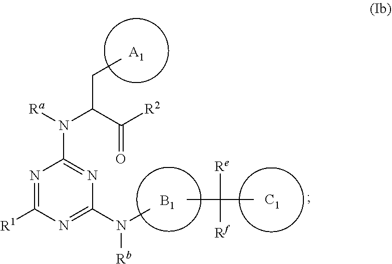
A₁ is a 3- to 14-membered ring optionally containing 1-4 heteroatoms selected from N, O, and S and optionally substituted with one or more R^{S3};
B₁ is a 3- to 14-membered ring optionally containing 1-4 heteroatoms selected from N, O, and S and optionally substituted with one or more R^{S4}; and
C₁ is a 3- to 14-membered ring optionally containing 1-4 heteroatoms selected from N, O, and S and optionally substituted with one or more R^{S5}.

23. The compound of claim 22, wherein A₁ is phenyl or 4-cyanophenyl.

24. (canceled)

25. The compound of claim 22, wherein B₁ is a 5- to 6-membered ring optionally containing 1-4 heteroatoms selected from N, O, and S and optionally substituted with one or more R^{S4}.

26. The compound of claim 25, wherein B₁ is phenyl.

27. (canceled)

28. The compound of claim 27, wherein C₁ is a 6-membered ring optionally containing 1-2 heteroatoms selected from N, O, and S and optionally substituted with one or more R^{S5}.

29. The compound of claim 28, wherein C₁ is

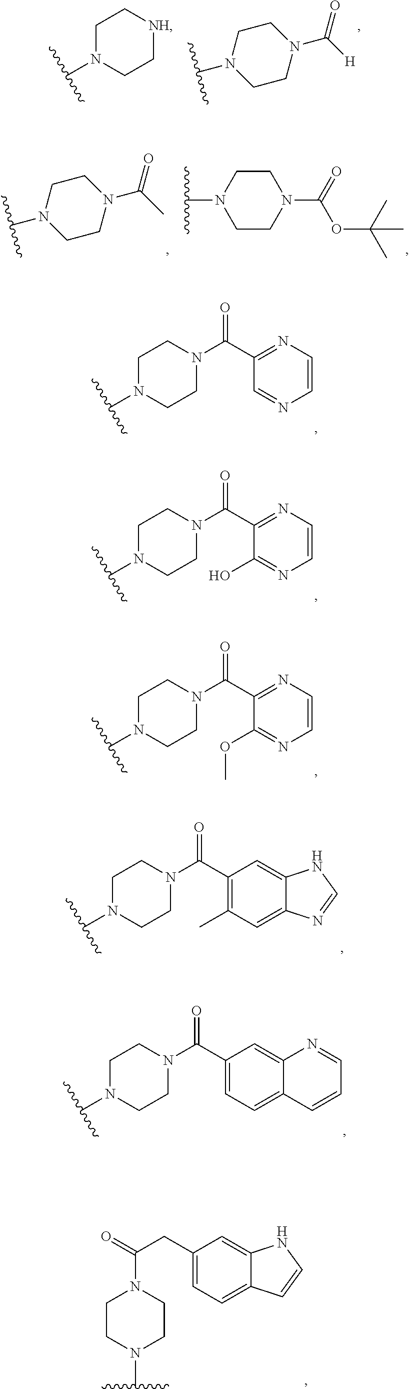

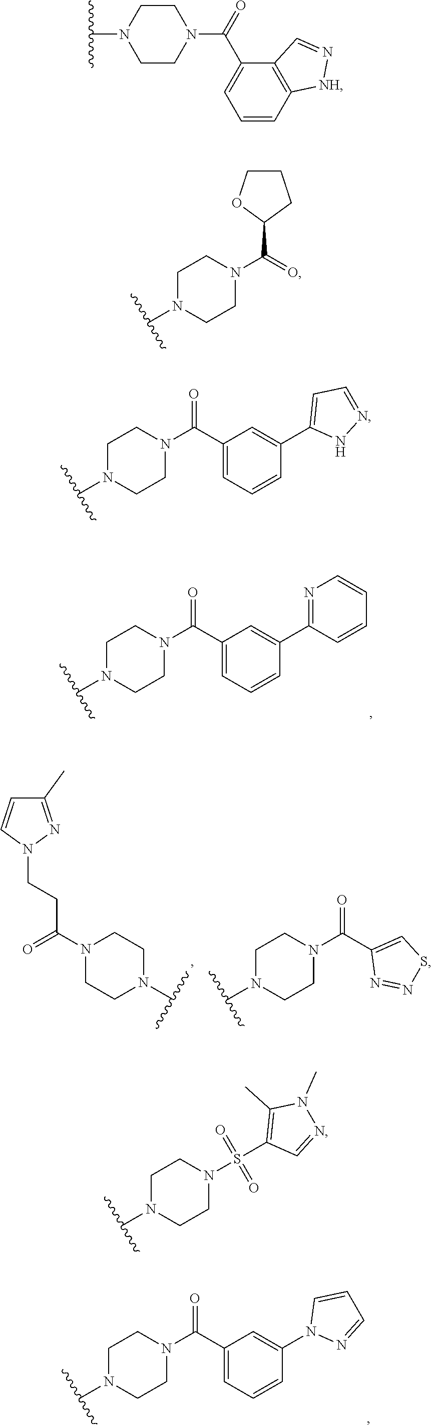

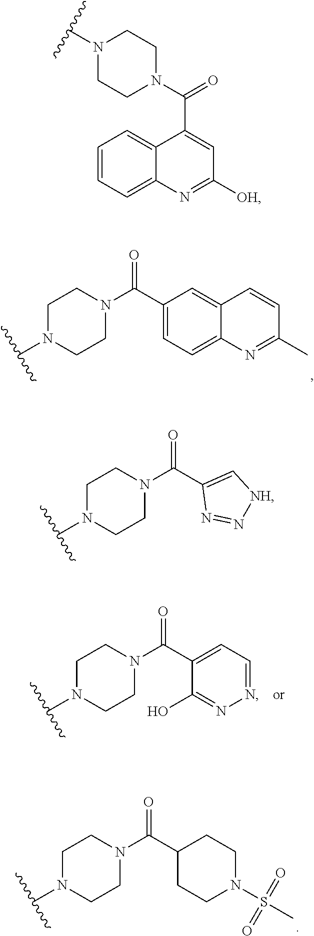 [SMILES: *N1CCN(C(=O)c2cc(O)nc3ccccc23)CC1 ; *N1CCN(C(=O)c2ccc3nc(C)ccc3c2)CC1 ; *N1CCN(C(=O)c2c[nH]nn2)CC1 ; *N1CCN(C(=O)c2ccnnc2O)CC1 ; *N1CCN(C(=O)C2CCN(S(C)(=O)=O)CC2)CC1]

30. The compound of claim 1, wherein the compound is of Formula (Ic): 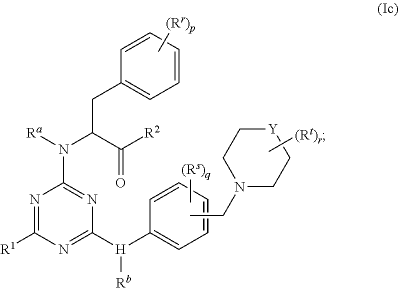
each R^{r} independently is cyano, halo, oxo, —OR, —S(═O)ₓR, —NRR′, and C₁-C₆ alkyl;
each R^{s} independently is cyano, halo, oxo, —OR, —S(═O)ₓR, —NRR′, and C₁-C₆ alkyl;
each R¹ independently is halo, cyano, —C(═O)R^{k}, —S(═O)ₓR^{k}, a nitrogen protecting group bound to a suitable nitrogen in the ring, or C₁-C₆ alkyl, wherein the alkyl is optionally substituted with one or more R^{S6};
Y is O, CH₂, CHR^{t}, NH, or NR^{t};
p is 0, 1, 2, 3, 4, or 5; and
each of q and r independently is 0, 1, 2, 3, or 4.

31. The compound of claim 30, wherein p is 0 or 1, and R^{r} is cyano.

32. (canceled)

33. The compound of claim 30, wherein q is 0, r is 0 and Y is NH or r is 1 and Y is NR^{t}.

34. (canceled)

35. (canceled)

36. (canceled)

37. (canceled)

38. The compound of claim 30, wherein R^{t} is C(═O)R^{k} and R^{k} is H or a 5- to 6-membered heteroaryl optionally substituted with one or more substituents independently selected from the group consisting of R^{S6}, C₁-C₆ alkyl, and C₁-C₆ alkyl substituted with one or more R^{S6}.

39. (canceled)

40. (canceled)

41. (canceled)

42. (canceled)

43. (canceled)

44. (canceled)

45. The compound of claim 30, wherein R^{k} is

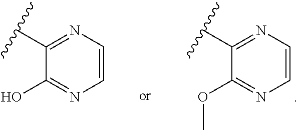

46. (canceled)

47. (canceled)

48. (canceled)

49. (canceled)

50. (canceled)

51. (canceled)

52. (canceled)

53. (canceled)

54. (canceled)

55. The compound of claim 30, wherein R¹ is NRⁱR^{j}.

56. (canceled)

57. (canceled)

58. The compound of claim 21, wherein R¹ is —NHCH₃,

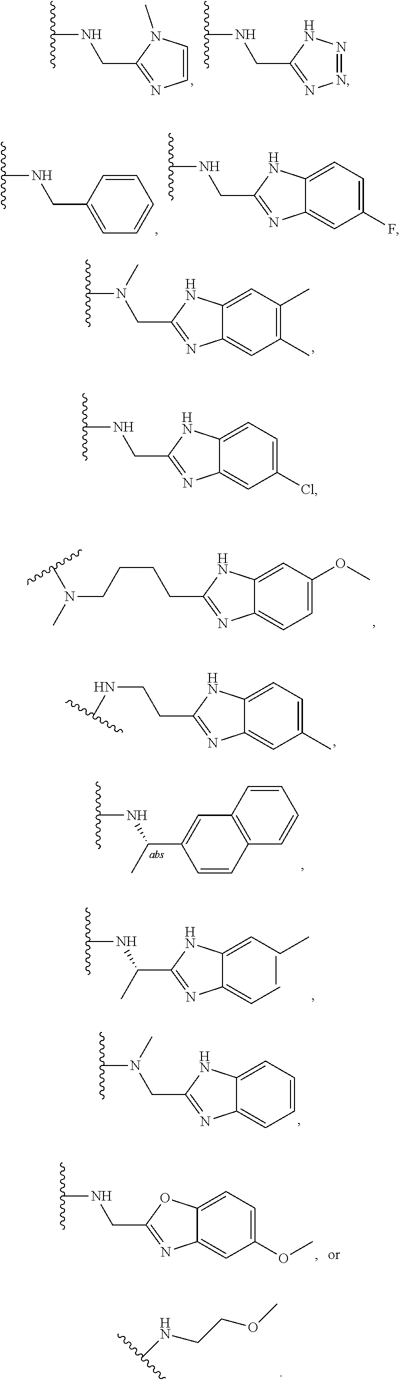

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. The compound of claim 30, wherein each of R^{a} and R^{b} is H.

73. The compound according to claim 1, wherein the compound is selected from the group consisting of

| Compound | |
|---|---|
| ID | Structure |
| TA1002 | 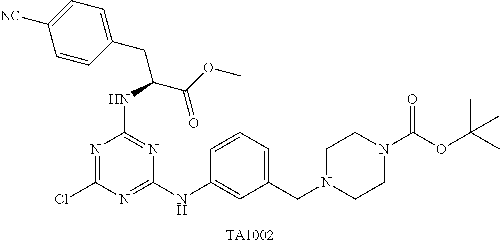 |
| | tert-butyl (S)-4-(3-(3-((4-chloro-6-((3-(4-cyanophenyl)-1-methoxy-1- |
| | oxopropan-2-yl)amino)-1,3,5-triazin-2-yl)amino)benzyl)piperazine- |
| | 1-carboxylate |
| TA1003 | 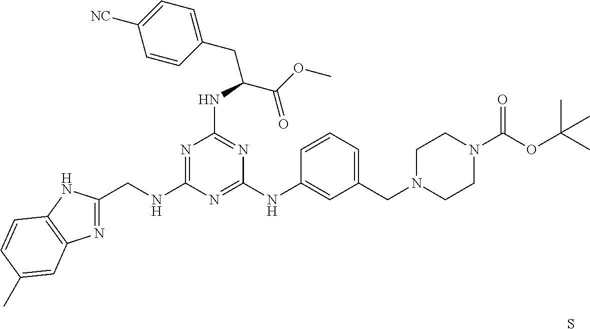 |
| | tert-butyl (S)-4-(3-((4-((3-(4-cyanophenyl)-1-methoxy-1-oxopropan- |
| | 2-yl)amino)-6-(((5-fluoro-1H-benzo[d]imidazol-2-yl)methyl)amino)- |
| | 1,3,5-triazin-2-yl)amino)benzyl)piperazine-1-carboxylate |
| TA1008 | 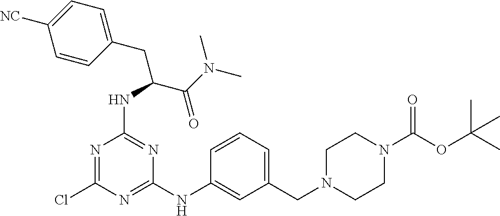 [SMILES: CN(C)C(=O)[C@H](Cc1ccc(C#N)cc1)Nc1nc(Cl)nc(Nc2cccc(CN3CCN(C(=O)OC(C)(C)C)CC3)c2)n1] |
| | tert-butyl (S)-4-(3-((4-chloro-6-((3-(4-cyanophenyl)-1- |
| | (dimethylamino)-1-oxopropan-2-yl)amino)-1,3,5-triazin-2- |
| | yl)amino)benzyl)piperazine-1-carboxylate |
| TA1009 | 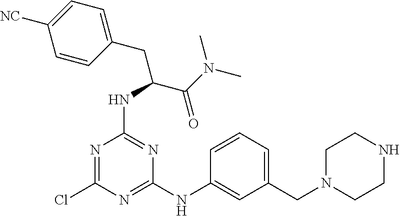 [SMILES: CN(C)C(=O)[C@H](Cc1ccc(C#N)cc1)Nc1nc(Cl)nc(Nc2cccc(CN3CCNCC3)c2)n1] |
| | (S)-2-((4-chloro-6-((3-(piperazin-1-ylmethyl)phenyl)amino)-1,3,5- |
| | triazin-2-yl)amino)-3-(4-cyanophenyl)-N,N-dimethylpropanamide |
| TA1010 | 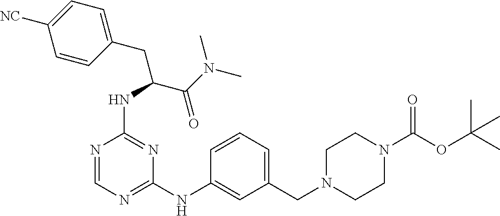 [SMILES: CN(C)C(=O)[C@H](Cc1ccc(C#N)cc1)Nc1ncnc(Nc2cccc(CN3CCN(C(=O)OC(C)(C)C)CC3)c2)n1] |
| | tert-butyl (S)-4-(3-((4-((3-(4-cyanophenyl)-1-(dimethylamino)-1- |
| | oxopropan-2-yl)amino)-1,3,5-triazin-2-yl)amino)benzyl)piperazine- |
| | 1-carboxylate |
| TA1011 | 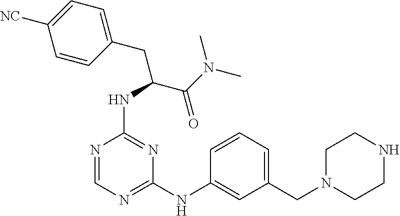 [SMILES: CN(C)C(=O)[C@H](Cc1ccc(C#N)cc1)Nc1ncnc(Nc2cccc(CN3CCNCC3)c2)n1] |
| | (S)-3-(4-cyanophenyl)-N,N-dimethyl-2-((4-((3-(piperazin-1- |
| | ylmethyl)phenyl)amino)-1,3,5-triazin-2-yl)amino)propanamide |
| TA1012 | 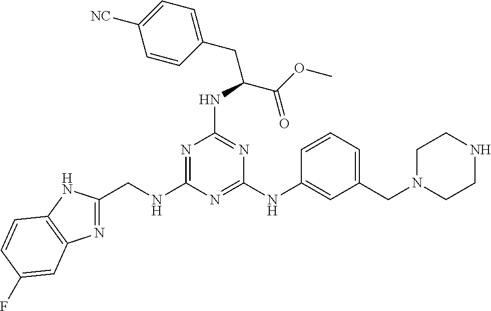 [SMILES: COC(=O)[C@H](Cc1ccc(C#N)cc1)Nc1nc(NCc2nc3cc(F)ccc3[nH]2)nc(Nc2cccc(CN3CCNCC3)c2)n1] |
| | methyl (S)-3-(4-cyanophenyl)-2-((4-(((5-fluoro-1H- |
| | benzo[d]imidazol-2-yl)methyl)amino)-6-((3-(piperazin-1- |
| | ylmethyl)phenyl)amino)-1,3,5-triazin-2-yl)amino)propanoate |
| TA1013 | 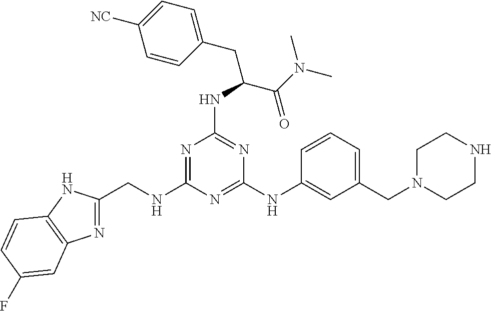 [SMILES: CN(C)C(=O)[C@H](Cc1ccc(C#N)cc1)Nc1nc(NCc2nc3cc(F)ccc3[nH]2)nc(Nc2cccc(CN3CCNCC3)c2)n1] |
| | (S)-3-(4-cyanophenyl)-2-((4-(((5-fluoro-1H-benzo[d]imidazol-2- |
| | yl)methyl)amino)-6-((3-(piperazin-1-ylmethyl)phenyl)amino)-1,3,5- |
| | triazin-2-yl)amino)-N,N-dimethylpropanamide |
| TA1014 | 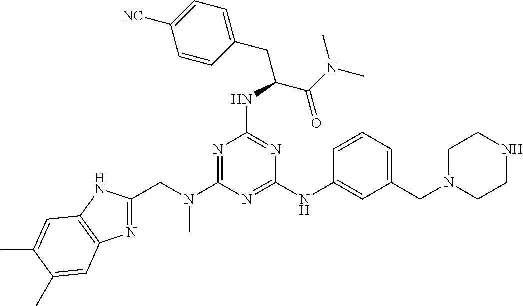 [SMILES: Cc1cc2nc(CN(C)c3nc(Nc4cccc(CN5CCNCC5)c4)nc(N[C@@H](Cc4ccc(C#N)cc4)C(=O)N(C)C)n3)[nH]c2cc1C] |
| | (S)-3-(4-cyanophenyl)-2-((4-(((5,6-dimethyl-1H-benzo[d]imidazol- |
| | 2-yl)methyl)(methyl)amino)-6-((3-(piperazin-1- |
| | ylmethyl)phenyl)amino)-1,3,5-triazin-2-yl)amino)-N,N- |
| | dimethylpropanamide |
| TA1015 | 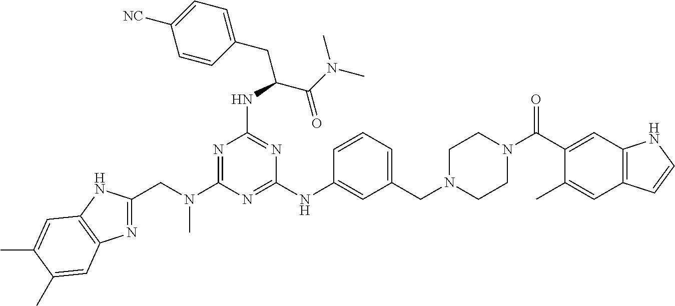 [SMILES: Cc1cc2nc(CN(C)c3nc(Nc4cccc(CN5CCN(C(=O)c6cc7[nH]ccc7cc6C)CC5)c4)nc(N[C@@H](Cc4ccc(C#N)cc4)C(=O)N(C)C)n3)[nH]c2cc1C] |
| | (S)-3-(4-cyanophenyl)-2-((4-(((5,6-dimethyl-1H-benzo[d]imidazol- |
| | 2-yl)methyl)(methyl)amino)-6-((3-((4-(5-methyl-1H- |
| | benzo[d]imidazole-6-carbonyl)piperazin-1- |
| | yl)methyl)phenyl)amino)-1,3,5-triazin-2-yl)amino)-N,N- |
| | dimethylpropanamide |
| TA1016 | 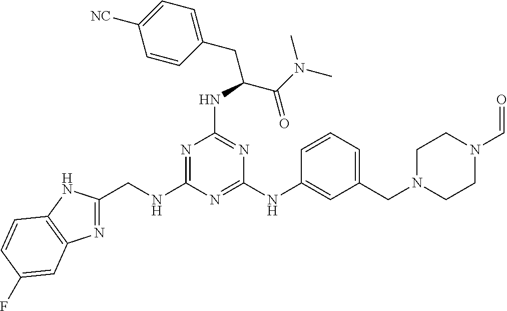 [SMILES: CN(C)C(=O)[C@H](Cc1ccc(C#N)cc1)Nc1nc(NCc2nc3cc(F)ccc3[nH]2)nc(Nc2cccc(CN3CCN(C=O)CC3)c2)n1] |
| | (S)-3-(4-cyanophenyl)-2-((4-(((5-fluoro-1H-benzo[d]imidazol-2- |
| | yl)methyl)amino)-6-((3-((4-formylpiperazin-1- |
| | yl)methyl)phenyl)amino)-1,3,5-triazin-2-yl)amino)-N,N- |
| | dimethylpropanamide |
| TA1017 | 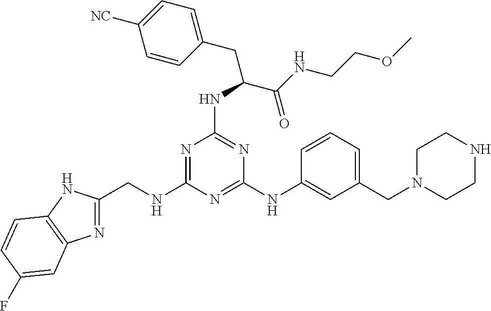 [SMILES: COCCNC(=O)[C@H](Cc1ccc(C#N)cc1)Nc1nc(NCc2nc3cc(F)ccc3[nH]2)nc(Nc2cccc(CN3CCNCC3)c2)n1] |
| | (S)-3-(4-cyanophenyl)-2-((4-(((5-fluoro-1H-benzo[d]imidazol-2- |
| | yl)methyl)amino)-6-((3-(piperazin-1-ylmethyl)phenyl)amino)-1,3,5- |
| | triazin-2-yl)amino)-N-(2-methoxyethyl)propanamide |
| TA1018 | 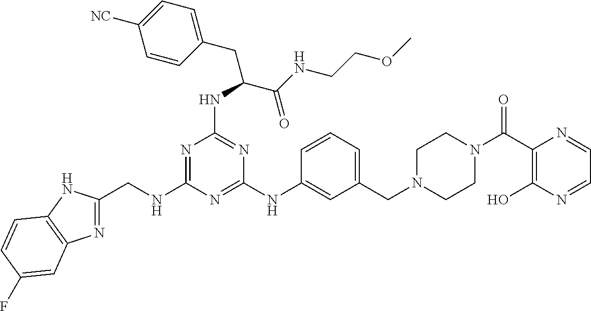 [SMILES: COCCNC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| | (S)-3-(4-cyanophenyl)-2-((4-(((5-fluoro-1H-benzo[d]imidazol-2- |
| | yl)methyl)amino)-6-((3-((4-(3-hydroxypyrazine-2- |
| | carbonyl)piperazin-1-yl)methyl)phenyl)amino)-1,3,5-triazin-2- |
| | yl)amino)-N-(2-methoxyethyl)propanamide |
| TA1019 | 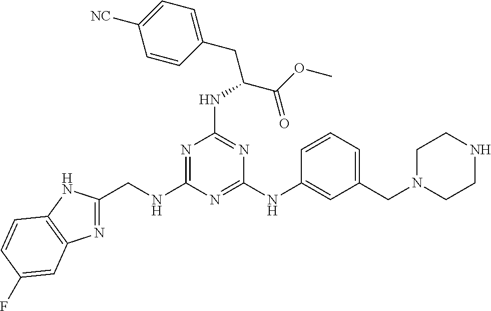 [SMILES: COC(=O)[C@@H](Cc1ccc(C#N)cc1)Nc1nc(NCc2nc3cc(F)ccc3[nH]2)nc(Nc2cccc(CN3CCNCC3)c2)n1] |
| | methyl (R)-3-(4-cyanophenyl)-2-((4-(((5-fluoro-1H- |
| | benzo[d]imidazol-2-yl)methyl)amino)-6-((3-(piperazin-1- |
| | ylmethyl)phenyl)amino)-1,3,5-triazin-2-yl)amino)propanoate |
| TA1020 | 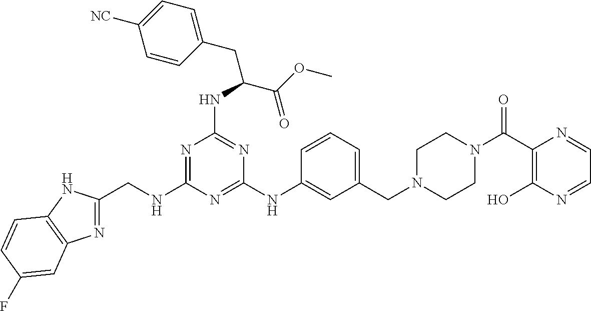 [SMILES: COC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| | methyl (S)-3-(4-cyanophenyl)-2-((4-(((5-fluoro-1H- |
| | benzo[d]imidazol-2-yl)methyl)amino)-6-((3-((4-(3-hydroxypyrazine- |
| | 2-carbonyl)piperazin-1-yl)methyl)phenyl)amino)-1,3,5-triazin-2- |
| | yl)amino)propanoate |
| TA1021 | 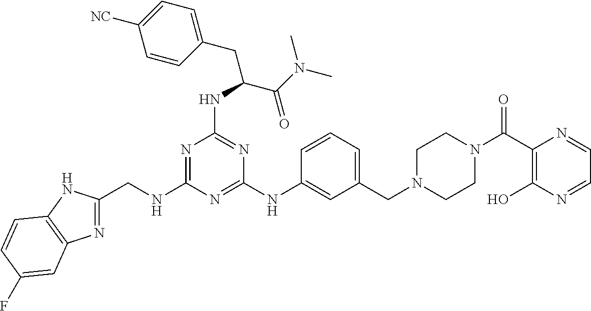 [SMILES: CN(C)C(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| | (S)-3-(4-cyanophenyl)-2-((4-(((5-fluoro-1H-benzo[d]imidazol-2- |
| | yl)methyl)amino)-6-((3-((4-(3-hydroxypyrazine-2- |
| | carbonyl)piperazin-1-yl)methyl)phenyl)amino)-1,3,5-triazin-2- |
| | yl)amino)-N,N-dimethylpropanamide |
| TA1022 | 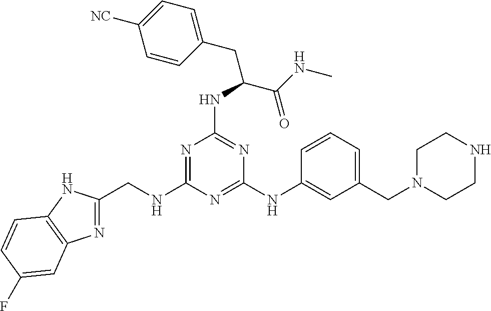 [SMILES: CNC(=O)[C@H](Cc1ccc(C#N)cc1)Nc1nc(NCc2nc3cc(F)ccc3[nH]2)nc(Nc2cccc(CN3CCNCC3)c2)n1] |
| | (S)-3-(4-cyanophenyl)-2-((4-(((5-fluoro-1H-benzo[d]imidazol-2- |
| | yl)methyl)amino)-6-((3-(piperazin-1-ylmethyl)phenyl)amino)-1,3,5- |
| | triazin-2-yl)amino)-N-methylpropanamide |
| TA1023 | 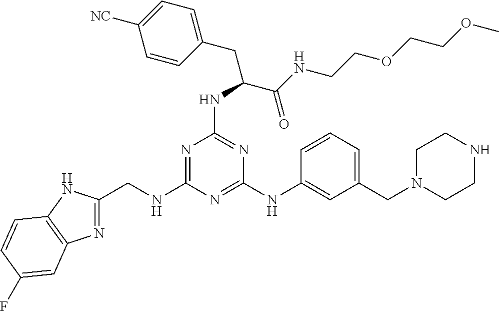 [SMILES: COCCOCCNC(=O)[C@H](Cc1ccc(C#N)cc1)Nc1nc(NCc2nc3cc(F)ccc3[nH]2)nc(Nc2cccc(CN3CCNCC3)c2)n1] |
| | (S)-3-(4-cyanophenyl)-2-((4-(((5-fluoro-1H-benzo[d]imidazol-2- |
| | yl)methyl)amino)-6-((3-(piperazin-1-ylmethyl)phenyl)amino)-1,3,5- |
| | triazin-2-yl)amino)-N-(2-(2-methoxyethoxy)ethyl)propanamide |
| TA1024 | 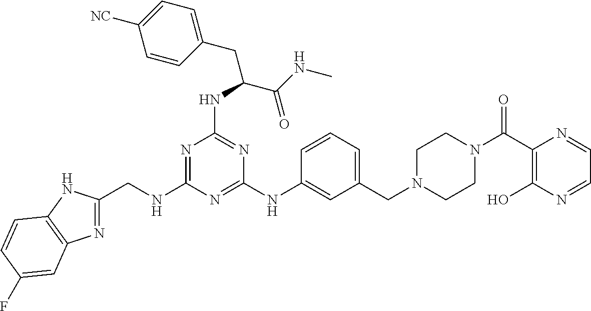 [SMILES: CNC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| | (S)-3-(4-cyanophenyl)-2-((4-(((5-fluoro-1H-benzo[d]imidazol-2- |
| | yl)methyl)amino)-6-((3-((4-(3-hydroxypyrazine-2- |
| | carbonyl)piperazin-1-yl)methyl)phenyl)amino)-1,3,5-triazin-2- |
| | yl)amino)-N-methylpropanamide |
| TA1025 | 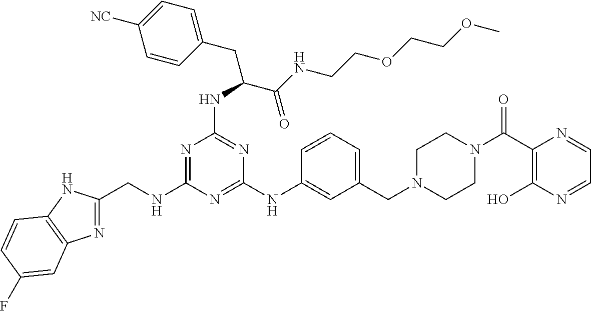 [SMILES: COCCOCCNC(=O)[C@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| | (S)-3-(4-cyanophenyl)-2-((4-(((5-fluoro-1H-benzo[d]imidazol-2- |
| | yl)methyl)amino)-6-((3-((4-(3-hydroxypyrazine-2- |
| | carbonyl)piperazin-1-yl)methyl)phenyl)amino)-1,3,5-triazin-2- |
| | yl)amino)-N-(2-(2-methoxyethoxy)ethyl)propanamide |
| TA1026 | 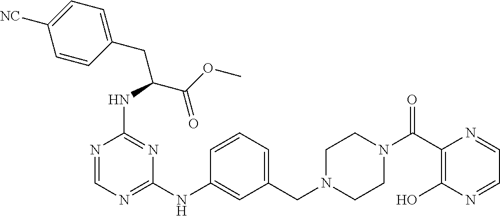 [SMILES: COC(=O)[C@H](Cc1ccc(C#N)cc1)Nc1ncnc(Nc2cccc(CN3CCN(C(=O)c4nccnc4O)CC3)c2)n1] |
| | methyl (S)-3-(4-cyanophenyl)-2-((4-((3-((4-(3-hydroxypyrazine-2- |
| | carbonyl)piperazin-1-yl)methyl)phenyl)amino)-1,3,5-triazin-2- |
| | yl)amino)propanoate |
| TA1027 | 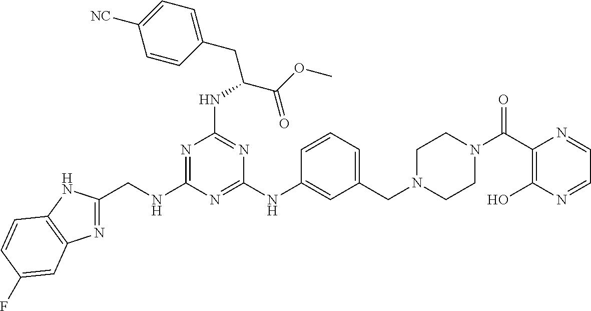 [SMILES: COC(=O)[C@@H](Cc1ccc(C#N)cc1)Nc1nc(NCc2nc3cc(F)ccc3[nH]2)nc(Nc2cccc(CN3CCN(C(=O)c4nccnc4O)CC3)c2)n1] |
| | methyl (R)-3-(4-cyanophenyl)-2-((4-(((5-fluoro-1H- |
| | benzo[d]imidazol-2-yl)methyl)amino)-6-((3-((4-(3-hydroxypyrazine- |
| | 2-carbonyl)piperazin-1-yl)methyl)phenyl)amino)-1,3,5-triazin-2- |
| | yl)amino)propanoate |
| TA1028 | 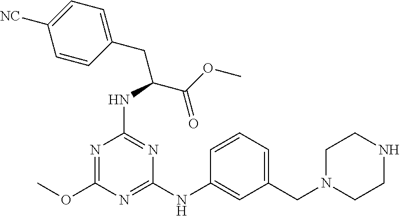 [SMILES: COC(=O)[C@H](Cc1ccc(C#N)cc1)Nc1nc(Nc2cccc(CN3CCNCC3)c2)nc(OC)n1] |
| | methyl (S)-3-(4-cyanophenyl)-2-((4-methoxy-6-((3-(piperazin-1- |
| | ylmethyl)phenyl)amino)-1,3,5-triazin-2-yl)amino)propanoate |
| TA1029 | 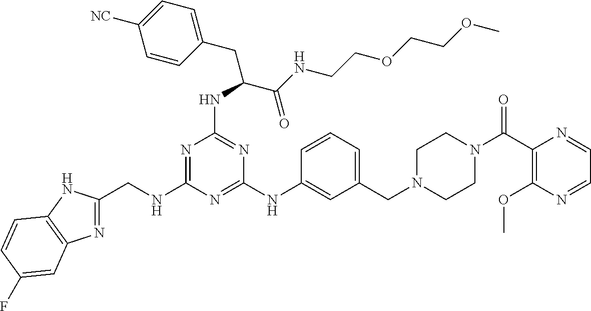 [SMILES: COCCOCCNC(=O)[C@H](Cc1ccc(C#N)cc1)Nc1nc(NCc2nc3cc(F)ccc3[nH]2)nc(Nc2cccc(CN3CCN(C(=O)c4nccnc4OC)CC3)c2)n1] |
| | (S)-3-(4-cyanophenyl)-2-((4-(((5-fluoro-1H-benzo[d]imidazol-2- |
| | yl)methyl)amino)-6-((3-((4-(3-methoxypyrazine-2- |
| | carbonyl)piperazin-1-yl)methyl)phenyl)amino)-1,3,5-triazin-2- |
| | yl)amino)-N-(2-(2-methoxyethoxy)ethyl)propanamide |
| TA1030 | 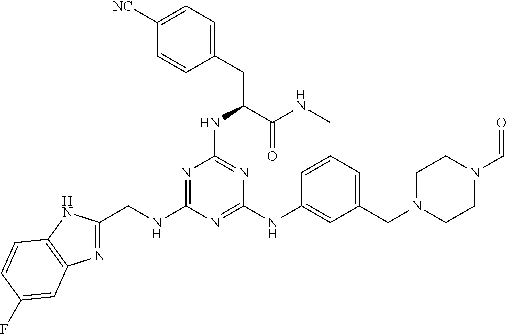 [SMILES: CNC(=O)[C@H](Cc1ccc(C#N)cc1)Nc1nc(NCc2nc3cc(F)ccc3[nH]2)nc(Nc2cccc(CN3CCN(C=O)CC3)c2)n1] |
| | (S)-3-(4-cyanophenyl)-2-((4-(((5-fluoro-1H-benzo[d]imidazol-2- |
| | yl)methyl)amino)-6-((3-((4-formylpiperazin-1- |
| | yl)methyl)phenyl)amino)-1,3,5-triazin-2-yl)amino)-N- |
| | methylpropanamide |
| TA1031 | 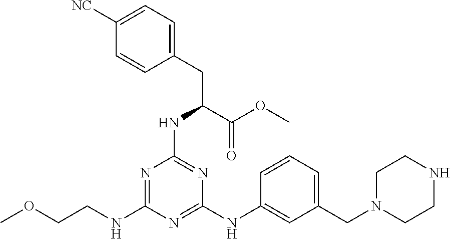 [SMILES: COCCNc1nc(Nc2cccc(CN3CCNCC3)c2)nc(N[C@@H](Cc2ccc(C#N)cc2)C(=O)OC)n1] |
| | methyl (S)-3-(4-cyanophenyl)-2-((4-((2-methoxyethyl)amino)-6-((3- |
| | (piperazin-1-ylmethyl)phenyl)amino)-1,3,5-triazin-2- |
| | yl)amino)propanoate |
| TA1032 | 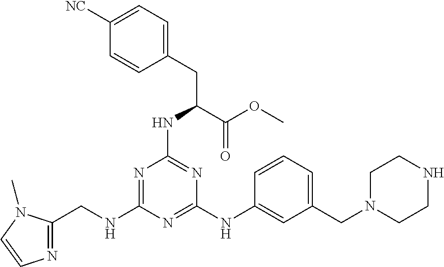 [SMILES: COC(=O)[C@H](Cc1ccc(C#N)cc1)Nc1nc(NCc2nccn2C)nc(Nc2cccc(CN3CCNCC3)c2)n1] |
| | methyl (S)-3-(4-cyanophenyl)-2-((4-(((1-methyl-1H-imidazol-2- |
| | yl)methyl)amino)-6-((3-(piperazin-1-ylmethyl)phenyl)amino)-1,3,5- |
| | triazin-2-yl)amino)propanoate |
| TA1033 | 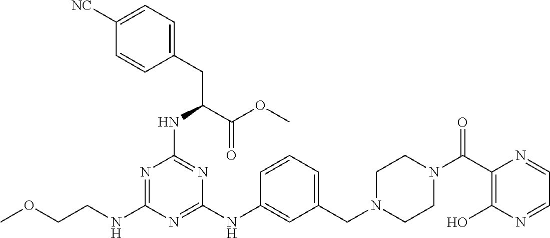 [SMILES: COCCNc1nc(Nc2cccc(CN3CCN(C(=O)c4nccnc4O)CC3)c2)nc(N[C@@H](Cc2ccc(C#N)cc2)C(=O)OC)n1] |
| | methyl (S)-3-(4-cyanophenyl)-2-((4-((3-((4-(3-hydroxypyrazine-2- |
| | carbonyl)piperazin-1-yl)methyl)phenyl)amino)-6-((2- |
| | methoxyethyl)amino)-1,3,5-triazin-2-yl)amino)propanoate |
| TA1034 | 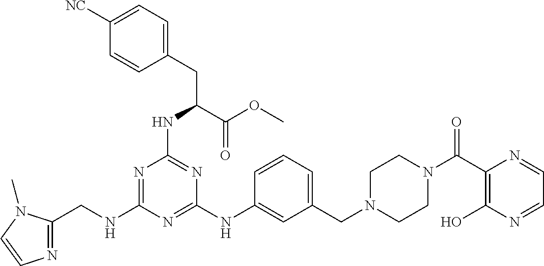 [SMILES: COC(=O)[C@H](Cc1ccc(C#N)cc1)Nc1nc(NCc2nccn2C)nc(Nc2cccc(CN3CCN(C(=O)c4nccnc4O)CC3)c2)n1] |
| | methyl (S)-3-(4-cyanophenyl)-2-((4-((3-((4-(3-hydroxypyrazine-2- |
| | carbonyl)piperazin-1-yl)methyl)phenyl)amino)-6-(((1-methyl-1H- |
| | imidazol-2-yl)methyl)amino)-1,3,5-triazin-2-yl)amino)propanoate |
| TA1035 | 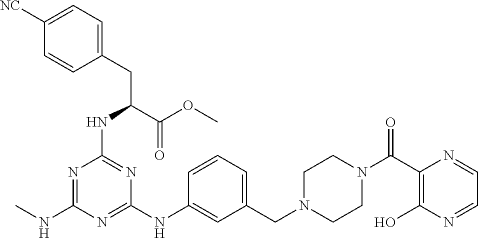 [SMILES: CNc1nc(Nc2cccc(CN3CCN(C(=O)c4nccnc4O)CC3)c2)nc(N[C@@H](Cc2ccc(C#N)cc2)C(=O)OC)n1] |
| | methyl (S)-3-(4-cyanophenyl)-2-((4-((3-((4-(3-hydroxypyrazine-2- |
| | carbonyl)piperazin-1-yl)methyl)phenyl)amino)-6-(methylamino)- |
| | 1,3,5-triazin-2-yl)amino)propanoate |
| TA1036 | 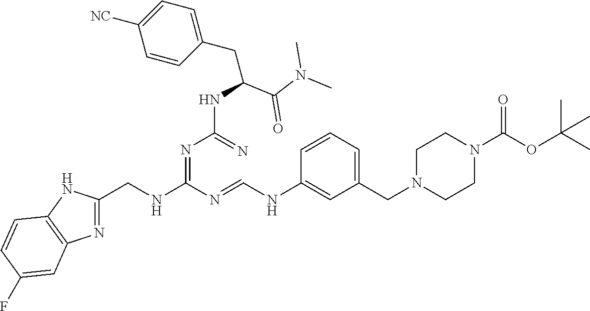 [SMILES: CN(C)C(=O)[C@H](Cc1ccc(C#N)cc1)NC(=N)/N=C(\N=C\Nc1cccc(CN2CCN(C(=O)OC(C)(C)C)CC2)c1)NCc1nc2cc(F)ccc2[nH]1] |
| | tert-butyl (S)-4-(3-((4-((3-(4-cyanophenyl)-1-(dimethylamino)-1- |
| | oxopropan-2-yl)amino)-6-(((5-fluoro-1H-benzo[d]imidazol-2- |
| | yl)methyl)amino)-1,3,5-triazin-2-yl)amino)benzyl)piperazine-1- |
| | carboxylate |
| TA1037 | 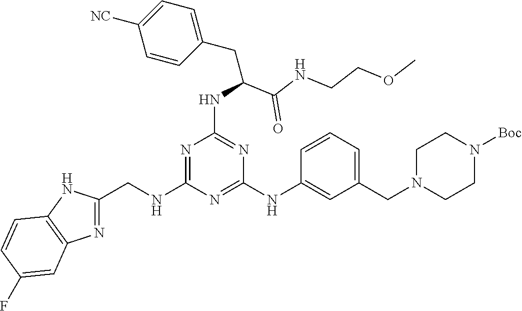 [SMILES: COCCNC(=O)[C@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1] |
| | tert-butyl (S)-4-(3-((4-((3-(4-cyanophenyl)-1-((2- |
| | methoxyethyl)amino)-1-oxopropan-2-yl)amino)-6-(((5-fluoro-1H- |
| | benzo[d]imidazol-2-yl)methyl)amino)-1,3,5-triazin-2- |
| | yl)amino)benzyl)piperazine-1-carboxylate |
| TA1038 | 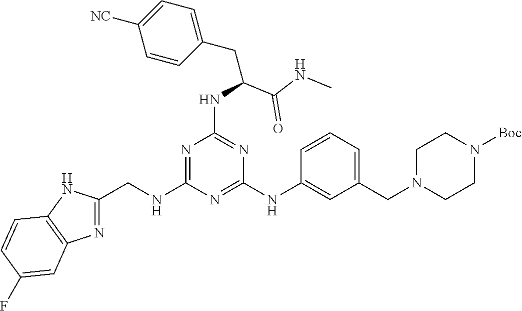 [SMILES: CNC(=O)[C@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1] |
| | tert-butyl (S)-4-(3-((4-((3-(4-cyanophenyl)-1-(methylamino)-1- |
| | oxopropan-2-yl)amino)-6-(((5-fluoro-1H-benzo[d]imidazol-2- |
| | yl)methyl)amino)-1,3,5-triazin-2-yl)amino)benzyl)piperazine-1- |
| | carboxylate |
| TA1039 | 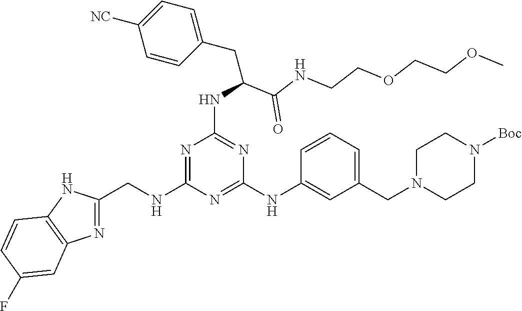 [SMILES: COCCOCCNC(=O)[C@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1] |
| | tert-butyl (S)-4-(3-((4-((3-(4-cyanophenyl)-1-((2-(2- |
| | methoxyethoxy)ethyl)amino)-1-oxopropan-2-yl)amino)-6-(((5- |
| | fluoro-1H-benzo[d]imidazol-2-yl)methyl)amino)-1,3,5-triazin-2- |
| | yl)amino)benzyl)piperazine-1-carboxylate |
| TA1040 | 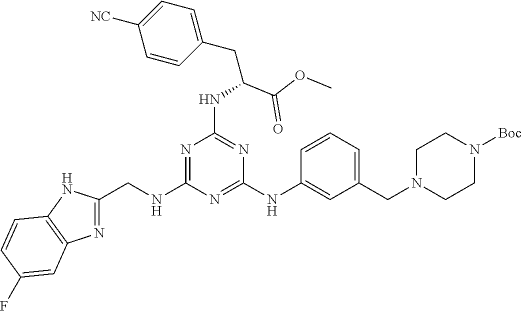 [SMILES: COC(=O)[C@@H](Cc1ccc(C#N)cc1)Nc1nc(NCc2nc3cc(F)ccc3[nH]2)nc(Nc2cccc(CN3CCN(C(=O)OC(C)(C)C)CC3)c2)n1] |
| | tert-butyl (R)-4-(3-((4-((3-(4-cyanophenyl)-1-methoxy-1-oxopropan- |
| | 2-yl)amino)-6-(((5-fluoro-1H-benzo[d]imidazol-2-yl)methyl)amino)- |
| | 1,3,5-triazin-2-yl)amino)benzyl)piperazine-1-carboxylate |
| TA1041 | 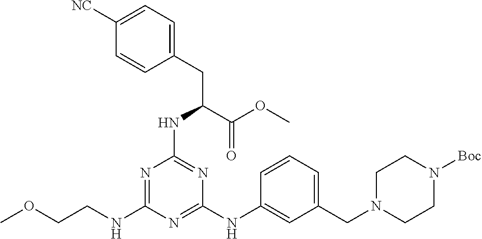 [SMILES: COCCNc1nc(Nc2cccc(CN3CCN(C(=O)OC(C)(C)C)CC3)c2)nc(N[C@@H](Cc2ccc(C#N)cc2)C(=O)OC)n1] |
| | tert-butyl (S)-4-(3-((4-((3-(4-cyanophenyl)-1-methoxy-1-oxopropan- |
| | 2-yl)amino)-6-((2-methoxyethyl)amino)-1,3,5-triazin-2- |
| | yl)amino)benzyl)piperazine-1-carboxylate |
| TA1042 | 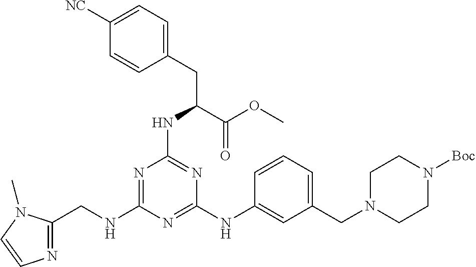 [SMILES: COC(=O)[C@H](Cc1ccc(C#N)cc1)Nc1nc(NCc2nccn2C)nc(Nc2cccc(CN3CCN(C(=O)OC(C)(C)C)CC3)c2)n1] |
| | tert-butyl (S)-4-(3-((4-((3-(4-cyanophenyl)-1-methoxy-1-oxopropan- |
| | 2-yl)amino)-6-(((1-methyl-1H-imidazol-2-yl)methyl)amino)-1,3,5- |
| | triazin-2-yl)amino)benzyl)piperazine-1-carboxylate |
| TA1043 | 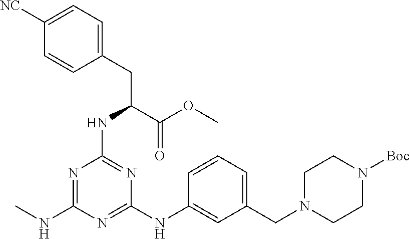 [SMILES: CNc1nc(Nc2cccc(CN3CCN(C(=O)OC(C)(C)C)CC3)c2)nc(N[C@@H](Cc2ccc(C#N)cc2)C(=O)OC)n1] |
| | tert-butyl (S)-4-(3-((4-((3-(4-cyanophenyl)-1-methoxy-1-oxopropan- |
| | 2-yl)amino)-6-(methylamino)-1,3,5-triazin-2- |
| | yl)amino)benzyl)piperazine-1-carboxylate |
| TA1044 | 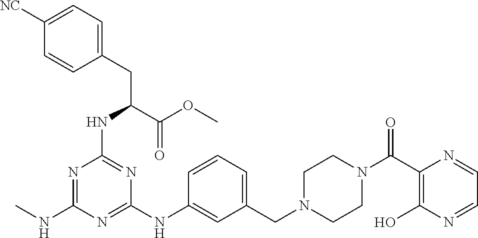 [SMILES: CNc1nc(Nc2cccc(CN3CCN(C(=O)c4nccnc4O)CC3)c2)nc(N[C@@H](Cc2ccc(C#N)cc2)C(=O)OC)n1] |
| | methyl (S)-3-(4-cyanophenyl)-2-((4-(methylamino)-6-((3-(piperazin- |
| | l-ylmethyl)phenyl)amino)-1,3,5-triazin-2-yl)amino)propanoate |
| TA1045 | 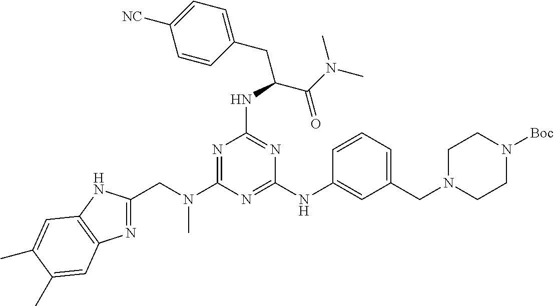 [SMILES: Cc1cc2nc(CN(C)c3nc(Nc4cccc(CN5CCN(C(=O)OC(C)(C)C)CC5)c4)nc(N[C@@H](Cc4ccc(C#N)cc4)C(=O)N(C)C)n3)[nH]c2cc1C] |
| | tert-butyl (S)-4-(3-((4-((3-(4-cyanophenyl)-1-(dimethylamino)-1- |
| | oxopropan-2-yl)amino)-6-(((5,6-dimethyl-1H-benzo[d]imidazol-2- |
| | yl)methyl)(methyl)amino)-1,3,5-triazin-2- |
| | yl)amino)benzyl)piperazine-1-carboxylate |
| TA1046 | 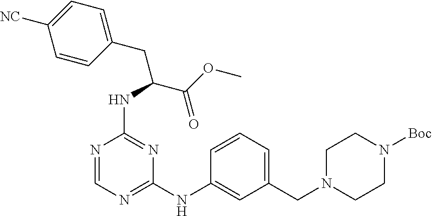 [SMILES: COC(=O)[C@H](Cc1ccc(C#N)cc1)Nc1ncnc(Nc2cccc(CN3CCN(C(=O)OC(C)(C)C)CC3)c2)n1] |
| | tert-butyl (S)-4-(3-((4-((3-(4-cyanophenyl)-1-methoxy-1-oxopropan- |
| | 2-yl)amino)-1,3,5-triazin-2-yl)amino)benzyl)piperazine-1- |
| | carboxylate |
| TA1047 | 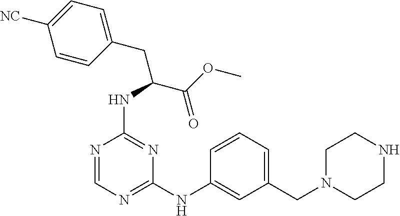 [SMILES: COC(=O)[C@H](Cc1ccc(C#N)cc1)Nc1ncnc(Nc2cccc(CN3CCNCC3)c2)n1] |
| | methyl (S)-3-(4-cyanophenyl)-2-((4-((3-(piperazin-1- |
| | ylmethyl)phenyl)amino)-1,3,5-triazin-2-yl)amino)propanoate |
| TA1048 | 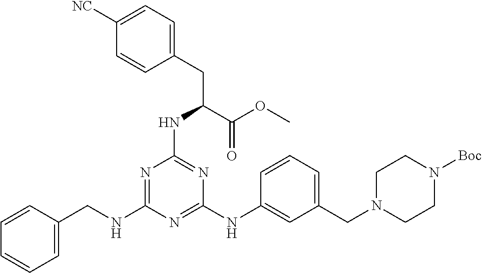 [SMILES: COC(=O)[C@H](Cc1ccc(C#N)cc1)Nc1nc(NCc2ccccc2)nc(Nc2cccc(CN3CCN(C(=O)OC(C)(C)C)CC3)c2)n1] |
| | tert-butyl (S)-4-(3-((4-(benzylamino)-6-((3-(4-cyanophenyl)-1- |
| | methoxy-1-oxopropan-2-yl)amino)-1,3,5-triazin-2- |
| | yl)amino)benzyl)piperazine-1-carboxylate |
| TA1049 | 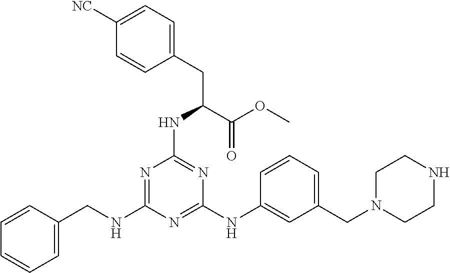 [SMILES: COC(=O)[C@H](Cc1ccc(C#N)cc1)Nc1nc(NCc2ccccc2)nc(Nc2cccc(CN3CCNCC3)c2)n1] |
| | methyl (S)-2-((4-(benzylamino)-6-((3-(piperazin-1- |
| | ylmethyl)phenyl)amino)-1,3,5-triazin-2-yl)amino)-3-(4- |
| | cyanophenyl)propanoate |
| TA1050 | 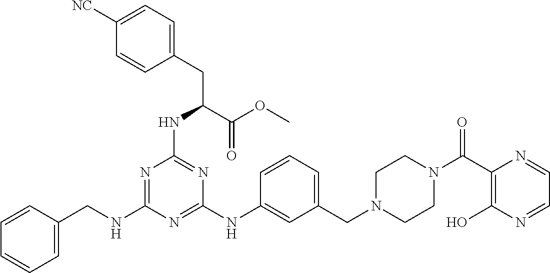 [SMILES: COC(=O)[C@H](Cc1ccc(C#N)cc1)Nc1nc(NCc2ccccc2)nc(Nc2cccc(CN3CCN(C(=O)c4nccnc4O)CC3)c2)n1] |
| | methyl (S)-2-((4-(benzylamino)-6-((3-((4-(3-hydroxypyrazine-2- |
| | carbonyl)piperazin-1-yl)methyl)phenyl)amino)-1,3,5-triazin-2- |
| | yl)amino)-3-(4-cyanophenyl)propanoate |
| TA1051 | 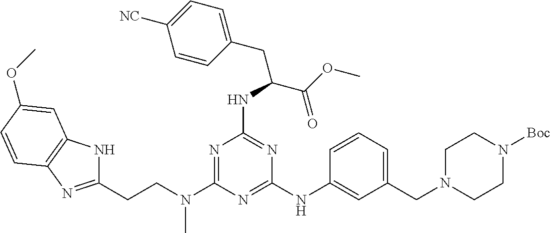 [SMILES: COC(=O)[C@H](Cc1ccc(C#N)cc1)Nc1nc(Nc2cccc(CN3CCN(C(=O)OC(C)(C)C)CC3)c2)nc(N(C)CCc2nc3ccc(OC)cc3[nH]2)n1] |
| | tert-butyl (S)-4-(3-((4-((3-(4-cyanophenyl)-1-methoxy-1-oxopropan- |
| | 2-yl)amino)-6-((2-(6-methoxy-1H-benzo[d]imidazol-2- |
| | yl)ethyl)(methyl)amino)-1,3,5-triazin-2-yl)amino)benzyl)piperazine- |
| | 1-carboxylate |
| TA1052 | 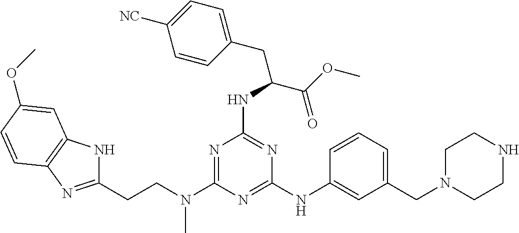 [SMILES: COC(=O)[C@H](Cc1ccc(C#N)cc1)Nc1nc(Nc2cccc(CN3CCNCC3)c2)nc(N(C)CCc2nc3ccc(OC)cc3[nH]2)n1] |
| | methyl (S)-3-(4-cyanophenyl)-2-((4-((2-(6-methoxy-1H- |
| | benzo[d]imidazol-2-yl)ethyl)(methyl)amino)-6-((3-(piperazin-1- |
| | ylmethyl)phenyl)amino)-1,3,5-triazin-2-yl)amino)propanoate |
| TA1053 | 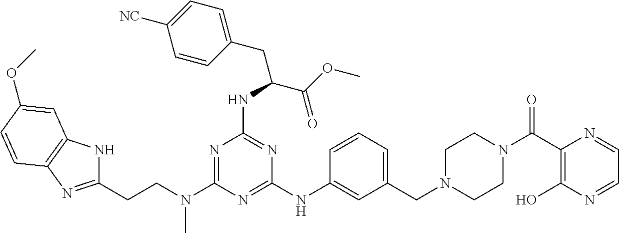 [SMILES: COC(=O)[C@H](Cc1ccc(C#N)cc1)Nc1nc(Nc2cccc(CN3CCN(C(=O)c4nccnc4O)CC3)c2)nc(N(C)CCc2nc3ccc(OC)cc3[nH]2)n1] |
| | methyl (S)-3-(4-cyanophenyl)-2-((4-((3-((4-(3-hydroxypyrazine-2- |
| | carbonyl)piperazin-1-yl)methyl)phenyl)amino)-6-((2-(6-methoxy- |
| | 1H-benzo[d]imidazol-2-yl)ethyl)(methyl)amino)-1,3,5-triazin-2- |
| | yl)amino)propanoate |
| TA1054 | 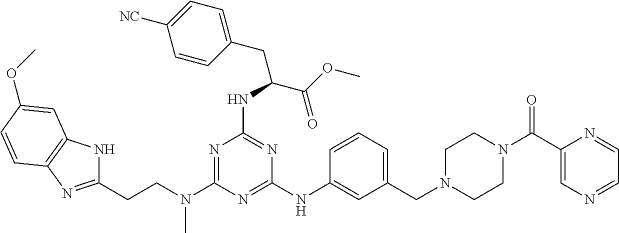 [SMILES: COC(=O)[C@H](Cc1ccc(C#N)cc1)Nc1nc(Nc2cccc(CN3CCN(C(=O)c4cnccn4)CC3)c2)nc(N(C)CCc2nc3ccc(OC)cc3[nH]2)n1] |
| | methyl (S)-3-(4-cyanophenyl)-2-((4-((2-(6-methoxy-1H- |
| | benzo[d]imidazol-2-yl)ethyl)(methyl)amino)-6-((3-((4-(pyrazine-2- |
| | carbonyl)piperazin-1-yl)methyl)phenyl)amino)-1,3,5-triazin-2- |
| | yl)amino)propanoate |
| TA1055 | 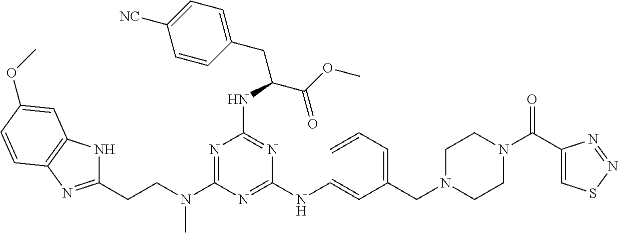 [SMILES: C=C/C=C(\C=C\Nc1nc(N[C@@H](Cc2ccc(C#N)cc2)C(=O)OC)nc(N(C)CCc2nc3ccc(OC)cc3[nH]2)n1)CN1CCN(C(=O)c2csnn2)CC1] |
| | methyl (S)-2-((4-((3-((4-(1,2,3-thiadiazole-4-carbonyl)piperazin-1- |
| | yl)methyl)phenyl)amino)-6-((2-(6-methoxy-1H-benzo[d]imidazol-2- |
| | yl)ethyl)(methyl)amino)-1,3,5-triazin-2-yl)amino)-3-(4- |
| | cyanophenyl)propanoate |
| TA1056 | 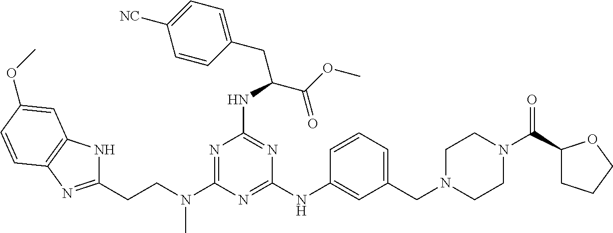 [SMILES: COC(=O)[C@H](Cc1ccc(C#N)cc1)Nc1nc(Nc2cccc(CN3CCN(C(=O)[C@@H]4CCCO4)CC3)c2)nc(N(C)CCc2nc3ccc(OC)cc3[nH]2)n1] |
| | methyl (S)-3-(4-cyanophenyl)-2-((4-((2-(6-methoxy-1H- |
| | benzo[d]imidazol-2-yl)ethyl)(methyl)amino)-6-((3-((4-((S)- |
| | tetrahydrofuran-2-carbonyl)piperazin-1-yl)methyl)phenyl)amino)- |
| | 1,3,5-triazin-2-yl)amino)propanoate |
| TA1057 | 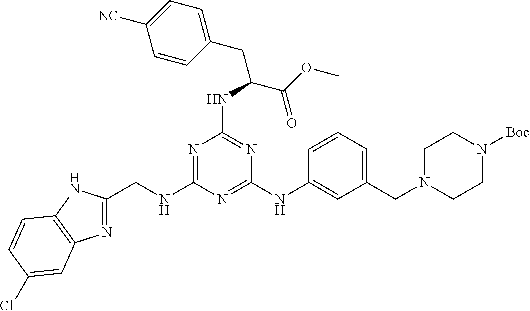 [SMILES: COC(=O)[C@H](Cc1ccc(C#N)cc1)Nc1nc(NCc2nc3cc(Cl)ccc3[nH]2)nc(Nc2cccc(CN3CCN(C(=O)OC(C)(C)C)CC3)c2)n1] |
| | tert-butyl (S)-4-(3-((4-(((5-chloro-1H-benzo[d]imidazol-2- |
| | yl)methyl)amino)-6-((3-(4-cyanophenyl)-1-methoxy-1-oxopropan-2- |
| | yl)amino)-1,3,5-triazin-2-yl)amino)benzyl)piperazine-1-carboxylate |
| TA1058 | 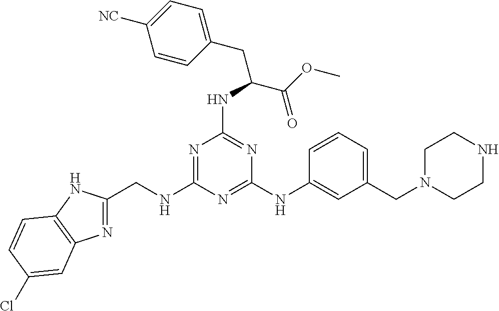 [SMILES: COC(=O)[C@H](Cc1ccc(C#N)cc1)Nc1nc(NCc2nc3cc(Cl)ccc3[nH]2)nc(Nc2cccc(CN3CCNCC3)c2)n1] |
| | methyl (S)-2-((4-(((5-chloro-1H-benzo[d]imidazol-2- |
| | yl)methyl)amino)-6-((3-(piperazin-1-ylmethyl)phenyl)amino)-1,3,5- |
| | triazin-2-yl)amino)-3-(4-cyanophenyl)propanoate |
| TA1059 | 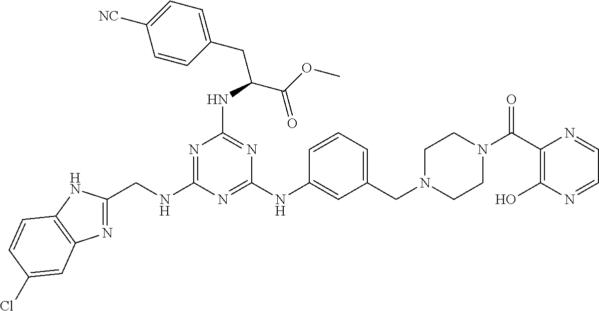 [SMILES: COC(=O)[C@H](Cc1ccc(C#N)cc1)Nc1nc(NCc2nc3cc(Cl)ccc3[nH]2)nc(Nc2cccc(CN3CCN(C(=O)c4nccnc4O)CC3)c2)n1] |
| | methyl (S)-2-((4-(((5-chloro-1H-benzo[d]imidazol-2- |
| | yl)methyl)amino)-6-((3-((4-(3-hydroxypyrazine-2- |
| | carbonyl)piperazin-1-yl)methyl)phenyl)amino)-1,3,5-triazin-2- |
| | yl)amino)-3-(4-cyanophenyl)propanoate |
| TA1060 | 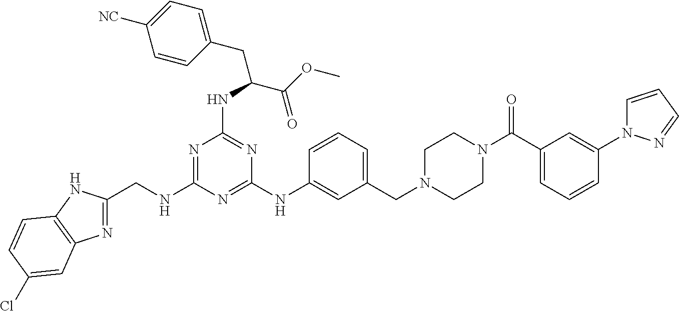 [SMILES: COC(=O)[C@H](Cc1ccc(C#N)cc1)Nc1nc(NCc2nc3cc(Cl)ccc3[nH]2)nc(Nc2cccc(CN3CCN(C(=O)c4cccc(-n5cccn5)c4)CC3)c2)n1] |
| | methyl (S)-2-((4-((3-((4-(3-(1H-pyrazol-1-yl)benzoyl)piperazin-1- |
| | yl)methyl)phenyl)amino)-6-(((5-chloro-1H-benzo[d]imidazol-2- |
| | yl)methyl)amino)-1,3,5-triazin-2-yl]amino)-3-(4- |
| | cyanophenyl)propanoate |
| TA1061 | 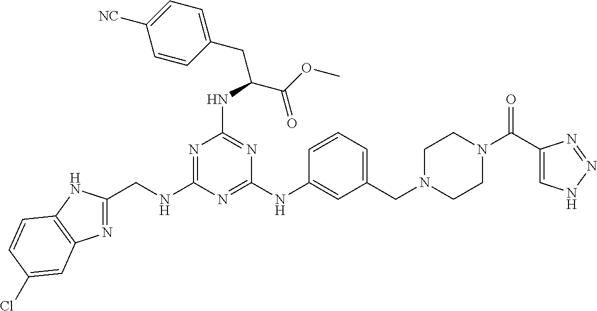 [SMILES: COC(=O)[C@H](Cc1ccc(C#N)cc1)Nc1nc(NCc2nc3cc(Cl)ccc3[nH]2)nc(Nc2cccc(CN3CCN(C(=O)c4c[nH]nn4)CC3)c2)n1] |
| | methyl (S)-2-((4-((3-((4-(1H-1,2,3-triazole-4-carbonyl)piperazin-1- |
| | yl)methyl)phenyl)amino)-6-(((5-chloro-1H-benzo[d]imidazol-2- |
| | yl)methyl)amino)-1,3,5-triazin-2-yl)amino)-3-(4- |
| | cyanophenyl)propanoate |
| TA1062 | 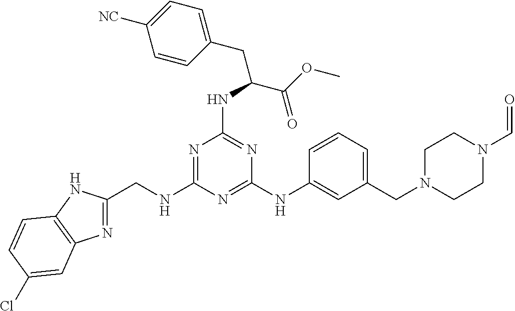 [SMILES: COC(=O)[C@H](Cc1ccc(C#N)cc1)Nc1nc(NCc2nc3cc(Cl)ccc3[nH]2)nc(Nc2cccc(CN3CCN(C=O)CC3)c2)n1] |
| | methyl (S)-2-((4-(((5-chloro-1H-benzo[d]imidazol-2- |
| | yl)methyl)amino)-6-((3-((4-formylpiperazin-1- |
| | yl)methyl)phenyl)amino)-1,3,5-triazin-2-yl)amino)-3-(4- |
| | cyanophenyl)propanoate |
| TA1063 | 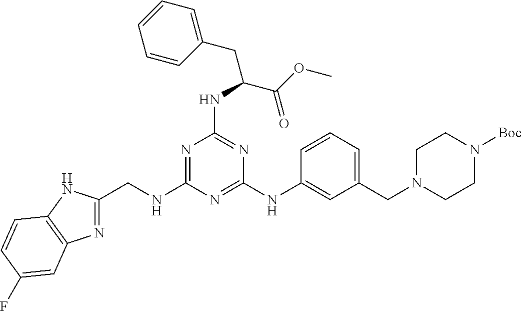 [SMILES: COC(=O)[C@H](Cc1ccccc1)Nc1nc(NCc2nc3cc(F)ccc3[nH]2)nc(Nc2cccc(CN3CCN(C(=O)OC(C)(C)C)CC3)c2)n1] |
| | tert-butyl (S)-4-(3-((4-(((5-fluoro-1H-benzo[d]imidazol-2- |
| | yl)methyl)amino)-6-((1-methoxy-1-oxo-3-phenylpropan-2- |
| | yl)amino)-1,3,5-triazin-2-yl)amino)benzyl)piperazine-1-carboxylate |
| TA1064 | 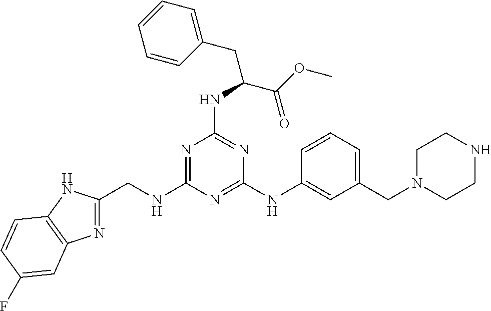 [SMILES: COC(=O)[C@H](Cc1ccccc1)Nc1nc(NCc2nc3cc(F)ccc3[nH]2)nc(Nc2cccc(CN3CCNCC3)c2)n1] |
| | methyl (4-(((5-fluoro-1H-benzo[d]imidazol-2-yl)methyl)amino)-6- |
| | ((3-(piperazin-1-ylmethyl)phenyl)amino)-1,3,5-triazin-2- |
| | yl)phenylalaninate |
| TA1065 | 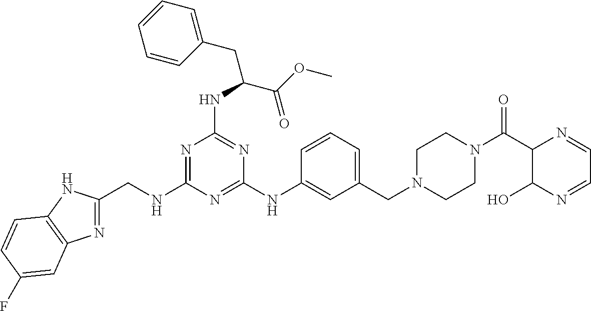 [SMILES: COC(=O)[C@H](Cc1ccccc1)Nc1nc(NCc2nc3cc(F)ccc3[nH]2)nc(Nc2cccc(CN3CCN(C(=O)C4N=CC=NC4O)CC3)c2)n1] |
| | methyl (4-(((5-fluoro-1H-benzo[d]imidazol-2-yl)methyl)amino)-6- |
| | ((3-((4-(3-hydroxypyrazine-2-carbonyl)piperazin-1- |
| | yl)methyl)phenyl)amino)-1,3,5-triazin-2-yl)-L-phenylalaninate |
| TA1066 | 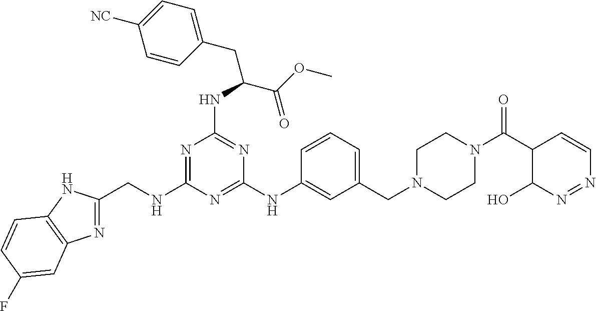 [SMILES: COC(=O)[C@H](Cc1ccc(C#N)cc1)Nc1nc(NCc2nc3cc(F)ccc3[nH]2)nc(Nc2cccc(CN3CCN(C(=O)C4C=CN=NC4O)CC3)c2)n1] |
| | methyl (S)-3-(4-cyanophenyl)-2-((4-(((5-fluoro-1H- |
| | benzo[d]imidazol-2-yl)methyl)amino)-6-((3-((4-(3- |
| | hydroxypyridazine-4-carbonyl)piperazin-1- |
| | yl)methyl)phenyl)amino)-1,3,5-triazin-2-yl)amino)propanoate |
| TA1067 | 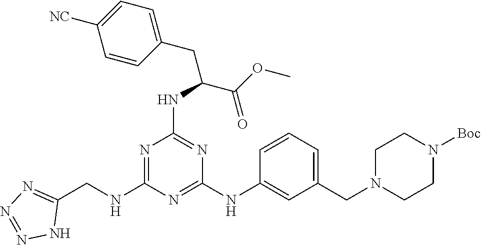 [SMILES: COC(=O)[C@H](Cc1ccc(C#N)cc1)Nc1nc(NCc2nnn[nH]2)nc(Nc2cccc(CN3CCN(C(=O)OC(C)(C)C)CC3)c2)n1] |
| | tert-butyl (S)-4-(3-((4-(((1H-tetrazol-5-yl)methyl)amino)-6-((3-(4- |
| | cyanophenyl)-1-methoxy-1-oxopropan-2-yl)amino)-1,3,5-triazin-2- |
| | yl)amino)benzyl)piperazine-1-carboxylate |
| TA1068 | 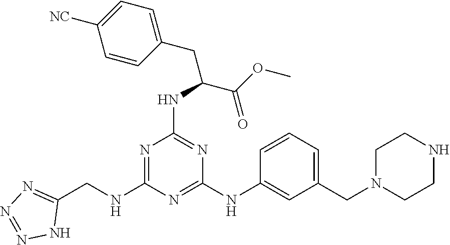 [SMILES: COC(=O)[C@H](Cc1ccc(C#N)cc1)Nc1nc(NCc2nnn[nH]2)nc(Nc2cccc(CN3CCNCC3)c2)n1] |
| | methyl (S)-2-((4-(((1H-tetrazol-5-yl)methyl)amino)-6-((3-(piperazin- |
| | 1-ylmethyl)phenyl)amino)-1,3,5-triazin-2-yl)amino)-3-(4- |
| | cyanophenyl)propanoate |
| TA1069 | 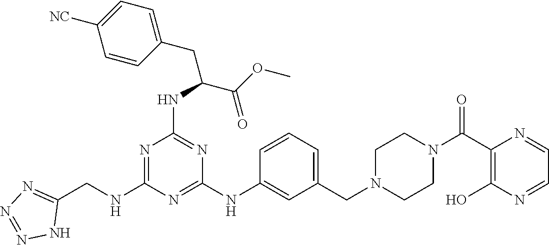 [SMILES: COC(=O)[C@H](Cc1ccc(C#N)cc1)Nc1nc(NCc2nnn[nH]2)nc(Nc2cccc(CN3CCN(C(=O)c4nccnc4O)CC3)c2)n1] |
| | methyl (S)-2-((4-(((1H-tetrazol-5-yl)methyl)amino)-6-((3-((4-(3- |
| | hydroxypyrazine-2-carbonyl)piperazin-1-yl)methyl)phenyl)amino)- |
| | 1,3,5-triazin-2-yl)amino)-3-(4-cyanophenyl)propanoate |
| TA1070 | 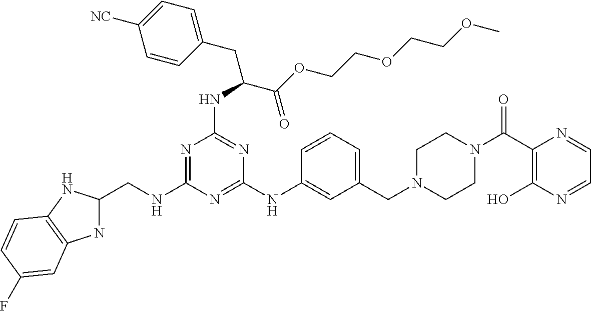 [SMILES: COCCOCCOC(=O)[C@H](Cc1ccc(C#N)cc1)Nc1nc(NCC2Nc3ccc(F)cc3N2)nc(Nc2cccc(CN3CCN(C(=O)c4nccnc4O)CC3)c2)n1] |
| | 2-(2-methoxyethoxy)ethyl (S)-3-(4-cyanophenyl)-2((4-(((5-fluoro- |
| | 1H-benzo[d]imidazol-2-yl)methyl)amino)-6-((3-((4-(3- |
| | hydroxypyrazine-2-carbonyl)piperazin-1-yl)methyl)phenyl)amino)- |
| | 1,3,5-triazin-2-yl)amino)propanoate |
| TA1071 | 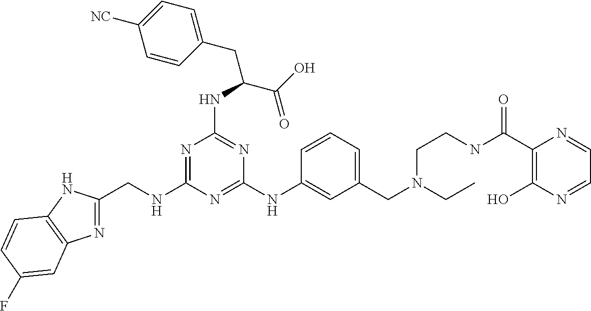 [SMILES: CCN(CCNC(=O)c1nccnc1O)Cc1cccc(Nc2nc(NCc3nc4cc(F)ccc4[nH]3)nc(N[C@@H](Cc3ccc(C#N)cc3)C(=O)O)n2)c1] |
| | (S)-3-(4-cyanophenyl)-2-((4-(((5-fluoro-1H-benzo[d]imidazol-2- |
| | yl)methyl)amino)-6-((3-((4-(3-hydroxypyrazine-2- |
| | carbonyl)piperazin-1-yl)methyl)phenyl)amino)-1,3,5-triazin-2- |
| | yl)amino)propanoic acid |
| TA1072 | 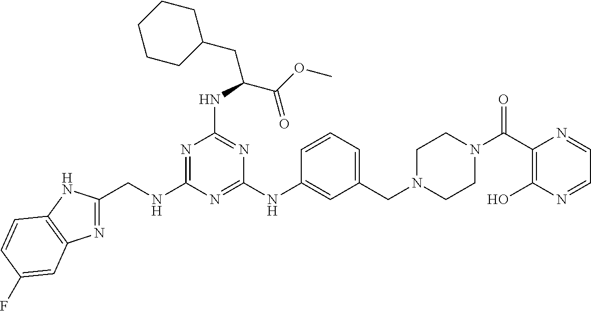 [SMILES: COC(=O)[C@H](CC1CCCCC1)Nc1nc(NCc2nc3cc(F)ccc3[nH]2)nc(Nc2cccc(CN3CCN(C(=O)c4nccnc4O)CC3)c2)n1] |
| | methyl (S)-3-cyclohexyl-2-((4-(((5-fluoro-1H-benzo[d]imidazol-2- |
| | yl)methyl)amino)-6-((3-((4-(3-hydroxypyrazine-2- |
| | carbonyl)piperazin-1-yl)methyl)phenyl)amino)-1,3,5-triazin-2- |
| | yl)amino)propanoate |
| TA1073 | 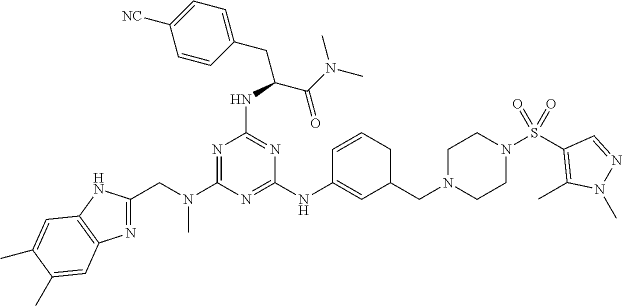 |
| | (S)-3-(4-cyanophenyl)-2-((4-(((5,6-dimethyl-1H-benzo[d]imidazol- |
| | 2-yl)methyl)(methyl)amino)-6-((3-((4-((1,5-dimethyl-1H-pyrazol-4- |
| | yl)sulfonyl)piperazin-1-yl)methyl)phenyl)amino)-1,3,5-triazin-2- |
| | yl)amino)-N,N-dimethylpropanamide |
| TA1074 | 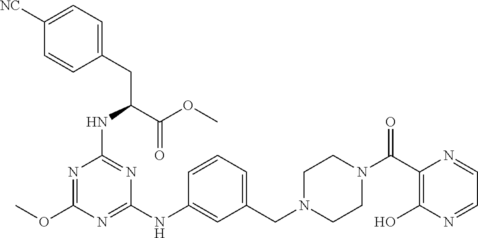 [SMILES: COC(=O)[C@H](Cc1ccc(C#N)cc1)Nc1nc(Nc2cccc(CN3CCN(C(=O)c4nccnc4O)CC3)c2)nc(OC)n1] |
| | methyl (S)-3-(4-cyanophenyl)-2-((4-((3-((4-(3-hydroxypyrazine-2- |
| | carbonyl)piperazin-1-yl)methyl)phenyl)amino)-6-methoxy-1,3,5- |
| | triazin-2-yl)amino)propanoate |
| TA1075 | 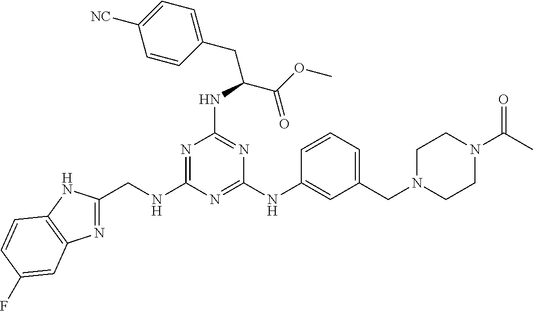 [SMILES: COC(=O)[C@H](Cc1ccc(C#N)cc1)Nc1nc(NCc2nc3cc(F)ccc3[nH]2)nc(Nc2cccc(CN3CCN(C(C)=O)CC3)c2)n1] |
| | methyl (S)-2-((4-((3-((4-acetylpiperazin-1-yl)methyl)phenyl)amino)- |
| | 6-(((5-fluoro-1H-benzo[d]imidazol-2-yl)methyl)amino)-1,3,5- |
| | triazin-2-yl)amino)-3-(4-cyanophenyl)propanoate |
| TA1076 | 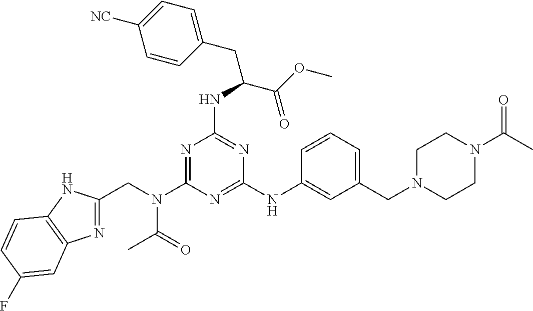 [SMILES: COC(=O)[C@H](Cc1ccc(C#N)cc1)Nc1nc(Nc2cccc(CN3CCN(C(C)=O)CC3)c2)nc(N(Cc2nc3cc(F)ccc3[nH]2)C(C)=O)n1] |
| | methyl (S)-2-((4-((3-((4-acetylpiperazin-1-yl)methyl)phenyl)amino)- |
| | 6-(N-((5-fluoro-1H-benzo[d]imidazol-2-yl)methyl)acetamido)-1,3,5- |
| | triazin-2-yl)amino)-3-(4-cyanophenyl)propanoate |
| TA1077 | 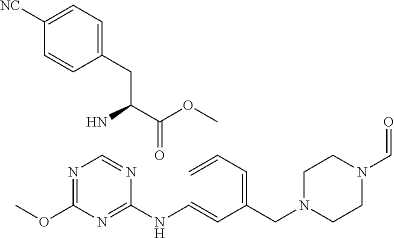 |
| | methyl (S)-3-(4-cyanophenyl)-2-((4-((3-((4-formylpiperazin-1- |
| | yl)methyl)phenyl)amino)-6-methoxy-1,3,5-triazin-2- |
| | yl)amino)propanoate |
| TA1078 | 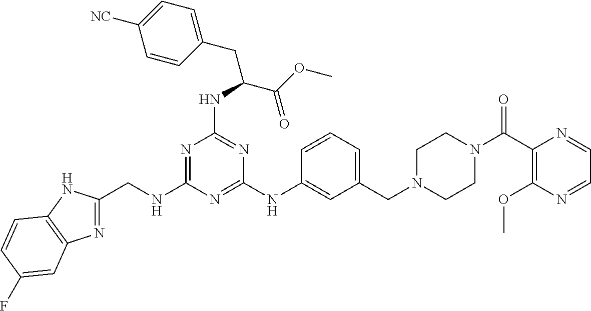 [SMILES: COC(=O)[C@H](Cc1ccc(C#N)cc1)Nc1nc(NCc2nc3cc(F)ccc3[nH]2)nc(Nc2cccc(CN3CCN(C(=O)c4nccnc4OC)CC3)c2)n1] |
| | methyl (S)-3-(4-cyanophenyl)-2-((4-(((5-fluoro-1H- |
| | benzo[d]imidazol-2-yl)methyl)amino)-6-((3-((4-(3- |
| | methoxypyrazine-2-carbonyl)piperazin-1-yl)methyl)phenyl)amino)- |
| | 1,3,5-triazin-2-yl)amino)propanoate |
| TA1079 | 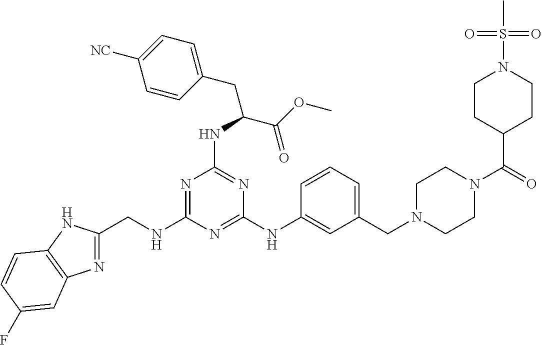 [SMILES: COC(=O)[C@H](Cc1ccc(C#N)cc1)Nc1nc(NCc2nc3cc(F)ccc3[nH]2)nc(Nc2cccc(CN3CCN(C(=O)C4CCN(S(C)(=O)=O)CC4)CC3)c2)n1] |
| | methyl (S)-3-(4-cyanophenyl)-2-((4-(((5-fluoro-1H- |
| | benzo[d]imidazol-2-yl)methyl)amino)-6-((3-((4-(1- |
| | (methylsulfonyl)piperidine-4-carbonyl)piperazin-1- |
| | yl)methyl)phenyl)amino)-1,3,5-triazin-2-yl)amino)propanoate |

74. A pharmaceutical composition comprising the compound of claim 1 or a tautomer, enantiomer, or salt thereof together with a pharmaceutically acceptable diluent or carrier.

75. A method of inhibiting cGAS in a cell, comprising contacting the cell with the compound of claim 1.

76. A method of treating a cGAS-mediated condition, comprising administering to a patient in need thereof an effective amount of a compound of claim 1.

77. The method of claim 76, wherein the cGAS-mediated condition is an autoimmune, inflammatory, or neurodegenerative condition, wherein the autoimmune condition is SIRS, sepsis, septic shock, atherosclerosis, celiac disease, interstitial cystitis, transplant rejection, Aicardi-Goutieres Syndrome, chilblain lupus erythematosus, systemic lupus erythematosus, idiopathic thrombocytopenic purpura, thrombotic thrombocytopenic purpura, autoimmune thrombocytopenia, spondyloenchondrodysplasia, psoriasis, Type 1 diabetes, Type 2 diabetes, or Sjogren's syndrome, and wherein the inflammatory condition is rheumatoid arthritis, juvenile rheumatoid arthritis, inflammatory bowel disease (ulcerative colitis, Crohn's disease), age-related macular degeneration, IgA nephropathy, glomerulonephritis, vasculitis, polymyositis, or Wegener's disease, and wherein the neurodegenerative condition is Alzheimer's disease, Parkinson's disease, multiple sclerosis, IgM polyneuropathies, or myasthenia gravis.

78. (canceled)

79. (canceled)

80. (canceled)

81. (canceled)

82. (canceled)

83. (canceled)
